(19) United States
(12) Patent Application Publication
(10) Pub. No.: US 2021/0139391 A1
(43) Pub. Date: May 13, 2021

(54) ETHYLENE-TO-LIQUIDS SYSTEMS AND METHODS

(71) Applicant: Lummus Technology LLC, The Woodlands, TX (US)

(72) Inventors: Raed Hasan Abudawoud, Dhahran (SA); Anthony Crisci, Oakland, CA (US); David C. Grauer, San Francisco, CA (US); William Michalak, Redwood City, CA (US); Greg Nyce, Pleasanton, CA (US); Tim A. Rappold, Oakland, CA (US); Aihua Zhang, Daly City, CA (US); Richard Black, Montgomery, TX (US); Peter Czerpak, San Francisco, CA (US); Bipinkumar Patel, Richmond, TX (US); Guido Radaelli, Pleasant Hill, CA (US)

(21) Appl. No.: 17/153,016

(22) Filed: Jan. 20, 2021

Related U.S. Application Data
(63) Continuation of application No. 16/751,999, filed on Jan. 24, 2020.
Application No. 16/751,999 is a continuation of application No. 15/826,997, filed on Nov. 30, 2017, now abandoned.
(60) Provisional application No. 62/522,049, filed on Jun. 19, 2017.
(60) Provisional application No. 62/483,852, filed on Apr. 10, 2017.
(60) Provisional application No. 62/475,108, filed on Mar. 22, 2017.
(60) Provisional application No. 62/429,244, filed on Dec. 2, 2016.

(51) Int. Cl. C07C 2/12; C07C 2/42; C07C 2/10; C07C 15/067; C07B 37/10; C07C 27/10; C07B 41/02; C07B 41/04; C07B 37/04; C07C 9/14; C07C 29/04; C10G 50/00; C10G 29/20; C10G 27/04; B01J 8/06; C10G 29/22

(52) U.S. Cl. CPC C07C 2/12; Y02P 20/10; C07C 2/10; C07C 15/067; C07B 37/10; C07C 27/10; C07B 41/02; C07B 41/04; C07B 37/04; C07C 9/14; C07C 29/04; C10G 50/00; C10G 29/205; C10G 27/04; B01J 8/067; C10G 29/22; C10G 2400/02; Y02P 20/52; C10G 2400/08; Y02P 20/582; C10G 2400/30; C10G 2300/1092; C10G 2400/04; C07C 2529/40; C07C 2/42

(57) ABSTRACT

The present disclosure provides petrochemical processing methods and systems, including ethylene conversion processes and systems, for the production of higher hydrocarbon compositions, for example liquid hydrocarbon compounds, with reduced amount of unsaturated hydrocarbons.

### CROSS-REFERENCE

This application is a continuation of U.S. patent application Ser. No. 16/751,999, filed Jan. 24, 2020, which is a continuation of U.S. patent application Ser. No. 15/826,997, filed Nov. 30, 2017, which claims the benefit of U.S. Provisional Patent Application No. 62/429,244, filed Dec. 2, 2016, U.S. Provisional Patent Application No. 62/475,108, filed Mar. 22, 2017, U.S. Provisional Patent Application No. 62/483,852, filed Apr. 10, 2017, and U.S. Provisional Patent Application No. 62/522,049, filed Jun. 19, 2017, each of which is incorporated herein by reference in its entirety.

### BACKGROUND

The modern petrochemical industry makes extensive use of cracking and fractionation technology to produce and separate various desirable compounds from crude oil. Cracking and fractionation operations are energy intensive and generate considerable quantities of greenhouse gases.

The gradual depletion of worldwide petroleum reserves and the commensurate increase in petroleum prices may place extraordinary pressure on refiners to minimize losses and improve efficiency when producing products from existing feedstocks, and also to seek viable alternative feedstocks capable of providing affordable hydrocarbon intermediates and liquid fuels to downstream consumers.

Ethylene-to-liquids (ETL) technology in its current form produces a liquid product rich in olefins. Federal and state specifications with respect to gasoline fuel may limit the amount of olefins that can be blended into gasoline, to be around 4-6 wt % in total, for example.

### SUMMARY

Recognized herein is a need for efficient and commercially viable systems and methods for converting ethylene to higher molecular weight hydrocarbons, including gasoline, diesel fuel, jet fuel, and aromatic chemicals, with olefin content reduced sufficiently to meet Federal and state specifications.

The present disclosure provides methods and systems for reducing olefin content in streams, for example, to meet various specifications. In some cases, ethylene is converted to higher hydrocarbon compounds in an ethylene-to-liquids (ETL) process. The ETL product can then be modified or further processed in one or more additional processes to produce an end product with olefins largely reduced to meet the specifications and product properties that maximize its utility.

In some cases, the higher molecular weight hydrocarbons can be produced from methane in an integrated process that converts methane to ethylene and the ethylene to the higher molecular weight compounds. An oxidative coupling of methane (“OCM”) reaction is a process by which methane can form one or more hydrocarbon compounds with two or more carbon atoms (also “C₂₊ compounds” herein), such as olefins like ethylene.

In an OCM process, methane can be oxidized to yield products comprising C₂₊ compounds, including alkanes (e.g., ethane, propane, butane, pentane, etc.) and alkenes (e.g., ethylene, propylene, etc.). Such alkane (also “paraffin” herein) products may not be suitable for use in downstream processes. Unsaturated chemical compounds, such as alkenes (or olefins), may be employed for use in downstream processes. Such compounds can be polymerized to yield polymeric materials, which can be employed for use in various commercial settings.

Oligomerization processes can be used to further convert ethylene into longer chain hydrocarbons useful as polymer components for plastics, vinyls, and other high value polymeric products. Additionally, these oligomerization processes may be used to convert ethylene to other longer hydrocarbons, such as C6, C7, C8 and longer hydrocarbons useful for fuels like gasoline, diesel, jet fuel and blendstocks for these fuels, as well as other high value specialty chemicals.

An aspect of the present disclosure provides a method for generating oxygenate compounds with five or more carbon atoms (C₅₊ oxygenates), comprising: (a) directing an unsaturated hydrocarbon feed stream comprising ethylene (C₂H₄) into an ethylene-to-liquids (ETL) reactor that converts the C₂H₄ in an ETL process to yield a product stream comprising compounds with five or more carbon atoms (C₅₊ compounds); and (b) directing at least a portion of the product stream from the ETL reactor into a hydration unit that reacts the C₅₊ compounds in the at least the portion of the product stream in a hydration process to yield an oxygenate product stream comprising the C₅₊ oxygenates.

In some embodiments, the C₅₊ compounds comprise olefins. In some embodiments, the olefins comprise di-olefins, acyclic olefins and cyclic olefins. In some embodiments, the method further comprises converting the olefins to the oxygenate product stream comprising the C₅₊ oxygenates. In some embodiments, at least 20 volume percent (vol %) of the olefins are converted to the C₅₊ oxygenates. In some embodiments, the olefins are substantially converted to the C₅₊ oxygenates. In some embodiments, the C₅₊ compounds comprise alkynes. In some embodiments, the C₅₊ oxygenates comprise alcohols comprising five or more carbon atoms (C₅₊ alcohols). In some embodiments, subsequent to (b), the product stream comprises at most about 10 wt % olefins. In some embodiments, the hydration unit comprises a hydration catalyst that facilitates a hydration reaction in the hydration process. In some embodiments, the hydration catalyst comprises an acid catalyst. In some embodiments, the acid catalyst is selected from the group consisting of water soluble acids, organic acids, metal organic frameworks (MOF), and solid acids. In some embodiments, the water soluble acids comprise HNO₃, HCl, H₃PO₄, H₂SO₄ and heteropoly acids. In some embodiments, the organic acids comprise one or more of acetic acid, tosylate acid, and perflorinated acetic acid. In some embodiments, the solid acids comprise one or more of ion exchange resin, acidic zeolite and metal oxide. In some embodiments, the hydration unit is operated at a temperature from about 50° C. to 300° C. In some embodiments, the hydration unit is operated at a pressure from about 10 PSI to 3,000 PSI. In some embodiments, (b) further comprises directing water into the hydration reactor, wherein the water reacts with the C₅₊ compounds in the hydration process to yield the C₅₊ oxygenates. In some embodiments, a molar ratio of the water to the C₅₊ compounds directed into the hydration unit is from about 0.1 to about 300. In some embodiments, the product stream further comprises compounds with four carbon atoms or less (C₄₋ compounds). In some embodiments, the method further comprises, prior to (b), directing the product stream comprising the C₄₋ compounds into a separation unit that (i) separates the C₄₋ compounds from the product stream and (ii) enriches the C₄₋ compounds in the product stream. In some embodiments, the method further comprise directing the C₄₋ compounds from the separation unit into an aromatization reactor that converts the C₄₋ compounds in an aromatization process to yield aromatic hydrocarbon products. In some embodiments, the method further comprises recovering from the aromatization reactor a liquid stream comprising the aromatic hydrocarbon products. In some embodiments, the aromatic hydrocarbon products comprise one or more of benzene, toluene, xylenes, and ethylbenzene. In some embodiments, the method further comprises (i) recovering from the aromatization reactor an additional stream comprising unconverted C₄₋ compounds and (ii) recycling at least a portion of the additional stream to the aromatization reactor and/or the ETL reactor. In some embodiments, the method further comprises directing hydrogen (H₂) or nitrogen (N₂) into the aromatization reactor. In some embodiments, the ETL process is operated at a first temperature and the aromatization process is operated at a second temperature that is higher than the first temperature. In some embodiments, a difference between the first temperature and the second temperature is between about 50° C. and 500° C. In some embodiments, the aromatization reactor is operated at a temperature between about 200° C. and 700° C. In some embodiments, the aromatization reactor is operated at a pressure between about 10 PSI bar and 1,500 PSI. In some embodiments, the aromatization reactor is a fixed-bed, a moving-bed, or a fluid bed reactor. In some embodiments, the method further comprises recovering one or more additional C₅₊ compounds from one or more additional units and directing at least a portion of the one or more additional C₅₊ compounds into the hydration unit that reacts the at least the portion of the one or more additional C₅₊ compounds in the hydration process to yield one or more additional C₅₊ oxygenates. In some embodiments, the one or more additional units are integrated and in fluidic communication with the ETL reactor and/or the hydration unit. In some embodiments, the one or more additional units are retrofitted into a system comprising the ETL reactor and/or the hydration unit. In some embodiments, the method further comprises recovering from the hydration unit the C₅₊ oxygenates and the one or more additional C₅₊ oxygenates. In some embodiments, the C₅₊ oxygenates and the one or more additional C₅₊ oxygenates comprise C₅₊ alcohols. In some embodiments, the C₅₊ alcohols comprise one or more of 1,5-pentanediol, 1,6-hexanediol, cyclohexanol, 3-hexanol, 4-methyl-2-pentanol, 3-methyl-3-pentanol, 3,3-dimethyl-2-butanol, 2-pentanol, 3-methyl-2-butanol, and tertiary amyl alcohol. In some embodiments, the one or more additional units are selected from the group consisting of a metathesis unit, fluid catalytic cracking (FCC) unit, thermal cracker unit, coker unit, methanol to olefins (MTO) unit, Fischer-Tropsch unit, and oxidative coupling of methane (OCM) unit, or any combination thereof. In some embodiments, the ETL reactor operates substantially adiabatically.

Another aspect of the present disclosure provides a method for generating aromatics products comprising eight carbon atoms (C₈ aromatics), comprising: (a) directing an unsaturated hydrocarbon feed stream comprising ethylene (C₂H₄) into an ethylene-to-liquids (ETL) reactor, wherein the ETL reactor comprises (i) an ETL catalyst that facilitates an ETL reaction and (ii) a transalkylation catalyst that facilitates a transalkylation reaction; and (b) in the ETL reactor, conducting (1) the ETL reaction to convert the C₂H₄ in the unsaturated hydrocarbon feed stream to yield higher hydrocarbon products, and (2) the transalkylation reaction to convert at least a portion of the higher hydrocarbon products to yield the C₈ aromatics.

In some embodiments, the ETL reaction and the transalkylation reaction are conducted substantially simultaneously. In some embodiments, the ETL reaction and the transalkylation reaction are conducted under substantially the same reaction conditions. In some embodiments, the transalkylation catalyst is intermixed with the ETL catalyst. In some embodiments, the ETL reactor comprises catalyst particles, wherein an individual catalyst particle comprises the ETL catalyst and the transalkylation catalyst. In some embodiments, the transalkylation catalyst and the ETL catalyst are in separate layers of the individual catalyst particle. In some embodiments, the transalkylation catalyst is sandwiched between layers of the ETL catalyst. In some embodiments, the transalkylation catalyst and the ETL catalyst are in the same layer of the individual catalyst particle. In some embodiments, the ETL catalyst is porous. In some embodiments, the ETL catalyst has pores with an average pore size between about 4 angstrom (Å) and 7 Å. In some embodiments, the transalkylation catalyst is porous. In some embodiments, the transalkylation catalyst has pores with an average pore size greater than or equal to about 7 Å. In some embodiments, the ETL catalyst comprises a zeolite. In some embodiments, the zeolite includes erionite, zeolite 4A and/or zeolite 5A. In some embodiments, the zeolite includes one or more of MFI topology zeolites. In some embodiments, the transalkylation catalyst comprises a zeolite. In some embodiments, the zeolite comprises mordenite. In some embodiments, the transalkylation catalyst further comprises one or more metal selected from the group consisting of rhenium, platinum, nickle, and any combination thereof. In some embodiments, the transalkylation catalyst comprises beta zeolite, zeolite Y, Ultrastable Y (USY), Dealuminized Y (Deal Y), mordenite, NU-87, ZSM-3, ZSM-4 (Mazzite), ZSM-12, ZSM-18, MCM-22, MCM-36, MCM-49, MCM-56, EMM-10, EMM-10-P and ZSM-20. In some embodiments, the ETL catalyst and transalkylation catalyst are porous, and an average pore size of the ETL catalyst is less than an average pore size of the transalkylation catalyst. In some embodiments, the higher hydrocarbon products comprise compounds with six or more carbon atoms. In some embodiments, the higher hydrocarbon products comprises compounds with six and seven carbon atoms (C₆/C₇ compounds) and compounds with nine or more carbon atoms (C₉₊ compounds). In some embodiments, in the transalkylation reaction, at least a portion of the C₉₊ compounds is reacted with at least a portion of the C₆/C₇ compounds to yield the C₈ aromatics. In some embodiments, the ETL catalyst in the ETL reactor has a lifetime that is greater than a lifetime of the ETL catalyst in the absence of the transalkylation catalyst in the ETL reactor. In some embodiments, the ETL catalyst in the ETL reactor has a lifetime that is at least 1.5 times greater than a lifetime of the ETL catalyst in the absence of the transalkylation catalyst in the ETL reactor. In some embodiments, the ETL reactor operates substantially adiabatically.

Another aspect of the present disclosure provides a method for generating compounds comprising five or more carbon atoms (C₅₊ compounds), comprising: (a) directing (i) an unsaturated hydrocarbon feed stream comprising ethylene (C₂H₄) and (ii) an oxygen (O₂) containing stream comprising O₂ into an ethylene-to-liquids (ETL) reactor, wherein the ETL reactor comprises an ETL catalyst that conducts an ETL reaction, and wherein the O₂ is directed into the ETL reactor at a concentration of less than about 1 volume percent (vol %) of the unsaturated hydrocarbon feed stream; and (b) in the ETL reactor, conducting the ETL reaction to convert, in the presence of the O₂, the C₂H₄ in the unsaturated hydrocarbon feed stream to yield a product stream comprising the C₅₊ compounds.

In some embodiments, the concentration of the O₂ is greater than or equal to about 0.005 vol % of the unsaturated hydrocarbon feed stream. In some embodiments, the concentration of the O₂ is selected to enhance a dehydrogenation activity of the ETL catalyst, as determined by a yield of the C₅₊ compounds in the presence of the O₂ at the concentration relative to a yield of the C₅₊ compounds in the absence of the O₂ at the concentration. In some embodiments, the concentration of the O₂ is selected to enhance a dehydrogenation activity of the ETL catalyst by a factor of at least 1.05, as determined by a yield of the C₅₊ compounds in the presence of the O₂ at the concentration relative to a yield of the C₅₊ compounds in the absence of the O₂ at the concentration. In some embodiments, the ETL reactor operates substantially adiabatically. In some embodiments, the method further comprises, prior to (a), directing methane and an oxidizing agent into an oxidative coupling of methane (OCM) reactor upstream of and in fluid communication with the ETL reactor, wherein the OCM reactor reacts the methane with the oxidizing agent to generate at least a portion of the unsaturated hydrocarbon feed stream comprising the C₂H₄. In some embodiments, the OCM reactor is integrated with the ETL reactor. In some embodiments, the OCM reactor is retrofitted into a system comprising the ETL reactor.

Another aspect of the present disclosure provides a system for generating oxygenate compounds with five or more carbon atoms (C₅₊ oxygenates), comprising: an ethylene-to-liquids (ETL) reactor that, during use, receives an unsaturated hydrocarbon feed stream comprising ethylene (C₂H₄) and converts the C₂H₄ in an ETL process to yield a product stream comprising compounds with five or more carbon atoms (C₅₊ compounds); and a hydration unit fluidically coupled to the ETL reactor, wherein during use, the hydration unit (i) receives at least a portion of the product stream from the ETL reactor and (ii) reacts the C₅₊ compounds in the at least the portion of the product stream in a hydration process to yield an oxygenate product stream comprising the C₅₊ oxygenates.

In some embodiments, the C₅₊ compounds comprise olefins. In some embodiments, the olefins comprise di-olefins, acyclic olefins and cyclic olefins. In some embodiments, the hydration unit converts the olefins to the oxygenate product stream comprising the C₅₊ oxygenates. In some embodiments, at least 20 volume percent (vol %) of the olefins are converted to the C₅₊ oxygenates. In some embodiments, the olefins are substantially converted to the C₅₊ oxygenates. In some embodiments, the C₅₊ compounds comprise alkynes. In some embodiments, the C₅₊ oxygenates comprise alcohols comprising five or more carbon atoms (C₅₊ alcohols). In some embodiments, after the oxygenate product stream is yielded, the product stream comprises at most about 10 wt % olefins. In some embodiments, the hydration unit comprises a hydration catalyst that facilitates a hydration reaction in the hydration process. In some embodiments, the hydration catalyst comprises an acid catalyst. In some embodiments, the acid catalyst is selected from the group consisting of water soluble acids, organic acids, metal organic frameworks (MOF), and solid acids. In some embodiments, the water soluble acids comprise HNO₃, HCl, H₃PO₄, H₂SO₄ and heteropoly acids. In some embodiments, the organic acids comprise one or more of acetic acid, tosylate acid, and perflorinated acetic acid. In some embodiments, the solid acids comprise one or more of ion exchange resin, acidic zeolite and metal oxide. In some embodiments, the hydration unit is operated at a temperature from about 50° C. to 300° C. In some embodiments, the hydration unit is operated at a pressure from about 10 PSI bar to 3,000 PSI. In some embodiments, the hydration reactor further receives water that reacts with the C₅₊ compounds in the hydration process to yield the C₅₊ oxygenates. In some embodiments, a molar ratio of the water to the C₅₊ compounds directed into the hydration unit is from about 0.1 to about 300. In some embodiments, the product stream further comprises compounds with four carbon atoms or less (C₄₋ compounds). In some embodiments, the system further comprises a separation unit fluidically coupled to the ETL reactor, wherein during use, the separation unit (i) receives the product stream comprising the C₄₋ compounds (ii) separates the C₄₋ compounds from the product stream and (iii) enriches the C₄₋ compounds in the product stream. In some embodiments, the system further comprises an aromatization reactor fluidically coupled to the separation unit, wherein during use, the aromatization reactor (i) receives the C₄₋ compounds from the separation unit and (ii) converts the C₄₋ compounds in an aromatization process to yield aromatic hydrocarbon products. In some embodiments, a liquid stream comprising the aromatic hydrocarbon products is recovered from the aromatization reactor. In some embodiments, the aromatic hydrocarbon products comprise one or more of benzene, toluene, xylene and ethylbenzene. In some embodiments, (i) an additional stream comprising unconverted C₄₋ compounds is recovered from the aromatization reactor and (ii) at least a portion of the additional stream is recycled to the aromatization reactor and/or the ETL reactor. In some embodiments, the aromatization reactor further receives hydrogen (H₂) or nitrogen (N₂). In some embodiments, the ETL process is operated at a first temperature and the aromatization process is operated at a second temperature that is higher than the first temperature. In some embodiments, a difference between the first temperature and the second temperature is between about 50° C. and 500° C. In some embodiments, the aromatization reactor is operated at a temperature between about 200° C. and 700° C. In some embodiments, the aromatization reactor is operated at a pressure between about 10 PSI and 1,500 PSI. In some embodiments, the aromatization reactor is a fixed-bed, a moving-bed, or a fluid bed reactor. In some embodiments, the system further comprises one or more additional units fluidically coupled to the hydration unit, wherein one or more additional C₅₊ compounds are recovered from the one or more additional units and at least a portion of the one or more additional C₅₊ compounds are directed into the hydration unit that reacts the at least the portion of the one or more additional C₅₊ compounds in the hydration process to yield one or more additional C₅₊ oxygenates. In some embodiments, the one or more additional units are integrated and in fluidic communication with the ETL reactor and/or the hydration unit. In some embodiments, the one or more additional units are retrofitted into a system comprising the ETL reactor and/or the hydration unit. In some embodiments, the C₅₊ oxygenates and the one or more additional C₅₊ oxygenates are recovered from the hydration unit. In some embodiments, the C₅₊ oxygenates and the one or more additional C₅₊ oxygenates comprise C₅₊ alcohols. In some embodiments, the C₅₊ alcohols comprise one or more of 1,5-pentanediol, 1,6-hexanediol, cyclohexanol, 3-hexanol, 4-methyl-2-pentanol, 3-methyl-3-pentanol, 3,3-dimethyl-2-butanol, 2-pentanol, 3-methyl-2-butanol, and tertiary amyl alcohol. In some embodiments, the one or more additional units are selected from the group consisting of a metathesis unit, fluid catalytic cracking (FCC) unit, thermal cracker unit, coker unit, methanol to olefins (MTO) unit, Fischer-Tropsch unit, and oxidative coupling of methane (OCM) unit, or any combination thereof. In some embodiments, the ETL reactor operates substantially adiabatically.

Another aspect of the present disclosure provides a system for generating aromatics products comprising eight carbon atoms (C₈ aromatics), comprising: an ethylene-to-liquids (ETL) reactor comprising (i) an ETL catalyst that facilitates an ETL reaction and (ii) a transalkylation catalyst that facilitates a transalkylation reaction; and a controller that directs an unsaturated hydrocarbon feed stream comprising ethylene (C₂H₄) into the ETL reactor to conduct (a) the ETL reaction to convert the C₂H₄ in the unsaturated hydrocarbon feed stream to yield higher hydrocarbon products, and (b) the transalkylation reaction to convert at least a portion of the higher hydrocarbon products to yield the C₈ aromatics.

In some embodiments, the ETL reaction and the transalkylation reaction are conducted substantially simultaneously. In some embodiments, the ETL reaction and the transalkylation reaction are conducted under substantially the same reaction conditions. In some embodiments, the transalkylation catalyst is intermixed with the ETL catalyst. In some embodiments, the ETL reactor comprises catalyst particles, wherein an individual catalyst particle comprises the ETL catalyst and the transalkylation catalyst. In some embodiments, the transalkylation catalyst and the ETL catalyst are in separate layers of the individual catalyst particle. In some embodiments, the transalkylation catalyst is sandwiched between layers of the ETL catalyst. In some embodiments, the transalkylation catalyst and the ETL catalyst are in the same layer of the individual catalyst particle. In some embodiments, the ETL catalyst is porous. In some embodiments, the ETL catalyst has pores with an average pore size between about 4 angstrom (Å) and 7 Å. In some embodiments, the transalkylation catalyst is porous. In some embodiments, the transalkylation catalyst has pores with an average pore size greater than or equal to about 7 Å. In some embodiments, the ETL catalyst comprises a zeolite. In some embodiments, the zeolite includes erionite, zeolite 4A and/or zeolite 5A. In some embodiments, the zeolite includes one or more of MFI topology zeolites. In some embodiments, the transalkylation catalyst comprises a zeolite. In some embodiments, the zeolite comprises mordenite. In some embodiments, the transalkylation catalyst further comprises one or more metal selected from the group consisting of rhenium, platinum, nickle, and any combination thereof. In some embodiments, the transalkylation catalyst comprises beta zeolite, zeolite Y, Ultrastable Y (USY), Dealuminized Y (Deal Y), mordenite, NU-87, ZSM-3, ZSM-4 (Mazzite), ZSM-12, ZSM-18, MCM-22, MCM-36, MCM-49, MCM-56, EMM-10, EMM-10-P and ZSM-20. In some embodiments, the ETL catalyst and transalkylation catalyst are porous, and wherein an average pore size of the ETL catalyst is less than an average pore size of the transalkylation catalyst. In some embodiments, the higher hydrocarbon products comprise compounds with six or more carbon atoms. In some embodiments, the higher hydrocarbon products comprises compounds with six and seven carbon atoms (C₆/C₇ compounds) and compounds with nine or more carbon atoms (C₉₊ compounds). In some embodiments, in the transalkylation reaction, at least a portion of the C₉₊ compounds is reacted with at least a portion of the C₆/C₇ compounds to yield the C₈ aromatics. In some embodiments, the ETL catalyst in the ETL reactor has a lifetime that is greater than a lifetime of the ETL catalyst in the absence of the transalkylation catalyst in the ETL reactor. In some embodiments, the ETL catalyst in the ETL reactor has a lifetime that is at least 1.5 times greater than a lifetime of the ETL catalyst in the absence of the transalkylation catalyst in the ETL reactor. In some embodiments, the ETL reactor operates substantially adiabatically.

Another aspect of the present disclosure provides a system for generating compounds comprising five or more carbon atoms (C₅₊ compounds), comprising: an ethylene-to-liquids (ETL) reactor comprising an ETL catalyst that conducts an ETL reaction; and a controller that directs to the ETL reactor (i) an unsaturated hydrocarbon feed stream comprising ethylene (C₂H₄) and (ii) an oxygen (O₂) containing stream comprising O₂ at a concentration of less than 1 volume percent (vol %) of the unsaturated hydrocarbon feed stream, to conduct the ETL reaction to convert, in the presence of the O₂, the C₂H₄ in the unsaturated hydrocarbon feed stream to yield a product stream comprising the C₅₊ compounds.

In some embodiments, the concentration of the O₂ is greater than or equal to about 0.005 vol % of the unsaturated hydrocarbon feed stream. In some embodiments, the concentration of the O₂ is selected to enhance a dehydrogenation activity of the ETL catalyst, as determined by a yield of the C₅₊ compounds in the presence of the O₂ at the concentration relative to a yield of the C₅₊ compounds in the absence of the O₂ at the concentration. In some embodiments, the concentration of the O₂ is selected to enhance a dehydrogenation activity of the ETL catalyst by a factor of at least 1.05, as determined by a yield of the C₅₊ compounds in the presence of the O₂ at the concentration relative to a yield of the C₅₊ compounds in the absence of the O₂ at the concentration. In some embodiments, the ETL reactor operates substantially adiabatically. In some embodiments, the system further comprises an oxidative coupling of methane (OCM) reactor upstream of and fluidically coupled to the ETL reactor, wherein during use, the OCM reactor (i) receives methane and an oxidizing agent and (ii) reacts the methane with the oxidizing agent to generate at least a portion of the unsaturated hydrocarbon feed stream comprising the C₂H₄. In some embodiments, the OCM reactor is integrated with the ETL reactor. In some embodiments, the OCM reactor is retrofitted into a system comprising the ETL reactor.

Another aspect of the present disclosure provides a method for generating hydrocarbon compounds with three or more carbon atoms (C₃₊ compounds), comprising: directing a feed stream comprising ethylene (C₂H₄) into an ethylene conversion reactor that converts the C₂H₄ in an ethylene conversion process to yield a product stream comprising the C₃₊ compounds, wherein the ethylene conversion reactor comprises at least one mesoporous catalyst disposed therein and configured to operate at a temperature greater than or equal to about 100° C. and a pressure greater than or equal to about 150 pounds per square inch (PSI) in the ethylene conversion process, and wherein the at least one mesoporous catalyst comprises a plurality of mesopores having an average pore size from about 1 nanometer (nm) to 500 nm.

In some embodiments, the C₃₊ compounds comprise hydrocarbon compounds with five or more carbon atoms (C₅₊ compounds). In some embodiments, the method further comprises directing at least a portion of the product stream from the ethylene conversion reactor into a hydration unit that reacts the C₅₊ compounds in the at least the portion of the product stream in a hydration process to yield an oxygenate product stream comprising oxygenate compounds with five or more carbon atoms (C₅₊ oxygenates). In some embodiments, the ethylene conversion reactor is an ethylene-to-liquids (ETL) reactor, and wherein the ethylene conversion process is an ETL process. In some embodiments, the temperature is greater than or equal to about 300° C. In some embodiments, the pressure is greater than or equal to about 250 PSI. In some embodiments, the pressure is less than or equal to about 900 PSI. In some embodiments, the average pore size is from 1 nm to 50 nm. In some embodiments, the average pore size is from 1 nm to 10 nm. In some embodiments, the at least one mesoporous catalyst comprises mesoporous zeolites. In some embodiments, the mesoporous zeolites comprise mesoporous ZSM-5. In some embodiments, the C₃₊ compounds are generated at a selectivity greater than about 50%.

Another aspect of the present disclosure provides a method for generating hydrocarbon compounds with three or more carbon atoms (C₃₊ compounds), comprising: directing a feed stream comprising ethylene (C₂H₄), hydrogen (H₂) and carbon dioxide (CO₂) at a C₂H₄/H₂ molar ratio from about 0.01 to 5, and a C₂H₄/CO₂ molar ratio from about 1 to 10, into an ethylene conversion reactor that converts the C₂H₄ in an ethylene conversion process to yield a product stream comprising the C₃₊ compounds, wherein the ethylene conversion reactor comprises at least one mesoporous catalyst disposed therein and configured to facilitate the ethylene conversion process, and wherein the at least one mesoporous catalyst comprises a plurality of mesopores having an average pore size from about 1 nanometer (nm) to 500 nm.

In some embodiments, the C₃₊ compounds comprise hydrocarbon compounds with five or more carbon atoms (C₅₊ compounds). In some embodiments, the ethylene conversion reactor is an ethylene-to-liquids (ETL) reactor, and wherein the ethylene conversion process is an ETL process. In some embodiments, the average pore size is from 1 nm to 50 nm. In some embodiments, the average pore size is from 1 nm to 10 nm. In some embodiments, the C₂H₄/H₂ molar ratio is between about 0.1 and about 2. In some embodiments, the C₂H₄/H₂ molar ratio is about 0.6. In some embodiments, the C₂H₄/CO₂ molar ratio is between about 5 and about 10. In some embodiments, the C₂H₄/CO₂ molar ratio is about 6.

Another aspect of the present disclosure provides a method for generating hydrocarbon compounds with three or more carbon atoms (C₃₊ compounds), comprising: directing a feed stream comprising ethylene (C₂H₄) into an ethylene conversion reactor that converts the C₂H₄ in an ethylene conversion process to yield a product stream comprising the C₃₊ compounds, wherein the ethylene conversion reactor comprising a catalyst disposed therein and configured to facilitate the ethylene conversion process, and wherein the catalyst comprises at least one crystalline catalytic material and at least one amorphous catalytic material.

In some embodiments, the C₃₊ compounds comprise hydrocarbon compounds with five or more carbon atoms (C₅₊ compounds). In some embodiments, the ethylene conversion reactor is an ethylene-to-liquids (ETL) reactor, and wherein the ethylene conversion process is an ETL process. In some embodiments, the at least one crystalline catalytic material comprises zeolite. In some embodiments, the at least one amorphous catalytic material comprise a mesoporous catalyst having a plurality of mesopores. In some embodiments, the plurality of mesopores has an average pore size from about 1 nanometer (nm) to about 500 nm. In some embodiments, the average pore size is from 1 nm to 50 nm. In some embodiments, the average pore size is from 1 nm to 10 nm. In some embodiments, the mesoporous catalyst is MCM-41. In some embodiments, the at least one crystalline catalytic material is intermixed with the at least one amorphous catalytic material. In some embodiments, the at least one crystalline catalytic material is modified prior to being intermixed with the at least one amorphous catalytic material. In some embodiments, the modified crystalline catalytic material is mesostructured. In some embodiments, the modified crystalline catalytic material comprises a plurality of mesopores having an average pore size from about 1 nanometer (nm) to 500 nm. In some embodiments, the average pore size is from 1 nm to 50 nm. In some embodiments, the average pore size is from 1 nm to 10 nm.

Another aspect of the present disclosure provides a method of forming a catalyst comprising a mesoporous zeolite, comprising: contacting a zeolite having a framework silicon-to-aluminum ratio (SAR) greater than 80 with a pH controlled solution, thereby forming the catalyst comprising the mesoporous zeolite, wherein the mesoporous zeolite comprises one or more mesopores, and wherein the one or more mesopores have an average pore size between about 1 nanometer (nm) and about 500 nm.

In some embodiments, the average pore size is from 1 nm to 50 nm. In some embodiments, the average pore size is from 1 nm to 10 nm. In some embodiments, the SAR is less than or equal to about 800. In some embodiments, the pH controlled solution comprises a surfactant. In some embodiments, the surfactant is a cationic surfactant, an anionic surfactant, a neutral surfactant, or any combination thereof. In some embodiments, the pH controlled solution is a basic solution. In some embodiments, the pH controlled solution is an acidic solution. In some embodiments, the zeolite comprises zeolite A, faujasites, mordenite, CHA, ZSM-5, ZSM-11, ZSM-12, ZSM-22, beta zeolite, synthetic ferrierite (ZSM-35), synthetic mordenite, functional variants or any combination thereof. In some embodiments, the faujasite is zeolite X. In some embodiments, the catalyst has a lifetime that is greater than a lifetime of the zeolite when subjected to reaction conditions in an ethylene conversion process. In some embodiments, the catalyst has a lifetime that is at least 1.5 times greater than a lifetime of the zeolite when subjected to reaction conditions in an ethylene conversion process. In some embodiments, the ethylene conversion process is an ethylene-to-liquids (ETL) process.

Another aspect of the present disclosure provides a method of forming a catalyst comprising a mesoporous zeolite, comprising: contacting a zeolite with a pH controlled solution comprising ions of one or more chemical elements, thereby forming the catalyst comprising the mesoporous zeolite, wherein the mesoporous zeolite has a modified framework comprising the at least one of the one or more chemical elements incorporated therein, and wherein the mesoporous zeolite comprises one or more mesopores having an average pore size between about 1 nanometer (nm) and about 500 nm.

In some embodiments, the average pore size is from 1 nm to 50 nm. In some embodiments, the average pore size is from 1 nm to 10 nm. In some embodiments, the ions comprise metal ions. In some embodiments, the metals ions comprise metal cations of an alkali, alkaline earth, transition, or rare earth metal. In some embodiments, the ions comprise nonmetal ions. In some embodiments, the one or more chemical elements comprise sodium, copper, iron, manganese, silver, zinc, nickel, gallium, titanium, phosphorus, boron, or any combination thereof. In some embodiments, the catalyst has a lifetime that is greater than a lifetime of the zeolite when subjected to reaction conditions in an ethylene conversion process. In some embodiments, the catalyst has a lifetime that is at least 1.5 times greater than a lifetime of the zeolite when subjected to reaction conditions in an ethylene conversion process. In some embodiments, the ethylene conversion process is an ethylene-to-liquids (ETL) process.

Another aspect of the present disclosure provides a catalyst comprising a mesoporous zeolite having a framework silicon-to-aluminum ratio (SAR) greater than about 60, wherein the mesoporous zeolite comprises one or more mesopores having an average pore size between about 1 nanometer (nm) and about 500 nm.

In some embodiments, the average pore size is from 1 nm to 50 nm. In some embodiments, the average pore size is from 1 nm to 10 nm. In some embodiments, the SAR is greater than or equal to about 80. In some embodiments, the SAR is less than or equal to about 800.

Another aspect of the present disclosure provides a catalyst comprising a mesoporous zeolite having a modified framework comprising silicon, aluminum and at least another chemical element, wherein the mesoporous zeolite comprises one or more mesopores having an average pore size between about 1 nanometer (nm) and about 500 nm.

In some embodiments, the average pore size is from 1 nm to 50 nm. In some embodiments, the average pore size is from 1 nm to 10 nm. In some embodiments, the at least another chemical element comprise sodium, copper, iron, manganese, silver, zinc, nickel, gallium, titanium, phosphorus, boron, or any combination thereof.

Another aspect of the present disclosure provides a method for generating hydrocarbon compounds with eight or more carbon atoms (C₈₊ compounds), comprising: (a) directing a feed stream comprising unsaturated hydrocarbon compounds with two or more carbon atoms (unsaturated C₂₊ compounds) into an oligomerization unit that permits at least a portion of the unsaturated C₂₊ compounds to react in an oligomerization process to yield an effluent comprising unsaturated higher hydrocarbon compounds; and (b) directing at least a portion of the effluent from the oligomerization unit and a stream comprising isoparaffins into an alkylation unit downstream of and separate from the oligomerization unit, which alkylation unit permits at least a portion of the unsaturated higher hydrocarbon compounds and the isoparaffins to react in an alkylation process to yield a product stream comprising the C₈₊ compounds.

In some embodiments, the C₈₊ compounds comprise saturated hydrocarbons. In some embodiments, at least 80 mol % of the C₈₊ compounds are saturated hydrocarbons. In some embodiments, at least 90 mol % of the C₈₊ compounds are saturated hydrocarbons. In some embodiments, the C₈₊ compounds comprise hydrocarbon compounds with eight to twelve carbon atoms (C₈-C₁₂ compounds). In some embodiments, the C₈₊ compounds comprise branched hydrocarbon compounds. In some embodiments, the product stream is an alkylate stream comprising an alkylate product. In some embodiments, the alkylate product comprises the C₈₊ compounds. In some embodiments, the alkylate product has a research octane number (RON) greater than about 95. In some embodiments, the alkylate product has a motor octane number (MON) greater than about 85. In some embodiments, the stream comprising the isoparaffins is external to the oligomerization unit. In some embodiments, the isoparaffins comprises isobutane. In some embodiments, the effluent comprises less than about 10 mol % of isoparaffins. In some embodiments, the oligomerization unit is an ethylene conversion unit. In some embodiments, the ethylene conversion unit is an ethylene-to-liquids (ETL) unit. In some embodiments, the oligomerization unit is a dimerization unit, and wherein the oligomerization process is a dimerization process. In some embodiments, the dimerization unit comprises a plurality of dimerization reactors. In some embodiments, individual reactors of the plurality of dimerization reactors are fluidically parallel to each other. In some embodiments, the dimerization process is operated at a temperature from about 40° C. to about 200° C. In some embodiments, the dimerization process is operated at a pressure from about 100 PSI to about 400 PSI. In some embodiments, the dimerization unit comprises a dimerization catalyst that facilitates the dimerization process. In some embodiments, the dimerization catalyst comprises at least one metal. In some embodiments, the at least one metal comprise one or more of nickel, palladium, chromium, vanadium, iron, cobalt, ruthenium, rhodium, copper, silver, rhenium, molybdenum, tungsten, manganese, and any combination thereof. In some embodiments, the dimerization catalyst further comprises one or more of zeolites, alumina, silica, carbon, titania, zirconia, silica/alumina, mesoporous silicas, and any combination thereof. In some embodiments, the alkylation unit comprises an alkylation catalyst that facilitates the alkylation process. In some embodiments, the alkylation catalyst comprises one or more of zeolites, sulfated zirconia, tungstated zirconia, chlorided alumina, aluminum chloride, silicon-aluminum phosphates, titaniosilicates, polyphosphoric acid, polytungstic acid, supported liquid acids, sulfuric acid on silica, hydrogen fluoride on carbon, antimony fluoride on silica, aluminum chloride (AlCl₃) on alumina (Al₂O₃), and any combination thereof. In some embodiments, the zeolites comprise one or more of zeolite Beta, BEA zeolites, MCM zeolites, faujasites, USY zeolites, LTL zeolites, mordenite, MFI zeolites, EMT zeolites, LTA zeolites, ITW zeolites, ITQ zeolites, SFO zeolites and any combination thereof.

In some embodiments, the faujasites comprise zeolite X and/or zeolite Y. In some embodiments, the method further comprises, before (a), directing the feed stream into an isomerization unit upstream of the oligomerization unit, which isomerization unit permits at least a portion of the unsaturated C₂₊ compounds to react in an isomerization process to yield a stream comprising a mixture of the unsaturated C₂₊ compounds and isomers thereof. In some embodiments, the method further comprises, between (a) and (b), directing the effluent into an isomerization unit downstream of the oligomerization unit, which isomerization unit permits at least a portion of the unsaturated higher hydrocarbon compounds to react in an isomerization process to yield a stream comprising a mixture of the unsaturated higher hydrocarbon compounds and isomers thereof. In some embodiments, the isomerization unit comprises an isomerization catalyst that facilitates the isomerization process. In some embodiments, the isomerization catalyst comprises alkaline oxides.

Another aspect of the present disclosure provides a method for generating hydrocarbon compounds with eight or more carbon atoms (C₈₊ compounds), comprising: directing a first stream comprising unsaturated hydrocarbon compounds with two or more carbon atoms (unsaturated C₂₊ compounds) and a second stream comprising isoparaffins into an alkylation unit that permits at least a portion of the unsaturated C₂₊ compounds and the isoparaffins to react in an alkylation process to yield a product stream comprising the C₈₊ compounds, wherein the first stream and the second stream are directed into the alkylation unit without passing through a dimerization unit.

In some embodiments, at least a portion of the first stream is an effluent generated in an ethylene conversion unit. In some embodiments, the ethylene conversion unit is an ethylene-to-liquids (ETL) unit. In some embodiments, the first stream is at least a portion of an effluent generated in an ethylene conversion unit. In some embodiments, the ethylene conversion unit is an ethylene-to-liquids (ETL) unit. In some embodiments, the method further comprises, directing an ETL feed stream into the ETL unit that permits at least a portion of the ETL feed stream to react in an ETL process to yield the unsaturated C₂₊ compounds. In some embodiments, the ETL unit comprises an ETL catalyst that facilitates the ETL process. In some embodiments, the ETL catalyst comprises at least one metal. In some embodiments, the at least one metal comprise one or more of nickel, palladium, chromium, vanadium, iron, cobalt, ruthenium, rhodium, copper, silver, rhenium, molybdenum, tungsten, manganese, gallium, platinum, and any combination thereof. In some embodiments, the ETL catalyst further comprises one or more of zeolites amorphous silica alumina, silica, alumina, mesoporous silica, mesoporous alumina, zirconia, titania, pillared clay, and any combination thereof. In some embodiments, the zeolites comprise ZSM-5, zeolite Beta, ZSM-11, functional variants or any combination thereof. In some embodiments, the method further comprises, directing an oxidizing agent and the ETL feed stream into the ETL unit. In some embodiments, the oxidizing agent reacts with at least a portion of hydrogen (H₂) in the ETL feed stream, thereby reducing hydrogenation of unsaturated hydrocarbon compounds over the ETL catalyst in the ETL unit. In some embodiments, the hydrogenation of unsaturated hydrocarbon compounds is reduced by at least about 20% as compared to hydrogenation of unsaturated hydrocarbon compounds in the ETL unit in the absence of the oxidizing agent. In some embodiments, the oxidizing agent comprises oxygen (O₂), air or water. In some embodiments, a molar ratio of the oxidizing agent to the ETL feed stream is from about 0.01 to about 10. In some embodiments, the method further comprises directing the ETL feed stream into a Fischer-Tropsch (FT) unit upstream of the ETL unit, which FT unit permits at least a portion of carbon monoxide (CO) and H₂ in the ETL feed stream to react in a FT process to yield an effluent comprising hydrocarbon compounds having one to four carbon atoms (C₁-C₄ compounds). In some embodiments, the method further comprises directing the ETL feed stream into a hydrotreating unit upstream of the ETL unit, the hydrotreating unit comprising a hydrotreating catalyst that facilitates a hydrotreating process for removing at least a portion of sulfur (S) from the ETL feed stream. In some embodiments, at least 50 mol % of S is removed from the ETL feed stream. In some embodiments, the ETL unit and hydrotreating unit are separate reactor zones in the same reactor. In some embodiments, the hydrotreating catalyst comprises CoMo-based catalyst, NiMo-based catalyst or any combination thereof. In some embodiments, the method further comprises, directing one or more additional feed streams comprising unsaturated hydrocarbon compounds with three or more carbon atoms (unsaturated C₃₊ compounds) into the alkylation unit. In some embodiments, the unsaturated C₃₊ compounds comprise unsaturated hydrocarbon compounds having three or four carbon atoms (unsaturated C₃₌/C₄₌ compounds). In some embodiments, the unsaturated C₃₊ compounds comprise unsaturated hydrocarbon compounds having five or six carbon atoms (unsaturated C₅₌/C₆₌ compounds). In some embodiments, the one or more additional feed streams are generated in one or more additional processing units. In some embodiments, the one or more processing units comprise fluid catalytic cracking (FCC) unit, methanol-to-olefins (MTO) unit, FT unit, delayed cokers, steam crackers, or any combination thereof. In some embodiments, the product stream is an alkylate stream comprising an alkylate product. In some embodiments, the alkylate product comprises the C₈₊ compounds. In some embodiments, the alkylate product has a research octane number (RON) greater than about 95. In some embodiments, the alkylate product has a motor octane number (MON) greater than about 85. In some embodiments, the alkylation unit comprises an alkylation catalyst that facilitates the alkylation process. In some embodiments, the alkylation catalyst comprises one or more of zeolites, sulfated zirconia, tungstated zirconia, chlorided alumina, aluminum chloride, silicon-aluminum phosphates, titaniosilicates, polyphosphoric acid, polytungstic acid, supported liquid acids, sulfuric acid on silica, hydrogen fluoride on carbon, antimony fluoride on silica, aluminum chloride (AlCl₃) on alumina (Al₂O₃), and any combination thereof. In some embodiments, the zeolites comprise one or more of zeolite Beta, BEA zeolites, MCM zeolites, faujasites, USY zeolites, LTL zeolites, mordenite, MFI zeolites, EMT zeolites, LTA zeolites, ITW zeolites, ITQ zeolites, SFO zeolites and any combination thereof. In some embodiments, the faujasites comprise zeolite X and/or zeolite Y.

Another aspect of the present disclosure provides a method for generating hydrocarbon compounds with eight or more carbon atoms (C₈₊ compounds), comprising: (a) directing a feed stream comprising ethylene (C₂H₄) into an ethylene conversion unit that permits at least a portion of the C₂H₄ to react in an ethylene conversion process to yield an effluent comprising (i) unsaturated higher hydrocarbon compounds with three or more carbon atoms (unsaturated C₃₊ compounds), and (ii) isoparaffins with four or more carbon atoms (C₄₊ isoparaffins); and (b) directing at least a portion of the effluent from the ethylene conversion unit into an alkylation unit downstream of the ethylene conversion unit, which alkylation unit permits at least a portion of the unsaturated C₃₊ compounds and the C₄₊ isoparaffins to react in an alkylation process to yield a product stream comprising the C₈₊ compounds, wherein the alkylation process is conducted in the absence of an additional feed stream of isoparaffins external to the ethylene conversion unit and the alkylation unit.

In some embodiments, the ethylene conversion unit is an ethylene-to-liquids (ETL) unit, and wherein the ethylene conversion process is an ETL process. In some embodiments, the at least a portion of the effluent is directed from the ETL unit into the alkylation unit without passing through a dimerization unit. In some embodiments, the method further comprises, before (b), directing the at least a portion of the effluent from the ETL unit into a separations unit that separates at least a portion of the unsaturated C₃₊ compounds and unreacted C₂H₄ from the at least a portion of the effluent. In some embodiments, the method further comprises, directing the at least a portion of the unsaturated C₃₊ compounds from the separations unit into a fractionation unit that (1) separates at least one impurities comprising saturated hydrocarbon compounds with three or more carbon atoms from the at least a portion of the unsaturated C₃₊ compounds, and (2) yields a first stream comprising the at least one impurities and a second stream comprising the at least a portion of the unsaturated C₃₊ compounds. In some embodiments, the method further comprises, directing the second stream comprising the at least a portion of the unsaturated C₃₊ compounds from the fractionation unit into the alkylation unit. In some embodiments, the method further comprises, directing the at least a portion of the effluent from the separations unit into an additional separations unit downstream of the separations unit that separates the C₄₊ isoparaffins from the at least a portion of the effluent. In some embodiments, the method further comprises, directing the C₄₊ isoparaffins from the additional separations unit into the alkylation unit. In some embodiments, the C₄₊ isoparaffins comprise isopentane. In some embodiments, the C₄₊ isoparaffins comprise at least 90 mol % isopentane. In some embodiments, the C₄₊ isoparaffins comprise less than about 5 mol % isobutane. In some embodiments, the method further comprises, directing one or more additional feed streams comprising unsaturated C₃₊ compounds into the alkylation unit. In some embodiments, the unsaturated C₃₊ compounds comprise unsaturated hydrocarbon compounds having three or four carbon atoms (unsaturated C₃₌/C₄₌ compounds). In some embodiments, the unsaturated C₃₊ compounds comprise unsaturated hydrocarbon compounds having five or six carbon atoms (unsaturated C₅₌/C₆₌ compounds). In some embodiments, the one or more additional feed streams are generated in one or more additional processing units. In some embodiments, the one or more processing units comprise fluid catalytic cracking (FCC) unit, methanol-to-olefins (MTO) unit, FT unit, delayed cokers, steam crackers, or any combination thereof. In some embodiments, the alkylation unit comprises an alkylation catalyst that facilitates the alkylation process. In some embodiments, the alkylation catalyst comprises one or more of zeolites, sulfated zirconia, tungstated zirconia, chlorided alumina, aluminum chloride, silicon-aluminum phosphates, titaniosilicates, polyphosphoric acid, polytungstic acid, supported liquid acids, sulfuric acid on silica, hydrogen fluoride on carbon, antimony fluoride on silica, aluminum chloride (AlCl₃) on alumina (Al₂O₃), and any combination thereof. In some embodiments, the zeolites comprise one or more of zeolite Beta, BEA zeolites, MCM zeolites, faujasites, USY zeolites, LTL zeolites, mordenite, MFI zeolites, EMT zeolites, LTA zeolites, ITW zeolites, ITQ zeolites, SFO zeolites and any combination thereof. In some embodiments, the faujasites comprise zeolite X and/or zeolite Y.

Another aspect of the present disclosure provides a method for generating alkyl aromatic hydrocarbon compounds, comprising: (a) directing a feed stream comprising ethylene (C₂H₄) into an ethylene conversion unit that permits at least a portion of the C₂H₄ to react in an ethylene conversion process to yield an effluent comprising higher hydrocarbon compounds with three or more carbon atoms (C₃₊ compounds); (b) directing at least a portion of the effluent from the ethylene conversion unit into a separations unit that separates the at least a portion of the effluent into (i) a first stream comprising hydrocarbon compounds with four or less carbon atoms (C₄₋ compounds) including unreacted C₂H₄, and (ii) a second stream comprising hydrocarbon compounds with five or more carbon atoms (C₅₊ compounds); (c) directing at least a portion of the second stream comprising the C₅₊ compounds from the separations unit into an aromatic extraction unit to yield an extraction effluent comprising aromatic hydrocarbon compounds with five or more carbon atoms (C₅₊ aromatics); and (d) directing at least a portion of the first stream comprising the C₄₋ compounds from the separations unit and at least a portion of the extraction effluent comprising the C₅₊ aromatics from the aromatic extraction unit into an alkylation unit that permits at least a portion of the C₄₋ compounds and the C₅₊ aromatics to react in an alkylation process to yield a product stream comprising the alkyl aromatic hydrocarbon compounds.

In some embodiments, the C₄₋ compounds comprise unsaturated hydrocarbon compounds with four or less carbon atoms (unsaturated C₄₋ compounds). In some embodiments, the C₄₋ compounds comprise at least 80 mol % unsaturated C₄₋ compounds. In some embodiments, the C₅₊ compounds comprise benzene. In some embodiments, the alkyl aromatic hydrocarbon compounds comprise xylene, ethylbenzene, isopropylbenzene, or any combination thereof. In some embodiments, the method further comprise, between (c) and (d), directing the extraction effluent comprising the C₅₊ aromatics from the aromatic extraction unit into an additional separations unit that separates the C₅₊ aromatics into (i) a first separations stream comprising benzene, and (ii) a second separations stream comprising aromatic hydrocarbon compounds with seven or more carbon atoms (C₇₊ aromatics). In some embodiments, the method further comprises, directing the first separations stream from the additional separations unit into the alkylation unit. In some embodiments, the method further comprises, directing the second separations stream into a product tank without further processing. In some embodiments, the at least a portion of the first stream comprising the C₄₋ compounds and the at least a portion of the extraction effluent comprising the C₅₊ aromatics are directed into the alkylation unit without passing through a dimerization unit. In some embodiments, the ethylene conversion unit is an ethylene-to-liquids (ETL) unit, and wherein the ethylene conversion process is an ETL process. In some embodiments, the alkylation unit comprises an alkylation catalyst that facilitates the alkylation process. In some embodiments, the alkylation catalyst comprises one or more of zeolites, sulfated zirconia, tungstated zirconia, chlorided alumina, aluminum chloride, silicon-aluminum phosphates, titaniosilicates, polyphosphoric acid, polytungstic acid, supported liquid acids, sulfuric acid on silica, hydrogen fluoride on carbon, antimony fluoride on silica, aluminum chloride (AlCl₃) on alumina (Al₂O₃), and any combination thereof. In some embodiments, the zeolites comprise one or more of zeolite Beta, BEA zeolites, MCM zeolites, faujasites, USY zeolites, LTL zeolites, mordenite, MFI zeolites, EMT zeolites, LTA zeolites, ITW zeolites, ITQ zeolites, SFO zeolites and any combination thereof. In some embodiments, the faujasites comprise zeolite X and/or zeolite Y.

Another aspect of the present disclosure provides a method for generating hydrocarbon compounds with fourteen or more carbon atoms (C₁₄₊ compounds), comprising: (a) directing a feed stream comprising ethylene (C₂H₄) into an ethylene conversion unit that permits at least a portion of the C₂H₄ to react in an ethylene conversion process to yield an effluent comprising higher hydrocarbon compounds with three or more carbon atoms (C₃₊ compounds); (b) directing at least a portion of the effluent from the ethylene conversion unit and a stream comprising isoparaffins into a first alkylation unit that permits at least a portion of the C₃₊ compounds and the isoparaffins to react in a first alkylation process to yield an alkylation product stream; (c) directing at least a portion of the alkylation product stream from the first alkylation unit into a separations unit to yield a separations product stream comprising higher hydrocarbon compounds with six or more carbon atoms (C₆₊ compounds); and (d) directing at least a portion of the separations product stream from the separations unit into a second alkylation unit that permits at least a portion of the C₆₊ compounds to react in a second alkylation process to yield a product stream comprising the C₁₄₊ compounds.

In some embodiments, the isoparaffins comprise isobutane, isopentane, or any combination thereof. In some embodiments, the C₆₊ compounds comprise (i) isoparaffins and (ii) unsaturated hydrocarbon compounds with six or more carbon atoms (unsaturated C₆₊ compounds). In some embodiments, the isoparaffins comprise isoparaffins with eight or more carbon atoms (C₈₊ isoparaffins). In some embodiments, the second alkylation unit permits at least a portion of the C₈₊ isoparaffins and the unsaturated C₆₊ compounds to react in the second alkylation process to yield the product stream. In some embodiments, the ethylene conversion unit is an ethylene-to-liquids (ETL) unit, and wherein the ethylene conversion process is an ETL process. In some embodiments, the first alkylation unit and the second alkylation unit are operated under the same condition. In some embodiments, the first alkylation unit and the second alkylation unit are operated under different conditions. In some embodiments, the first alkylation unit comprises a first alkylation catalyst and the second alkylation unit comprises a second alkylation catalyst. In some embodiments, the first alkylation catalyst is different from the second alkylation catalyst. In some embodiments, the first alkylation catalyst is the same as the second alkylation catalyst. In some embodiments, at least one of the first alkylation catalyst and the second alkylation catalyst comprises one or more of zeolites, sulfated zirconia, tungstated zirconia, chlorided alumina, aluminum chloride, silicon-aluminum phosphates, titaniosilicates, polyphosphoric acid, polytungstic acid, supported liquid acids, sulfuric acid on silica, hydrogen fluoride on carbon, antimony fluoride on silica, aluminum chloride (AlCl₃) on alumina (Al₂O₃), and any combination thereof. In some embodiments, the zeolites comprise one or more of zeolite Beta, BEA zeolites, MCM zeolites, faujasites, USY zeolites, LTL zeolites, mordenite, MFI zeolites, EMT zeolites, LTA zeolites, ITW zeolites, ITQ zeolites, SFO zeolites and any combination thereof. In some embodiments, the faujasites comprise zeolite X and/or zeolite Y.

Another aspect of the present disclosure provides a method for generating hydrocarbon compounds with five or more carbon atoms (C₅₊ compounds), the method comprising: (a) injecting a stream containing methane into an oxidative coupling of methane (OCM) reactor to produce an OCM product stream containing olefins; (b) injecting the OCM product stream and a water recovery stream into an ethylene-to-liquids (ETL) reactor to produce an ETL product stream containing hydrocarbons with four carbon atoms (C₄ compounds), hydrocarbons with five or more carbon atoms (C₅₊ compounds), and water; (c) injecting the ETL product stream into a first separations unit to generate a first stream containing the C₄ compounds and a second stream containing the C₅₊ compounds and the water; and (d) injecting the second stream into a second separations unit to produce a C₅₊ stream containing the C₅₊ compounds) and the water recovery stream.

In some embodiments, the method further comprises injecting an effluent stream generated in a fluidized catalytic cracking (FCC) unit into the ETL reactor. In some embodiments, the method further comprises injecting the first stream generated in (c) into a fractionation unit to produce a first fractionation product stream containing olefins with between two and four carbon atoms (C₂-C₄ olefins) and a second fractionation product stream containing methane and ethane. In some embodiments, the method further comprises injecting the first fractionation product stream into the ETL reactor. In some embodiments, the method further comprises injecting the second fractionation product into the OCM reactor. In some embodiments, the method further comprises injecting an additional amount of water into the water recovery stream. In some embodiments, the additional amount of water is less than or equal to about 30% of an amount of water in the water recovery stream. In some embodiments, the first separations unit is a distillation column. In some embodiments, the method further comprises injecting the second stream generated in (c) into a hydration unit to convert at least a portion of the C₅₊ compounds into oxygenates with five or more carbon atoms (C₅₊ oxygenates). In some embodiments, the hydration unit operates at a temperature between about 100° C. and about 200° C. In some embodiments, the hydration unit operates at a pressure between about 1 bar and 100 bar. In some embodiments, the hydration unit operates with a feed composition having at least about 50 mole percent water and less than about 50 mole percent hydrocarbons. In some embodiments, the hydration unit contains a hydration catalyst. In some embodiments, the hydration catalyst comprises an acid catalyst. In some embodiments, the acid catalyst is selected from the group consisting of water soluble acids, organic acids, solid acids, and any combination thereof. In some embodiments, the ETL reactor contains an ETL catalyst. In some embodiments, the ETL catalyst is a zeolite. In some embodiments, the zeolite comprises ZSM-5, ZSM-11, ZSM-12, ZSM-35, ZSM-38, Beta, Mordinite, or any combination thereof. In some embodiments, the ETL reactor operates with a feed composition between about 0.5 mole water per mole olefins and about 16 mole water per mole olefins.

Another aspect of the present disclosure provides a method for generating hydrocarbons having six or more carbon atoms (C₆₊ hydrocarbons) via catalytic distillation, the method comprising: (a) injecting a stream containing ethylene (into a catalytic distillation vessel comprising an oligomerization catalyst; and (b) reacting at least a portion of the stream in the catalytic distillation vessel using the oligomerization catalyst under reaction conditions that yield a vapor stream comprising hydrocarbons having four carbon atoms (C₄ hydrocarbons) and a liquid stream comprising C₆₊ hydrocarbons, wherein at least a portion of the ethylene in the stream is generated in an oxidative coupling of methane (OCM) process.

In some embodiments, the method further comprises injecting at least a portion of the vapor stream into a condenser to liquefy the C₄ hydrocarbons and directing the C₄ hydrocarbons liquefied in the condenser as a recycle stream into the catalytic distillation vessel. In some embodiments, the method further comprises injecting at least a portion of the liquid stream into a reboiler to produce a gaseous stream comprising the C₆₊ hydrocarbons and directs the gaseous stream as a recycle stream into the catalytic distillation vessel. In some embodiments, the oligomerization catalyst is a metal or a combination of metals on a catalyst support. In some embodiments, the metal comprises Ni, Pd, Cr, V, Fe, Co, Ru, Rh, Cu, Ag, Re, Mo, W, Mn, and Pt, or any combination thereof. In some embodiments, the catalyst support comprises zeolite, amorphous silica alumina, silica, alumina, mesoporous silica, mesoporous alumina, zirconia, titania, pillared clay, or any combination thereof. In some embodiments, the zeolite comprises ZSM-5, Beta, ZSM-11, or any combination thereof.

Another aspect of the present disclosure provides a method for generating hydrocarbons having six or more carbon atoms (C₆₊ hydrocarbons) via catalytic distillation, the method comprising: (a) injecting a stream containing ethylene into a catalytic distillation vessel comprising an oligomerization catalyst; (b) reacting at least a portion of the stream in the catalytic distillation vessel using the oligomerization catalyst under reaction conditions that yield a vapor stream comprising unconverted ethylene and a liquid stream comprising hydrocarbons having four or more carbon atoms (C₄₊ hydrocarbons); and (c) injecting at least a portion of the liquid stream into a distillation column to generate a vapor effluent stream comprising hydrocarbons having four carbon atoms (C₄ hydrocarbons) and a liquid effluent stream comprising hydrocarbons having six or more carbon atoms (C₆₊ hydrocarbons), wherein at least a portion of the ethylene in the stream is generated in an oxidative coupling of methane (OCM) process.

In some embodiments, the oligomerization catalyst is a metal or a combination of metals on a catalyst support. In some embodiments, the metal comprises Ni, Pd, Cr, V, Fe, Co, Ru, Rh, Cu, Ag, Re, Mo, W, Mn, and Pt, or any combination thereof. In some embodiments, the catalyst support comprises zeolite, amorphous silica alumina, silica, alumina, mesoporous silica, mesoporous alumina, zirconia, titania, pillared clay, or any combination thereof. In some embodiments, the zeolite comprises ZSM-5, Beta, ZSM-11, or any combination thereof. In some embodiments, the catalytic distillation vessel operates at a pressure of at least about 10 bar. In some embodiments, the catalytic distillation vessel operates at a temperature of at least about 50° C. In some embodiments, the pressure is at least about 20 bar. In some embodiments, the temperature is at least about 100° C.

Another aspect of the present disclosure provides a method for etherification of olefins having five or more carbon atoms (C₅₊ olefins) via catalytic distillation, the method comprising: (a) injecting a stream containing ethylene into an ethylene-to-liquids (ETL) reactor to produce an ETL product stream containing the C₅₊ olefins; (b) injecting at least a portion of the ETL product stream and an alcohol stream containing an alcohol into a catalytic distillation vessel comprising an etherification catalyst to produce hydrocarbon compounds containing hydrocarbons having four carbon atoms (C₄ hydrocarbons) and oxygenates having six or more carbon atoms (C₆₊ oxygenates), wherein the catalytic distillation vessel operates under conditions that yield a vapor stream comprising the C₄ hydrocarbons and a liquid stream comprising the C₆₊ oxygenates.

In some embodiments, the ethylene is at least partially generated in an oxidative-coupling of methane (OCM) process. In some embodiments, the alcohol is methanol. In some embodiments, the method further comprises injecting at least a portion of the C₄ hydrocarbons into a reflux condenser to produce a liquid C₄ stream that is recycled into the catalytic distillation vessel. In some embodiments, the method further comprises injecting at least a portion of the C₆₊ oxygenates into a reboiler to produce a vapor C₆₊ stream that is recycled into the catalytic distillation vessel. In some embodiments, a molar ratio of the C₅₊ olefins to the alcohol fed into the catalytic distillation vessel is between about 0.01 and about 20. In some embodiments, a temperature in the catalytic distillation vessel is between about 50° C. and about 400° C. In some embodiments, a contact time of the reacting C₅₊ olefin and the etherification catalyst is between about 0.1 h⁻¹ and about 20 h⁻¹. In some embodiments, the etherification catalyst comprises a solid acid catalyst. In some embodiments, the solid acid catalyst comprises ionic exchange resins, acidic zeolites, metal oxides, or any combination thereof.

Another aspect of the present disclosure provides a method for hydration of olefins having five or more carbon atoms (C₅₊ olefins) via catalytic distillation, the method comprising: (a) injecting a stream containing ethylene into an ethylene-to-liquids (ETL) reactor to produce an ETL product stream containing the C₅₊ olefins; (b) injecting at least a portion of the ETL product stream and a water stream containing water into a catalytic distillation vessel comprising a hydration catalyst to produce hydrocarbon compounds containing hydrocarbons having four carbon atoms (C₄ hydrocarbons) and oxygenates having five or more carbon atoms (C₅₊ oxygenates), wherein the catalytic distillation vessel operates under conditions that yield a vapor stream comprising the C₄ hydrocarbons and a liquid stream comprising the C₅₊ oxygenates.

In some embodiments, the ethylene is at least partially generated in an oxidative-coupling of methane (OCM) process. In some embodiments, the method further comprises injecting at least a portion of the C₄ hydrocarbons into a reflux condenser to produce a liquid C₄ stream that is recycled into the catalytic distillation vessel. In some embodiments, the method further comprises injecting at least a portion of the C₅₊ oxygenates into a reboiler to produce a vapor C₅₊ stream that is recycled into the catalytic distillation vessel. In some embodiments, a molar ratio of the C₅₊ olefins to the water fed into the catalytic distillation vessel is between about 0.01 and about 20. In some embodiments, a temperature in the catalytic distillation vessel is between about 50° C. and about 400° C. In some embodiments, a pressure in the catalytic distillation vessel is between about 1 bar and about 100 bar. In some embodiments, a contact time of the reacting C₅₊ olefin and the hydration catalyst is between about 0.1 h⁻¹ and about 20 h⁻¹. In some embodiments, the hydration catalyst comprises a solid acid catalyst. In some embodiments, the solid acid catalyst comprises ionic exchange resins, acidic zeolites, metal oxides, or any combination thereof.

Another aspect of the present disclosure provides a method for producing oxygenates having six or more carbon atoms (C₆₊ oxygenates), the method comprising: injecting an ethylene stream containing ethylene and an alcohol stream containing an alcohol into a catalytic distillation vessel comprising an ethylene-to-liquids (ETL) catalyst bed and an etherification catalyst bed below the ETL catalyst bed, wherein the ethylene stream is injected into or below the ETL catalyst bed and the alcohol stream is injected into or below the etherification catalyst bed, and wherein the catalytic distillation vessel operates under reaction conditions that yield a vapor stream comprising ethylene and a liquid stream comprising the C₆₊ oxygenates.

In some embodiments, the ethylene at least partially converts into olefins having five or more carbon atoms (C₅₊ olefins) within the ETL catalyst bed. In some embodiments, the C₅₊ olefins generated within the ETL catalyst bed move down the catalytic distillation vessel into the etherification catalyst bed. In some embodiments, the method further comprises injecting the vapor stream into a condenser to produce a first stream containing hydrocarbons having four carbon atoms (C₄ hydrocarbons) and a second stream containing the ethylene. In some embodiments, the method further comprises recycling at least a portion of the second stream into the catalytic distillation vessel. In some embodiments, the method further comprises recycling at least a portion of the first stream into the catalytic distillation vessel. In some embodiments, a temperature in the catalytic distillation vessel is between about 100° C. and about 200° C. In some embodiments, a pressure in the catalytic distillation vessel is between about 10 bar and about 80 bar. In some embodiments, a ratio of molar flow rates of the alcohol stream to the ethylene stream is between about 0.01 and about 20. In some embodiments, a contact time between the reacting C₅₊ olefin and an etherification catalyst in the etherification catalyst bed is between about 0.1 h⁻¹ and about 20 h⁻¹. In some embodiments, a contact time between the reacting ethylene and an ETL catalyst in the ETL catalyst bed is between about 0.1 h⁻¹ and about 20 h⁻¹. In some embodiments, the ETL catalyst bed comprises an ETL catalyst comprising a metal and a catalyst support. In some embodiments, the metal comprises Ni, Pd, Cr, V, Fe, Co, Ru, Rh, Cu, Ag, Re, Mo, W, Mn, Pt, or any combination thereof. In some embodiments, the catalyst support comprises zeolite, amorphous silica alumina, silica, alumina, mesoporous silica, mesoporous alumina, zirconia, titania, pillared clay, or any combination thereof. In some embodiments, the zeolite comprises ZSM-5, Beta, ZSM-11, or any combination thereof. In some embodiments, the alcohol is methanol.

Another aspect of the present disclosure provides a method for producing oxygenates having five or more carbon atoms (C₅₊ oxygenates), the method comprising: injecting an ethylene stream containing ethylene and a water stream containing water into a catalytic distillation vessel comprising an ethylene-to-liquids (ETL) catalyst bed and a hydration catalyst bed below the ETL catalyst bed, wherein the ethylene stream is injected into or below the ETL catalyst bed and the alcohol stream is injected into or below the hydration catalyst bed, and wherein the catalytic distillation vessel operates under conditions that yield a gas stream comprising ethylene and a liquid stream comprising the C₅₊ oxygenates.

In some embodiments, the ethylene at least partially converts into olefins having five or more carbon atoms (C₅₊ olefins) within the ETL catalyst bed. In some embodiments, the C₅₊ olefins generated within the ETL catalyst bed move down the catalytic distillation vessel into the hydration catalyst bed. In some embodiments, the method further comprises injecting the gas stream into a condenser to produce a first stream containing hydrocarbons having four carbon atoms (C₄ hydrocarbons) and a second stream containing the ethylene. In some embodiments, the method further comprises recycling at least a portion of the second stream into the catalytic distillation vessel. In some embodiments, the method further comprises recycling at least a portion of the first stream into the catalytic distillation vessel. In some embodiments, a temperature in the catalytic distillation vessel is between about 100° C. and about 200° C. In some embodiments, a pressure in the catalytic distillation vessel is between about 10 bar and about 80 bar. In some embodiments, a ratio of molar flow rates of the water stream to the ethylene stream is between about 0.01 and about 20. In some embodiments, a contact time between the reacting C₅₊ olefin and an etherification catalyst in the etherification catalyst bed is greater than 0.1 h⁻¹ and less than 20 h⁻¹. In some embodiments, a contact time between the reacting ethylene and an ETL catalyst in the ETL catalyst bed is between about 0.1 h⁻¹ and about 20 h⁻¹. In some embodiments, the ETL catalyst bed comprises an ETL catalyst comprising a metal and a catalyst support. In some embodiments, the metal comprises Ni, Pd, Cr, V, Fe, Co, Ru, Rh, Cu, Ag, Re, Mo, W, Mn, Pt, or any combination thereof. In some embodiments, the catalyst support comprises zeolite, amorphous silica alumina, silica, alumina, mesoporous silica, mesoporous alumina, zirconia, titania, pillared clay, or any combination thereof. In some embodiments, the zeolite comprises ZSM-5, Beta, ZSM-11, or any combination thereof.

Another aspect of the present disclosure provides a method for producing hydrocarbon compounds with three or more carbon atoms (C₃₊ compounds), the method comprising: (a) directing a feed stream comprising unsaturated hydrocarbon compounds with two or more carbon atoms (unsaturated C₂₊ compounds) into a chemical reactor, wherein the chemical reactor converts at least a portion of the unsaturated C₂₊ compounds to C₃₊ compounds, thereby producing a product stream comprising the C₃₊ compounds; (b) fractionating the C₃₊ compounds to produce (i) a light product stream comprising hydrocarbon compounds having two to four carbon atoms (C₂-C₄ compounds) and (ii) a heavy product stream comprising hydrocarbon compounds having five or more carbons atoms (C₅₊ compounds); and (c) combining a portion of the light product stream with the feed stream and/or directing the portion of the light product stream back to the chemical reactor, wherein the portion of the light product stream is selected such that a concentration of unsaturated C₂₊ compounds entering the chemical reactor is less than about 15 mol %.

In some embodiments, the method further comprises cooling the product stream in a heat exchanger; directing the product stream from the heat exchanger to a flash drum to condense the product stream, thereby producing the light product stream and the heavy product stream; directing the light product stream to a compressor to compress the light product stream; and directing the light product stream from the compressor to the chemical reactor, thereby reacting at least a portion of the C₂-C₄ compounds in the light product stream to produce additional C₃₊ compounds. In some embodiments, the chemical reactor is substantially adiabatic. In some embodiments, the chemical reactor comprises an unsaturated C₂₊ conversion catalyst. In some embodiments, the unsaturated C₂₊ conversion catalyst is selected from the group consisting of a zeolite, a sulfated zirconia, a tungstated zirconia, a chlorided alumina, silica-aluminum phosphates, titanosilicates, amorphous silica alumina, supported liquid acids, Metal Organic Framework (MOF), and any combination thereof. In some embodiments, the zeolite comprises a Beta zeolite, a BEA zeolites, MCM zeolites, faujasites, USY zeolites, LTL zeolites, mordenite, MFI zeolites, EMT zeolites, LTA zeolites, ITW zeolites, ITQ zeolites, SFO zeolites, CHA zeolites, or any combination thereof. In some embodiments, the MFI zeolite is a ZSM-5 with a silica/alumina ratio greater than or equal to about 50. In some embodiments, the MFI zeolite is mesoporous. In some embodiments, supported liquid acids comprise solid phosphoric acid, silicotungstic acid, sulfuric acid on silica, or any combination thereof. In some embodiments, the MOF comprises a hydrocarbon unit containing a chemical functional group, and wherein the chemical functional group is selected from the group consisting of a carboxylate, carboxylic acid, alcohol, imidazole, triazole, and any combination thereof. In some embodiments, the unsaturated C₂₊ conversion catalyst comprises metal ions, and wherein the metal ions are selected from the group consisting of sodium, copper, iron, manganese, silver, zinc, nickel, gallium, titanium, nickel, cobalt, palladium, chromium, copper, vanadium, zirconium, and any combination thereof. In some embodiments, the feed stream further comprises hydrogen. In some embodiments, the feed stream comprises less than or equal to about 40 mol % of hydrogen. In some embodiments, the method further comprises prior to (a), directing at least a portion of the feed stream to a hydrogen removal unit upstream of the chemical reactor, which hydrogen removal unit removes at least a portion of the hydrogen from the feed stream.

Another aspect of the present disclosure provides a method for producing hydrocarbon compounds with three or more carbons (C₃₊ compounds), the method comprising: (a) directing a feed stream comprising unsaturated hydrocarbon compounds with two or more carbon atoms (unsaturated C₂₊ compounds) into a chemical reactor, wherein the chemical reactor converts at least a portion of the unsaturated C₂₊ compounds in the feed stream to C₃₊ compounds, thereby producing a product stream comprising the C₃₊ compounds; and (b) directing a first portion of the product stream back to the chemical reactor, wherein the first portion of the product stream is selected such that a difference between a temperature of the feed stream and a temperature of the product stream is less than or equal to about 300° C.

In some embodiments, the first portion of the product stream comprises hydrocarbons having two to four carbon atoms (C₂-C₄ compounds). In some embodiments, the method further comprises fractionating the product stream to produce (i) a light product stream comprising hydrocarbons having two to four carbon atoms (C₂-C₄ compounds) and (ii) a heavy product stream comprising hydrocarbons having five or more carbon atoms (C₅₊ compounds), wherein the first portion of the product stream is a portion of the light product stream. In some embodiments, the method further comprises cooling the product stream in a heat exchanger; directing the product stream from the heat exchanger to a flash drum to condense the product stream, thereby producing the light product stream and the heavy product stream; directing the light product stream to a compressor to compress the light product stream; and directing the light product stream from the compressor to the chemical reactor, thereby reacting a portion of the C₂-C₄ compounds in the light product stream to produce additional C₃₊ compounds.

In some embodiments, the chemical reactor is substantially adiabatic. In some embodiments, the chemical reactor comprises an unsaturated C₂₊ conversion catalyst. In some embodiments, the unsaturated C₂₊ conversion catalyst is selected from the group consisting of a zeolite, a sulfated zirconia, a tungstated zirconia, a chlorided alumina, silica-aluminum phosphates, titanosilicates, amorphous silica alumina, supported liquid acids, Metal Organic Framework (MOF), and any combination thereof. In some embodiments, the zeolite comprises a Beta zeolite, a BEA zeolites, MCM zeolites, faujasites, USY zeolites, LTL zeolites, mordenite, MFI zeolites, EMT zeolites, LTA zeolites, ITW zeolites, ITQ zeolites, SFO zeolites, CHA zeolites, or any combination thereof. In some embodiments, the MFI zeolites comprise ZSM-5 with a silica/alumina ratio greater than or equal to about 50. In some embodiments, the MFI zeolites are mesoporous. In some embodiments, the supported liquid acids include solid phosphoric acid, silicotungstic acid, sulfuric acid on silica, or any combination thereof. In some embodiments, the MOF comprises a hydrocarbon unit containing a chemical functional group, and wherein the chemical functional group is selected from the group consisting of a carboxylate, carboxylic acid, alcohol, imidazole, triazole, and any combination thereof. In some embodiments, the unsaturated C₂₊ conversion catalyst comprises metal ions, and wherein the metal ions are selected from the group consisting of sodium, copper, iron, manganese, silver, zinc, nickel, gallium, titanium, nickel, cobalt, palladium, chromium, copper, vanadium, zirconium, and any combination thereof. In some embodiments, the feed stream further comprises hydrogen. In some embodiments, the feed stream comprises less than or equal to about 40 mol % of hydrogen. In some embodiments, the method further comprises prior to (a), directing at least a portion of the feed stream to a hydrogen removal unit upstream of the chemical reactor, which hydrogen removal unit removes at least a portion of the hydrogen from the feed stream.

Another aspect of the present disclosure provides a method for producing hydrocarbon compounds with three or more carbon atoms (C₃₊ compounds), the method comprising: (a) directing a feed stream comprising unsaturated hydrocarbon compounds with two or more carbon atoms (unsaturated C₂₊ compounds) into a chemical reaction module to convert at least a portion of the unsaturated C₂₊ compounds and to yield a product stream containing the C₃₊ compounds, wherein the feed stream has a temperature of less than or equal to about 225° C. when entering the chemical reaction module; and (b) optionally directing a first portion of the product stream back to the chemical reaction module such that at least a portion of the first portion of the product stream reacts to yield additional C₃₊ compounds.

In some embodiments, the chemical reaction module comprises at least two chemical reactors in series. In some embodiments, a portion of the unsaturated C₂₊ compounds are directed to a first chemical reactor to yield a first effluent containing unsaturated hydrocarbon compounds having two to four carbon atoms (unsaturated C₂-C₄ compounds). In some embodiments, the first effluent is directed to a second chemical reactor in fluidic connection in series to the first chemical reactor, which second chemical reactor yields a second effluent comprising hydrocarbon compounds having five or more carbon atoms (C₅₊ compounds). In some embodiments, the first effluent has a temperature of less than or equal to about 300° C. In some embodiments, the method further comprises cooling the first effluent stream in a heat exchanger; and directing the first effluent stream from the heat exchanger to a second chemical reactor in series to the first chemical reactor. In some embodiments, the first chemical reactor and the second chemical reactor are substantially adiabatic. In some embodiments, the chemical reaction module comprises an unsaturated C₂₊ conversion catalyst. In some embodiments, the unsaturated C₂₊ conversion catalyst is selected from the group consisting of a zeolite, a sulfated zirconia, a tungstated zirconia, a chlorided alumina, silica-aluminum phosphates, titanosilicates, amorphous silica alumina, supported liquid acids, Metal Organic Framework (MOF), and any combination thereof. In some embodiments, the zeolite comprises a Beta zeolite, a BEA zeolites, MCM zeolites, faujasites, USY zeolites, LTL zeolites, mordenite, MFI zeolites, EMT zeolites, LTA zeolites, ITW zeolites, ITQ zeolites, SFO zeolites, CHA zeolites, or any combination thereof. In some embodiments, the MFI zeolites include ZSM-5 with a silica/alumina ratio greater than or equal to about 50. In some embodiments, the MFI zeolites are mesoporous. In some embodiments, the supported liquid acids include solid phosphoric acid, silicotungstic acid, sulfuric acid on silica, or any combination thereof. In some embodiments, the MOF comprises a hydrocarbon unit containing a functional group, and wherein the functional group is selected from the group consisting of a carboxylate, carboxylic acid, alcohol, imidazole, triazole, and any combination thereof. In some embodiments, the unsaturated C₂₊ conversion catalyst comprises metal ions, and wherein the metal ions are selected from the group consisting of sodium, copper, iron, manganese, silver, zinc, nickel, gallium, titanium, nickel, cobalt, palladium, chromium, copper, vanadium, zirconium, and any combination thereof. In some embodiments, the feed stream further comprises hydrogen. In some embodiments, the feed stream comprises less than or equal to about 40 mol % of hydrogen. In some embodiments, the method further comprises prior to (a), directing at least a portion of the feed stream to a hydrogen removal unit upstream of the chemical reactor, which hydrogen removal unit removes at least a portion of the hydrogen from the feed stream.

Another aspect of the present disclosure provides a method for producing hydrocarbon compounds with three or more carbon atoms (C₃₊ compounds), the method comprising: (a) directing a feed stream comprising unsaturated hydrocarbon compounds with two or more carbon atoms (unsaturated C₂₊ compounds) into a chemical reactor, wherein a concentration of unsaturated C₂₊ compounds is less than or equal to about 20 mol %, and wherein the chemical reactor converts at least a portion of the unsaturated C₂₊ compounds in the feed stream to the C₃₊ compounds; and (b) cooling the chemical reactor with a cooling medium.

In some embodiments, the cooling medium is a portion of the feed stream. In some embodiments, the cooling medium is a steam having a temperature between about 200 and about 300° C. In some embodiments, the chemical reactor comprises an unsaturated C₂₊ conversion catalyst. In some embodiments, the unsaturated C₂₊ conversion catalyst is selected from the group consisting of a zeolite, a sulfated zirconia, a tungstated zirconia, a chlorided alumina, silica-aluminum phosphates, titanosilicates, amorphous silica alumina, supported liquid acids, Metal Organic Framework (MOF), and any combination thereof. In some embodiments, the zeolite comprises a Beta zeolite, BEA zeolites, MCM zeolites, faujasites, USY zeolites, LTL zeolites, mordenite, MFI zeolites, EMT zeolites, LTA zeolites, ITW zeolites, ITQ zeolites, SFO zeolites, CHA zeolites, or any combination thereof. In some embodiments, the MFI zeolites include ZSM-5 with a silica/alumina ratio greater than or equal to about 50. In some embodiments, the MFI zeolites are mesoporous. In some embodiments, the supported liquid acids include solid phosphoric acid, silicotungstic acid, sulfuric acid on silica, or any combination thereof. In some embodiments, the MOF comprises a hydrocarbon unit containing a functional group, and wherein the functional group is selected from the group consisting of a carboxylate, carboxylic acid, alcohol, imidazole, triazole, and any combination thereof. In some embodiments, the unsaturated C₂₊ conversion catalyst comprises metal ions, and wherein the metal ions are selected from the group consisting of sodium, copper, iron, manganese, silver, zinc, nickel, gallium, titanium, nickel, cobalt, palladium, chromium, copper, vanadium, zirconium, and any combination thereof. In some embodiments, the feed stream further comprises hydrogen. In some embodiments, the feed stream comprises less than or equal to about 40 mol % of hydrogen. In some embodiments, the method further comprises prior to (a), directing at least a portion of the feed stream to a hydrogen removal unit upstream of the chemical reactor, which hydrogen removal unit removes at least a portion of the hydrogen gas from the feed stream before the chemical reactor.

Another aspect of the present disclosure provides a method for producing hydrocarbons with five or more carbon atoms (C₅₊ hydrocarbons), the method comprising: injecting an isobutane stream containing isobutane and an olefin stream containing olefins into a catalytic distillation column comprising a dimerization catalyst bed and an alkylation catalyst bed, wherein the catalytic distillation column operates under conditions that yield a vapor stream comprising butane and a liquid stream comprising the C₅₊ hydrocarbons.

In some embodiments, the gas stream comprises isobutane. In some embodiments, the gas stream is condensed in a condenser and recycled to the catalytic distillation column. In some embodiments, the isobutane stream is injected above the olefin stream. In some embodiments, the dimerization catalyst bed comprises a dimerization catalyst. In some embodiments, the dimerization catalyst comprises Ni, Pd, Cr, V, Fe, Co, Ru, Rh, Cu, Ag, Re, Mo, W, Mn, Pt, or any combination thereof. In some embodiments, the alkylation catalyst bed comprises an alkylation catalyst. In some embodiments, the alkylation catalyst includes zeolites, sulfated zirconia, tungstated zirconia, chlorided alumina, aluminum chloride (AlCls), silicon-aluminum phosphates, titaniosilicates, polyphosphoric acid, polytungstic acid, supported liquid acids, sulfuric acid on silica, hydrogen fluoride on carbon, antimony fluoride on silica, aluminum chloride (AlCls) on alumina (Al2O3), or any combination thereof. In some embodiments, the method further comprises injecting at least a portion of the liquid stream into a reboiler to generate a vapor stream. In some embodiments, the method further comprises recycling at least a portion of the vapor stream into the catalytic distillation column. In some embodiments, the catalytic distillation column operates at a temperature greater than or equal to about 100° C. In some embodiments, the catalytic distillation column operates at a pressure greater than or equal to about 10 bar.

Another aspect of the present disclosure provides a method for generating hydrocarbons with 14 or more carbon atoms (C₁₄₊ hydrocarbons), the method comprising: (a) injecting a stream containing ethylene into an ethylene-to-liquids (ETL) subsystem to generate an ETL effluent stream; (b) injecting the ETL effluent stream into a catalytic distillation column comprising two alkylation catalyst beds, the catalytic distillation column operating under conditions such that butane is a vapor and moves up the catalytic distillation column and hydrocarbons having six or more carbon atoms (C₆₊ hydrocarbons) are liquids that move down the column; and (c) recovering a product stream containing the C₁₄₊ hydrocarbons from the catalytic distillation column.

In some embodiments, the method further comprises injecting an isobutane stream containing isobutane into the catalytic distillation column. In some embodiments, the isobutene stream is injected into the catalytic distillation column above the ETL effluent stream. In some embodiments, the method further comprises injecting at least a portion of the product stream into a reboiler to produce a vapor stream. In some embodiments, the method further comprises injecting at least a portion of the vapor stream into the catalytic distillation column. In some embodiments, the method further comprises injecting an olefin stream into the catalytic distillation column. In some embodiments, the olefin stream is generated in a fluidized catalytic cracking, methanol-to-olefins, Fischer-Tropshe, delayed coker, or steam cracker subsystem. In some embodiments, the alkylation catalyst beds comprise an alkylation catalyst. In some embodiments, the alkylation catalyst comprises zeolites, sulfated zirconia, tungstated zirconia, chlorided alumina, aluminum chloride (AlCls), silicon-aluminum phosphates, titaniosilicates, polyphosphoric acid, polytungstic acid, supported liquid acids, sulfuric acid on silica, hydrogen fluoride on carbon, antimony fluoride on silica, aluminum chloride (AlCls) on alumina (Al₂O₃), or any combination thereof.

Another aspect of the present disclosure provides a method for generating fuel gas and hydrocarbons having five or more carbon atoms (C₅₊ hydrocarbons), the method comprising: (a) injecting an offgas stream containing hydrogen, methane, and olefins into an ethylene-to-liquids (ETL) subsystem to convert at least a portion of the olefins comprised in the offgas stream into the C₅₊ hydrocarbons, thereby generating an ETL effluent stream; (b) injecting the ETL effluent stream into a separations subsystem to generate a fuel gas stream and a stream containing the C₅₊ hydrocarbons.

In some embodiments, the offgas stream is from a fluidized catalytic cracking (FCC) unit. In some embodiments, the offgas stream is from a delayed coker unit (DCU). In some embodiments, the offgas stream is from a propane dehydrogenation unit. In some embodiments, the offgas stream is from an oxidative dehydrogenation unit. In some embodiments, the offgas stream is a refinery offgas. In some embodiments, a concentration of the olefins in the offgas stream is at least about 5 mol %. In some embodiments, a concentration of the olefins in the offgas stream is at least about 10 mol %. In some embodiments, an olefin concentration in the fuel gas stream is less than about 1 mol %. In some embodiments, an olefin concentration in the fuel gas stream is less than about 0.1 mol %. In some embodiments, the method further comprises prior to (a), injecting at least a portion of the offgas stream into a pretreatment bed to remove sulfur-containing species from the offgas stream. In some embodiments, the method further comprises injecting at least a portion of the ETL effluent stream into a drying unit to remove water from the ETL effluent stream and to produce a dry ETL effluent stream. In some embodiments, the separations subsystem includes one or more distillation columns. In some embodiments, the separations subsystem includes a deethanizer column. In some embodiments, the deethanizer column operates under conditions that yield a gas stream comprising ethane and a liquid stream comprising the C₅₊ hydrocarbons.

Another aspect of the present disclosure provides a method for producing fuel gas and hydrocarbons having five or more carbon atoms (C₅₊ hydrocarbons), the method comprising: (a) injecting a stream containing methane into an oxidative coupling of methane (OCM) subsystem that converts methane into ethylene to produce an OCM effluent stream; (b) injecting the OCM effluent stream and an offgas stream containing hydrogen, methane, and olefins into an ethylene-to-liquids (ETL) subsystem that converts the olefins into the C₅₊ hydrocarbons to generate an ETL effluent stream; (c) injecting the ETL effluent stream into a separations subsystem that generates a fuel gas stream, an ethane stream, a propane stream, and a C₅₊ hydrocarbon stream; and (d) injecting at least a portion of the ethane stream and at least a portion of the propane stream into the OCM subsystem.

In some embodiments, the stream containing methane is natural gas. In some embodiments, the stream containing methane is offgas from a fluidized catalytic cracking (FCC) unit. In some embodiments, the stream containing methane is offgas from a delayed coker unit (DCU). In some embodiments, the stream containing methane is refinery offgas. In some embodiments, the offgas stream is offgas from a fluidized catalytic cracking (FCC) unit. In some embodiments, the offgas stream is offgas from a delayed coker unit (DCU). In some embodiments, the offgas stream is offgas from a propane dehydrogenation unit. In some embodiments, the offgas stream is refinery offgas.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 schematically illustrates differentially cooled tubular reactor systems;

FIG. 2 schematically illustrates a reactor system with two or more tubular reactors;

FIG. 3 schematically illustrates an example ethylene-to-liquids (ETL) reactor system for producing higher molecular weight hydrocarbons with reduced olefin content;

FIG. 4 schematically illustrates an example oxidative coupling of methane (OCM)-ETL system comprising OCM and ETL units for use in producing higher molecular weight hydrocarbons comprising aromatic chemicals;

FIGS. 5A and 5B schematically illustrate an example OCM-ETL system comprising OCM/ETL units and one or more additional processing units for use in producing higher molecular weight hydrocarbons;

FIG. 6 schematically illustrates a computer system that is programmed or otherwise configured to implement systems and methods of the present disclosure;

FIG. 7 shows an example method for preparing mesostructured catalysts;

FIGS. 8A-8C shows acidity of sample mesostructured catalysts measured by thermogravimetric analysis (TGA);

FIGS. 9A-9C illustrate X-ray diffraction (XRD) spectra of sample mesostructured catalysts;

FIGS. 10A-10C illustrate performance of sample mesostructured catalysts in an ETL reaction at a temperature of 400° C., pressure of 300 psig, and weight hourly space velocity (WHSV) of 1.03 hr⁻¹;

FIGS. 11A-11C illustrate performance of sample mesostructured catalysts in an ETL reaction at a temperature of 400° C., pressure of 300 psig, and WHSV of 1.10 hr⁻¹;

FIG. 12 shows a list of sample mesostructured catalysts subjected to steaming conditions prior to use;

FIGS. 13A-13C illustrate performance of sample steamed mesostructured catalysts in an ETL reaction at a temperature of 400° C., pressure of 300 psig, and WHSV of 1.07 hr⁻¹;

FIGS. 14A-14C illustrate performance of sample steamed mesostructured catalysts in an ETL reaction at a temperature of 400° C., pressure of 300 psig, and WHSV of 1.05 hr⁻¹;

FIG. 15 schematically illustrates an example system for producing hydrocarbon compounds including alkylate;

FIG. 16 schematically illustrates an example ethylene conversion system for producing hydrocarbon compounds including alkylate;

FIG. 17 schematically illustrates an example ethylene conversion system for producing hydrocarbon compounds including alkylate;

FIG. 18 schematically illustrates an example ethylene conversion system for producing hydrocarbon compounds including alkylate using isoparaffins generated in the ethylene conversion system;

FIG. 19 schematically illustrates an example system for producing aromatic hydrocarbon compounds;

FIG. 20 schematically illustrates an example system for producing higher hydrocarbon compounds;

FIG. 21 schematically illustrates an example system for producing hydrocarbons using a water recycle stream;

FIG. 22 schematically illustrates an example system for producing hydrocarbons using a water recycle stream and the gas from a fluidized catalytic cracker (FCC) unit;

FIG. 23 schematically illustrates an example system for producing oxygenates using a water recovery stream;

FIG. 24 shows a schematic of a catalytic distillation column;

FIG. 25 shows a schematic for conducting catalytic distillation under elevated pressures;

FIG. 26 shows a process scheme for C₅₊ etherification via catalytic distillation;

FIG. 27 shows a schematic for C₅₊ hydration via catalytic distillation;

FIG. 28 shows an ETL process based on the initial step of oligomerization and catalytic distillation;

FIG. 29 shows a process for catalytic distillation hydration and oligomerization with ETL;

FIG. 30 shows a schematic of dimerization/alkylation via catalytic distillation;

FIG. 31 shows a schematic for 2-bed dimerization followed by alkylation via catalytic distillation;

FIG. 32 shows a schematic that demonstrates a possible process scheme for a catalytic distillation and oligomerization;

FIG. 33 shows a single pass oligomerization process;

FIG. 34 shows an oligomerization process that is configured with a recycle loop and process gas dryer before the separations module;

FIG. 35 shows an oligomerization process that is configured with a recycle loop coupled to a vapor/liquid separator before the dryer module and separations module;

FIG. 36 shows an oligomerization process that is configured with a recycle loop coupled to a vapor/liquid separator and a guard bed module comprising a H₂ removal unit;

FIG. 37 shows an in-situ catalyst regeneration process that is configured with a recycle loop coupled to a vapor/liquid separator with a dryer unit before or after the compressor/blower;

FIG. 38 shows a process by which clean fuel gas and C₅₊ hydrocarbons can be generated from FCC or DCU offgas;

FIG. 39 shows a process in which ETL and OCM are used with refinery offgas as a feedstock;

FIG. 40 shows a schematic for alkylation and dimerization via catalytic distillation; and

FIG. 41 shows a schematic for ETL-based oligomerization followed by alkylation via catalytic distillation.

### DETAILED DESCRIPTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The term “OCM process,” as used herein, generally refers to a process that employs or substantially employs an oxidative coupling of methane (OCM) reaction. An OCM reaction can include the oxidation of methane to a higher hydrocarbon (e.g., higher molecular weight hydrocarbon or higher chain hydrocarbon) and water, and involves an exothermic reaction. In an OCM reaction, methane can be partially oxidized to one or more C₂₊ compounds, such as ethylene, propylene, butylenes, etc. In an example, an OCM reaction is 2CH₄+O₂→C₂H₄+2H₂O. An OCM reaction can yield C₂₊ compounds. An OCM reaction can be facilitated by a catalyst, such as a heterogeneous catalyst. Additional by-products of OCM reactions can include CO, CO₂, H₂, as well as hydrocarbons, such as, for example, ethane, propane, propene, butane, butene, and the like.

The term “non-OCM process,” as used herein, generally refers to a process that does not employ or substantially employ an oxidative coupling of methane reaction. Examples of processes that may be non-OCM processes include non-OCM hydrocarbon processes, such as, for example, non-OCM processes employed in hydrocarbon processing in oil refineries, a natural gas liquids separations processes, steam cracking of ethane, steam cracking or naphtha, Fischer-Tropsch processes, and the like.

The term “ethylene-to-liquids” (ETL), as used herein, generally refers to any device, system, method (or process) that can convert an olefin (e.g., ethylene) to higher molecular weight hydrocarbons, which can be in liquid form.

The term “non-ETL process,” as used herein, generally refers to a process that does not employ or substantially employ the conversion of an olefin to a higher molecular weight hydrocarbon through oligomerization. Examples of processes that may be non-ETL processes include processes employed in hydrocarbon processing in oil refineries, a natural gas liquids separations processes, steam cracking of ethane, steam cracking or naphtha, Fischer-Tropsch processes, and the like.

The terms “C₂₊” and “C₂₊ compound,” as used herein, generally refer to a compound comprising two or more carbon atoms, e.g., C₂, C₃ etc. C₂₊ compounds include, without limitation, alkanes, alkenes, alkynes and aromatics containing two or more carbon atoms. In some cases, C₂₊ compounds include aldehydes, ketones, esters and carboxylic acids. Examples of C₂₊ compounds include ethane, ethene, acetylene, propane, propene, butane, butene, etc.

The term “non-C₂₊ impurities,” as used herein, generally refers to material that does not include C₂₊ compounds. Examples of non-C₂₊ impurities, which may be found in certain OCM reaction product streams, include nitrogen (N₂), oxygen (O₂), water (H₂O), argon (Ar), hydrogen (H₂) carbon monoxide (CO), carbon dioxide (CO₂) and methane (CH₄).

The term “weight hourly space velocity” (WHSV), as used herein, generally refers to the mass flow rate of olefins in a feed divided by the mass of a catalyst, which can have units of inverse time (e.g., hr⁻¹).

The term “slate,” as used herein, generally refers to distribution, such as product distribution.

The term “oligomerization,” as used herein, generally refers to a reaction in which hydrocarbons are combined to form larger chain hydrocarbons.

The term “catalyst,” as used herein, generally refers to a substance that alters the rate of a chemical reaction. A catalyst may either increase the chemical reaction rate (i.e. a “positive catalyst”) or decrease the reaction rate (i.e. a “negative catalyst”). A catalyst can be a heterogeneous catalyst. Catalysts can participate in a reaction in a cyclic fashion such that the catalyst is cyclically regenerated. “Catalytic” generally means having the properties of a catalyst.

The term “salt,” as used herein, generally refers to a compound comprising negative and positive ions. Salts are generally comprised of cations and counter ions. Under appropriate conditions, e.g., the solution also comprises a template, the metal ion (Mⁿ⁺) and the anion (X^{m-}) bind to the template to induce nucleation and growth of a nanowire of MₘXₙ on the template. “Anion precursor” thus is a compound that comprises an anion and a cationic counter ion, which allows the anion (X^{m-}) to dissociate from the cationic counter ion in a solution. Specific examples of the metal salt and anion precursors are described in further detail herein.

The term “oxide,” as used herein, generally refers to a metal or semiconductor compound comprising oxygen. Examples of oxides include, but are not limited to, metal oxides (MₓO_{y}), metal oxyhalides (MₓO_{y}X_{z}), metal hydroxyhalides (M_{X}OH_{y}X_{z}), metal oxynitrates (MₓO_{y}(NO₃)_{z}), metal phosphates (Mₓ(PO₄)_{y}), metal oxycarbonates (MₓO_{y}(CO₃)_{z}), metal carbonates (Mₓ(CO₃)_{z}), metal sulfates (Mₓ(SO₄)_{z}), metal oxysulfates (MₓO_{y}(SO₄)_{z}), metal phosphates (Mₓ(PO₄)_{z}), metal acetates (Mₓ(CH₃CO₂)_{z}), metal oxalates (Mₓ(C₂O₄)_{z}), metal oxyhydroxides (MₓO_{y}(OH)_{z}), metal hydroxides (Mₓ(OH)_{z}), hydrated metal oxides (MₓO_{y})—(H₂O)_{z} and the like, wherein X is independently, at each occurrence, fluoro, chloro, bromo or iodo, and x, y and z are independently numbers from 1 to 100.

The term “mixed oxide” or “mixed metal oxide,” as used herein, generally refers to a compound comprising two or more metals and oxygen (i.e., M1ₓM2_{y}O_{z}, wherein M1 and M2 are the same or different metal elements, O is oxygen and x, y and z are numbers from 1 to 100). A mixed oxide may comprise metal elements in various oxidation states and may comprise more than one type of metal element. For example, a mixed oxide of manganese and magnesium comprises oxidized forms of magnesium and manganese. Each individual manganese and magnesium atom may or may not have the same oxidation state. Mixed oxides comprising 2, 3, 4, 5, 6 or more metal elements can be represented in an analogous manner. Mixed oxides also include oxy-hydroxides (e.g., M_{X}O_{y}OH_{z}, wherein M is a metal element, O is oxygen, x, y and z are numbers from 1 to 100 and OH is hydroxy). Mixed oxides may be represented herein as M1-M2, wherein M1 and M2 are each independently a metal element.

The term “dopant” or “doping agent,” as used herein, generally refers to a material (e.g., impurity) added to or incorporated within a catalyst to alter (e.g., optimize) catalytic performance (e.g. increase or decrease catalytic activity). As compared to the undoped catalyst, a doped catalyst may increase or decrease the selectivity, conversion, and/or yield of a reaction catalyzed by the catalyst.

The term “OCM catalyst,” as used herein, generally refers to a catalyst capable of catalyzing an OCM reaction.

“Group 1” elements include lithium (Li), sodium (Na), potassium (K), rubidium (Rb), cesium (Cs), and francium (Fr).

“Group 2” elements include beryllium (Be), magnesium (Mg), calcium (Ca), strontium (Sr), barium (Ba), and radium (Ra).

“Group 3” elements include scandium (Sc) and yttrium (Y).

“Group 4” elements include titanium (Ti), zirconium (Zr), hafnium (Hf), and rutherfordium (Rf).

“Group 5” elements include vanadium (V), niobium (Nb), tantalum (Ta), and dubnium (Db).

“Group 6” elements include chromium (Cr), molybdenum (Mo), tungsten (W), and seaborgium (Sg).

“Group 7” elements include manganese (Mn), technetium (Tc), rhenium (Re), and bohrium (Bh).

“Group 8” elements include iron (Fe), ruthenium (Ru), osmium (Os), and hassium (Hs).

“Group 9” elements include cobalt (Co), rhodium (Rh), iridium (Ir), and meitnerium (Mt).

“Group 10” elements include nickel (Ni), palladium (Pd), platinum (Pt) and darmistadium (Ds).

“Group 11” elements include copper (Cu), silver (Ag), gold (Au), and roentgenium (Rg).

“Group 12” elements include zinc (Zn), cadmium (Cd), mercury (Hg), and copernicium (Cn).

“Metal element” or “metal” is any element, except hydrogen, selected from Groups 1 through 12, lanthanides, actinides, aluminum (Al), gallium (Ga), indium (In), tin (Sn), thallium (T), lead (Pb), and bismuth (Bi). Metal elements include metal elements in their elemental form as well as metal elements in an oxidized or reduced state, for example, when a metal element is combined with other elements in the form of compounds comprising metal elements. For example, metal elements can be in the form of hydrates, salts, oxides, as well as various polymorphs thereof, and the like.

The term “non-metal element,” as used herein, generally refers to an element selected from carbon (C), nitrogen (N), oxygen (O), fluorine (F), phosphorus (P), sulfur (S), chlorine (Cl), selenium (Se), bromine (Br), iodine (I), and astatine (At).

The term “higher hydrocarbon,” or “higher molecular weight compounds,” as used herein, generally refers to a higher molecular weight and/or higher chain hydrocarbon. A higher hydrocarbon can have a higher molecular weight and/or carbon content that is higher or larger relative to starting material in a given process (e.g., OCM or ETL). A higher hydrocarbon can be a higher molecular weight and/or chain hydrocarbon product that is generated in an OCM or ETL process. For example, ethylene is a higher hydrocarbon product relative to methane in an OCM process. As another example, a C₃₊ hydrocarbon is a higher hydrocarbon relative to ethylene in an ETL process. As another example, a C₅₊ hydrocarbon is a higher hydrocarbon relative to ethylene in an ETL process. In some cases, a higher hydrocarbon is a higher molecular weight hydrocarbon.

The present disclosure is generally directed to processes and systems for use in the production of higher hydrocarbon compositions. These processes and systems may be characterized in that they derive the hydrocarbon compositions from ethylene that may be derived from methane, for example as is present in natural gas. The processes and systems may comprise an ethylene-to-liquids (ETL) process and system which converts ethylene to one or more higher hydrocarbons, which in turn, may be further converted to commercially valuable products including gasoline, diesel fuel, jet fuel and aromatics, in one or more additional processes and sub-systems. The one or more additional subsystems may be integrated with the ETL system or retrofitted into a system that comprises the ETL system.

In some cases, disclosed processes and systems are further characterized in that the process for conversion of methane to ethylene is integrated with one or more processes or systems for converting ethylene to one or more higher hydrocarbon products, which, in some embodiments, comprise liquid hydrocarbon compositions. By converting the methane present in natural gas to a liquid material, one can eliminate one of the key hurdles involved in exploitation of the world's vast natural gas reserves, namely transportation. In particular, exploitation of natural gas resources may require extensive and costly pipeline infrastructures for movement of gas from the wellhead to its ultimate destination. By converting that gas to a liquid material, more conventional transportation systems become available, such as truck, rail car, tanker ship, and the like.

In some embodiments, processes and systems provided herein include multiple (i.e., two or more) ethylene conversion process paths integrated into the overall processes or systems, in order to produce multiple different higher hydrocarbon compositions from the single original methane source. Further advantages are gained by providing the integration of these multiple conversion processes or systems in a switchable or selectable architecture whereby a portion or all of the ethylene containing product of the methane to ethylene conversion system is selectively directed to one or more different process paths, for example two, three, four, five or more different process paths to yield as many different products.

### Ethylene-to-Liquids (ETL) Systems

Ethylene-to-liquids (ETL) systems and methods of the present disclosure can be used to form various products, including hydrocarbon products. Products and product distributions can be tailored to a given application, such as products for use as fuel (e.g., jet fuel or automobile fuels such as diesel or gasoline).

The present disclosure provides reactors for the conversion of unsaturated hydrocarbons (e.g., olefins) to higher molecular weight hydrocarbons, which can be in liquid form. Such reactors can be ETL reactors, which can be used to convert ethylene and/or other olefins to higher molecular weight hydrocarbons.

An ETL system (or sub-system) can include one or more reactors. An ETL system can include at least 1, 2, 3, 4, 5, 6, 7, 8, 9, 10, 11, 12, 13, 14, 15, 16, 17, 18, 19, or 20 ETL reactors, which can be in a parallel, serial, or a combination of parallel and serial configurations.

An ETL reactor can be in the form of a tube, packed bed, moving bed or fluidized bed. An ETL reactor can include a single tube or multiple tubes, such as a tube in a shell. A multi-tubular reactor can be used for highly exothermic conversions, such as the conversion of ethylene to other hydrocarbons. Such a design can allow for an efficient management of thermal fluxes and the control of reactor and catalyst bed temperatures.

An ETL reactor can be an isothermal or adiabatic reactor. An ETL reactor can have one or more of the following: 1) multiple cooling zones and arrangements within the reactor shell in which the temperature within each cooling zone may be independently set and controlled; 2) multiple residence times of the reactants as they traverse the tubular reactor from the inlet of the individual tubes to the outlet; and 3) multiple pass design in which the reactants may make several passes within the reactor shell from the inlet of the reactor to the outlet. In some cases, the ETL reactor operates substantially adiabatically, that is, under conditions such that substantially no heat (e.g., less than or equal to 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1% or less of the heat needed for ETL reaction) is added to the reactor during the ETL reaction.

Multi-tubular reactors of the present disclosure can be used to convert ethylene to liquid hydrocarbons in a variety of ways. In some cases, the disclosed multi-tubular ETL reactors can result in smaller reactors and gas compressors compared to adiabatic ETL designs. The ETL hydrocarbon reaction is exothermic and thus reaction heat management may be important for reaction control and improved product selectivity. In adiabatic ETL reactor designs, there is an upper limit to the ethylene concentration that is flowed through reactor due to the amount of heat released and subsequent temperature rise inside the reactor. To control the heat of reaction, adiabatic reactors can use a large amount of diluent gas to mitigate the temperature rise in the reactor bed. In some cases, the heat of reaction can be managed using multiple reactors with cooling between reactors and limited conversion between reactors (i.e., at least about 20%, about 30%, about 40%, about 50%, about 60%, or about 70% conversion in one reactor), cooling of the product effluent, and converting the remaining feedstock in one or more subsequent reactors. The use of diluent gas can result in larger catalyst beds, reactors, and gas compressors. The multi-tubular reactors described herein can allow for significantly greater ethylene concentrations while controlling the reactor bed temperature, since heat can be removed at the reactor wall. As a consequence, for a targeted rate of production, smaller catalyst beds, reactors, and gas compressors may be used.

In addition to smaller ETL reactors, the disclosed multi-tubular ETL reactors can result in smaller downstream liquid-gas product separation equipment due to less diluent gas needed to cool the reactor.

Multi-tubular ETL reactors can result in more favorable process conditions toward higher carbon number hydrocarbon liquids compared to an adiabatic ETL design. Relative to adiabatic reactors where ethylene feed can be diluted to control reaction temperature, the multi-tubular designs can allow for more concentrated ethylene feed into the reactor while maintaining good reactor temperature control. Higher ethylene concentration in the reactor can facilitate the formation for higher hydrocarbon liquids such as jet and/or diesel fuel since reactant concentration is important process parameter to yield higher hydrocarbon oligomers. In some cases, olefinic liquids of specific carbon number range and types can also be recycled into the reactor bed to further generate higher carbon number liquids (e.g., jet/diesel).

Multi-tubular reactors can have multiple temperature zones and offer multiple residence times. This can allow a wide range of process flexibility to target a particular product slate. As an example, a reactor can have multiple temperature zones and/or residence times. One use of this design can be to make jet and/or diesel fuel from ethylene. Ethylene oligomerization can require a relatively high reaction temperature. The temperature required to react ethylene, to start the oligomerization process may not be compatible with jet or diesel products, due to the rapid cracking and/or disproportionation of these jet/diesel products at elevated temperatures. Multiple reactor temperature zones can allow for a separate and higher temperature zone to start ethylene oligomerization while having another lower temperature zone to facilitate further oligomerization into jet/diesel fuel while discouraging cracking and disproportionation side reactions.

The use of multiple temperature zones may require different residence times within a reactor bed. In the jet/diesel example, the residence time for the ethylene reaction can be different than the residence time for a lower temperature finishing step to form jet/diesel. To maximize jet/diesel liquid yield, the ethylene oligomerization reaction bed temperature may need to be higher but with a lower residence time than the step to make jet/diesel which can require a lower temperature but higher residence time. In adiabatic ETL reactors, multi-temperature processes may occur over multiple reactor beds with a different temperature associated with each reactor. The multi-temperature zone approach disclosed herein can obviate the need for multiple reactors, as in the adiabatic ETL case, since multiple temperature zones can be achieved within a single reactor and thus lower capital outlay for reactor deployment.

Catalyst aging can be an important design constraint in ETL reaction engineering. ETL catalysts can deactivate over time until the catalyst bed is no longer able to sustain high ethylene conversion. A slower catalyst deactivation rate may be desired since more ethylene can be converted per catalyst bed before the catalyst bed can need to be taken off-line and regenerated. The catalyst may deactivate due to “coke”, deposits of carbonaceous material, which results in decreasing catalyst performance upon coke build-up. The rate of “coke” build-up is attributable to many different parameters. In ETL adiabatic reactors, the formation of catalyst bed “hot-spots” can play an important role in causing catalyst coking. “Hot-spots” favor aromatic compound formation, which are precursors to coke formation. “Hot-spots” are a result of temperature non-uniformities within the catalyst bed due to inadequate heat transfer. The localized “hot-spots” increase the rate of catalyst coking/deactivation. The disclosed multi-tubular design can decrease localized “hot-spots” due to better heat transfer properties of the multi-tubular design relative to the adiabatic design. It is anticipated that the decrease in catalyst “hot-spots” can slow catalyst deactivation.

The product slate of the ETL slate is a result of many factors. An important factor is the catalyst bed temperature. For example, higher temperatures catalyst bed temperatures can skew the product slate, for some catalysts, to aromatic products. In large adiabatic reactors, controlling “hot spot” formation is challenging and inhomogeneities in the catalyst bed temperature profiles lead a wider distribution of products. The multi-tubular design can significantly reduce catalyst bed temperature inhomogeneities/“hot spots” due to better heat transfer characteristics relative to the adiabatic design. As a result, a narrower product distribution can be more readily achieved than with adiabatic reactor design. While the multi-tubular design provides excellent catalyst bed temperature uniformity, catalyst bed temperature bed uniformity can be further enhanced by injection of “trim gas” and/or “trim liquid.”

The heat capacity of “trim gas” can be used to fine-tune the catalyst bed to a target temperature. Trim gas composition can be inert/high heat capacity gas for example: ethane, propane, butane, and other high heat capacity hydrocarbons.

The present disclosure also provides reactor systems for carrying out ethylene conversion processes. A number of ethylene conversion processes can involve exothermic catalytic reactions where substantial heat is generated by the process. Likewise, for a number of these catalytic systems, the regeneration processes for the catalyst materials likewise involve exothermic reactions. As such, reactor systems for use in these processes can generally be configured to effectively manage excess thermal energy produced by the reactions, in order to control the reactor bed temperatures to most efficiently control the reaction, prevent deleterious reactions, and prevent catalyst or reactor damage or destruction.

Tubular reactor configurations that may present high wall surface area per unit volume of catalyst bed may be used for reactions where thermal control is desirable or otherwise required, as they can permit greater thermal transfer out of the reactor. Reactor systems that include multiple parallel tubular reactors may be used in carrying out the ethylene conversion processes described herein. In particular, arrays of parallel tubular reactors each containing the appropriate catalyst for one or more ethylene conversion reaction processes may be arrayed with space between them to allow for the presence of a cooling medium between them. Such cooling medium may include any cooling medium appropriate for the given process. For example, the cooling medium may be air, water or other aqueous coolant formulations, steam, oil, upstream of reaction feed or for very high temperature reactor systems, molten salt coolants.

In some cases, reactor systems are provided that include multiple tubular reactors segmented into one, two, three, four or more different discrete cooling zones, where each zone is segregated to contain its own, separately controlled cooling medium. The temperature of each different cooling zone may be independently regulated through its respective cooling medium and an associated temperature control system, e.g., thermally connected heat exchangers, etc. Such differential control of temperature in different reactors can be used to differentially control different catalytic reactions, or reactions that have catalysts of different age. Likewise, it allows for the real time control of reaction progress in each reactor, in order to maintain a more uniform temperature profile across all reactors, and therefore synchronize catalyst lifetimes, regeneration cycles and replacement cycles.

Differentially cooled tubular reactor systems are schematically illustrated in FIG. 1. As shown, an overall reactor system 100 includes multiple discrete tubular reactors 102, 104, 106 and 108 contained within a larger reactor housing 110. Within each tubular reactor disposed is a catalyst bed for carrying out a given catalytic reaction. The catalyst bed in each tubular reactor may be the same or it may be different from the catalyst in the other tubular reactors, e.g., optimized for catalyzing a different reaction, or for catalyzing the same reaction under different conditions. As shown, the multiple tubular reactors 102, 104, 106 and 108 share a common manifold 112 for the delivery of reactants to the reactors. However, each individual tubular reactor or subset of the tubular reactors may alternatively include a single reactant delivery conduit or manifold for delivering reactants to that tubular reactor or subset of reactors, while a separate delivery conduit or manifold is provided for delivery of the same or different reactants to the other tubular reactors or subsets of tubular reactors.

As an alternative or in addition to, the reactor systems used in conjunction with the olefin (e.g., ethylene) conversion processes described herein can provide for variability in residence time for reactants within the catalytic portion of the reactor. Residence time within a reactor can be varied through the variation of any of a number of different applied parameters, e.g., increasing or decreasing flow rates, pressures, catalyst bed lengths, etc. However, a single reactor system may be provided with variable residence times, despite sharing a single reactor inlet, by varying the volume of different reactor tubes or reactor tube portions within a single reactor unit (“catalyst bed length”). As a result of varied volumes among reactor tubes or reactor tube portions into which reactants are being introduced at a given flow rate, residence times for those reactants within those varied volume reactor tubes or reactor tube portions, can be consequently varied.

Variation of reactor volumes may be accomplished through a number of approaches. By way of example, varied volume may be provided by including two or more different reactor tubes into which reactants are introduced at a given flow rate, where the two or more reactor tubes each have different volumes, e.g., by providing varied diameters. As will be appreciated, the residence time of gases being introduced at the same flow rate into two or more different reactors having different volumes can be different. In particular, the residence time can be greater in the higher volume reactors and shorter in the smaller volume reactors. The higher volume within two different reactors may be provided by providing each reactor with different diameters. Likewise, one can vary the length of the reactors catalyst bed, in order to vary the volume of the catalytic portion.

Alternatively or additionally, the volume of an individual reactor tube can be varied by varying the diameter of the reactor along its length, effectively altering the volume of different segments of the reactor. Again, in the wider reactor segments, the residence time of gas being introduced into the reactor tube can be longer in the wider reactor segments than in the narrower reactor segments.

Varied volumes can also be provided by routing different inlet reactant streams to different numbers of similarly sized reactor conduits or tubes. In particular, reactants, e.g., gases, may be introduced into a single reactor tube at a given flow rate to yield a particular residence time within the reactor. In contrast, reactants introduced at the same flow rate into two or more parallel reactor tubes can have a much longer residence time within those reactors.

FIG. 2 schematically illustrates a reactor system 200 in which two or more tubular reactors 202 and 204 are disposed, each having its own catalyst bed, 206 and 208, respectively, disposed therein. The two reactors are connected to the same inlet manifold such that the flow rate of reactants being introduced into each of reactors 202 and 204 are the same. Due to a larger volume that reactor 204 has (shown as a wider diameter), the reactants can be retained within catalyst bed 208 for a longer period. In particular, as shown in the figure, reactor 204 has a larger diameter, resulting in a slower linear velocity of reactants through the catalyst bed 208, than the reactants passing through catalyst bed 206. As noted above, one can similarly increase residence time within the catalyst bed of reactor 204 by providing a longer reactor. However, such longer reactor bed may be required to have similar back pressure as a shorter reactor to ensure reactants are introduced at the same flow rate as the shorter reactor.

The residence time of reactants within reactor systems can be controlled by varying the diameter of the ETL reactor along the path of fluid flow. In some cases, the reactor system can include multiple different reactor tubes, where each reactor tube includes a catalyst bed disposed therein. Differing residence times may be employed in catalyzing different catalytic reactions, or catalyzing the same reactions under differing conditions. In particular, it may be desirable to vary residence time of a given set of reactants over a single catalyst system, in order to catalyze a reaction more completely, catalyze a different or further reaction, or the like. Likewise, different reactors within the system may be provided with different catalyst systems that may benefit from differing residence times of the reactants within the catalyst bed to catalyze the same or different reactions from each other.

Alternatively or additionally, residence time of reactants within catalyst beds may be configured to optimize thermal control within the overall reactor system. In particular, residence time may be longer at a zone in the reactor system in which removal of excess thermal energy is less critical or more easily managed, e.g., because the overall reaction has not yet begun generating excessive heat. In contrast, in other zones of the reactor, e.g., where removal of excess thermal energy is more difficult due to rapid exothermic reactivity, the reactor portion may only maintain the reactants for a much shorter time, by providing a narrower reactor diameter. As can be appreciated, thermal management becomes easier due to the shorter period of time that the reactants are present and reacting to produce heat. Likewise, the reduced volume of a tubular reactor within a reactor housing also provides for a greater volume of cooling media, to more efficiently remove thermal energy.

Systems and methods of the present disclosure can employ fixed bed reactors. Fixed bed reactors can be adiabatic reactors. Fixed bed adiabatic ETL reactors can provide for simplicity of the reactor design. No active external cooling mechanism of the reactor may be necessary. To control the reactor temperature, profile dilution of the reactive olefin or other feedstocks (e.g., ethylene, propylene, butenes, pentenes, etc.) may be necessary. The diluent gas can be any material that is non-reactive or non-poisonous to the ETL catalyst, but may have a high heat capacity to moderate the temperature rise within the catalyst bed. Examples of diluent gases include nitrogen (N₂), argon, methane, ethane, propane and helium. The reactive part of the feedstock can be diluted directly or diluted indirectly in the reactor by recycling process gas to dilute the feedstock to an acceptable concentration. Temperature profile can also be controlled by internal reactor heat exchangers that can actively control the heat within the catalyst bed. Catalyst bed temperature control can also be achieved by limiting feedstock conversion within the catalyst bed. To achieve full feedstock conversion in this scenario, fixed bed adiabatic reactors are placed in series with heat exchangers between reactors to moderate temperature rise reactor over reactor. Partial conversion occurs in each reactor with inter-stage cooling to achieve the desired conversion and selectivity for the ETL process.

Since ETL catalysts can deactivate over time through coke deposition, the fixed bed reactors can be taken off-line and regenerated, such as by an oxidative or non-oxidative process. Once regenerated to full activity the ETL reactors can be put back on-line to process more feedstock.

Systems and methods of the present disclosure can employ the use of ETL continuous catalyst regeneration reactors. Continuous catalyst regeneration reactors (CCRR) can be attractive for processes where the catalyst deactivates over time and need to be taken off-line to be regenerated. By regenerating the catalyst in a continuous fashion less catalyst, fewer reactors for the process as well as fewer required operations are to regenerate the catalyst. There are two classes of deployments for CCRR reactors: (1) moving bed reactors and (2) fluidized bed reactors. In moving bed CCRR design, the pelletized catalyst bed moves along the reactor length and is removed and regenerated in a separate vessel. Once the catalyst is regenerated the catalyst pellets are put back in the ETL conversion reactor to process more feedstock. The online/regeneration process can be continuous and can maintain a constant flow of active catalyst in the ETL reactor. In fluidized bed ETL reactors, ETL catalyst particles are “fluidized” by a combination of ETL process gas velocity and catalyst particle weight. During bed fluidization, the bed expands, swirls, and agitates during reactor operation. The advantages of an ETL fluidized bed reactor are excellent mixing of process gas within the reactor, uniform temperature within the reactor, and the ability to continuously regenerate the coked ETL catalyst.

### Catalysts for the Conversion of Olefins to Liquids

The present disclosure also provides catalysts and catalyst compositions for ethylene conversion processes, in accordance with the processes described herein. In some embodiments, the disclosure provides zeolite, modified zeolite catalysts and/or catalyst compositions for carrying out a number of desired ethylene conversion reaction processes. In some cases, provided are impregnated or ion exchanged zeolite catalysts useful in conversion of ethylene to higher hydrocarbons, such as gasoline or gasoline blendstocks, diesel and/or jet fuels, as well as a variety of different aromatic compounds. For example, where one is using ethylene conversion processes to convert OCM product gases to gasoline or gasoline feedstock products or aromatic mixtures, one may employ modified ZSM catalysts, such as ZSM-5 catalysts that may be modified with Ga, Zn, Al, or mixtures thereof. In some cases, Ga, Zn and/or Al modified ZSM-5 catalysts are employed for use in converting ethylene to gasoline or gasoline feedstocks. Modified catalyst base materials other than ZSM-5 may also be employed in conjunction with the present disclosure, including, e.g., Y, ferrierite, mordenite, and additional catalyst base materials described herein.

In some cases, ZSM catalysts, such as ZSM-5 are modified with Co, Fe, Ce, or mixtures of these and are used in ethylene conversion processes using dilute ethylene streams that include both carbon monoxide and hydrogen components (See, e.g., Choudhary, et al., Microporous and Mesoporous Materials 2001, 253-267, which is incorporated herein by reference). In particular, these catalysts can be capable of co-oligomerizing the ethylene and H₂ and CO components into higher hydrocarbons, and mixtures useful as gasoline, diesel or jet fuel or blendstocks of these. In such embodiments, a mixed stream that includes dilute or non-dilute ethylene concentrations along with CO/H₂ gases can be passed over the catalyst under conditions that cause the co-oligomerization of both sets of feed components. Use of ZSM catalysts for conversion of syngas to higher hydrocarbons can be described in, for example, Li, et al., Energy and Fuels 2008, 22:1897-1901, which is incorporated herein by reference in its entirety.

The present disclosure provides various catalysts for use in converting olefins to liquids. Such catalysts can include an active material on a solid support. The active material can be configured to catalyze an ETL process to convert olefins to higher molecular weight hydrocarbons.

ETL reactors of the present disclosure can include various types of ETL catalysts. In some cases, such catalysts are zeolite and/or amorphous catalysts. Examples of zeolite catalysts include, but not limited to, ZSM-5, Zeolite Y, erionite, Beta zeolite (or zeolite beta), MFI topology zeolite and Mordenite. Examples of amorphous catalysts include solid phosphoric acid and amorphous aluminum silicate. Such catalysts can be doped, such as using metallic and/or semiconductor dopants. Examples of dopants include, without limitation, Ni, Pd, Pt, Zn, B, Al, Ga, In, Be, Mg, Ca and Sr. Such dopants can be situated at the surfaces, in the pore structure of the catalyst and/or bulk regions of such catalysts.

Catalyst can be doped with materials that are selected to effect a given or predetermined product distribution. For example, a catalyst doped with Mg or Ca can provide selectivity towards olefins for use in gasoline. As another example, a catalyst doped with Zn or Ga (e.g., Zn-doped ZSM-5 or Ga-doped ZSM-5) can provide selectivity towards aromatics. As another example, a catalyst doped with Ni (e.g., Ni-doped zeolite Y) can provide selectivity towards diesel or jet fuel.

Catalysts can be situated on solid supports. Solid supports can be formed of insulating materials, such as TiOx or AlOx, wherein ‘x’ is a number greater than zero, or ceramic materials.

Catalyst of the present disclosure can have various cycle lifetimes (e.g., the average period of time between catalyst regeneration cycles). In some cases, ETL catalysts can have lifetimes of at least about 50 hours, 100 hours, 110 hours, 120 hours, 130 hours, 140 hours, 150 hours, 160 hours, 170 hours, 180 hours, 190 hours, 200 hours, 210 hours, 220 hours, 230 hours, 240 hours, 250 hours, 300 hours, 350 hours, or 400 hours. At such cycle lifetimes, olefin conversion efficiencies less than about 90%, 85%, 80%, 75%, 70%, 65%, or 60% may be observed.

Catalysts of the present disclosure can be regenerated through various regeneration procedures, as described elsewhere herein. Such procedures can increase the total lifetimes of catalysts (e.g., length of time before the catalyst is disposed of). An example of a catalyst regeneration process is provided in Lubo Zhou, “BP-UOP Cyclar Process,” Handbook of Petroleum Refining Processes, The McGraw-Hill Companies (2004), pages 2.29-2.38, which is entirely incorporated herein by reference.

In some embodiments, ETL catalysts can be comprised of base materials (first active components) and dopants (second active components). The dopants can be introduced to the base materials through appropriate methods and procedures, such as vapor or liquid phase deposition. Dopants can be selected from a variety of elements, including metallic, non-metallic or amphoteric in forms of elementary substance, ions or compounds. A few representative doping elements are Ga, Zn, Al, In, Ni, Mg, B and Ag. Such dopants can be provided by dopant sources. For example, silver can be provided by way of AgCl or sputtering. The selection of doping materials can depend on the target product nature, such as product distribution. For example, Ga is favorable for aromatics-rich liquid production while Mg is favorable for aromatics-poor liquid production.

Base materials can be selected from crystalline zeolite materials, such as ZSM-5, ZSM-11, ZSM-22, Y, beta, mordenite, L, ferrierite, MCM-41, SAPO-34, SAPO-11, TS-1, SBA 15 or amorphous porous materials, such as amorphous silicoaluminate (ASA) and solid phosphoric acid catalysts. The cations of these materials can be NH₄, H or others. The surface areas of these materials can be in a range of 1 m²/g to 10000 m²/g, 10 m²/g to 5000 m²/g, or 100 m²/g to 1000 m²/g. The base materials can be directly used for synthesis or undergo some chemical treatment, such as desilication (de-Si) or dealumination (de-Al) to further modify the functionalities of these materials.

The base materials can be directly used for synthesis or undergo chemical treatment, such as desilication (de-Si) or dealumination (de-Al), to get derivatives of the base materials. Such treatment can improve the catalyst lifetime performance by creating larger pore volumes, such as pores having diameters greater than or equal to about 1 nanometer (nm), 2 nm, 3 nm, 4, nm, 5 nm, 10 nm, 20 nm, 30 nm, 40 nm, 50 nm, or 100 nm. In some cases, mesopores having diameters between about 1 nm and 100 nm, or 2 nm and 50 nm are created. In some examples, silica or alumina, or a combination of silica and alumina, can be etched from the base material to make a larger pore structure in the base catalyst that can enhance diffusion of reactants and products into the catalyst material. Pore diameter(s) and volume, in addition to porosity, can be as determined by adsorption or desorption isotherms (e.g., Brunauer-Emmett-Teller (BET) isotherm), such as using the method of Barrett-Joyner-Halenda (BJH). See Barrett E. P. et al., “The determination of pore volume and area distributions in porous substances. I. Computations from nitrogen isotherms,” J. Am. Chem. Soc. 1951. V. 73. P. 373-380. Such method can be used to calculate material porosity and mesopore volumes, in some cases volumes that are 3-7 times larger than their original materials. In general, any changes in catalyst structure, composition and morphology can be measured by technologies of BET, SEM and TEM, etc.

There are various approaches for doping catalysts. In an example, the doping components can be added to the base materials and their derivatives through impregnation, in some cases using incipient wetness impregnation (IWI), ion exchange or framework substitution in a zeolite synthesis operation. In some cases, IWI can include i) mixing a salt solution of the doping component with base material, for which the amount of salt is calculated based on doping level, ii) drying the mixture in an oven, and iii) calcining the product at a certain temperature for a certain time, for example 550-650° C., 6-10 hours. Ion exchange catalyst synthesis can include i) mixing a salt solution, which can contain at least about 1.5, 2, 3, 4, 5, 6, 7, 8, 9, or 10 times excess amount of the doping component, with base material, ii) heating the mixture, such as, for example, at a temperature from about 50° C. to 100° C., 60° C. to 90° C., or 70° C. to 80° C. for a time period of at least about 10 minutes, 30 minutes, 1 hour, 2 hours, 3 hours, 4 hours, 5 hours, 6 hours, 7 hours, 8 hours, 9 hours, 10 hours, 11 hours, or 12 hours, to conduct a first ion exchange, iii) separating the first ion exchange mother solution, iv) adding a new salt solution and repeating ii) and iii) to conduct a second ion exchange, v) washing the wet solid with deionized water to remove or lower the concentration of soluble components, vi) drying the raw product, such as air drying or in an oven, and vii) calcining the raw product at a temperature from about 450° C. to 800° C., 500° C. to 750° C., or 550° C. to 650° C. for a time period from about 1 hour to 24 hours, 4 hours to 12 hours, or 6 hours to 10 hours.

In some situations, powder catalysts prepared according to methods of the present disclosure may need to be formed prior to prepared in predetermined forms (or form factors) prior to use. In some examples, the forms can be selected from cylinder extrudates, rings, trilobe, and pellets. The sizes of the forms can be determined by reactor size. For example, for a 1″-2″ internal diameter (ID) reactor, 1.7 mm to 3.0 mm extrudates or equivalent size for other forms can be used. Larger forms can be used for different commercial scales (such as 5 mm forms). The ETL reactor inner diameter (ID) can be any diameter, including ranging from 2 inches to 10 feet, from 1 foot to 6 feet, and from 3 feet to 4 feet. In commercial reactors, the diameters of the catalyst (e.g., extrudate) can be greater than about 3 mm, greater than about 4 mm, greater than about 5 mm, greater than about 7 mm, greater than about 10 mm, greater than about 15 mm, or greater than about 20 mm. Binding materials (binder) can be used for forming the catalysts and improving catalyst particle strength. Various solid materials that are inert towards olefins (e.g., ethylene), such as Boehmite, alumina, silicate, Bentonite, or kaolin, can be used as binders.

A wide range of catalyst:binder ratio can be used, such as, from about 95:5 to 30:70, or 90:10 to 50:50. In some cases, a ratio of 80:20 is used for bench scale and pilot reactor catalyst synthesis. For formed catalysts, the crush strengths can be in the range of about 1 N/mm to 60 N/mm, 5 N/mm to 30 N/mm, or 7 N/mm to 15 N/mm.

Catalysts prepared according to methods of the present disclosure can be tested for the production of various hydrocarbon products, such as gasoline and/or aromatics production. In some cases, such catalysts are tested for the production of both gasoline and aromatics.

In an example, a short-term test condition for gasoline production is 300° C., atmospheric pressure, WHSV=0.65 hr⁻¹, N₂ 50% and C₂H₄ 50%, two hour runs. In another example, a short-term test condition for aromatics production is 450° C., atmospheric pressure, WHSV=1.31 hr⁻¹, N₂ 50% and C₂H₄ 50%, two hour runs. In addition to conducting the two hour short-term test to obtain the initial catalytic activity data, for some selected catalysts, the long-term test (lifetime test) are also performed to obtain data of catalyst lifetime, catalyst capacity as well as average product composition over the lifetime runs.

In an example, the results on an initial catalytic activity test at gasoline production conditions is C₂H₄ conversion greater than about 99%, C₅₊ C mole selectivity greater than about 65% (e.g., 65%-70%), and C₅₊ C mole yield greater than about 65% (e.g., 65%-70%). Catalyst lifetime performance in one cycle run at gasoline conditions can be at least about 189 hours, cut at conversion down to 80%; catalyst capacity is about 182 g-C₂H₄ converted per g-catalyst with C mole yield of products (e.g., C₅₊, C₃₌, C₄₌) greater than about 70%. With recycling, C₃₌ and C₄₌ can be accounted as liquid products.

In another example, the results on an initial catalytic activity at aromatics production conditions is C₂H₄ conversion greater than about 99%, C₅₊ C mole selectivity greater than about 75% (e.g., 75-80%), C₅₊ C mole yield greater than about 75% (e.g., 75-80%) and aromatics in C₅₊ greater than about 90%. Catalyst lifetime performance in one cycle run at aromatics production conditions can be at least about 228 hours, cut at conversion down to 82%, catalyst capacity 143 g-C₂H₄ converted/g-catalyst with average C₅₊ yield around 72% and aromatics yield around 62%.

An ETL catalyst can be porous and have an average pore size that is selected to optimize catalyst performance, including selectivity, lifetime, and product output, for use in production of specific products. The average pore size of an ETL catalyst can be greater than or equal to about 1 Angstroms (Å), 2 Å, 3 Å, 4 Å, 5 Å, 6 Å, 7 Å, 8 Å, 9 Å, 10 Å, 12 Å, 14 Å, 16 Å, 18 Å, 20 Å or more. In some cases, the average pore size of an ETL catalyst is less than or equal to about 1 micrometer (μm), 800 nanometers (nm), 600 nm, 400 nm, 200 nm, 100 nm, 80 nm, 60 nm, 40 nm, 20 nm, 10 nm, 8 nm, 6 nm, 4 nm, 2 nm, 1 nm, 8 Å, 6 Å, 4 Å, 2 Å, 1 Å or less. In some cases, the average pore size of an ETL catalyst is between any of the two values described above, for example, from 0.01 nm to 500 nm, from 0.1 nm to 100 nm, from 1 nm to 10 nm, or from 4 Å to 7 Å. The average pore size, pore structures, pore size distribution and porosity of a given catalyst can be characterized by a variety of techniques, including, but not limited to, scanning electron microscope (SEM), transmission electron microscope (TEM), small-angle scattering of X-rays (SAXS), neutrons (SANS), gas adsorption (e.g., nitrogen adsorption), mercury porosimetry, and a combination thereof. An ETL catalyst can have a base material with a set of pores that have an average pore size (e.g., diameter) from about 4 Å to 100 nm, or 4 Å to 10 nm, or 4 Å to 10 Å.

The catalytic materials may also be employed in any number of forms. In this regard, the physical form of the catalytic materials may contribute to their performance in various catalytic reactions. In particular, the performance of a number of operating parameters for a catalytic reactor that impact its performance can be significantly impacted by the form in which the catalyst is disposed within the reactor. The catalyst may be provided in the form of discrete, individual particles, e.g., pellets, extrudates or other formed aggregate particles, or it may be provided in one or more monolithic forms, e.g., blocks, honeycombs, foils, lattices, etc. These operating parameters include, for example, thermal transfer, flow rate and pressure drop through a reactor bed, catalyst accessibility, catalyst lifetime, aggregate strength, performance, and manageability.

In some cases, it is also desirable that the catalyst forms used will have crush strengths that meet the operating parameters of the reactor systems. In particular, a catalyst particle crush strength should generally support both the pressure applied to that particle from the operating conditions, e.g., gas inlet pressure, as well as the weight of the catalyst bed. A catalyst particle may have a crush strength that is greater than or equal to about 1 N/mm², 5 N/mm², 10 N/mm², 20 N/mm², 30 N/mm², 40 N/mm², 50 N/mm², or 100 N/mm². As will be appreciated, crush strength may be increased through the use of catalyst forms that are more compact, e.g., having lower surface to volume ratios. However, adopting such forms may adversely impact performance. Accordingly, forms are chosen that provide the above described crush strengths within the desired activity ranges, pressure drops, etc. Crush strength is also impacted though use of binder and preparation methods (e.g., extrusion or pelleting).

For example, in some embodiments the catalytic materials are in the form of an extrudate or pellet. Extrudates may be prepared by passing a semi-solid composition comprising the catalytic materials through an appropriate orifice or using molding or other appropriate techniques. Pellets may be prepared by pressing a solid composition comprising the catalytic materials under pressure in the die of a tablet press. Other catalytic forms include catalysts supported or impregnated on a support material or structure. In general, any support material or structure may be used to support the active catalyst. The support material or structure may be inert or have catalytic activity in the reaction of interest. For example, catalysts may be supported or impregnated on a monolith support. In some particular embodiments, the active catalyst is actually supported on the walls of the reactor itself, which may serve to minimize oxygen concentration at the inner wall or to promote heat exchange by generating heat of reaction at the reactor wall exclusively (e.g., an annular reactor in this case and higher space velocities).

The stability of the catalytic materials is defined as the length of time a catalytic material will maintain its catalytic performance without a significant decrease in performance (e.g., a decrease >20%, >15%, >10%, >5%, or greater than 1% in hydrocarbon or soot combustion activity). In some cases, the catalytic materials have stability under conditions required for the hydrocarbon combustion reaction of longer than or equal to about 1 hour (hr), 5 hrs, 10 hrs, 20 hrs, 50 hrs, 80 hrs, 90 hrs, 100 hrs, 150 hrs, 200 hrs, 250 hrs, 300 hrs, 350 hrs, 400 hrs, 450 hrs, 500 hrs, 550 hrs, 600 hrs, 650 hrs, 700 hrs, 750 hrs, 800 hrs, 850 hrs, 900 hrs, 950 hrs, 1,000 hrs, 2,000 hrs, 3,000 hrs, 4,000 hrs, 5,000 hrs, 6,000 hrs, 7,000 hrs, 8,000 hrs, 9,000 hrs, 10,000 hrs, 11,000 hrs, 12,000 hrs, 13,000 hrs, 14,000 hrs, 15,000 hrs, 16,000 hrs, 17,000 hrs, 18,000 hrs, 19,000 hrs, 20,000 hrs, 1 year (yr), 2 yrs, 3 yrs, 4 yrs, 5 yrs or more.

### Mesostructured Catalyst

Also provided herein is a method for generating higher hydrocarbon compounds (e.g., hydrocarbon compounds with three or more carbon atoms (C₃₊ compounds)), the method comprising directing a hydrocarbon feed stream comprising unsaturated hydrocarbons (e.g., ethylene (C₂H₄)) into an ethylene conversion reactor. The ethylene conversion reactor can be configured to convert the unsaturated hydrocarbons in an ethylene conversion process to yield a product stream comprising one or more C₃₊ compounds. In some cases, the product stream may further comprise hydrocarbon compounds having greater than or equal to 4, 5, 6, 7, 8, 9, 10, 11, 12, 13, 14, 15, 16, 17, 19, 20, 22, 24, 26, 28, 30, 32, 34, 36, 38, 40 or more carbon atoms. The hydrocarbon compounds generated in ethylene conversion process may be saturated and/or unsaturated, linear or branched.

In some cases, the ethylene conversion reactor comprises at least one catalyst disposed therein. The catalyst may be mesostructured (e.g., mesoporous catalyst). The catalyst may be configured to facilitate the ethylene conversion process and to operate at a variety of reaction conditions, depending upon, for example, desired composition of or type(s) of hydrocarbon compounds included in the product stream. For example, in some cases, the catalyst is configured to operate at a pressure less than or equal to about 50 PSI to maximize production of aromatics in the product stream. Alternatively or additionally, the catalyst may be configured to operate in an ethylene conversion process at a temperature higher than or equal to about 150° C. and a pressure less than or equal to about 1,000 PSI to maximize diesel/jet production.

In some cases, the catalyst is configured to operate at a temperature that is greater than or equal to about 50° C., 60° C., 70° C., 80° C., 90° C., 100° C., 110° C., 120° C., 130° C., 140° C., 150° C., 160° C., 170° C., 180° C., 190° C., 200° C., 220° C., 240° C., 260° C., 280° C., 300° C., 350° C., 400° C., 450° C., 500° C., 550° C., 600° C., 800° C. or higher. In some cases, the catalyst is configured to operate at a temperature that is less than or equal to about 2,000° C., 1,800° C., 1,600° C., 1,400° C., 1,200° C., 1,000° C., 900° C., 850° C., 800° C., 750° C., 700° C., 650° C., 600° C., 500° C., 400° C., 300° C., 200° C., 180° C., 160° C., 140° C., 120° C., 100° C., 80° C., 60° C., or lower. In some cases, the catalyst is configured to operate at a temperature that is between any of the two values described above, for example, 125° C.

In some cases, the catalyst is configured to operate at a pressure that is greater than or equal to about 10 pounds per square inch (PSI) (absolute), 20 PSI, 40 PSI, 60 PSI, 80 PSI, 100 PSI, 110 PSI, 120 PSI, 130 PSI, 140 PSI, 150 PSI, 160 PSI, 180 PSI, 200 PSI, 250 PSI, 300 PSI, 350 PSI, 400 PSI, 450 PSI, 500 PSI, 600 PSI, 700 PSI, 800 PSI, 900 PSI, or higher. In some cases, the catalyst is configured to operate at a pressure that is less than or equal to about 2,000 PSI, 1,800 PSI, 1,600 PSI, 1,400 PSI, 1,200 PSI, 1,000 PSI, 950 PSI, 850 PSI, 750 PSI, 650 PSI, 550 PSI, 450 PSI, 350 PSI, 250 PSI, 150 PSI, 100 PSI, 85 PSI, 75 PSI, 65 PSI, 55 PSI, 45 PSI, 35 PSI, 25 PSI, or lower. In some cases, the catalyst is configured to operate at a pressure that is between any of the two values described above, for example, 14.7 PSI.

As discussed above, the at least one catalyst may be mesostructured. The mesostructured catalyst may be a mesoporous catalyst. The mesoporous catalyst may comprise mesoporous zeolites such as mesoporous ZSM-5. The mesoporous catalyst may comprise a plurality of mesopores which has an average pore size that is greater than or equal to about 0.1 nanometers (nm), 0.2 nm, 0.3 nm, 0.4 nm, 0.5 nm, 0.6 nm, 0.7 nm, 0.8 nm, 0.9 nm, 1 nm, 1.5 nm, 2 nm, 2.5 nm, 3 nm, 3.5 nm, 4 nm, 4.5 nm, 5 nm, 5.5 nm, 6 nm, 6.5 nm, 7 nm, 7.5 nm, 8 nm, 8.5 nm, 9 nm, 9.5 nm, 10 nm, 11 nm, 12 nm, 13 nm, 14 nm, 15 nm, 16 nm, 17 nm, 18 nm, 19 nm, 20 nm, 30 nm, 40 nm, 50 nm, 60 nm, 70 nm, 80 nm, 90 nm, 100 nm, 200 nm, 300 nm, 400 nm, 500 nm, or more. In some cases, the average pore size of the mesopores is less than or equal to about 1,000 nm, 900 nm, 800 nm, 700 nm, 600 nm, 500 nm, 400 nm, 300 nm, 200 nm, 100 nm, 85 nm, 75 nm, 65 nm, 55 nm, 45 nm, 35 nm, 25 nm, 15 nm, 10 nm, 8 nm, 6 nm, 4 nm, 2 nm, 1 nm or less. In some cases, the average pore size of the mesopores is between any of the two values described above, for example, from about 1 nm to 500 nm, from about 1 nm to 50 nm, or from about 1 nm to 10 nm.

The mesostructured catalyst may be configured to facilitate an ethylene conversion process to yield a hydrocarbon compound (e.g., C₃₊, C₄₊, C₅₊, C₆₊, C₇₊, C₅₊, C₉₊, C₁₀₊ compounds) at a selectivity that is greater than or equal to about 25%, 30%, 35%, 40%, 45%, 50%, 55%, 60%, 65%, 70%, 75%, 80%, 85%, 90%, 95%, 96%, 97%, 98%, 99% or more.

In some cases, the ethylene conversion reactor comprises a plurality of ethylene conversion reactors, each of which may operate at the same or a different reaction conditions. In some cases, the ethylene conversion reactor comprises at least one ETL reactor which is adapted to conduct an ETL process. Suitable ETL reactor of the present disclosure is described above and elsewhere herein.

In some cases, the product stream generated in the ethylene conversion reactor is directed to one or more other processing units for further reaction or conversion. The product stream may be selectively directed from the ethylene conversion reactor in whole or in part to any one of the processing units. For example, at any given time, all of the product stream generated in the ethylene conversion rector may be directed therefrom to a single processing unit. Alternatively, only a portion of the product stream yielded in the ethylene conversion process may be routed to a first processing unit, and some or all of the remaining product stream may be directed to one, two, three, four, five, or more processing units or system. As an example, a portion of the product stream can be directed from the ethylene conversion reactor to a hydration unit that converts such portion of the product stream in a hydration process to generate an oxygenate product stream comprising oxygenates (e.g., C₅₊ oxygenates). Non-limiting examples of processing units include separation unit, cracking unit, hydration unit, methanation unit, metathesis unit, fluid catalytic cracking (FCC) unit, thermal cracker unit, coker unit, methanol to olefins (MTO) unit, Fischer-Tropsch unit, oxidative coupling of methane (OCM) unit, and combinations thereof.

Another aspect of the disclosure provided a method for generating higher hydrocarbon compounds (e.g., hydrocarbon compounds with three or more carbon atoms (C₃₊ compounds)), comprising directing a feed stream into an ethylene conversion reactor that converts unsaturated hydrocarbons including ethylene (C₂H₄) in the feed stream in an ethylene conversion process to yield a product stream comprising one or more higher hydrocarbons. The feed stream may comprise ethylene (C₂H₄), hydrogen (H₂) and carbon dioxide (CO₂). Molar ratios between each two components in the feed stream may vary. For example, the feed stream may have a C₂H₄/H₂ molar ratio greater than or equal to about 0.01, 0.03, 0.05, 0.07, 0.09, 0.1, 0.2, 0.3, 0.4, 0.5, 0.6, 0.7, 0.8, 0.9, 1, 1.2, 1.4, 1.6, 1.8, 2, 2.5, 3, 4, 5, 6, 7, 8, 9, 10, or higher. In some cases, the feed stream may have a C₂H₄/H₂ molar ratio less than or equal to about 20, 18, 16, 14, 12, 10, 8, 6, 4, 2, 1, 0.8, 0.7, 0.6, 0.5, 0.4, 0.3, 0.2, 0.1 or lower. In some cases, the feed stream has a C₂H₄/H₂ molar ratio that is between any of the values described above, for example, from about 0.01 to 5, or from about 0.1 to 2.

Additionally or alternatively, the feed stream may have a C₂H₄/CO₂ molar ratio greater than or equal to about 0.1, 0.3, 0.5, 0.7, 0.9, 1, 2, 3, 4, 5, 6, 7, 8, 9, 10, 11, 12, 13, 14, 15, 16, 17, 18, 19, 20 or higher. In some cases, the feed stream may have a C₂H₄/CO₂ molar ratio less than or equal to about 50, 45, 40, 35, 30, 25, 20, 18, 16, 14, 12, 10, 9, 8, 7, 6, 5, 4, 3, 2, 1, 0.5 or lower. In some cases, the feed stream has a C₂H₄/CO₂ molar ratio that falls within a range between any of the two values described above, for example, from about 1 to 10, or from about 5 to 10. In some examples, the feed stream comprising C₂H₄, H₂ and CO₂ has a C₂H₄/H₂/CO₂ molar ratio of 12:20:2.

As described above and elsewhere herein, the ethylene conversion reactor may comprise at least one catalyst disposed therein and configured to facilitate the ethylene conversion process. The catalyst may be mesostructured. The mesostructured catalyst may comprise mesoporous catalyst which comprises a plurality of mesopores. Depending upon, e.g., reaction conditions (e.g., temperature, pressure, reaction time, WHSV), composition of feed stream, desired composition of product stream, one or more mesoporus catalysts each having a different average pore size may be utilized.

Also provided herein is a method for generating higher hydrocarbon compounds (e.g., hydrocarbon compounds with three or more carbon atoms (C₃₊ compounds)), comprising directing a hydrocarbon feed stream comprising unsaturated hydrocarbons (e.g., C₂H₄) into an ethylene conversion reactor that is configured to conduct an ethylene conversion process to yield a product stream comprising one or more higher hydrocarbon compounds. The ethylene conversion reactor may comprise one or more catalysts that facilitate the ethylene conversion process. The one or more catalysts may comprise crystalline catalytic materials, amorphous catalytic materials, or combinations thereof. In some cases, the catalysts comprise at least one crystalline catalytic material and at least one amorphous catalytic material. The at least one crystalline catalytic material and at least one amorphous catalytic material may be intermixed with each other prior to use.

The crystalline catalytic materials may have a crystalline content that is at least about 80%, 85%, 90%, 91%, 92%, 93%, 94%, 95%, 96%, 97%, 98%, 99%, or more, as measured by X-ray diffraction (XRD). The crystalline catalytic materials may comprise zeolites. Non-limiting examples of zeolites may include, zeolite A, faujasite (zeolites X and Y; “FAU”), mordenite (“MOR”), CHA, ZSM-5 (“MFI”), ZSM-11, ZSM-12, ZSM-22, beta zeolite, synthetic ferrierite (“ZSM-35”), synthetic mordenite, USY (e.g., USY CBV 500), NH₄Y (e.g., NH₄Y CBV 300), NaY (e.g., NaY CBV 100), a rare earth ion zeolite Y, Low Silica X zeolite (LSX), and combinations or mixtures thereof.

The amorphous catalytic materials, on the other hand, may comprise a mesostructured catalyst. The mesostructured catalyst may be a mesoporous catalyst. The mesoporous catalyst may comprise a plurality of mesopores having an average pore size that is greater than or equal to about 0.1 nanometers (nm), 0.2 nm, 0.3 nm, 0.4 nm, 0.5 nm, 0.6 nm, 0.7 nm, 0.8 nm, 0.9 nm, 1 nm, 1.5 nm, 2 nm, 2.5 nm, 3 nm, 3.5 nm, 4 nm, 4.5 nm, 5 nm, 5.5 nm, 6 nm, 6.5 nm, 7 nm, 7.5 nm, 8 nm, 8.5 nm, 9 nm, 9.5 nm, 10 nm, 11 nm, 12 nm, 13 nm, 14 nm, 15 nm, 16 nm, 17 nm, 18 nm, 19 nm, 20 nm, 30 nm, 40 nm, 50 nm, 60 nm, 70 nm, 80 nm, 90 nm, 100 nm, 200 nm, 300 nm, 400 nm, 500 nm, or more. In some cases, the average pore size of the mesopores is less than or equal to about 1,000 nm, 900 nm, 800 nm, 700 nm, 600 nm, 500 nm, 400 nm, 300 nm, 200 nm, 100 nm, 85 nm, 75 nm, 65 nm, 55 nm, 45 nm, 35 nm, 25 nm, 15 nm, 10 nm, 8 nm, 6 nm, 4 nm, 2 nm, 1 nm or less. In some cases, the average pore size of the mesopores is between any of the two values described above, for example, from about 1 nm to 500 nm, from about 1 nm to 50 nm, or from about 1 nm to 10 nm. In some cases, the amorphous catalytic materials comprise MCM-41 type materials (e.g., Aluminum-MCM-41 (Al-MCM-41) and Titanium-MCM-41 (Ti-MCM-41)), or composites thereof.

In some cases, the crystalline catalytic materials are modified prior to use. Modified catalytic materials may have a crystalline content that is at least about 1%, 5%, 10%, 15%, 20%, 25%, 30%, 35%, 40%, 45%, 50%, 55%, 60%, 65%, 70%, 80%, 90%, or 95% less than a crystalline content of unmodified materials. The modified catalytic materials may be mesostructured. The mesostructured catalytic materials may have a plurality of mesopores. The mesopores may have an average pore size that is greater than, less than or equal to an average pore size of mesopores in the amorphous catalytic materials. In some cases, the ethylene conversion reactor comprises a plurality of the crystalline catalytic materials and/or the amorphous catalytic materials, each of which may have the same or a different average pore size.

Methods for forming a catalytic material comprise at least one mesostructured zeolite are also provided herein. The methods may comprise contacting a zeolite with a pH controlled solution, thereby forming the mesostructured zeolite. The zeolite, prior to contacting with pH controlled solution, may have a framework silicon-to-aluminum ratio (SAR) (or a framework silica-to-alumina ratio) that is greater than or equal to about 10, 20, 30, 35, 40, 45, 50, 55, 60, 65, 70, 75, 80, 85, 90, 95, 100, 120, 140, 160, 180, 200, 250, 300, 350, 400, 450, 500, 550, 600, 650, 700, 750, 800, 900, 1,000, 1,100, 1,200, 1,300, 1,400, 1,500, 1,600, 1,700, 1,800, 1,900, 2,000, 2,500, 3,000 or more. In some cases, the SAR (or the framework silica-to-alumina ratio) is less than or equal to about 3,000, 2,500, 2,000, 1,500, 1,000, 900, 850, 800, 700, 600, 500, 400, 300, 200, 100, 90, 80, 70, 60, 50, 40, 30 or lower. In some cases, the SAR (or the framework silica-to-alumina ratio) is between any of the two values described above, for example, about 280, or 140. Non-limiting examples of zeolites may include, zeolite A, faujasite (zeolites X and Y; “FAU”), mordenite (“MOR”), CHA, ZSM-5 (“MFI”), ZSM-11, ZSM-12, ZSM-22, beta zeolite, synthetic ferrierite (“ZSM-35”), synthetic mordenite, USY (e.g., USY CBV 500), NH4Y (e.g., NH4Y CBV 300), NaY (e.g., NaY CBV 100), a rare earth ion zeolite Y, Low Silica X zeolite (LSX), and combinations or mixtures thereof.

The framework silica-to-alumina ratio may be two times the SAR values described herein. For example, for a SAR of 10, the silica-to-alumina ratio is 20.

The pH controlled solution may comprise a surfactant. The surfactant may comprise a cationic surfactant, an anionic surfactant, a neutral surfactant (or non-ionic surfactant), or combinations thereof. Non-limiting examples of surfactants may include, behentrimonium chloride, benzalkonium chloride, benzethonium chloride, bronidox, cetrimonium bromide, cetrimonium chloride, dimethyldioctadecylammonium bromide, dimethyldioctadecylammonium chloride, cetyltrimethylammonium bromide, cetyltrimethylammonium chloride, lauryl methyl gluceth-10 hydroxypropyl dimonium chloride, octenidine dihydrochloride, olaflur, n-oleyl-1,3-propanediamine, stearalkonium chloride, tetramethylammonium hydroxide, thonzonium bromide, 2-acrylamido-2-methylpropane sulfonic acid, ammonium lauryl sulfate, ammonium perfluorononanoate, docusate, magnesium laureth sulfate, perfluorobutanesulfonic acid, perfluorononanoic acid, perfluorooctanesulfonic acid, perfluorooctanoic acid, phospholipid, potassium lauryl sulfate, soap, soap substitute, sodium alkyl sulfate, sodium dodecyl sulfate, sodium dodecylbenzenesulfonate, sodium laurate, sodium laureth sulfate, sodium lauroyl sarcosinate, sodium myreth sulfate, sodium nonanoyloxybenzenesulfonate, sodium pareth sulfate, sodium stearate, sulfolipid, alkyl polyglycoside, cetomacrogol 1000, cetostearyl alcohol, cetyl alcohol, cocamide diethanolamine, cocamide monoethanolamine, decyl glucoside, decyl polyglucose, disodium cocoamphodiacetate, glycerol monostearate, IGEPAL CA-630, Isoceteth-20, lauryl glucoside, maltosides, monolaurin, mycosubtilin, narrow-range ethoxylate, nonidet p-40, nonoxynol-9, nonoxynols, np-40, octaethylene glycol monododecyl ether, N-Octyl beta-D-thioglucopyranoside, octyl glucoside, oleyl alcohol, peg-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, Tween 80, and combinations thereof.

Quantity of the surfactant may vary, according to, for example, the surfactant and the zeolite that are mixed. For example, in some cases, the weight of surfactant is about equal to the weight of zeolite added to the solution. Alternatively, the weight of surfactant can be at least about 1%, 2%, 3%, 4%, 5%, 6%, 7%, 8%, 9%, 10%, 15%, 20%, 25%, 30%, 35%, 40%, 45%, 50%, 55%, 60%, 70%, 80%, 90%, 100%, 150%, 200%, or more of the weight of zeolite added to the solution.

The pH controlled solution can be a basic solution with a pH value greater than or equal to about 7, 8, 9, 10, 11, 12, 13 or 14. A variety of bases can be employed to prepare the pH controlled solution. Depending upon the desired pH value of the solution, strength, type and concentration of the bases may vary. For example, in some cases, the solution comprises a base at a concentration greater than or equal to about 0.001 mol/L (M), 0.002 M, 0.004 M, 0.006 M, 0.008 M, 0.01 M, 0.02 M, 0.03M, 0.04M, 0.05M, 0.06M, 0.07M, 0.08M, 0.09M, 0.1M, 0.2M, 0.3M, 0.4M, 0.5M, 0.6 M, 0.7 M, 0.8 M, 0.9 M, 1M, 1.5 M, 2 M or higher. In some cases, the solution comprises a base at a concentration less than or equal to about 5 M, 4 M, 3 M, 2 M, 1 M, 0.95 M, 0.85 M, 0.75 M, 0.65M, 0.55M, 0.45M, 0.35M, 0.25M, 0.15M, 0.1M, 0.08M, 0.06M, 0.04M, 0.02M, 0.01M, or lower. In some cases, the solution comprises a base at a concentration between any of the two values described herein, for example, from about 0.1 M to 0.5 M.

In some cases, the bases may comprise hydroxides of the alkali metals or alkaline earth metals. Non-limiting examples of bases may include, lithium hydroxide (LiOH), sodium hydroxide (NaOH), potassium hydroxide (KOH), rubidium hydroxide (RbOH), cesium hydroxide (CsOH), magnesium hydroxide (Mg(OH)₂), calcium hydroxide (Ca(OH)₂), strontium hydroxide (Sr(OH)₂), barium hydroxide (Ba(OH)₂), or combinations thereof.

Alternatively, the pH controlled solution can be an acidic solution with a pH lower than equal to about 7, 6, 5, 4, 3, 2, 1, or 0. Non-limiting examples of acids that may be employed in the methods include, mineral acids such as hydrofluoric acid (HF), hydrochloric acid (HCl), hydrobromic acid (HBr), hydroiodic acid (HI), halogen oxoacids: hypochlorous acid (HClO), chlorous acid (HClO₂), chloric acid (HClO₃), perchloric acid (HClO₄), hypofluorous acid (HFO), sulfuric acid (H₂SO₄), fluorosulfuric acid (HSO₃F), nitric acid (HNO₃), phosphoric acid (H₃PO₄), fluoroantimonic acid (HSbF₆), fluoroboric acid (HBF₄), hexafluorophosphoric acid (HPF₆), chromic acid (H₂CrO₄), boric acid (H₃BO₃); sulfonic acids such as methanesulfonic acid (or mesylic acid, CH₃SO₃H), ethanesulfonic acid (or esylic acid, CH₃CH₂SO₃H), benzenesulfonic acid (or besylic acid, C₆H₅SO₃H), p-Toluenesulfonic acid (or tosylic acid, CH₃C₆H₄SO₃H), trifluoromethanesulfonic acid (or triflic acid, CF₃SO₃H), polystyrene sulfonic acid (sulfonated polystyrene, [CH₂CH(C₆H₄)SO₃H]ₙ); carboxylic acids such as Acetic acid (CH₃COOH), citric acid (C₆H₈O₇), formic acid (HCOOH), gluconic acid HOCH₂—(CHOH)₄—COOH, lactic acid (CH₃—CHOH—COOH), oxalic acid (HOOC—COOH), tartaric acid (HOOC—CHOH—CHOH—COOH), fluoroacetic acid, trifluoroacetic acid, chloroacetic acid, dichloroacetic acid, trichloroacetic acid, or combinations thereof.

Concentration of the acid(s) in the solution may vary. In some cases, the solution comprises an acid at a concentration greater than or equal to about 0.001 mol/L (M), 0.002 M, 0.004 M, 0.006 M, 0.008M, 0.01M, 0.02M, 0.03M, 0.04M, 0.05M, 0.06M, 0.07M, 0.08M, 0.09M, 0.1M, 0.2 M, 0.3 M, 0.4 M, 0.5 M, 0.6 M, 0.7 M, 0.8 M, 0.9 M, 1M, 1.5 M, 2 M or higher. In some cases, the solution comprises an acid at a concentration less than or equal to about 5 M, 4 M, 3 M, 2 M, 1 M, 0.95M, 0.85M, 0.75M, 0.65M, 0.55M, 0.45M, 0.35M, 0.25M, 0.15M, 0.1M, 0.08M, 0.06M, 0.04 M, 0.02 M, 0.01 M, or lower. In some cases, the solution comprises an acid at a concentration that is between any of the two values described herein, for example, from about 0.1 M to 0.5 M.

The zeolites and surfactants can be added to the solution simultaneously, sequentially, or alternatively. In cases where the zeolite and surfactants are added sequentially, (e.g., the zeolites/surfactants are added after all the surfactants/zeolites have been added and dissolved in the pH controlled solution), pH value of the solution may vary during the process. In addition, during and/or after the addition of zeolites (and/or surfactants) to the pH controlled solution, the pH controlled solution may be subject to heat and maintained at a temperature that is greater than or equal to about 30° C., 35° C., 40° C., 45° C., 50° C., 55° C., 60° C., 65° C., 70° C., 75° C., 80° C., 85° C., 90° C., 95° C., 100° C., or higher, for at least about 10 minutes (min), 20 min, 30 min, 40 min, 50 min, 1 hour (hr), 1.5 hrs, 2 hrs, 2.5 hrs, 3 hrs, 3.5 hrs, 4 hrs, 4.5 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 22 hrs, 24 hrs, 26 hrs, 28 hrs, 30 hrs, 35 hrs, 40 hrs, 45 hrs, 50 hrs, 55 hrs, 60 hrs, 65 hrs, 70 hrs, 75 hrs, 80 hrs, 90 hrs, 100 hrs, or more.

Alternatively or additionally, methods for forming a catalytic material comprise at least one mesostructured zeolite may comprise contacting a zeolite with a pH controlled solution comprising ions of one or more chemical elements, thereby forming the mesostructured zeolite. The mesostructured zeolite may be mesoporous zeolite which comprises a plurality of mesopores. Further, the mesostructured zeolite may have a modified framework which comprises the one or more chemical elements. In some cases, the one or more chemical elements do not comprise silicon and aluminum. In some cases, the modified framework comprises at least about 0.001%, 0.005%, 0.01%, 0.05%, 0.1%, 0.20%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25% (mol %), or more chemical elements other than silicon and aluminum.

In some cases, the ions comprise metal ions. The metal ions may comprise cations of an alkali, alkaline earth, transition or rare earth metal. In some cases, the ions comprise nonmetal ions. In some cases, the one or more chemical elements comprise sodium, copper, iron, manganese, silver, zinc, nickel, gallium, titanium, phosphorus, boron, or combinations thereof.

The catalytic material produced by the methods of the present disclosure may have a lifetime that is greater than a lifetime of a catalytic material without being treated using the method when subjected to reaction conditions in an ethylene conversion process as described above and elsewhere herein. In some cases, the catalytic material may have a lifetime that is at least about 1.1, 1.2, 1.3, 1.4, 1.5, 1.6, 1.7, 1.8, 1.9, 2, 2.2, 2.4, 2.6, 2.8, 3, 3.5, 4, 4.5, 5, 5.5, 6, 6.5, 7, 7.5, 8, 8.5, 9, 9.5, 10, 12, 14, 16, 18, 20, 25, 30, 35, 40 times greater than a lifetime of a catalytic material without being treated using the method. In some cases, catalyst lifetime in an ethylene conversion process is expressed as (g of C₂H₄ converted)/(g of catalyst at an ethylene conversion level of 75%).

In some cases, the resulting catalytic materials are further subject to one or more additional processing steps such as steaming, calcination, reduction, impregnation (e.g., incipient wetness impregnation (IWI) or combinations thereof prior to use.

Also provided herein are catalytic materials produced by the methods of the present disclosure. The catalytic materials may comprise a mesostructured catalyst such as mesoporous zeolites. The zeolites may have an initial framework silicon-to-aluminum ratio (SAR) (or a framework silica-to-alumina ratio) that is greater than or equal to about 10, 20, 30, 35, 40, 45, 50, 55, 60, 65, 70, 75, 80, 85, 90, 95, 100, 120, 140, 160, 180, 200, 250, 300, 350, 400, 450, 500, 550, 600, 650, 700, 750, 800, 900, 1,000, 1,100, 1,200, 1,300, 1,400, 1,500, 1,600, 1,700, 1,800, 1,900, 2,000, 2,500, 3,000 or more. In some cases, the initial SAR (or the framework silica-to-alumina ratio) of the zeolites is less than or equal to about 3,000, 2,500, 2,000, 1,500, 1,000, 900, 850, 800, 700, 600, 500, 400, 300, 200, 100, 90, 80, 70, 60, 50, 40, 30 or lower.

Upon treatment or modification by the methods as described above, the modified zeolites (i.e., the mesoporous zeolites) may have a framework silicon-to-aluminum ratio (SAR) (or a framework silica-to-alumina ratio) that is greater than, lower than, or equal to the initial framework silicon-to-aluminum ratio (SAR) (or a framework silica-to-alumina ratio). For example, the mesoporous zeolites may have a framework SAR (or a framework silica-to-alumina ratio) that is greater than or equal to about 10, 20, 30, 35, 40, 45, 50, 55, 60, 65, 70, 75, 80, 85, 90, 95, 100, 120, 140, 160, 180, 200, 250, 300, 350, 400, 450, 500, 550, 600, 650, 700, 750, 800, 900, 1,000, 1, 100, 1,200, 1,300, 1,400, 1,500, 1,600, 1,700, 1,800, 1,900, 2,000, 2,500, 3,000 or more. In some cases, the mesoporous zeolites have an SAR (or the framework silica-to-alumina ratio) less than or equal to about 3,000, 2,500, 2,000, 1,500, 1,000, 900, 850, 800, 700, 600, 500, 400, 300, 200, 100, 90, 80, 70, 60, 50, 40, 30 or less. In some cases, the mesoporous zeolites have an SAR (or the framework silica-to-alumina ratio) that is at least about 1%, 5%, 10%, 15%, 20%, 25%, 30%, 35%, 40%, 45%, 50%, 60%, 70%, 80%, 90%, or 95% higher or lower than the initial SAR (or the framework silica-to-alumina ratio).

In some cases, the mesoporous zeolites have a modified framework comprising silicon, aluminum and at least another chemical element, such as sodium, copper, iron, manganese, silver, zinc, nickel, gallium, titanium, phosphorus, boron, or combinations thereof.

The catalytic materials of the present disclosure can be used in a variety of fields. For example, the catalytic materials may be employed in processing operations including gas and liquid-phase adsorption, separation, catalysis, catalytic cracking, catalytic hydrocracking, catalytic isomerization, catalytic hydrogenation, hydrosulfurization, oligomerization, catalytic hydroformilation, catalytic alkylation, catalytic acylation, ion-exchange, water treatment, pollution remediation, ethylene conversion such as ETL, OCM or combinations thereof.

### Systems and Methods for Producing Hydrocarbons Including Alkylate

Also provided in the present disclosure are methods and systems for producing hydrocarbon compounds. The produced hydrocarbon compounds may comprise hydrocarbon compounds with greater than or equal to about 2, 3, 4, 5, 6, 7, 8, 9, 10, 11, 12, 13, 14, 15, 16, 17, 18, 19, 20 or more carbon atoms. In some cases, the produced hydrocarbon compounds may comprise alkylate. The systems and methods may first comprise directing a feed stream into an oligomerization unit. The feed stream may comprise unsaturated and/or saturated hydrocarbons. The unsaturated and/or saturated hydrocarbons may comprise greater than or equal to about 2, 3, 4, 5, 6, 7, 8, 9, 10 or more carbon atoms, such as ethylene (C₂H₄). The oligomerization unit may permit at least a portion of one or more unsaturated and/or saturated hydrocarbons contained in the feed stream to react in an oligomerization process to yield a product stream (or an effluent). The effluent may comprise higher hydrocarbon compounds. The higher hydrocarbon compounds may be saturated and/or unsaturated, linear and/or branched.

During or after the yield of the product stream (or the effluent) in the oligomerization unit, at least a portion of the effluent may be directed from the oligomerization unit to an alkylation unit(s). The alkylation unit(s) may be in fluidic and/or thermal communication with the oligomerization unit. The alkylation unit(s) may be upstream of and/or downstream of the oligomerization unit. A separate stream comprising hydrocarbon compounds may be directed into the alkylation unit(s) along with the effluent from the oligomerization unit. The stream may be external to the oligomerization unit. The stream may comprise saturated or unsaturated hydrocarbons and/or isomers thereof. In some cases, the stream comprises isoparaffins (e.g., isobutane). The stream may be directed into the alkylation unit(s) substantially simultaneously, sequentially or alternately with the effluent. The alkylation unit(s) may permit at least a portion of hydrocarbon compounds contained in the effluent from the oligomerization unit and hydrocarbon compounds in the stream to react in one or more alkylation reactions to yield a product stream. The product stream may comprise one or more hydrocarbon compounds, saturated and/or unsaturated, linear and/or branched. In some examples, the effluent from the oligomerization unit comprises unsaturated higher hydrocarbons and the stream comprises isoparaffins. The alkylation unit(s) may be configured to perform an alkylation reaction that converts the unsaturated higher hydrocarbons and isoparaffins into a product stream. As discussed above, the product stream may comprise hydrocarbons with greater than or equal to about 2, 3, 4, 5, 6, 7, 8, 9, 10, 11, 12, 13, 14, 15, 16, 17, 18, 19, 20 or more carbon atoms. In some cases, the product stream may comprise hydrocarbons with carbon atoms falling in a range between any of the two values described herein, for example, C₅-C₁₀ or C₈-C₁₂. The hydrocarbons generated in the alkylation unit(s) may comprise saturated or unsaturated compounds. In some cases, the hydrocarbons generated in the alkylation unit(s) comprise at least about 5%, 10%, 20%, 30%, 40%, 50%, 55%, 60%, 65%, 70%, 75%, 80%, 85%, 90%, 95%, 99% (wt % or mol %) or more saturated and/or unsaturated hydrocarbons.

A molar ratio of hydrocarbon compounds in the stream (e.g., isoparaffins) to the hydrocarbons compounds in the effluent that are directed into the alkylation unit(s) may vary. In some cases, the molar ratio of hydrocarbon compounds in the stream (e.g., isoparaffins) to the hydrocarbons compounds in the effluent is greater than or equal to about 0.01, 0.05, 0.1, 0.5, 1, 5, 10, 20, 30, 40, 50, 60, 70, 80, 90, 100, 200, 300, 400, 500, 600, 700, 800, 900, 1,000 or higher. In some cases, the molar ratio of hydrocarbon compounds in the stream (e.g., isoparaffins) to the hydrocarbons compounds in the effluent is less than or equal to 2,000, 1,000, 800, 600, 400, 200, 100, 75, 50, 25, 10, 5, 4, 3, 2, 1, 0.5, 0.1, 0.05, 0.01 or less. In some cases, the molar ratio of hydrocarbon compounds in the stream (e.g., isoparaffins) to the hydrocarbons compounds in the effluent is between any of the two values described herein, for example, about 125.

In some cases, the product stream of the alkylation unit(s) is an alkylate stream. The alkylate stream may comprise an alkylate product. The alkylate product may comprise hydrocarbon compounds with eight or more carbon atoms (C₈₊ compounds). The alkylate product may comprise saturated hydrocarbons and/or isomers thereof. The alkylate product may comprise at least about 50%, 60%, 70%, 75%, 80%, 85%, 90%, 95%, 99% (wt % or mol %) or more saturated hydrocarbons and/or isomers thereof. The alkylated product may have a research octane number (RON) greater than or equal to about 70, 80, 90, 91, 92, 93, 94, 95, 96, 97, 98 or more. The alkylate product may have a motor octane number (MON) greater than or equal to about 50, 60, 70, 80, 81, 82, 83, 84, 85, 86, 87, 88, 89, 90, 91, 92, 93, 94, 95 or more.

The oligomerization unit may be an ethylene conversion unit. The ethylene conversion unit may comprise an ethylene-to-liquids (ETL) unit. Suitable ETL units that can be employed in the systems and methods of the present disclosure have been discussed above and elsewhere herein. The ETL unit can comprise a plurality of ETL reactors, each of which may comprise one or more ETL catalysts that may facilitate an ETL process.

The oligomerization unit may comprise a dimerization unit(s). The oligomerization process may comprise a dimerization process. The dimerization unit may comprise one or more dimerization reactors, for example, greater than or equal to about 2, 3, 4, 5, 6, 7, 8, 9, 10, 11, 12, 13, 14, 15, 16, 17, 18, 19, 20 or more dimerization rectors. Individual reactors may be in fluidic and/or thermal communication with each other. In some cases, the individual reactors are parallel to each other (fluidically and/or structurally). In some cases, each individual reactor has its own feed. In some cases, one or more reactors have a common feed. In cases where more than one dimerization reactors are employed, each individual reactor may be operated at the same or different conditions. Within a single reactor, the dimerization process may be operated at constant or varying conditions, depending upon, for example, compositions of feed stream, desired composition of product stream etc.

In some cases, the dimerization process is operated at a temperature that is greater than or equal to about 20° C., 30° C., 35° C., 40° C., 45° C., 50° C., 55° C., 60° C., 65° C., 70° C., 75° C., 80° C., 85° C., 90° C., 95° C., 100° C., 110° C., 120° C., 130° C., 140° C., 150° C., 160° C., 170° C., 180° C., 190° C., 200° C., or more. In some cases, the dimerization process is operated at a temperature that is less than or equal to about 350° C., 300° C., 250° C., 200° C., 180° C., 160° C., 140° C., 120° C., 100° C., 90° C., 80° C., 70° C., 60° C., 50° C., 40° C., 30° C., or less. In some cases, the dimerization process is operated at a temperature that is between any of the two values described above, for example, about 45° C., or about 75° C.

In some cases, the dimerization process is operated at a pressure that is greater than or equal to about 100 pounds per square inch (PSI) (absolute), 150 PSI, 200 PSI, 220 PSI, 240 PSI, 260 PSI, 280 PSI, 300 PSI, 320 PSI, 340 PSI, 360 PSI, 380 PSI, 400 PSI, 450 PSI, 500 PSI, 550 PSI, 600 PSI, or more. In some cases, the dimerization process is operated at a pressure that is less than or equal to about 1,000 PSI, 800 PSI, 600 PSI, 500 PSI, 450 PSI, 400 PSI, 390 PSI, 370 PSI, 350 PSI, 330 PSI, 310 PSI, 290 PSI, 270 PSI, 250 PSI, 230 PSI, 210 PSI, 190 PSI, 170 PSI, 150 PSI, 130 PSI, 110 PSI, 80 PSI, 60 PSI, or less. In some cases, the dimerization process is operated at a pressure that is between any of the two values described above, for example, 415 PSI.

The dimerization unit may comprise one or more catalyst. The one or more catalyst may facilitate the dimerization process. The catalyst may comprise one or more different components. In some cases, the catalyst may comprise at least one metal. Non-limiting examples of the metals may include, nickel, palladium, chromium, vanadium, iron, cobalt, ruthenium, rhodium, copper, silver, rhenium, molybdenum, tungsten, manganese, and combinations thereof. Alternatively or additionally, the catalyst may comprise one or more materials including e.g., zeolites, alumina, silica, carbon, titania, zirconia, silica/alumina, mesoporous silicas, and combinations thereof. Such materials may be employed as a support for the at least metal in the catalyst. In some cases, the catalyst comprises at least about 0.001%, 0.005%, 0.01%, 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15% (wt % or mol %), or more metals. In some cases, the catalyst comprises less than or equal to about 25%, 20%, 18%, 16%, 14%, 12%, 10%, 9%, 8%, 7%, 6%, 5%, 4%, 3%, 2%, 11%, 0.5%, 0.1% (wt % or mol %), or less metals.

In some cases, the dimerization catalyst comprises one or more materials that are configured to facilitate regeneration of the catalyst. The one or more materials may comprise a hydrogenation catalytic material, such as a hydrogenation catalyst. The hydrogenation catalytic material may comprise a metal such as, nickel, platinum, palladium, or combinations thereof.

The alkylation unit may comprise one or more alkylation reactors. The one or more alkylation reactors may be in fluidic and/or thermal communication with each other. The one or more alkylation reactors may be connected in series and/or in parallel. Each individual may or may not have a separate feed. In some cases, at least a certain percentage (e.g., at least about 10%, 20%, 30%, 40%, 50%, 60%, 70%, 80%, 90%, or more) of the reactors shares a common feed.

The alkylation unit may comprise an alkylation catalyst. The alkylation catalyst may facilitate (e.g., accelerate or promote) the alkylation process. The alkylation catalyst may comprise one or more materials. Non-limiting examples of the materials that may be employed in the alkylation catalyst include, tungstated zirconia, chlorided alumina, titaniosilicates (e.g., VTM zeolite), aluminum chloride (AlCl₃), polyphosphoric acid (e.g., solid phosphoric acid, or SPA, catalysts, which may be made by reacting phosphoric acid with diatomaceous earth), zeolites, silicon-aluminum phosphates, sulfated zirconia, polytungstic acid, and supported liquid acids such as triflic acid on silica, sulfuric acid on silica, hydrogen fluoride on carbon, antimony fluoride on silica, aluminum chloride (AlCl₃) on alumina (Al₂O₃), and combinations thereof. In some cases, zeolites comprise zeolite Beta, LTL zeolites, mordenite, MFI zeolites, BEA zeolites, MCM zeolites, faujasites (e.g., zeolite X, zeolite Y), USY zeolites, EMT zeolites, LTA zeolites, ITW zeolites, ITQ zeolites, SFO zeolites and combinations thereof.

The alkylation unit may be operated under constant or varying conditions. In some cases, the alkylation unit is operated at a temperature that is greater than or equal to about 20° C., 30° C., 35° C., 40° C., 45° C., 50° C., 55° C., 60° C., 65° C., 70° C., 75° C., 80° C., 85° C., 90° C., 95° C., 100° C., 110° C., 120° C., 130° C., 140° C., 150° C., 160° C., 170° C., 180° C., 190° C., 200° C., 250° C., 300° C., or more. In some cases, the alkylation unit is operated at a temperature that is less than or equal to about 500° C., 400° C., 300° C., 250° C., 200° C., 180° C., 160° C., 140° C., 120° C., 100° C., 90° C., 80° C., 70° C., 60° C., 50° C., 40° C., 30° C., or less. In some cases, the alkylation unit is operated at a temperature that is between any of the two values described above, for example, about 45° C., or about 75° C.

In some cases, the alkylation unit is operated at a pressure that is greater than or equal to about 100 pounds per square inch (PSI) (absolute), 150 PSI, 200 PSI, 220 PSI, 240 PSI, 260 PSI, 280 PSI, 300 PSI, 320 PSI, 340 PSI, 360 PSI, 380 PSI, 400 PSI, 450 PSI, 500 PSI, 550 PSI, 600 PSI, or more. In some cases, the alkylation unit is operated at a pressure that is less than or equal to about 1,000 PSI, 800 PSI, 600 PSI, 500 PSI, 450 PSI, 400 PSI, 390 PSI, 370 PSI, 350 PSI, 330 PSI, 310 PSI, 290 PSI, 270 PSI, 250 PSI, 230 PSI, 210 PSI, 190 PSI, 170 PSI, 150 PSI, 130 PSI, 110 PSI, 80 PSI, 60 PSI, or less. In some cases, the alkylation unit is operated at a pressure that is between any of the two values described above, for example, 375 PSI.

In some cases, systems and methods of the present disclosure further comprise, prior to the oligomerization process, directing the feed stream into an isomerization unit. The isomerization unit may be in fluidic and/or thermal communication with the oligomerization unit. The isomerization unit may be upstream of and/or downstream of the oligomerization unit. The isomerization unit may permit at least a portion of hydrocarbon compounds (e.g., unsaturated C₂₊ compounds) in the feed stream to react in an isomerization process. The isomerization process may convert the hydrocarbon compounds to their isomers, thereby producing a product stream comprising a mixture of the hydrocarbon compounds and isomers thereof.

Alternatively or additionally, at least a portion of effluent which is generated in the oligomerization unit may be directed into an isomerization unit. The isomerization unit may be in fluidic and/or thermal communication with the oligomerization unit. The isomerization unit may be upstream of and/or downstream of the oligomerization unit. The isomerization unit may permit at least a portion of hydrocarbons contained in the effluent (e.g., unsaturated higher hydrocarbons) to react in an isomerization process. The isomerization process may convert the unsaturated higher hydrocarbons to their respective isomers, and thus yield a product stream comprising a mixture of the unsaturated higher hydrocarbons and isomers thereof.

The isomerization unit may comprise one or more isomerization reactors. The one or more isomerization reactors may be connected in series and/or in parallel. The isomerization unit may comprise at least one isomerization catalyst. The at least one isomerization catalyst may facilitate the isomerization process. The isomerization catalyst may comprise alkaline oxides.

FIG. 15 shows an example system and method for producing hydrocarbons. The produced hydrocarbons may comprise alkylate. As shown in the figure, a feed stream 1501 (e.g., one of or a mixture of any of C₂-C₅ olefins) may be introduced to a dimerization unit 1502 where production of higher olefins can be effected. The effluent from the dimerization unit 1502 may then be routed to an alkylation unit 1503, along with a steam of isoparaffins 1504 (e.g., isobutane) such that alkylation may be effected to produce a product stream comprising hydrocarbon compounds 1505 such as alkylate. Alternatively or additionally, an isomerization unit (e.g., an olefin isomerization unit) (not shown in the figure) may be used such that at least a portion of the feed stream can be isomerized to yield a stream comprising a mixture of olefin isomers (e.g., 1-butene and cis-2-butene, and trans-2-butene). The isomerization unit may be upstream or downstream of the dimerization unit and/or the alkylation unit.

In some cases, systems and methods for producing hydrocarbon compounds may comprise, firstly, directing a first feed stream and a second stream into an alkylation unit. The first stream may comprise unsaturated hydrocarbons, e.g., unsaturated hydrocarbons with two or more carbon atoms (unsaturated C₂₊ compounds). The second stream, on the other hand, may comprise saturated hydrocarbons such as isoparaffins. As discussed above and elsewhere herein, the alkylation unit may be configured to perform an alkylation process. In the alkylation process, at least a portion of unsaturated hydrocarbons in the first stream and at least a portion of the saturated hydrocarbons in the second stream react with each other to yield a product stream. The product stream may comprise higher hydrocarbon compounds (e.g., hydrocarbon compounds with eight or more carbon atoms, or C₈₊ compounds). The first stream and the second stream may be directed into the alkylation unit without passing through an oligomerization unit (e.g., a dimerization unit).

In some cases, at least a portion of the first stream (e.g., at least about 5%, 10%, 15%, 20%, 25%, 30%, 35%, 40%, 45%, 50%, 55%, 60%, 65%, 70%, 75%, 80%, 85%, 90%, 95% (wt % or mol %) or more) is a product stream (or an effluent) from an ethylene conversion unit. In some cases, the first stream is at least a portion of the product stream (or an effluent) (e.g., at least about 5%, 10%, 15%, 20%, 25%, 30%, 35%, 40%, 45%, 50%, 55%, 60%, 65%, 70%, 75%, 80%, 85%, 90%, 95% (wt % or mol %) or more) from an ethylene conversion unit. The ethylene conversion unit may comprise an ETL unit. The ETL unit may comprise an ETL catalyst that facilitates the ETL process. The ETL catalyst, as discussed above and elsewhere herein, may comprise at least one metal. Non-limiting examples of the metals may include nickel, palladium, chromium, vanadium, iron, cobalt, ruthenium, rhodium, copper, silver, rhenium, molybdenum, tungsten, manganese, gallium, platinum, or combinations thereof. In some cases, the ETL catalyst further comprises one or more of zeolites amorphous silica alumina, silica, alumina, mesoporous silica, mesoporous alumina, zirconia, titania, pillared clay, and combinations thereof. The zeolites may comprise ZSM-5, zeolite Beta, ZSM-11, functional variants or combinations thereof.

In some cases, the methods further comprise, directing a feed stream into the ethylene conversion unit. The ethylene conversion unit may permit at least a portion of the feed stream to react in an ethylene conversion process. The ethylene conversion process may yield a product stream comprising at least a portion of the unsaturated hydrocarbons (e.g., unsaturated C₂₊ compounds) contained in the first stream.

Alternatively or additionally, the methods may further comprise, directing an oxidizing agent and the ethylene conversion feed stream into the ethylene conversion unit. The oxidizing agent may comprise oxygen (O₂), air, water or combination thereof. The oxidizing agent may react with at least a portion of hydrogens (H₂) in the ethylene conversion feed stream. Such reaction may result in a reduction of hydrogenation of unsaturated compounds over ethylene conversion catalyst in the ethylene conversion unit. In some cases, the hydrogenation of unsaturated compounds is reduced by at least about 5%, 10%, 15%, 20%, 25%, 30%, 35%, 40%, 45%, 50%, 55%, 60%, 65%, 70%, 75%, 80%, 85%, or more, as compared to hydrogenation of unsaturated compounds in the absence of the oxidizing agent when operated under the same conditions.

A molar ratio of the oxidizing agent to the ethylene conversion feed stream may vary. In some cases, the molar ratio may be greater than or equal to about 0.001, 0.005, 0.01, 0.05, 0.1, 0.2, 0.4, 0.6, 0.8, 1, 2, 3, 4, 5, 6, 7, 8, 9, 10, 12, 14, 16, 18, 20 or more. In some cases, the molar ratio may be less than or equal to about 50, 40, 30, 20, 18, 16, 14, 12, 10, 8, 6, 4, 2, 1, 0.5, 0.1, 0.05, 0.01 or less. In some cases, the molar ratio is between any of the two values described herein, for example, from about 0.01 to about 10.

In some cases, the ethylene conversion feed stream may be directed into a Fischer-Tropsch (FT) unit prior to being routed to the ethylene conversion unit. The FT unit may be in fluidic and/or thermal communication with the ethylene conversion unit. The FT unit may be upstream or downstream of the ethylene conversion unit. The FT unit may permit at least a portion of carbon monoxide (CO) and H₂ contained in the ethylene conversion feed stream to react in a FT process.

The FT process may then yield an effluent which may comprise hydrocarbon compounds with one to four carbons atoms (C₁-C₄ compounds).

Additionally or alternatively, the ethylene conversion feed stream may be directed into a hydrotreating unit. The hydrotreating unit may be in fluidic and/or thermal communication with the ethylene conversion unit. The hydrotreating unit may be upstream of and/or downstream of the ethylene conversion unit. The hydrotreating unit may comprise a hydrotreating catalyst. The hydrotreating catalyst may comprise CoMo-based catalyst, NiMo-based catalyst, or combinations thereof. The hydrotreating catalyst may be configured to facilitate a hydrotreating process. The hydrotreating process may remove at least a portion of sulfur (S) from the ethylene conversion feed stream. In some cases, after hydrotreating process, at least about 10%, 20%, 30%, 40%, 50%, 60%, 70%, 80%, 90%, 95% (wt % or mol %), or more S is removed from the ethylene conversion feed stream. The ethylene conversion unit and the hydrotreating unit may be separate reactor zones in the same reaction unit. The ethylene conversion unit and the hydrotreating unit may be individual reactors or reaction units that are separate from each other.

In some cases, the systems and methods of the present disclosure may further comprise directing one or more additional feed streams into the alkylation unit. The one or more additional feed streams may comprise e.g., unsaturated hydrocarbon compounds. The unsaturated hydrocarbon compounds may comprise, e.g., at least about 2, 3, 4, 5, 6, 7, 8, 9, 10, 11, 12, 13, 14, 15, 16, 17, 18, 19, 20 or more carbon atoms. In some cases, the unsaturated hydrocarbon compounds comprise unsaturated hydrocarbon compounds having three or four carbon atoms (unsaturated C₃₌/C₄₌ compounds). In some cases, the unsaturated hydrocarbon compounds comprise unsaturated hydrocarbon compounds having five or six carbon atoms (unsaturated C₅₌/C₆₌ compounds). The one or more additional feed streams may be generated in one or more additional processing units. Non-limiting examples of the additional processing units may include fluid catalytic cracking (FCC) unit, methanol-to-olefins (MTO) unit, FT unit, delayed cokers, steam crackers, or combinations thereof.

In some cases, the product stream generated in the alkylation unit comprises an alkylate stream. The alkylate stream may comprise an alkylate product. The alkylate product may comprise hydrocarbon compounds with eight or more carbon atoms (C₈₊ compounds). The alkylate product may comprise saturated hydrocarbons and/or isomers thereof. The alkylate product may comprise at least about 50%, 60%, 70%, 75%, 80%, 85%, 90%, 95%, 99% (wt % or mol %) or more saturated hydrocarbons and/or isomers thereof. The alkylated product may have a research octane number (RON) greater than or equal to about 90, 91, 92, 93, 94, 95, 96, 97, 98 or more. The alkylate product may have a motor octane number (MON) greater than or equal to about 80, 81, 82, 83, 84, 85, 86, 87, 88, 89, 90, 91, 92, 93, 94, 95 or more.

The alkylation unit may comprise an alkylation catalyst. The alkylation catalyst may facilitate (e.g., accelerate or promote) the alkylation process. The alkylation catalyst may comprise one or more different materials. Non-limiting examples of the materials that may be employed in the alkylation catalyst include, tungstated zirconia, chlorided alumina, titaniosilicates (e.g., VTM zeolite), aluminum chloride (AlCl₃), polyphosphoric acid (e.g., solid phosphoric acid, or SPA, catalysts, which may be made by reacting phosphoric acid with diatomaceous earth), zeolites, silicon-aluminum phosphates, sulfated zirconia, polytungstic acid, and supported liquid acids such as triflic acid on silica, sulfuric acid on silica, hydrogen fluoride on carbon, antimony fluoride on silica, aluminum chloride (AlCl₃) on alumina (Al₂O₃), and combinations thereof. In some cases, zeolites comprise zeolite Beta, LTL zeolites, mordenite, MFI zeolites, BEA zeolites, MCM zeolites, faujasites (e.g., zeolite X, zeolite Y), USY zeolites, EMT zeolites, LTA zeolites, ITW zeolites, ITQ zeolites, SFO zeolites and combinations thereof.

FIG. 16 illustrates an example system and method for producing hydrocarbons which may comprise alkylate. As shown in the figure, the system may comprise an ethylene conversion unit 1604. The ethylene conversion unit may be configured to perform an ethylene conversion process (e.g., an ETL process). The ethylene conversion process may permit oligomerization of light olefins (e.g. ethylene, propylene, and/or butenes) into higher olefins, with minimal conversion to hydrocarbons other than olefins (e.g. paraffins, isoparaffins, naphthenes, and aromatics). The ethylene conversion unit may comprise one or more catalysts that facilitate the ethylene conversion process. In some cases, the catalysts are geared towards oligomerization at moderate process conditions (e.g., mild temperature, moderate pressure etc.). The product stream from the ethylene conversion unit may be routed to an alkylation unit 1603, along with a stream of isoparaffins 1617 (e.g., isobutane) such that alkylation can be effected to produce a product stream 1618. The product stream 1618 may comprise alkylate. In some cases, at least a portion of the product stream generated in the ethylene conversion unit is routed 1619 as raw materials for further use (e.g., C₅₊ olefins generated in the ethylene conversion unit are routed as a gasoline blendstock). In some cases, at least a portion of the product stream generated in the ethylene conversion unit is subject to one or more further processing stages (as described above and elsewhere herein) for producing one or more different product streams such as alcohols, aldehydes, saturates, ethers, aromatics, epoxidation, or combinations thereof.

In some cases, at least a portion of the feed stream directed into the alkylation unit (e.g., unsaturated hydrocarbons including C₃ and C₄ olefins) is from one or more additional processing units 1606 (e.g., refinery and/or petrochemical units such as fluid catalytic cracking (FCC), methanol-to-olefins (MTO), Fischer-Tropsch (FT), delayed cokers, steam crackers, or combinations thereof). In some cases, an oxidizing agent 1610, such as O₂, air, or water, is fed along with the ethylene conversion feed (which may contain H₂), such as to minimize/limit the extent of hydrogenation of unsaturated hydrocarbons in the ethylene conversion feed over the oligomerization catalysts and thus to reduce the yield of oligomers. The oxidizing agent 1610 may be directed from a separate processing unit 1601 upstream of the ethylene conversion unit. In some cases, the processing unit 1601 is an OCM unit. Carbon monoxide (CO) contained in ethylene conversion feeds may be converted in a FT reaction (not shown in the figure) with H₂ into C₁-C₄ paraffins, so as to minimize the adverse impact it can have over the metal-containing oligomerization catalyst (e.g., Ni) such as etching.

Alternatively or additionally, a hydrotreating catalyst layer (or separate reaction zone) (not shown in the figure) upstream of the ethylene conversion unit can be employed to remove sulfur from certain feeds to the ethylene conversion unit. The hydrotreating catalyst can be in the form of a hydrotreating catalyst layer, composed of a CoMo and/or NiMo based catalyst which may react sulfur and not saturate olefins in the feed over the used process conditions, or in the form of a separate and upstream hydrotreating unit, which can comprise a mercaptan oxidation (MEROX) type unit employing a liquid catalyst or a CoMo/NiMo based unit. In some cases, one or more additional processing units such as a separations unit 1605, a fractionation and product recovery unit 1602, are included in the system. The one or more additional processing units may be utilized to further separate the feed(s) or product stream(s) prior to directing them into the other units of the system, such as the ethylene conversion unit and/or the alkylation unit.

FIG. 17 illustrates an example system similar to the system shown in FIG. 16. The system may comprise an ethylene conversion unit 1704, an alkylation unit 1703, one or more of an OCM unit 1701, a refinery/petrochemical unit 1706, a separations unit (e.g., a debutanizer) 1705, and a fractionation and/or product recovery unit 1702. In some instances, the one or more OCM units 1701 can be precluded. The ethylene conversion unit may have effluent including C₄₊ compounds routed to the alkylation unit, where isoparaffins may react with olefins in an alkylation reaction to yield higher hydrocarbons 1716 (e.g., alkylates). Additional C₃-C₆ olefin-containing streams 1715 may be directed into the alkylation unit from one or more additional sources 1706 including FCC, MTO, FT, delayed coker, hydrotreated steam cracking pyrolysis gasoline, or combinations thereof. An oxidizing agent 1710, such as O₂, air, or water, may be directed into the ethylene conversion unit along with the ethylene conversion feed (which may contain H₂) to minimize/limit the extent of hydrogenation of unsaturated hydrocarbons in the ethylene conversion feed over the oligomerization catalysts thereby reducing yield of oligomers.

Another aspect of the present disclosure provides systems and methods for producing hydrocarbon compounds. The systems and methods may comprise directing a feed stream into an ethylene conversion unit. The feed stream may comprise, e.g., unsaturated hydrocarbons such as C₂H₄. The ethylene conversion unit may permit at least a portion of the unsaturated hydrocarbons in the feed stream to react in an ethylene conversion process. The ethylene conversion process may then yield an ethylene conversion product stream (or effluent). The effluent may comprise multiple components (e.g., different types of hydrocarbon compounds). For example, the effluent may comprise unsaturated higher hydrocarbon compounds with e.g., greater than or equal to about 3, 4, 5, 6, 7, 8, 9, 10, or more carbon atoms. In some cases, the effluent comprises saturated hydrocarbons (e.g., paraffins including isoparaffins) with e.g., greater than or equal to about 3, 4, 5, 6, 7, 8, 9, 10, or more carbon atoms.

Next, a least a portion of the effluent from the ethylene conversion unit may be directed into an alkylation unit. The alkylation unit may be in fluidic and/or in thermal communication with the ethylene conversion unit. The alkylation unit may be upstream of and/or downstream of the ethylene conversion unit. The alkylation unit may be configured to perform an alkylation process or reaction. The alkylation unit may permit at least a portion of the unsaturated higher hydrocarbon (e.g., unsaturated hydrocarbon compounds with three or more carbon atoms or unsaturated C₃₊ compounds) and the saturated hydrocarbons (e.g., isoparaffins) contained in the effluent to react in the alkylation process. The alkylation process may yield a product stream comprising higher hydrocarbon compounds (e.g., hydrocarbon compounds with eight or more carbon atoms or C₈₊ compounds). The alkylation process may be conducted in the absence of an additional stream which comprise unsaturated hydrocarbons such as isoparaffins and is external to the ethylene conversion unit and the alkylation unit. In such situations, substantially all (i.e., at least about 90%, 91%, 92%, 93%, 94%, 95%, 96%, 97%, 98%, 99 mol % or more) of the saturated hydrocarbons consumed in the alkylation process may be generated in and/or directed from the ethylene conversion unit.

The ethylene conversion unit may comprise an ETL unit. The ETL unit may comprise one or more ETL reactors. The ETL unit may comprise at least one ETL catalyst that facilitates an ETL process. The effluent from the ethylene conversion unit may be directed into the alkylation unit without passing through a dimerization unit. In some cases, at least about 50%, 55%, 60%, 65%, 70%, 75%, 80%, 85%, 90%, 91%, 92%, 93%, 94%, 95%, 96%, 97%, 98%, 99% (wt % or mol %) or more of the effluent is directed into the alkylation unit without passing through a dimerization unit.

In some cases, the systems and methods further comprise directing at least a portion of the effluent from the ethylene conversion unit into a separations unit, before sending it to the alkylation unit. The separations unit may separate at least a portion of unsaturated C₃₊ compounds and at least a portion of unreacted C₂H₄ from the at least a portion of the effluent. Subsequently, at least a portion of such separated unsaturated C₃₊ compounds may be directed from the separations unit into a fractionation unit. The fractionation unit may separate at least one impurities from the unsaturated C₃₊ compounds. The at least one impurities may comprise saturated hydrocarbon compounds, such as saturated hydrocarbon compounds with three or more carbon atoms. In addition, the fractionation unit may yield one or more product streams (or effluent). For example, the fractionation unit may produce a first stream and a second stream. The first stream may comprise at least a portion of the at least one impurities. The second stream may comprise at least a portion of unsaturated C₃₊ compounds with reduced concentration of the at least one impurities. In some cases, the second stream comprising unsaturated C₃₊ compounds may be directed from the fractionation unit into the alkylation unit.

In some cases, the systems and methods further comprise, directing at least a portion of the effluent from the separations unit into an additional separations unit(s). The additional separations unit may be in fluidic and/or thermal communication with the separations unit, the fractionation unit, the ethylene conversion unit and/or the alkylation unit. The additional separations unit may be upstream of and/or downstream of one or more of the separations unit, the fractionation unit, the ethylene conversion unit and the alkylation unit. The additional separations unit may be configured to separate one or more desired compounds from the effluent. In some cases, the additional separations unit separates isoparaffins from the effluent. The isoparaffins separated in the additional separations unit may then be directed therefrom to the alkylation unit for further reaction. The isoparaffins may comprise isobutane, isopentane, or combinations thereof. In some cases, the isoparaffins comprise at least about 70%, 75%, 80%, 85%, 90%, 91%, 92%, 93%, 94%, 95%, 96%, 97%, 98%, 99% (wt %, or mol %) or more isopentane. In some cases, the isoparaffins comprise less than or equal to about 20%, 18%, 16%, 14%, 12%, 10%, 9%, 85, 7%, 6%, 5%, 4%, 3%, 2%, 1% (wt %, or mol %) or less isobutane.

In some cases, the systems and methods of the present disclosure may further comprise directing one or more additional feed streams into the alkylation unit. The one or more additional feed streams may comprise e.g., unsaturated hydrocarbon compounds. The unsaturated hydrocarbon compounds may comprise, e.g., at least about 2, 3, 4, 5, 6, 7, 8, 9, 10, 11, 12, 13, 14, 15, 16, 17, 18, 19, 20 or more carbon atoms. In some cases, the unsaturated hydrocarbon compounds comprise unsaturated hydrocarbon compounds having three or four carbon atoms (unsaturated C₃₌/C₄₌ compounds). In some cases, the unsaturated hydrocarbon compounds comprise unsaturated hydrocarbon compounds having five or six carbon atoms (unsaturated C₅₌/C₆₌ compounds). The one or more additional feed streams may be generated in one or more additional processing units. Non-limiting examples of the additional processing units may include fluid catalytic cracking (FCC) unit, methanol-to-olefins (MTO) unit, FT unit, delayed cokers, steam crackers, or combinations thereof.

The alkylation unit may comprise an alkylation catalyst. The alkylation catalyst may facilitate (e.g., accelerate or promote) the alkylation process. The alkylation catalyst may comprise one or more different materials. Non-limiting examples of materials that may be employed in the alkylation catalyst include, tungstated zirconia, chlorided alumina, titaniosilicates (e.g., VTM zeolite), aluminum chloride (AlCl₃), polyphosphoric acid (e.g., solid phosphoric acid, or SPA, catalysts, which may be made by reacting phosphoric acid with diatomaceous earth), zeolites, silicon-aluminum phosphates, sulfated zirconia, polytungstic acid, and supported liquid acids such as triflic acid on silica, sulfuric acid on silica, hydrogen fluoride on carbon, antimony fluoride on silica, aluminum chloride (AlCl₃) on alumina (Al₂O₃), and combinations thereof. In some cases, zeolites comprise zeolite Beta, LTL zeolites, mordenite, MFI zeolites, BEA zeolites, MCM zeolites, faujasites (e.g., zeolite X, zeolite Y), USY zeolites, EMT zeolites, LTA zeolites, ITW zeolites, ITQ zeolites, SFO zeolites and combinations thereof.

FIG. 18 shows an example system and method for producing hydrocarbon compounds including alkylate using isoparaffins generated in one or more processing/reaction units contained in the system. The system may comprise an ethylene conversion unit 1804, an alkylation unit 1803, one or more of an OCM unit 1801, a first separations unit 1805 (e.g., a debutanizer), a second separations unit 1806 (a depentanizer), a fractionation and/or product recovery unit 1802, and a refinery/petrochemical unit 1807 (e.g., FCC). In some cases, the OCM unit 1801 is precluded.

As the figure shows, effluent (including C₃₊, C₄₊ compounds) from the ethylene conversion unit may firstly be routed to the first and second separations units (e.g., debutanizer and depentanizer columns), so that C₄₋ 1813, C₅ 1818, and C₆₊ 1819 streams may be separated and recovered. The C₆₊ stream 1819 may be sent to a gasoline pool 1821. The C₅ stream, which may include iC₅, may be directed to the alkylation unit. C₂, C₃, and C₄ olefins 1814 may be recovered via multiple fractionation and recovery units 1802 (including e.g., a selective adsorption unit to separate iC₄ from C₄s and a membrane unit to separate nC₄ from C₄₌), and routed along with C₃₌ (light catalytically cracked naphtha from FCC) and C₅₌ (from hydrotreated light pygas from a steam cracker, a delayed coker, an FT unit, and/or an MTO unit) streams 1817, to the alkylation unit to produce alkylate product 1820.

Another aspect of the present disclosure provides systems and methods for generating aromatic hydrocarbon compounds. The aromatic hydrocarbon compounds may comprise alkyl aromatic hydrocarbon compounds. The systems and methods may comprise directing a feed stream into an ethylene conversion unit. The feed stream may comprise unsaturated hydrocarbons such as C₂H₄. The ethylene conversion unit may permit at least a portion of the unsaturated hydrocarbons to react in an ethylene conversion process. The ethylene conversion process may yield an ethylene conversion product stream or effluent. The effluent may comprise higher hydrocarbon compounds such as higher hydrocarbon compounds with three or more carbon atoms (C₃₊ compounds). The ethylene conversion unit may comprise an ETL unit. The ETL unit may comprise one or more catalysts that facilitate an ETL process.

Next, at least a portion of the effluent may be directed from the ethylene conversion unit into a separations unit. The separations unit may be in fluidic and/or in thermal communication with the ethylene conversion unit. The separations unit may be upstream of or downstream of the ethylene conversion unit. The separations unit may separate the effluent into multiple streams (e.g., 2, 3, 4, 5, 6, 7, 8, 9, 10, or more streams). For example, the separations unit may separate the ethylene conversion effluent into a first stream and a second stream. The first stream may comprise light hydrocarbons, e.g., hydrocarbon compounds with four or less carbon atoms (C₄₋ compounds). The C₄₋ compounds may comprise unreacted C₂H₄. The second stream may comprise higher hydrocarbons, e.g., hydrocarbon compounds with five or more carbon atoms (C₅₊ compounds).

Following the separations process, at least a portion of one or more of the separated streams may be directed into an aromatic extraction unit. The aromatic extraction unit may extract, from the streams, one or more aromatic hydrocarbon compounds. For example, in the above example, at least a portion of the second stream may be directed into the aromatic extraction unit. The aromatic extraction unit may be configured to perform an aromatic extraction process. The aromatic process may yield an effluent comprising aromatic hydrocarbon compounds with five or more carbon atoms (C₅₊ aromatics).

Subsequently, at least a portion of one or more of the streams produced in the separations unit and at least a portion of extraction effluent may be directed from the separations unit and the aromatic extraction unit, respectively, into an alkylation unit. The streams may be directed into the alkylation unit without passing through a dimerization unit. As discussed above and elsewhere herein, the alkylation unit may be configured to perform an alkylation process. The alkylation process may produce a product stream comprising higher hydrocarbons such as aromatic hydrocarbons. In one example, at least a portion of the first stream produced in the separations unit which comprises the C₄₋ compounds and at least a portion of the extraction effluent comprising the C₅₊ aromatics may be directed from the separations unit and the aromatic extraction unit respectively, into the alkylation unit. The alkylation unit may permit at least a portion of the C₄₋ compounds and the C₅₊ aromatics to react in an alkylation process to yield a product stream. The product stream may comprise alkyl aromatic hydrocarbon compounds. The alkyl aromatic hydrocarbon compounds may comprise xylene, ethylbenzene, isopropylbenzene, or combinations thereof.

In some cases, the C₄₋ compounds comprise unsaturated hydrocarbon compounds with four or less carbon atoms (unsaturated C₄₋ compounds). In some cases, the C₄₋ compounds comprise at least about 50%. 60%, 705, 75%, 80%, 85%, 90%, 95% (wt % or mol %), or more unsaturated C₄₋ compounds. In some cases, the C₅₊ aromatics comprise benzene. In some cases, the C₅₊ aromatics comprise at least about 20%, 30%, 40%, 50%, 60%, 705, 75%, 80%, 85%, 90%, 95% (wt % or mol %), or more benzene.

In some cases, the systems and methods further comprise, directing at least a portion of the extraction effluent from the aromatic extraction unit into one or more additional separations units. The one or more additional separations units may separate e.g., the C₅₊ aromatics into multiple streams (e.g., 2, 3, 4, 5, 6, 7, 8, 9, 10 or more stream each comprising a different composition). In some examples, the one or more additional separations units may separate the C₅₊ aromatics into two streams, a first stream and a second stream. The first stream may comprise benzene. The second stream may comprise aromatic compounds with seven or more carbon atoms (C₇₊ aromatics). The first stream may subsequently be routed from the additional separations unit to the alkylation unit and subject to further reaction. The second stream, on the other hand, may be directed to a product tank without any further processing.

The alkylation unit may comprise an alkylation catalyst. The alkylation catalyst may facilitate (e.g., accelerate or promote) the alkylation process. The alkylation catalyst may comprise one or more different materials. Non-limiting examples of materials that may be employed in the alkylation catalyst include, tungstated zirconia, chlorided alumina, titaniosilicates (e.g., VTM zeolite), aluminum chloride (AlCl₃), polyphosphoric acid (e.g., solid phosphoric acid, or SPA, catalysts, which may be made by reacting phosphoric acid with diatomaceous earth), zeolites, silicon-aluminum phosphates, sulfated zirconia, polytungstic acid, and supported liquid acids such as triflic acid on silica, sulfuric acid on silica, hydrogen fluoride on carbon, antimony fluoride on silica, aluminum chloride (AlCl₃) on alumina (Al₂O₃), and combinations thereof. In some cases, zeolites comprise zeolite Beta, LTL zeolites, mordenite, MFI zeolites, BEA zeolites, MCM zeolites, faujasites (e.g., zeolite X, zeolite Y), USY zeolites, EMT zeolites, LTA zeolites, ITW zeolites, ITQ zeolites, SFO zeolites and combinations thereof.

FIG. 19 illustrates an example system and method for producing hydrocarbon compounds including aromatics. The aromatics may be branched or linear, saturated or unsaturated, substituted or unsubstituted. As shown in the figure, the system may comprise an ethylene conversion unit 1904, an alkylation unit 1903, one or more of an OCM unit 1901, a first separations unit 1905 (e.g., a debutanizer), an aromatic extraction unit 1906, a second separations unit 1907 (a dehexanizer), a fractionation and/or product recovery unit 1902, and a refinery/petrochemical unit 1908 (e.g., FCC). In some cases, the OCM unit 1901 is precluded. Effluent(s) (including C3+, C₄₊, C₅₊ compounds) from the ethylene conversion unit may firstly be routed to the first separations unit and the aromatics extraction unit prior to being sent to the alkylation unit. Raffinate stream 1918 from the aromatics extraction unit may be routed to a gasoline pool 1923. Extracted aromatics 1917 may be sent to the second separations unit (e.g., a benzene column). The second separations unit may separate out benzene 1919 and recover C₇₊ aromatics 1922 as a final product which can be used in the gasoline pool 1923 or further processed in aromatic complexes to produce benzene and/or xylene. Benzene 1919, along with C₃₌ and/or C₂=streams 1915 produced in the ethylene conversion unit, may be directed to the alkylation unit(s), where aromatic hydrocarbons (e.g., cumene and/or ethylbenzene) may be selectively produced. Additional C₃₌ compounds (e.g., propylene) 1920 may be sourced from other refineries/petrochemical units 1908 such as FCC, FT, delayed cokers, MTO, steam crackers, metathesis etc., and routed to the alkylation unit for further reaction.

Also provided herein are systems and methods for producing higher hydrocarbon compounds such as hydrocarbon compounds having greater than or equal to about 8, 9, 10, 11, 12, 13, 14, 15, 16, 17, 18, 19, 20 or more carbon atoms. The systems and methods may comprise directing a feed stream into an ethylene conversion unit. The feed stream may comprise one or more unsaturated hydrocarbons such as C₂H₄. The ethylene conversion unit may be configured to perform an ethylene conversion process. The ethylene conversion unit may permit at least a portion of the feed stream to react in the ethylene conversion process to yield an ethylene conversion product stream or effluent. The effluent may comprise higher hydrocarbon compounds, for example, hydrocarbon compounds with three or more carbon atoms (C₃₊ compounds).

Following the ethylene conversion process, at least a portion of the effluent may be directed from the ethylene conversion unit, along with a stream comprising saturated hydrocarbons (e.g., isoparaffins) into a first alkylation unit. The effluent and the stream comprising saturated hydrocarbons may be directed into the alkylation unit substantially simultaneously, sequentially or alternately. The first alkylation unit may permit at least a portion of higher hydrocarbon compounds (e.g., C₃₊ compounds) in the effluent and the saturated hydrocarbons (e.g., isoparaffins such as isobutane, isopentane or combinations thereof) in the stream to react in a first alkylation process. The first alkylation process may produce an alkylation product stream.

Next, at least a portion of the alkylation product stream may be directed from the first alkylation unit into a separations unit. The separations unit may be configured to perform a separations reaction or process. The separations reaction or process may yield a separations product stream. The separations product stream may comprise higher hydrocarbon compounds with six or more carbon atoms (C₆₊ compounds). The C₆₊ compounds may comprise saturated (saturated C₆₊ compounds) or unsaturated compounds (e.g., unsaturated C₆₊ compounds). The saturated compounds may comprise a mixture of compounds and isomers thereof. The C₆₊ compounds may comprise isoparaffins. The isoparaffins may have greater than 6, 7, 8, 9, 10, or more carbon atoms. In some cases, the isoparaffins comprise isoparaffins with eight or more carbon atoms (C₈₊ isoparaffins).

Subsequently, at least a portion of the separations product stream may be directed into a second alkylation unit. The second alkylation unit may permit at least a portion of the C₆₊ compounds to react in a second alkylation process. The second alkylation process may yield a product stream comprising higher hydrocarbon compounds. The higher hydrocarbon compounds comprised in the product stream may include hydrocarbon compounds with fourteen or more carbon atoms (C₁₄₊ compounds). In some examples, the C₆₊ compounds comprise C₈₊ isoparaffins and unsaturated C₆₊ compounds. The second alkylation unit may permit at least a portion of the C₈₊ isoparaffins and unsaturated C₆₊ compounds to react in the second alkylation process to yield a product stream comprising the C₁₄₊ compounds.

As provided herein, the first alkylation unit and the second alkylation unit may be operated under the same conditions, such as an alkylation reaction condition as discussed above or elsewhere herein. In some cases, the first alkylation unit and the second alkylation unit are operated under different conditions (e.g., different temperatures, pressures etc.). The first alkylation unit may comprise an alkylation catalyst. The second alkylation unit may comprise an alkylation catalyst. The alkylation catalysts in the first and second alkylation units may be the same or different. One or both of the alkylation catalysts in the first alkylation unit and second alkylation unit may be configured to facilitate the first and/or the second alkylation processes. In some cases, at least one of the catalysts employed in the first and/or second alkylation units comprise one or more different materials. Non-limiting examples of materials that may be employed in the alkylation catalyst include, tungstated zirconia, chlorided alumina, titaniosilicates (e.g., VTM zeolite), aluminum chloride (AlCl₃), polyphosphoric acid (e.g., solid phosphoric acid, or SPA, catalysts, which may be made by reacting phosphoric acid with diatomaceous earth), zeolites, silicon-aluminum phosphates, sulfated zirconia, polytungstic acid, and supported liquid acids such as triflic acid on silica, sulfuric acid on silica, hydrogen fluoride on carbon, antimony fluoride on silica, aluminum chloride (AlCl₃) on alumina (Al₂O₃), and combinations thereof. In some cases, zeolites comprise zeolite Beta, LTL zeolites, mordenite, MFI zeolites, BEA zeolites, MCM zeolites, faujasites (e.g., zeolite X, zeolite Y), USY zeolites, EMT zeolites, LTA zeolites, ITW zeolites, ITQ zeolites, SFO zeolites and combinations thereof.

FIG. 20 illustrates an example system and method of the present disclosure for producing hydrocarbon compounds including alkylate and/or diesel. The system, as shown in the figure, may comprise an ethylene conversion unit 2004, one or more alkylation units 2003 & 2006, one or more of an OCM unit 2001, a separations unit 2005 (e.g., a debutanizer), a fractionation and/or product recovery unit 2002, and a refinery/petrochemical unit 2007 (e.g., FCC). In some cases, the OCM unit 2001 is precluded.

The ethylene conversion unit may be configured to permit a feed stream comprising lighter hydrocarbons (e.g. ethylene, propylene, and/or butenes) to react in an ethylene conversion process to yield effluent comprising higher hydrocarbons (e.g., C₃₊/C₄₊/C₅₊ compounds). The ethylene conversion process may comprise one or more catalysts as described above or elsewhere herein. The ethylene conversion process may be configured to convert the lighter hydrocarbons into higher ones with minimal conversion to hydrocarbons other than olefins (e.g. paraffins, isoparaffins, naphthenes, and aromatics). The olefin effluent from the ethylene conversion unit may be routed through the separations unit 2015 and/or the fractionation/recovery unit 2013/2014 to the first alkylation unit 2003. A stream comprising isoparaffins 2016 (e.g., isobutane) may be directed into the first alkylation unit 2003 simultaneously or sequentially with the olefin effluent 2013/2014 such that alkylation reaction may be effected to produce a product stream 2019 comprising e.g., alkylate stream. At least a portion of the product stream 2019 may be routed to the separations unit 2005 to recover iC₈ along with unsaturated higher hydrocarbons (e.g., C₆₊ olefins) produced in the ethylene conversion process 2020. At least a portion of the recovered compounds (i.e., iC₈ and C₆₊ olefins) may subsequently be directed to a second alkylation unit 2006. The second alkylation unit may be configured to permit the at least a portion of the iC₈ and unsaturated higher hydrocarbons (e.g., C₆₊ olefins) to yield a product stream comprising C₁₄₊ isoparaffins 2021 which may be suitable for blending into jet fuel and/or diesel fuel. In some cases, one or more additional stream 2018 comprising unsaturated hydrocarbons (e.g., C₃ and C₄ olefins) can be sourced from adjacent refinery/petrochemical units 2007 (such as FCC, MTO, FT, delayed cokers, or steam crackers) to constitute additional feed into the first alkylation unit, thereby increasing gasoline/jet/diesel fuel production of out the process scheme.

In some cases, an oxidizing agent 2011, such as O₂, air, or water, is fed along with the ethylene conversion feed (which may contain H₂) into the ethylene conversion unit, so as to minimize/limit the extent of hydrogenation of unsaturated hydrocarbons in the ethylene conversion feed over the oligomerization catalysts and to reduce the yield of oligomers. The oxidizing agent 2011 may be directed from the OCM unit 2001 which is upstream of and in fluidic communication with the ethylene conversion unit. Carbon monoxide (CO) contained in feed stream of the ethylene conversion unit may be converted in a FT unit (not shown in the figure) with H₂ into C₁-C₄ paraffins, so as to minimize the adverse impact it may have over the metal-containing oligomerization catalyst (e.g., Ni) such as etching.

Alternatively or additionally, a hydrotreating catalyst layer (or separate reaction zone) (not shown in the figure) upstream of the ethylene conversion unit can be employed to remove sulfur from certain feeds to the ethylene conversion unit. The hydrotreating catalyst can be in the form of a hydrotreating catalyst layer, composed of a CoMo and/or NiMo based catalyst which may react sulfur and not saturate olefins in the feed over the used process conditions, or in the form of a separate and upstream hydrotreating unit, which can comprise a mercaptan oxidation (MEROX) type unit employing a liquid catalyst or a CoMo/NiMo based unit.

FIG. 21 illustrates an example system for producing hydrocarbons using a water recovery stream 2100. A source containing methane 2101 is injected into an oxidative coupling of methane (OCM) reactor 2102. The OCM reactor may convert a portion of the methane into olefins. The olefins produced in the OCM reactor and a water recovery stream may be injected into an ethylene-to-liquids (ETL) reactor 2103. The ETL reactor may be configured to convert a portion of the olefins into a stream containing hydrocarbons with at least five carbon atoms (C₅₊ compounds), hydrocarbons with four carbon atoms (C₄ compounds), and water. The stream containing hydrocarbons with at least five carbon atoms (C₅₊ compounds), hydrocarbons with four carbon atoms (C₄ compounds), and water may be injected into a separation unit 2104 to separate the components into a first stream containing hydrocarbons with five or more carbon atoms (C₅₊ compounds) and water, and a second stream containing hydrocarbons with four carbon atoms (C₄ compounds). The second stream may be injected into a fractionation unit 2106. The fractionation unit may separate components in the second stream to produce a stream containing olefins with between two and four carbon atoms (C₂-C₄ olefins), a stream containing methane and ethane, and a stream containing CO₂. The stream containing methane and ethane may be injected into the OCM reactor 2102. The first stream containing the C₅₊ compounds and water may be injected into a unit 2105. The unit 2105 may be configured to separate the components into a stream containing water and a stream containing C₅₊ compounds. The stream containing water may be the water recovery stream that is injected into the ETL reactor 2103.

FIG. 22 illustrates an example system for producing hydrocarbons using a water recovery stream and a gas stream from a fluidized catalytic cracker (FCC) 2200. A source containing methane 2101 is injected into an oxidative coupling of methane (OCM) reactor 2102 to convert a portion of the methane into olefins. The olefins produced in the OCM reactor, a water recycle stream, and a source of gas from a fluidized catalytic cracker (FCC) 2203 may be injected into an ethylene-to-liquids reactor 2104 to convert a portion of the olefins into a stream containing hydrocarbons with at least five carbon atoms (C₅₊ compounds), hydrocarbons with four carbon atoms (C₄ compounds), and water. The stream containing hydrocarbons with at least five carbon atoms (C₅₊ compounds), hydrocarbons with four carbon atoms (C₄ compounds), and water may be injected into a separation unit 2105 that separates the components into a stream containing hydrocarbons with five or more carbon atoms (C₅₊ compounds) and water, and a stream containing hydrocarbons with four carbon atoms (C₄ compounds). The stream containing hydrocarbons with four carbon atoms (C₄ compounds) may be injected into a fractionation unit 2107, that separates components in the stream to produce a stream containing olefins with between two and four carbon atoms (C₂-C₄ olefins), a stream containing methane and ethane, and a stream containing CO₂. The stream containing methane and ethane may be injected into the oxidative coupling of methane (OCM) reactor 2102. The stream containing hydrocarbons with five or more carbon atoms (C₅₊ compounds) and water may be injected into a unit 2106 that separates the components into a stream containing water and a stream containing hydrocarbons with five or more carbon atoms (C₅₊ compounds). The stream containing water may be the water recovery stream that is injected into the ethylene-to-liquids (ETL) reactor 2104.

FIG. 23 schematically illustrates an example system for producing oxygenates using a water recycle stream. A source containing methane 2301 may be injected into an oxidative coupling of methane (OCM) reactor 2302 to produce a stream containing olefins. The stream containing olefins and a water recovery stream may be injected into an ethylene-to-liquids (ETL) reactor 2303 to produce a stream containing hydrocarbons with four carbon atoms (C₄ compounds), hydrocarbons with five or more carbon atoms (C₅₊ compounds), and water. The stream containing hydrocarbons with four carbon atoms (C₄ compounds), hydrocarbons with five or more carbon atoms (C₅₊ compounds), and water may be injected into a separation unit 2304 that produces a stream containing hydrocarbons with four carbon atoms (C₄ compounds) and a stream containing hydrocarbons with five or more carbon atoms (C₅₊ compounds and water. The stream containing hydrocarbons with four carbon atoms (C₄ compounds) may be injected into a fractionation unit 2306 that separates the components in the incoming stream to produce a stream containing olefins with between two and four carbon atoms (C₂-C₄ olefins), a stream containing methane and ethane, and a stream containing CO₂. The stream containing methane and ethane may be injected into the oxidative coupling of methane (OCM) reactor 2302. The stream containing olefins with between two and four carbon atoms (C₂-C₄ olefins) may be injected into the ethylene-to-liquids (ETL) reactor 2303. The stream containing hydrocarbons with five or more carbon atoms (C₅₊ compounds) and water may be injected into a hydration unit 2305 that converts a portion of the C₅₊ compounds into oxygenates with five or more carbon atoms (C₅₊ oxygenates) to produce a stream containing oxygenates with five or more carbon atoms (C₅₊ oxygenates) and water. The stream containing oxygenates with five or more carbon atoms (C₅₊ oxygenates) and water may be injected into a separation unit that produces a stream containing water and a stream containing oxygenates with five or more carbon atoms (C₅₊ oxygenates). The stream containing water may be the water recovery stream and can be injected into the hydration unit 2305, the ethylene-to-liquids (ETL) reactor 2303, or both.

An additional amount of water can be added to the water recovery stream. The additional amount of water can be less than or equal to about 95%, 90%, 85%, 0%, 75%, 70%, 65%, 60%, 55%, 50%, 45%, 40%, 35%, 30%, 25%, 20%, 15%, 10%, 5% of the water recovery stream or less.

The hydration unit can operate at a temperature between about 50° C. and about 300° C., between about 75° C. and about 300° C., between about 100° C. and about 300° C., between about 100° C. and about 250° C., between about 100° C. and about 200° C., or between about 120° C. and about 180° C.

The hydration unit can operate at a pressure between about 1 bar and about 200 bar, between about 1 bar and about 150 bar, between about 1 bar and about 100 bar, between about 1 bar and about 80 bar, between about 1 bar and about 60 bar, between about 1 bar and about 40 bar, or between about 1 bar and about 20 bar.

The hydration unit can operate at a feed composition that is at least about 50 mole percent water and less than about 50 mole percent hydrocarbons, at least about 75 mole percent water and less than about 25 mole percent hydrocarbons, at least about 85 mole percent water and less than about 15 mole percent hydrocarbons, at least about 90 mole percent water and less than about 10 mole percent hydrocarbons, at least about 95 mole percent water and less than about 5 mole percent hydrocarbons, or at least about 98 mole percent water and less than about 2 mole percent hydrocarbons.

The hydration unit can contain a hydration catalyst. The hydration catalyst can comprise water soluble acids (e.g. HCl, H₃PO₄, H₂SO₄, heteropoly acids), organic acids (e.g. acetic acit, tosylate acid, perflorinatidd acetic acid), solid acids (e.g. ionic exchange resins, acidic zeolite, metal oxide), or combinations thereof. The ethylene-to-liquids (ETL) reactor can contain an ethylene-to-liquids (ETL) catalyst. The ethylene-to-liquids (ETL) catalyst can be a zeolite. The zeolite can comprise ZSM-5, ZSM-11, ZSM-12, ZSM-35, ZSM-38, Beta, Mordinite, or combinations thereof.

The ethylene-to-liquids (ETL) reactor can operate with a feed composition that is between about 0.5 mole water per mole olefins and about 16 mole water per mole olefins, about 1 mole water per mole olefins and about 16 mole water per mole olefins, about 1 mole water per mole olefins and about 10 mole water per mole olefins, about 2 mole water per mole olefins and about 10 mole water per mole olefins, or about 2 mole water per mole olefins and about 5 mole water per mole olefins.

### ETL Processes and Operating Conditions

The present disclosure provides methods for operating ETL reactors to effect a given or predetermined product distribution or selectivity. The process conditions can be applied across a single or plurality of ETL reactors in series and/or parallel.

Hydrocarbon streams into or out of an ETL reactor can include various other non-hydrocarbon material. In some cases, hydrocarbon streams can include one or more elements leached from an OCM catalyst (e.g., La, Nd, Sr, W) or ETL catalyst (e.g., Ga dopant).

Reactor conditions can be selected to provide a given selectivity and product distribution. In some cases, for catalyst selectivity towards aromatics, an ETL reactor can be operated at a temperature greater than or equal to about 300° C., 350° C., 400° C., 410° C., 420° C., 430° C., 440° C., 450° C., or 500° C., and a pressure greater than or equal to about 150 pounds per square inch (PSI) (absolute), 200 PSI, 250 PSI, 300 PSI, 350 PSI or 400 PSI. For catalyst selectivity towards jet or diesel fuel, an ETL reactor can be operated at a temperature greater than or equal to about 100° C., 150° C., 200° C., 210° C., 220° C., 230° C., 240° C., 250° C., or 300° C., and a pressure greater than or equal to about 350 PSI, 400 PSI, 450 PSI, or 500 PSI. For catalyst selectivity towards gasoline, an ETL reactor can be operated at a temperature greater than or equal to about 200° C., 250° C., 300° C., 310° C., 320° C., 330° C., 340° C., 350° C., or 400° C., and a pressure greater than or equal to about 250 PSI, 300 PSI, 350 PSI, or 400 PSI.

In some cases, the operating conditions of an ETL process are substantially determined by one or more of the following parameters: process temperature range, weight-hourly space velocity (mass flow rate of reactant per mass of solid catalyst), partial pressure of a reactant at the reactor inlet, concentration of a reactant at the reactor inlet, and recycle ratio and recycle split. The reactant can be an (light) olefin—e.g., an olefin that has a carbon number in the range C₂-C₇, C₂-C₆, or C₂-C₅.

Temperatures used in a gasoline process can be from about 150 to 600° C., 220° C. to 520° C., or 270° C. to 450° C. Lower temperature can result in insufficient conversion while higher temperatures can result in excessive coking and cracking of product. In an example, the WHSV can be between about 0.5 hr⁻¹ and 3 hr⁻¹, partial pressures can be between about 0.5 bar (absolute) and 3 bar, and concentrations at the reactor inlet can be between about 2% and 30%. Higher concentrations can yield difficult-to-manage temperature excursions, while lower concentrations can make it difficult to achieve sufficiently high partial pressures and separation of the products. A process can achieve longer catalyst lifetime and higher average yields when a portion of the effluent is recycled. The recycle can be determined by a recycle ratio (e.g., volume of recycle gas/volume of make-up feed) and the post-reactor vapor-liquid split which determines the composition of the recycle stream. There may be several degrees of freedom to the recycle split, but in some cases the composition of the recycle stream may be important, which may be achieved by post-reactor separation (e.g., carbon number/boiling point range that is recycled vs. the carbon number/boiling point ranges that are removed by product and/or secondary process streams.

To achieve longer average chain lengths and to avoid cracking of elongated chains such as those found in jet fuel and distillates, ETL can be performed at reactor operating temperatures from about 150° C. to 500° C., 180° C. to 400° C., or 200° C. to 350° C. The slower kinetics may suggest a lower minimum WHSV of about 0.1 hr⁻¹. Longer chain lengths may be favored by high partial pressures, so the upper end for jet/distillates may be higher than for gasoline, in some cases as high as about 30 bar (absolute), 20 bar, 15 bar, or 10 bar.

More consistent production of aromatics can be achieved at high temperature ranges, such as a temperature up to about 200° C., 250° C., 300° C., 350° C., 400° C., 450° C., or 500° C. In an adiabatic or even in a pseudo-isothermal reactor, the ethylene/olefin feed can be diluted by an inert gas (e.g., N₂, Ar, methane, ethane, propane, butane or He). The inert gas can serve to moderate the temperature increase in the reactor bed, and maintain and stabilize contact time. The olefin concentration at the reactor inlet can be less than about 50%, 40%, 30%, 20%, or 10%. In some cases, the higher the molar heat capacity of the diluent, the higher the inlet concentration of olefins can be to achieve the same temperature rise.

The following is a list of suitable compounds that may be found in significant quantities in the process. Such compounds are listed in the order of increasing heat capacity: nitrogen, carbon dioxide, methane, ethane, propane, n-butane, iso-butane.

In some cases, a continuous process for making mixtures of hydrocarbons from (light) olefins by oligomerization comprises feeding a stream of unsaturated hydrocarbons including olefinic compounds (e.g., acyclic olefins, cyclic olefins, or di-olefins) to a reaction zone of an ETL reactor. The reactor zone can contain a heterogeneous catalyst. One or more inert gases can be co-fed to the reactor inlet, making up from about 50% (volume %) to 99%, 60% to 98%, or 70% to 98% of the feedstock. The mixture can be comprised at least one of the following compounds: nitrogen, carbon dioxide, methane, ethane, propane, n-butane, iso-butane. The process (e.g., ETL reactor) temperature can be between about 150° C. and 600° C., 180° C. and 550° C., or 200° C. and 500° C. The partial pressure of olefins in the feed can be between about 0.1 bar (absolute) to 30 bar, 0.1 bar to 15 bar, or 0.2 bar to 10 bar. The total pressure can be between about 1 bar (absolute) to 100 bar, 5 bar to 50 bar, or 10 bar to 50 bar. The weight hourly space velocity can be between about 0.05 hour⁻¹ (hr⁻¹) to 20 hr⁻¹, 0.1 hr⁻¹ to 10 hr⁻¹, or 0.1 hr⁻¹ to 5 hr⁻¹.

An effluent or product stream from an ETL reactor can be characterized by low water content. For example, an ETL product stream can comprise less than about 60 wt %, 56 wt %, 55 wt %, 50 wt %, 45 wt %, 40 wt %, 39 wt %, 35 wt %, 30 wt %, 25 wt %, 20 wt %, 15 wt %, 10 wt %, 5 wt %, 3 wt %, or 1 wt % water.

In some cases, at least a portion (e.g., greater than or equal to about 1%, 5%, 10%, 20%, 30%, 40%, or 50%) of the reactor effluent is recycled to the reactor. As an alternative, at most a portion (e.g., less than or equal to about 90%, 80%, 70%, 60%, 40%, 20% or 10%) of the reactor effluent is recycled to the reactor inlet. The volumetric recycle ratio (i.e., flow rate of the recycle gas stream divided by flow rate of the make-up gas stream (i.e., fresh feed)) can be at least about 0.1, 0.5, 1, 5, 10, 30, 30, 40, 50 or higher, or between about 0.1 and 30, 0.3 and 20, or 0.5 and 10.

A continuous process for making mixtures of hydrocarbons for use as gasoline can comprise feeding a stream of unsaturated hydrocarbons including olefinic compounds to a reaction zone of an ETL reactor. The ETL reactor can include a catalyst that is selected for gasoline production, as described elsewhere herein. The process temperature can be at least about 200° C., 300° C., 400° C., 500° C., 600° C., 700° C., 800° C. or higher, or between about 200° C. and 600° C., 250° C. and 500° C., or 300° C. and 450° C. The partial pressure of olefins in the feed can be between about 0.1 bar (absolute) to 10 bar, 0.3 bar to 5 bar, or 0.5 bar to 3 bar. The total pressure can be between about 1 bar (absolute) to 100 bar, 5 bar to 50 bar, or 10 bar to 50 bar. The weight hourly space velocity can be between about 0.1 hr⁻¹ to 20 hr⁻¹, 0.3 hr⁻¹ to 10 hr⁻¹, or 0.5 hr⁻¹ to 3 hr⁻¹.

For products in the distillate range (e.g., C₁₀₊ molecules, which can exclude gasoline in some cases), the catalyst composition can be selected as described elsewhere herein. The process temperature can be at least about 100° C., 200° C., 300° C., 400° C., 500° C., 600° C. or higher, or between about 100° C. and 600° C., 150° C. and 500° C., or 200° C. and 375° C. The partial pressure of olefins in the feed can be between about 0.5 bar (absolute) to 30 bar, 1 bar to 20 bar, or 1.5 bar to 10 bar. The total pressure can be between about 1 bar (absolute) to 100 bar, 5 bar to 50 bar, or 10 bar to 50 bar. The weight hourly space velocity can be between about 0.05 hr⁻¹ to 20 hr⁻¹, 0.1 hr⁻¹ to 10 hr-1, or 0.1 hr⁻¹ to 1 hr⁻¹.

For products comprising mixtures of hydrocarbons substantially comprised of aromatics, the catalyst composition can be selected as described elsewhere herein. The process temperature can be at least about 200° C., 300° C., 400° C., 500° C., 600° C., 700° C., 800° C. or higher, or between about 200° C. and 800° C., 300° C. and 600° C., or 400° C. and 500° C. The partial pressure of olefins in the feed can be between about 0.1 bar (absolute) to 10 bar, 0.3 bar to 5 bar, or 0.5 bar to 3 bar. The total pressure can be between about 1 bar (absolute) to 100 bar, 5 bar to 50 bar, or 10 bar to 50 bar. The weight hourly space velocity can be between about 0.05 hr⁻¹ to 20 hr⁻¹, 0.1 hr⁻¹ to 10 hr⁻¹, or 0.2 hr⁻¹ to 1 hr⁻¹.

The ETL process can generate a variety of long-chain hydrocarbons, including normal and isoparaffins, napthenes, aromatics and olefins, which may not be present in the feed to the ETL reactor. The catalyst can deactivate due to the deposition of carbonaceous deposits (“coke”) on the surfaces of the catalyst. As the deactivation progresses, the conversion of the process changes until a point is reached when the catalyst can be regenerated.

In some cases, in the early stages of a reaction cycle, the product distribution can contain large fractions of aromatics and short-chained alkanes. Later stages can feature increased fractions of olefins. All stages can feature various amounts isoparaffins, n-paraffins, naphthenes, aromatics, and olefins, including olefins other than feed olefins. The change in selectivity with time can be exploited by separating products. For example, the aromatics-rich effluent characteristic of the early stages of a reaction cycle may be readily separated from the effluent of a catalyst bed in a later stage of its cycle. This can result in high selectivities of individual products.

The ETL process can generate various byproducts, such as carbon-containing byproducts (e.g., coke) and hydrogen. The selectivity for coke can be on the order of at least about 1%, 2%, 3%, 4%, or 5% over the course of an ETL process. Hydrogen production can vary with time, and the amount of hydrogen generated can be correlated with aromatics production.

In some cases, the time-averaged product of the process can yield a liquid with a composition that meets the specification of reformulated gasoline blendstock for oxygen blending (RBOB). In some cases, RBOB has at least about an 93 octane rating using the (RON+MON)/2 method, has less than about 1.3 vol % benzene as measured by ASTM D3606, has less than about 50 vol % aromatics as measured by ASTM D5769, has less than about 25 vol % olefins as measured by ASTM D1319 and/or D6550, has less than 80 ppm (wt) sulfur as measured by ASTM D2622, or any combination thereof. Such liquid can be employed for use as fuel or other combustion settings. This liquid can be partially characterized by the content of aromatics. In some cases, this liquid has an aromatics content from 10% to 80%, 20% to 70%, or 30% to 60%, and an olefins content from 1% to 60%, 5% to 40%, or 10% to 30%. Gasoline can comprise about 60% to 95%, 70% to 90%, or 80-90% of such liquid, with the remainder in some cases being an alcohol, such as ethanol.

In some situations, an ETL process is used to generate a mixture of hydrocarbons from light olefin compounds (e.g., ethylene). The mixture can be liquid at room temperature and atmospheric pressure. The process can be used to form a mixture of hydrocarbons having a hydrocarbon content that can be tailored for various uses. For example, mixtures that may be characterized as gasoline or distillate (e.g., kerosene, diesel) blend stock, or aromatic compounds, can contribute at least 30%, 40%, 50%, 60%, or 70% by weight to the final fuel product.

The product selectivity of the ETL process can change with time. With such changes in selectivity, the product can include varying distributions of hydrocarbons. Separations units can be used to generate a product distribution which can be suitable for given end uses, such as gasoline.

Products of ETL processes of the present disclosure can include other elements or compounds that may be leached from reactors or catalysts of the system (e.g., OCM and/or ETL reactors). Examples of OCM catalysts and the elements comprising the catalyst that can be leached into the product can be found in U.S. Patent Publication No. 2013/0165728 or U.S. Provisional Patent Application 61/988,063, each of which is incorporated by reference in its entirety. Such elements can include transition metals and lanthanides. Examples include, but are not limited to Mg, La, Nd, Sr, W, Ga, Al, Ni, Co, Ga, Zn, In, B, Ag, Pd, Pt, Be, Ca, and Sr. The concentration of such elements or compounds can be at least about 0.01 parts per billion (ppb), 0.05 ppb, 0.1 ppb, 0.2 ppb, 0.3 ppb, 0.4 ppb, 0.5 ppb, 0.6 ppb, 0.7 ppb, 0.8 ppb, 0.9 ppb, 1 ppb, 5 ppb, 10 ppb, 50 ppb, 100 ppb, 500 ppb, 1 part per million (ppm), 5 ppm, 10 ppm, or 50 ppm as measured by inductively coupled plasma mass spectrometry (ICPMS).

The composition of ETL products from a system can be consistent over several cycles of catalyst use and regeneration. A reactor system can be used and regenerated for at least about 10, 20, 30, 40, 50, 60, 70, 80, 90, or 100 cycles. After a number of regeneration cycles, the composition of the ETL product stream can differ from the composition of the first cycle ETL product stream by no more than about 0.1%, 0.2%, 0.3%, 0.4%, 0.5%, 0.6%, 0.7%, 0.8%, 0.9%, 1%, 2%, 3, 4%, 5%, 6%, 7%, 8%, 9%, 10%, 11%, 12%, 13%, 14%, 15%, 16%, 17%, 18%, 19%, or 20%.

### ETL Process Feedstock

The feedstock to an ETL reactor can have an effect on the product distribution out of the ETL reactor. The product distribution can be related to the concentration of olefins into the ETL reactor, such as ethylene, propylene, butene(s) and pentene(s). The feedstock concentration can impact ETL catalyst efficiency. A feedstock of unsaturated hydrocarbons having an olefin concentration that is greater than or equal to about 5%, 10%, 15%, 20%, 25%, 30%, or 40% can be efficient at generating higher molecular weight hydrocarbons. In some cases, the optimum olefin concentration can be less than or equal to about 80%, 85%, 75%, 70%, 60% or 50%. The ETL feedstock can be characterized based on the ethylene to ethane molar ratio of the feedstock, which can be at least about 2:1, 3:1, 4:1, 5:1, 6:1, 7:1, or 8:1.

The presence of other C₂₊ compounds and non-C₂₊ impurities (e.g., CO, C₂, H₂O and H₂) can have an impact on ETL selectivity and/or product distribution. For instance, the presence of acetylene and/or dienes in a feedstock to an ETL reactor can have a significant impact on ETL selectivity and/or product distribution, since acetylene may be a deactivator and coke accelerator.

### ETL-containing Methods and Systems

Also provided herein are ETL-containing methods and systems for generating oxygenate compounds with five or more carbon atoms (C₅₊ oxygenates). The oxygenate compounds may be any oxygenated chemicals which contain oxygen as a part of their chemical structure. Examples of oxygenate compounds include, but are not limited to, alcohols, glycols, ethers, esters, ketones, aldehydes, diols, carboxylic acids, acid anhydrides, amides, and combinations thereof. The methods may comprise directing a feed stream comprising ethylene (C₂H₄) into an ETL system comprising an ETL reactor. The feed stream can comprise unsaturated hydrocarbons (i.e., hydrocarbons that have double or triple covalent bonds between adjacent carbon atoms). The ETL reactor may convert the C₂H₄ in an ETL process to yield a product stream. The product stream may comprise various compounds including e.g., saturated and unsaturated hydrocarbons. In some cases, the product stream comprises compounds with five or more carbon atoms (C₅₊ compounds) which may be olefins such as acyclic olefins, cyclic olefins or di-olefins, and/or alkynes such as acyclic or cyclic alkynes, or a combination thereof.

Subsequently, the generated product stream can be directed from the ETL reactor into one or more (e.g., at least about 1, 2, 3, 4, 5, 6, 7, 8, 9, 10, 12, 14, 16, 18, or 20) various processing units or systems fluidically connected to the ETL system for reacting or converting the product stream in multiple different conversion processes to multiple different products. The product stream may be selectively directed from the ETL system in whole or in part to any one of the processing units for further reaction. For example, at any given time, all of the product stream generated in the ETL rector may be directed therefrom to a single processing unit. Alternatively, only a portion of the product stream yielded in the ETL process may be routed to a first processing unit, and some or all of the remaining product stream may be directed to one, two, three, four, five, or more processing units or system. As an example, a portion of the product stream can be directed from the ETL reactor to a hydration unit or system which is fluidically coupled to the ETL reactor, and the hydration unit can convert such portion of the product stream in a hydration process to generate an oxygenate product stream comprising e.g., C₅₊ oxygenates.

As described above and elsewhere herein, the one or more separate processing units or systems can be fluidically coupled to and integrated with the ETL reactor in an integrated system. As used herein, fluid integration generally refers to a persistent fluid connection between two systems within an overall system or facility. Such persistent fluid connection or communication generally refers to an interconnected pipeline network coupling one system to another. Such interconnected pipelines can also include additional elements between two systems, such as control elements, e.g., heat exchangers, pumps, valves, compressors, turbo-expanders, sensors, as well as other fluid or gas transport and/or storage systems, e.g., piping, manifolds, storage vessels, and the like, but are generally entirely closed systems, as distinguished from two systems where materials are conveyed from one to another through any non-integrated component, e.g., railcar or truck transport, or systems that are not co-located in the same facility or immediately adjacent facilities. As used herein, fluid connection and/or fluid coupling includes complete fluid coupling, e.g., where all effluent from a given point such as an outlet of a reactor, is directed to the inlet of another unit with which the reactor is fluidly connected. Also included within such fluid connections or couplings are partial connections, e.g., where only a portion of the effluent from a given first unit is routed to a fluidly connected second unit. Further, although stated in terms of fluid connections, it will be appreciated that such connections include connections for conveying either or both of liquids and/or gas.

While feed stream being directed into the ETL reactor may range anywhere from trace concentrations of ethylene to pure or substantially pure ethylene (e.g., approaching 100% ethylene). In some cases, the feed stream comprises greater than or equal to about 1%, 5%, 10%, 15%, 20%, 25%, 30%, 35%, 40%, 45%, 50%, 60%, 70%, 80%, 90% (volume percent (vol %), weight percent (wt %) or mole percent (mol %)), or more ethylene. In some cases, the feed stream comprises less than or equal to about 100%, 90%, 80%, 70%, 60%, 50%, 40%, 30%, 20%, 10%, 5% or less ethylene. In some cases, the feed stream is characterized as having anywhere between about 1% and about 50%, between about 5% and about 25% ethylene or, between about 10% and about 25% ethylene, in addition to other components. In some cases, the feed stream employed in the ETL processes further comprise one or more gases including e.g., CO₂, CO, H₂, H₂O, C₂H₆, CH₄ and hydrocarbons with three or more carbon atoms (C₃₊ hydrocarbons).

FIG. 3 shows an example ETL-containing system 300 for use in producing oxygenates compounds. The system comprises an ETL unit 304, a fractionation unit (e.g., demethanizers, deethanizers, debutanizers, depropanizers etc.) 306, a hydration unit 312 and a regeneration unit 314. The direction of fluid flow is indicated by the arrows. The ETL unit takes the incoming feed stream 302 which comprises ethylene. The ETL unit can comprise one or more ETL reactors which can conduct an ethylene conversion reaction that converts ethylene to a product stream. The generated product stream may comprise higher molecular weight hydrocarbons. At least a part of the product stream may be directed into the fractionation unit 306 downstream of and fluidically connected to the ETL unit to separate the product stream into multiple different compounds. In some cases, the fractionation unit 306 is a debutanizer which splits the product stream into a first product stream 310 comprising short chain hydrocarbons (i.e., C1-C4 compounds) and a second product stream 308 comprising C₅₊ compounds. The first product stream 310 may be directed from the fractionation unit 306 to one or more additional processing units (not shown in the figure) for further reaction or product recovery. Additionally or alternatively, the first product stream may be recycled to the ETL unit or the unit that stores or generates the ETL feed stream (e.g., an OCM unit). The second product stream generated in the fractionation unit 306 may be directed therefrom into the hydration unit 312, and subsequently the regeneration unit 314, from which water is recovered 318 and an end product stream 316 is produced. The end product stream can comprise one or more higher molecular weight hydrocarbons such as gasoline, diesel fuel, jet fuel, and aromatic chemicals. In the hydration unit 312, the C₅₊ compounds is reacted with water under conditions sufficient to convert unsaturated C₅₊ compounds (e.g., olefins) to C₅₊ oxygenates (e.g., C₅₊ alcohols), thereby generating a stream of C₅₊ compounds with reduced olefin content that is in line with the Federal or state specifications. In some cases, a separate stream of water is directed into the hydration unit 312 and reacts with the C₅₊ compounds.

The hydration process of the present disclosure can be carried out under liquid phase, vapor phase, supercritical dense phase, or mixtures of these phases in semi-batch or continuous manner using a stirred tank reactor or fixed bed flow reactor. In some example, reaction times of from about 20 minutes to about 20 hours when operating in batch and a LHSV (i.e., reactant liquid flow rate/reactor volume) of from about 0.1 to about 10 when operating continuously are suitable. In some cases, unreacted unsaturated hydrocarbons (e.g., olefins) are recycled to the reactor for further reaction.

In some examples, the hydration unit 312 is operated under such conditions that at least about 10%, 15%, 20%, 25%, 30%, 35%, 40%, 45%, 50%, 55%, 60%, 65%, 70%, 75%, 80%, 85%, 90%, 95% (volume percent (vol %), weight percent (wt %) or mole percent (mol %)) or more unsaturated C₅₊ compounds are converted to C₅₊ oxygenates. In some cases, after hydration process, the amount of unsaturated compounds (e.g., olefins) included in the end product stream 316 is less than or equal to about 50%, 40%, 30%, 20%, 15%, 10%, 9%, 8%, 7%, 6%, 5%, 4%, 3%, 2%, 1% (volume percent (vol %), weight percent (wt %) or mole percent (mol %)) or less.

The hydration unit may comprise a hydration catalyst that facilitates a hydration process (or reaction) in the hydration unit. The hydration catalyst may comprise an acid catalyst. In some cases, the hydration catalyst is selected from acid catalyst groups comprising water soluble acids (e.g., HNO₃, HCl, H₃PO₄, H₂SO₄, hetoropoly acids), organic acids (e.g., acetic acid, tosylate acid, perflorinated acetic acid), metal organic frameworks (MOF), and solid acids (e.g., ion exchange resins, acidic zeolite, metal oxide).

Reaction conditions of the hydration unit can be selected to provide a given selectivity and product distribution. In some cases, a hydration unit can be operated at a temperature that is greater than or equal to about 50° C., 100° C., 150° C., 200° C., 250° C., 300° C., 350° C., 400° C., 450° C. or higher, or between any of the two values described herein, e.g., 100° C.-200° C. The pressure may be greater than or equal to about 100 PSI, 200 PSI, 300 PSI, 400 PSI, 500 PSI, 600 PSI, 700 PSI, 800 PSI, 900 PSI, 1,000 PSI, 1,500 PSI, 2,000 PSI, 2,500 PSI, 3,000 PSI, 3,500 PSI, 4,000 PSI or more, or between any of the two values described herein (e.g., 500-2,000 PSI). The molar ratio of water to C₅₊ compounds may vary. In some cases, the water to C₅₊ compounds mole ratio is at least about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 50, 60, 70, 80, 90, 100, 150, 200, 250, or 300. In some cases, the water to C₅₊ compounds mole ratio falls into a range between any of the two values described herein, for example, about 0.3-5. Contact time of the unsaturated hydrocarbons and the hydration catalyst can be at least about 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 22, 24, 26, 28, 30 hr⁻¹, or more. As an example, the hydration unit is operated at a temperature of 100° C. to 200° C., a pressure ranging from 10-1500 PSI, and water to hydrocarbon mole ratio of 1-200. Contact time of the reacting C₅₊ olefin and the hydration catalyst can be from 0.1-20 hr⁻¹.

As described above, the fractionation unit may split the product stream generated in an ETL reactor into a first product stream comprising shorter chain hydrocarbons (i.e., C1-C4 compounds) and a second product stream comprising longer hydrocarbons (e.g., C₅₊ compounds). The first product stream may be purged in some situations. In some cases, at least a portion of the first product stream is further processed and recycled to the ETL unit and/or a different unit which is upstream of and in fluidic communication with the ETL unit (e.g., an OCM unit). FIG. 4 illustrates such an example system 400 where the stream of shorter chain hydrocarbons (i.e., C1-C4 compounds) is sent to one or more additional processing units to generate additional product streams which may comprise different hydrocarbon products.

As shown in FIG. 4, similar to system 300, system 400 comprises an ETL unit 404, a fractionation unit 406, a hydration unit 412 and a regeneration unit 414. The direction of fluid flow is indicated by the arrows. The ETL unit takes the incoming feed stream 402 which comprises ethylene. The feed stream may be generated in whole or in part in an OCM reactor of an OCM unit.

The OCM unit and the ETL unit may be integrated with each other. Such integration can advantageously enable the formation of products that can be tailored for various uses, such as, for example fuel. Such integration can enable the conversion of ethylene in a C₂₊ product stream from an OCM reactor to be converted to higher molecular weight hydrocarbons. Examples of OCM methods and systems are described in U.S. Pat. Nos. 9,334,204, and 9,469,577, each of which is entirely incorporated herein by reference.

The ETL unit comprises at least one ETL reactor which can react the feed stream 402 in an ETL process to generate a product stream comprising higher molecular weight hydrocarbons (e.g., C₅₊ compounds). The product stream is then directed from the ETL unit into a separation unit 406 for separating C₄₋ compounds and C₅₊ compounds 410 from the remainder of ETL product stream. Similar to the system 300 shown in FIG. 3, the C₅₊ compounds 410 are sent to a hydration unit 412 along with water 418, and an oxygenate-rich C₅₊ stream is produced and sent to the gasoline pool 416. In some cases, water from the hydration unit may be recovered 414 and recycled to the hydration unit 412.

The C₄₋ compounds may be routed to a different processing unit (e.g., an aromatization unit 420) which converts the C₄₋ compounds to different hydrocarbon compounds (e.g., aromatic hydrocarbon compounds). In some cases, the C₄₋ compounds are further heated in a fired heater 408 prior to being sent to the aromatization unit 420 so as to reach a desirable aromatization temperature for an aromatization reaction in the aromatization unit. One example of an aromatization process is the Cyclar process which converts liquefied petroleum gas (LPG) directly into a liquid aromatics product in a single operation.

In some cases, the aromatization unit is operated at a temperature that is higher than the operating temperature of the ETL unit and a difference between the operating temperatures of the aromatization unit and the ETL unit is at least about 10° C., 20° C., 30° C., 40° C., 50° C., 60° C., 70° C., 80° C., 90° C., 100° C., 150° C., 200° C., 250° C., 300° C., 400° C., or 550° C. In addition to the operating temperature, other reaction/operation conditions in the aromatization unit may vary. For example, the aromatization unit may be operated at a pressure that is greater than or equal to about 10 PSI, 20 PSI, 30 PSI, 40 PSI, 50 PSI, 60 PSI, 70 PSI, 80 PSI, 90 PSI, 1,000 PSI, or higher, or between any of the two values described herein (e.g., 10-300 PSI), with a hydrogen (H₂) to hydrocarbon mole ratio of at least about 0.001, 0.005, 0.01, 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5, or more, or between any of the values described therein (e.g., 0.01-2).

Additional hydrogen (H₂) and/or inert gases (e.g., nitrogen (N₂) or noble gases) 428 may be added to the stream as desired to regulate pressures, to control H₂/hydrocarbon ratio, and/or to suppress the coke formation over catalysts in the aromatization unit. In the aromatization unit, the C₄₋ compounds are reacted under conditions that yield hydrocarbon compounds comprising aromatics. The aromatics may comprise one or more of benzene, toluene, xylene, ethylbenzene, and combinations thereof. The reactions in the aromatization unit can progress until the C₄₋ compounds are substantially (e.g., at least 80%, 85%, 90%, 95% or more (vol %, wt %, or mol %)) converted. The aromatization unit may comprise at least one aromatization reactor which may be a fixed-bed, moving-bed or fluid bed reactor in configuration. The aromatization reactor may comprise a catalyst that facilitates an aromatization reaction. The aromatization catalyst may comprise a zeolite-type alumino-, gallo- or boro-silicate (e.g., ZSM-5 or ZSM-11) which has gallium, aluminum and/or zinc incorporated into the structure and has been treated with rhenium and a metal selected from nickel, palladium, platinum, rhodium and iridium. The aromatization catalyst may comprise an MFI structure zeolite, which contains silicon and aluminium, as well as at least one noble metal from the platinum family, to which may be added metals chosen from the group consisting of tin, germanium, indium and lead. The aromatization catalyst may comprise a catalyst composition which is resistant to sulfur or a sulfur compound containing a zeolite, cerium or cerium oxide, and a Group VIII metal or metal oxide, such as platinum or platinum oxide. An amorphous matrix can be added to the catalyst with a view to the shaping thereof. During the aromatization reaction, contact time of the hydrocarbons with the aromatization catalyst may be greater than or equal to about 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 22, 24, 26, 28, 30 hr⁻¹, or more. As an example, the aromatization reaction is conducted at a temperature in the range of 350-600° C., and pressures ranging from 10-300 PSI, with a H₂ to hydrocarbon ratio of 0.01-2.0 mol/mol. Contact time of the hydrocarbons and the aromatization catalyst is from 0.1-20 h⁻¹.

The product stream generated in the aromatization unit may be fractionated into benzene, toluene and xylenes (BTX) 422 and other aromatics as well as unconverted C₄₋ hydrocarbons. In some cases, the unconverted C₄₋ hydrocarbons are sent to a separation unit comprising a de-ethanizer 424 and a fractionation unit 432. The separation unit can separate and recycle all or some of C₂₌/C₂₋ compounds 430 (including e.g., methane, ethane, and ethylene) to the ETL reactor 434, and/or to an OCM reactor 436 upstream of the ETL reactor. The remaining C₃ and C₄ hydrocarbons 426 produced from the aromatization reactor may be routed to the aromatization reactor as a recycle stream. Hydrogen from the aromatization reactor can also be recovered using a PSA unit or the like and recycled back into the aromatization reactor.

ETL systems of the present disclosure can be integrated or retrofitted in various existing systems, such as petroleum refineries and/or petrochemical complexes. Such integration can be with or without OCM systems. The integrated system may comprise one or more sub-systems (or units) including, but are not limited to, a metathesis unit, fluid catalytic cracking (FCC) unit, thermal cracker unit, coker unit, methanol to olefins (MTO) unit, Fischer-Tropsch unit, and oxidative coupling of methane (OCM) unit, and combinations thereof.

FIGS. 5A and 5B illustrate an example integrated ETL-containing system 500. The system comprises, an ETL unit 504, an OCM unit 538 upstream of the ETL unit, and a debutanizer 506 and a hydration unit 512 downstream of the ETL unit. The system further comprises a steam cracker unit 540 and a FCC unit 542 upstream of and in fluidic connection with ETL unit, as well as a metathesis unit (e.g., Lummus Olefin Conversion Technology (OCT)) 530. The steam cracker unit 540 and the FCC unit 542 can generate product streams that are rich in unsaturated hydrocarbons as at least a part of feed stream 502 to the ETL reactor. The feed stream may comprise additional reaction products, unreacted feed gases, or other reactor effluents from an ethylene production process, e.g., OCM, such as methane, ethane, propane, propylene, CO, CO₂, O₂, N₂, H₂, and/or water. The feed stream 502 directed into the ETL reactor is reacted in an ETL process to generate an ETL product stream comprising higher molecular weight hydrocarbons, which can be directed to the debutanizer 506 for splitting the ETL product stream into a first stream comprising C₄₋ compounds 508 and a second stream comprising C₅₊ compounds 510. Next, the second stream comprising C₅₊ compounds may be routed to the hydration unit 512 which reacts unsaturated hydrocarbons (e.g., C₅₊ olefins) included in the second stream with water in a hydration reaction to yield hydrocarbon compounds 516 with high content of C₅₊ oxygenates (e.g., alcohols). In some cases, water from the hydration unit may be recovered in a water recovery unit 514 and recycled to the hydration unit 512.

In some cases, the C₄₋ compounds from the debutanizer 506 is directed into an additional fractionation unit 520 for separation. The C₄₋ compounds may be separated into different streams comprising C₂₋ 536, C₂₌/C₃₌ 524, and C₂₌ 528 compounds respectively. In some cases, at least a portion of the C₂₋ 536 and the C₂₌/C₃₌ 524 compounds are recycled to the OCM unit and the ETL unit for further use. The metathesis unit 530 may take in a feed stream comprising C₂₌ compounds 528 and raff-1/raff-2 butenes 526 and converts the feed stream into hydrocarbons comprising propylene. In some cases, at least a portion of the metathesis feed stream is received from the FCC and/or steam cracker units and integration of the metathesis unit with the FCC and/or steam cracker units maximizes the production of propylene. The produced hydrocarbons from the metathesis unit may be fractionated into C₃₌ compounds 534 and C₅₊ compounds 532, which C₅₊ compounds 532 may be directed into the hydration unit 512 for producing C₅₊ oxygenates.

There may be other sources of C₅₊ streams that contain hydratable unsaturated hydrocarbons (e.g., olefins, di-olefins, cyclic olefins, and/or acetylenes), which include steam cracker pyrolysis gasoline 548, FCC light cracked naphtha 550, delayed coker light naphtha, Fischer Tropsch C5+ olefins, and Methanol to Olefins (MTO) C₅₊ olefins 552. One or a combination of these C₅₊ hydratable streams can be directed into the hydration unit 512 which converts the unsaturated hydrocarbons substantially (at least about 50%, 55%, 60%, 65%, 70%, 75%, 80%, 85%, 90%, 95% (vol %, wt %, or mol %), or more) to oxygenates compounds. In some cases, the oxygenates compounds comprise one or more of 1,5-pentanediol, 1,6-hexanediol, cyclohexanol, 3-hexanol, 4-methyl-2-pentanol, 3-methyl-3-pentanol, 3,3-dimethyl-2-butanol, 2-pentanol, 3-methyl-2-butanol, tertiary amyl alcohol, and combinations thereof. In some cases, the product stream from the hydration unit is further passed through one or more separation units 554 for separating the product stream into one or more end products such as gasoline 516 and C₅/C₆ oxygenates 556.

### Transalkylation Process

Also provided in the present disclosure are methods and systems for generating higher molecular weight aromatics with reduced amount of aromatic species that may at least partially deactivate at least a portion of the ETL catalyst. In some cases, such generated higher molecular weight aromatics comprises aromatics with eight hydrocarbons (C₈ aromatics). As described above and elsewhere herein, in an ETL process, unsaturated hydrocarbons (e.g., C₂H₄) are converted to higher molecular weight hydrocarbons with the aid of an ETL catalyst. The resulted higher molecular weight hydrocarbons may comprise aromatics with five or more carbon atoms (C₅₊ aromatics) including e.g., C₆, C₇, C₈ and C₉₊ aromatics. In some instances, the C₉₊ aromatic species are precursors to catalyst deactivation due to coke formation and pore blockage, and methods and systems to minimize/remove the C₉₊ aromatics from the reaction are expected to prolong the ETL catalyst life. In a transalkylation process, C₉₊ aromatics can be reacted with C₆/C₇ aromatics to selectively form C₈ aromatics and minimize the formation of heavy aromatics.

In some cases, the methods comprise directing an unsaturated hydrocarbon feed stream comprising C₂H₄ into an ETL unit which reacts the C₂H₄ in an ETL process to yield higher hydrocarbon products. The yielded higher hydrocarbon products may comprise saturated and unsaturated higher hydrocarbons (e.g., aromatics). The ETL unit may comprise one or more ETL reactors. Each of the ETL reactors may comprise an ETL catalyst that facilitates an ETL reaction. In some cases, the ETL reactors may further comprise a transalkylation catalyst which facilitates a transalkylation reaction in the ETL reactors. During the transalkylation reaction, at least a portion of the higher hydrocarbon products generated in the ETL reaction is further reacted to minimize the formation of C₉₊ aromatics and to produce C₈ aromatics. ETL product stream generated in the reactor may comprise C₈ aromatics at concentrations that are increased relative to the respective concentrations of C₈ aromatics in ETL product stream produced in the absence of the transalkylation catalyst. In some cases, the concentration of C₈ aromatics (e.g., among total aromatics in the ETL product) in the ETL product stream is increased by at least about 5%, 10%, 12%, 14%, 16%, 18%, 20%, 22%, 24%, 26%, 28%, 30%, 35%, 40%, 45%, 50% or more, as compared to the concentration of C₈ aromatics in ETL product stream generated without using the transalkylation catalyst.

The ETL reaction and the transalkylation reaction can be conducted sequentially or substantially simultaneously. The ETL reaction and the transalkylation reaction are conducted substantially simultaneously where the transalkylation reaction starts as soon as higher hydrocarbon products are generated in the ETL reaction. In some cases, the transalkylation reaction starts less than or equal to about 1 hour, 50 minutes (min), 40 min, 30 min, 25 min, 20 min, 15 min, 10 min, 9 min, 8 min, 7 min, 6 min, 5 min, 4 min, 3 min, 2 min, 1 min or less after the higher hydrocarbon products are generated in the ETL reaction. In some cases, ETL reaction and the transalkylation reaction are conducted under substantially the same reaction condition. For example, both reactions are performed in the same ETL reactor which is operated under the same conditions, including e.g., temperature, pressure, and residence time.

Alternatively or additionally, an ETL reactor may be a multi-tubular reactor which comprises multiple zones and arrangements within the reactor shell and reaction conditions within each zone may be independently set and controlled. In cases where a multi-tubular reactor is utilized, ETL reaction and transalkylation reaction may be conducted under different conditions. As an example, multiple reactor temperature zones can allow for a first temperature zone to start ETL reaction while having another zone operated under a different temperature to facilitate the transalkylation reaction of higher hydrocarbons generated in the ETL reaction.

ETL catalysts used in the methods and systems can be any types of ETL catalysts or oligomerization catalysts as described above and elsewhere herein. For example, the ETL catalysts can comprise zeolites such as erionite, zeolite 4A, zeolite 5A and MFI topology of zeolite. Non-limiting examples of ETL catalysts may include ZSM-5, ZSM-11, ZSM-12, ZSM-21, ZSM-23, ZSM-35, ZSM-38, and mixtures thereof. The zeolites can be doped or undoped. For example, the ETL catalysts may be ZSM-5 comprising undoped ZSM-5, ZSM-5 doped with W, ZSM-5 doped with Mo, ZSM-5 doped with Ga, ZSM-5 doped with La, ZSM-5 doped with Ni, ZSM-5 doped with Fe, ZSM-5 doped with Co, and ZSM-5 doped with combinations of multiple dopants.

Any catalyst that can facilitate a transalkylation reaction can be used as transalkylation catalyst in the present disclosure. The transalkylation catalyst may comprise zeolites such as zeolites containing 12-ring channel systems. In some cases, the zeolites comprise beta-zeolite and mordenite. The transalkylation catalyst may further comprise one or more metals including rhenium, platinum, nickle, and combinations thereof. Examples of transalkylation catalysts include, but are not limited to beta zeolite, zeolite X, zeolite Y, Ultrastable Y (USY), Dealuminized Y (Deal Y), mordenite, NU-87, ZSM-3, ZSM-4 (Mazzite), ZSM-12, ZSM-18, MCM-22, MCM-36, MCM-49, MCM-56, EMM-10, EMM-10-P and ZSM-20.

ETL catalysts and transalkylation catalysts may or may not be of the same type. The transalkylation catalyst may be physically admixed with the ETL catalyst. Physical admixtures of the catalysts may be in the form of individual particles. The catalyst particles may comprise multiple layers and the ETL catalyst and the transalkylation catalyst may be in the same layer of the catalyst particles. In some cases, the ETL catalyst and the transalkylation catalyst are in separate layers of the catalyst particles. In some cases, the transalkylation catalyst is sandwiched between layers of the ETL catalyst.

One or both of the ETL catalyst and transalkylation catalyst may be porous. The average pore size of the ETL catalyst may or may not be the same as that of the transalkylation catalyst. In some cases, the ETL catalyst has a smaller average pore size than the transalkylation catalyst. The average pore size of the ETL catalyst may be greater than or equal to about 1 angstrom (Å), 2 Å, 3 Å, 4 Å, 5 Å, 6 Å, 7 Å, 8 Å, 9 Å, 10 Å or more. In some cases, the ETL catalyst has an average pore size that falls between any of the two values described herein, for example, between 4 Å and 7 Å, and between 6 Å and 9 Å. The average pore size of the transalkylation catalyst may vary. For example, in some cases, the average pore size of the transalkylation catalyst is at least about 4 Å, 5 Å, 6 Å, 7 Å, 8 Å, 9 Å, 10 Å, 11 Å, 12 Å, or more. In some cases, the average pore size of the transalkylation catalyst is between two values described herein, for example, between 7 Å and 9 Å.

With the presence of transalkylation catalyst in the reactor, ETL catalyst may have a lifetime that is greater than a lifetime of the ETL catalyst in the absence of transalkylation catalyst. In some cases, the ETL catalyst has a lifetime that is at least about 1.1 times, 1.2 times, 1.3 times, 1.4 times, 1.5 times, 1.6 times, 1.7 times, 1.8 times, 1.9 times, 2 times, 2.2 times, 2.3 times, 2.4 times, 2.5 times, 2.6 times, 2.7 times, 2.8 times, 2.9 times, 3 times, 3.5 times, 4 times, 4.5 times, or 5 times greater than the lifetime of the ETL catalyst in the absence of transalkylation catalyst in the ETL reactor.

### ETL Process Using Oxygen Containing Feed Stream

In ETL process, hydrogen molecules can be adsorbed and dissociated by an ETL catalyst comprising metals (e.g., a gallium-loaded acid support ZSM-5 zeolite). The migration of hydrogen atoms from the metal catalyst onto the nonmetal support or adsorbate comprises the spillover phenomenon, which occurs over strong hydrogenation/dehydrogenation metals in the presence of hydrogen. It may cause hydrogen gas to dissociate into hydrides that are easily bound to the metal site, thereby inhibiting the site's ability to dehydrogenate/hydrogenate hydrocarbons, and reduces the available metal sites for activating hydrogenation/dehydrogenation reactions.

Provided herein are methods and systems for enhancing dehydrogenation activities of ETL catalysts and generating higher hydrocarbon compounds using the ETL catalysts in an ETL process. The methods may comprise directing an unsaturated hydrocarbon feed stream comprising ethylene, as well as an oxygen (O₂) containing stream into an ETL reactor which, in the presence of O₂, converts the ethylene in an ETL reaction to yield a product stream comprising one or more higher hydrocarbon compounds. The concentration of O₂ may vary. The O₂ containing stream may comprise O₂ at a concentration that is selected to enhance a dehydrogenation activity of the ETL catalyst. The enhanced dehydrogenation activity of the ETL catalyst may be determined by a yield of the ETL product stream in the presence of O₂ relative to a yield of the product stream in the absence of O₂ at the same concentration. In some cases, the concentration of O₂ is selected so as to enhance the dehydrogenation activity of a given catalyst by a factor of at least about 1.01, 1.02, 1.03, 1.04, 1.05, 1.06, 1.07, 1.08, 1.09, 1.10, 1.20, 1.30, 1.40, 1.50, 1.60, 1.70, 1.80, 1.90, 2.00, 2.20, 2.40, 2.60, 2.80, 3.00, 3.50, 4.00, 4.50, 5.00, 6.00, 7.00, 8.00, 9.00, 10.0 or higher. In some cases, O₂ is at a concentration less than or equal to about 5%, 4%, 3%, 2%, 1%, 0.9%, 0.8%, 0.7%, 0.6%, 0.5%, 0.4%, 0.3%, 0.2%, 0.1%, 0.075%, 0.05%, 0.025%, 0.01%, 0.0075%, 0.005%, 0.0025%, 0.001% or less (vol %) of ethylene (or ETL feed stream) directed into the ETL reactor. In some cases, the concentration of O₂ is between any of the two values described herein, for example, between about 0.005 and 1 vol % of ethylene (or ETL feed stream) which is fed into the ETL reactor.

In some cases, at least a portion of ETL feed stream and/or O₂ is generated in and received from one or more different processing units (or systems) that are in fluidic communication with the ETL unit, for example, an OCM unit. As an example, the methods and systems of the present disclosure may further comprise one or more OCM units. The OCM units may be configured to receive methane and an oxidizing agent (e.g., O₂) and react the methane and the oxidizing agent in an OCM process to generate an OCM product stream comprising ethylene. At least a portion of ethylene generated in the OCM units may be directed into the ETL reactor for producing higher hydrocarbon compounds. Additionally or alternatively, unreacted O₂ from the OCM units may be routed to the ETL unit along with the stream of ethylene. The OCM units may be integrated with the ETL unit. In some cases, the OCM units are retrofitted into an existing system comprising the ETL unit. In some cases, both the OCM units and ETL units are retrofitted into an existing system which comprises one or more additional processing units including, e.g., metathesis units, fluid catalytic cracking (FCC) units, thermal cracker units, coker units, methanol to olefins (MTO) units, Fischer-Tropsch units, and a combination thereof.

### ETL Processes Including Catalytic Distillation

### I. Ni-Based ETL Via Catalytic Distillation

ETL technology can be used to take OCM effluent or refinery offgas streams as feedstocks for the manufacture of higher hydrocarbons from the stream's light olefins (e.g. ethylene and propylene). The higher hydrocarbon product stream can comprise paraffins, isoparaffins, olefins, naphthenes, aromatics, or combinations thereof.

Ways to increase process versatility by altering the choice of product stream can improve process flexibility. One potential way is to gear the ETL process such as to maximize olefins production, where later the higher olefins can be used downstream for multiple uses (e.g. to alcohols, ethers, epoxides, aldehydes etc).

Concurrently, methodologies to reduce capital cost and the number of unit operations associated with the ETL process are described herein, as this can add to the technology competitiveness, diversity, and flexibility. One such methodology lies in catalytic distillation, which combines reaction and separation of products in the same vessel, and enables a high level of conversion of reactants due to continuous removal of products (as per Le Chatelier's principle), which drives the equilibrium of the reaction towards the products.

One aspect of the present invention provides an ETL process that is based on the initial step of oligomerization of light olefins (e.g. ethylene, propylene, and/or butenes) into higher olefins, with minimal conversion to hydrocarbons other than olefins (e.g. paraffins, isoparaffins, naphthenes, and aromatics). This may be accomplished over supported catalysts geared towards oligomerization at moderate process conditions. Simultaneously, the reaction step of oligomerizing ethylene into C₄₊ olefins and the separation of olefins into a C₄ cut and a C₆₊ cut can be accomplished over a catalytic distillation unit, as shown in FIG. 24.

FIG. 24 shows a schematic of a catalytic distillation column 2400. In this schematic, a stream containing ethylene 2401 enters as feed into a catalytic distillation column 2402 where it may be put into contact with an oligomerization catalyst, reacts, and forms C₄₊ olefins. The temperature and pressure of the column are selected such that formed C₆₊ olefins condense into a liquid that move downward in the column while C₄ vapors move upward. Unconverted ethylene 2403 may be routed back into the stream containing ethylene 2401 and butane product may be partially condensed in a condenser 2404 and refluxed back into the column to help maintain a liquid/vapor equilibrium/mixture as well as absorb any C₆₊ olefins entrained with the vapor stream. The C₆₊ product stream 2405 may be partially vaporized in a reboiler 2406 and refluxed back as vapor stream that helps maintaining the vapor/liquid equilibrium/mixture in the column as well as strip any liquid C₄ that may be falling below the reaction zone of the column. Refluxing higher amounts of C4 back into the column may increase the residence time of butane around the oligomerization catalyst, which may lead to higher conversion of butenes into higher olefins, potentially eliminating butenes production from the overall process (when operating in full-reflux mode).

In some embodiments, at least some of the stream containing ethylene 2401 may be generated in an oxidative coupling of methane (OCM) system.

The temperature in the column can range from about 10° C. to about 400° C., about 50° C. to about 400° C., about 100° C. to about 400° C., about 150° C. to about 400° C., about 50° C. to about 300° C., about 10° C. to about 250° C., or about 50° C. to about 200° C. The pressure in the column can range from about 1 bar to about 20 bar, about 1 bar to about 15 bar, about 1 bar to about 10 bar, about 1 bar to about 5 bar, or about 0.5 bar to about 10 bar.

In some embodiments, a higher pressure is employed in the catalytic distillation column, such that butenes as well as C₆₊ may be condensed once formed through oligomerization, and exit into a second column where separation of C₄ and C₆₊ may be accomplished. This can allow for a smaller oligomerization catalyst bed since higher pressures may favor an increased conversion of ethylene into higher olefins. Options to maximize the conversion of butenes into higher olefins may also be possible in this configuration by regulating the amount of C₄ reflux (vapor and/or liquid) back into the catalytic distillation column.

FIG. 25 shows a schematic for conducting catalytic distillation under elevated pressures 2500. A source containing ethylene 2501 is injected into a catalytic distillation tower 2502 to generate a stream containing unconverted ethylene and a stream containing C₄ and C₆₊ components.

The stream containing unconverted ethylene 2503 can be injected into the stream containing ethylene and/or recycled to the catalytic distillation column. Some of the stream containing C₄ and C₆₊ can be injected into a reboiler 2506 and injected into the catalytic distillation tower. The remainder of the stream containing C₄ and C₆₊ may be injected into a second distillation tower 2507 to produce a stream containing butane and a stream containing C₆₊ hydrocarbons 2505. The stream containing butane can be injected into a condenser 2504 that condenses butane vapor. The liquid butane product from the condenser can then be injected into the catalytic distillation tower.

In some embodiments, an oxidizing agent, such as O₂, air, water, or combinations thereof, can be fed along with the column feed (which typically contains H₂), such as to minimize/limit the extent of ethylene/propylene hydrogenation over the oligomerization catalysts—a phenomenon that may take place over highly active oligomerization catalysts resulting in loss of olefins into paraffins, thereby reducing oligomer yield.

In some cases, CO contained in ETL feeds can convert readily via Fischer-Tropsch reactions with H₂ into C₁-C₄ paraffins, minimizing the adverse impact it can have over the oligomerization metal (such as Ni) such as etching.

In some cases, a hydrotreating catalyst layer (or separate reaction zone) upstream of the ETL reactor/column can be employed to remove sulfur from certain ETL feeds. This can be in the form of a hydrotreating catalyst layer, composed of CoMo- or NiMo-based catalyst (which can react sulfur and not saturate olefins in the feed over the used process conditions), or in the form of a separate and upstream hydrotreating unit, or a CoMo/NiMo based unit as described for the case of hydrotreating layer above.

The choice of active metal for effecting oligomerization of light olefins into higher olefins can be any one or combination of Ni, Pd, Cr, V, Fe, Co, Ru, Rh, Cu, Ag, Re, Mo, W, Mn, and Pt, and with up to a total loading of 20% by weight of catalyst mass—Catalyst support can range between one or any combination of zeolites (such as ZSM-5, Beta, and ZSM-11), amorphous silica alumina, silica, alumina, mesoporous silica, mesoporous alumina, zirconia, titania, and pillared clay. The operating conditions of the ETL unit to suit optimal conversion and high olefin yield out of the ETL reactor/column may be in the range of 50-200° C. and 10-80 bar while effecting the condensation of part or all of formed higher olefins.

I. ETL with C₅₊ Etherification Via Catalytic Distillation

In some cases, ETL technology produces a C₅₊ liquid product that is rich in olefins, where around 20-35 wt % of the product may be constituted by olefins. Federal and state specifications with respect to gasoline fuel limit the amount of olefins that can be blended into gasoline, to be around 4-6 wt % in total. Hence, a cost-effective solution can be developed where the olefin amount is reduced to meet specifications.

In addition, there are other sources of C₅₊ streams that may contain hydratable olefins, di-olefins, cyclic olefins, and/or acetylenes, including steam cracker pyrolysis gasoline, FCC light cracked naphtha, delayed coker light naphtha, Fischer Tropsch C₅₊ olefins, and Methanol to Olefins (MTO) C₅₊ olefins. One or a combination of the aforementioned C₅₊ unsaturated streams can be available at any given time when OCM/ETL is deployed, presenting an opportunity to boost the production of an ether-containing C₅₊ liquid product.

FCC light cracked naphtha can contain about 60% olefins, and can be subject to a hydrotreating step to minimize olefins so as to meet gasoline specifications.

Steam cracker pyrolysis gasoline can contain up to about 75% of olefins, di-olefins, cyclic olefins, and triple bond hydrocarbons. The stream can go through two steps of hydrogenation to saturate triple bond and di-olefinic molecules. The etherification of pyrolysis gasoline C₅₊ molecules (without hydrotreating) can result in formation of C₆₊ ethers.

C₆₊ ethers can be considered potentially superior oxygenates to conventional ones such as ethanol, since they contain less oxygen per unit mass or volume, allowing blending more of them compared to ethanol before reaching the maximum oxygen limit of gasoline. Also, some of the smaller ethers such as MTBE have had concerns associated with their contamination of underground water, promoting its ban in the USA. Finally, some of the higher ethers may be usable as diesel fuel additives.

Etherifying C₅₊ olefins, di-olefins, cyclic olefins, and/or acetylenic compounds originating from FCC light naphtha, steam cracking pyrolysis gas, metathesis, ETL, delayed coker light naphtha, MTO, or Fischer-Tropsch units may substantially increase the amount of C₆₊ ethers that are blendable into gasoline/diesel, thereby increasing gasoline/diesel volumes.

Concurrently, methodologies to reduce capital cost and the number of unit operations associated with the ETL process can be introduced, as this can add to the technology competitiveness, diversity, and flexibility. One such methodology lies in catalytic distillation, which combines reaction and separation of products in the same vessel, and enables a high level of conversion of reactants due to continuous removal of products (as per Le Chatelier's principle), which drives the equilibrium of the reaction towards the products.

An aspect of invention provides an ETL process modification/add-on, wherein the C₅₊ effluent, which may be composed of paraffins, isoparaffins, olefins, naphthenes, and aromatics, may be sent to an etherification catalytic distillation unit operating at etherification conditions, where the stream may contact an alcohol (such as methanol, isopropanol, glycerol etc.) such that C₅₊ olefins may substantially convert to C₆₊ ethers. In addition, C₅₊ olefins, di-olefins, cyclic olefins, and/or acetylenic compounds produced from FCC, steam cracker, metathesis, coker, MTO, or FT units may also be sent to the same etherification reactor/column, thereby boosting gasoline/diesel production.

FIG. 26 shows a process scheme for C₅₊ etherification via catalytic distillation 2600. In this schematic, unsaturated C₅₊ hydrocarbon stream 2601 enters as feed into the catalytic distillation column 2602 where it may be placed into contact with the etherification catalyst along with an alcohol stream 2603 that is concurrently introduced to the column, reacts with the alcohol and forms C₆₊ oxygenates. The temperature and pressure of the column may be selected such that formed C₆₊ oxygenates may condense into a liquid that moves downward in the column while unreacted C₅₊ hydrocarbon vapors may move up (the alcohol may be consumed completely). Some of the unconverted C₅₊ hydrocarbon product may be condensed and refluxed back into the column to help maintain a liquid/vapor equilibrium/mixture as well as absorb any C₆₊ oxygenates entrained with the vapor stream using a reflux condenser 2604. The C₆₊ oxygenates product stream may be partially vaporized in a reboiler 2605 and refluxed back as vapor stream that helps maintaining the vapor/liquid equilibrium/mixture in the column as well as strip any liquid C₅₊ hydrocarbon that may fall below the reaction zone of the column. Refluxing higher amounts of C₅₊ hydrocarbons back into the column may increase the residence time of C₅₊ olefins around the etherification catalyst, which can lead to higher conversion of olefins with alcohol and into C₆₊ oxygenates.

The etherification temperature can be selected from the range of 20 to 400° C., 50 to 400° C., 75 to 400° C., 100 to 400° C., 100 to 350° C., or 100 to 300° C. The etherification pressures can range from 1 to 100 bar. The alcohol to olefin mole ratio can be in the range of 0.01 to 20. Contact time of the reacting C₅₊ olefin and the etherification catalyst can be from 0.1 to 20 h⁻¹. The etherification catalyst can be a solid acid catalyst (e.g. ionic exchange resin, acidic zeolite, metal oxide).

As explained above, the temperature, pressure, alcohol/unsaturate ratio, choice of etherification catalyst, and contact time can be varied to reach an acceptable level of conversion into C₆₊ oxygenates from the process. Operation of the reboiler and condenser units such as to regulate the reflux ratios of C₅₊ hydrocarbon liquid/vapor and C₆₊ oxygenates vapor back into the catalytic distillation column can be varied. The number of trays and/or height of packed catalyst bed used inside the column can be varied. The location of the catalyst bed inside the column can be varied. The location of the C₅₊ and alcohol feeds into the column can be varied. The location of the column top product draw can be varied. The location of introducing the condenser reflux stream(s) back into the column can be varied. The location of the column bottom product draw can be varied. The location of introducing the reboiler reflux stream(s) back into the column can be varied.

III. ETL Process with C₅₊ Hydration Via Catalytic Distillation

In some cases, ETL produces a C₅₊ liquid product that is rich in olefins, where around 20-35 wt % of the product is constituted by olefins. Federal and state specifications with respect to gasoline fuel limit the amount of olefins that can be blended into gasoline, to be around 4-6 wt % in total. Hence, a cost-effective solution can be developed where the olefin amount is reduced to meet specifications.

In addition, there are other sources of C₅₊ streams that contain hydratable olefins, di-olefins, cyclic olefins, and/or acetylenes, including steam cracker pyrolysis gasoline, FCC light cracked naphtha, delayed coker light naphtha, Fischer Tropsch C₅₊ olefins, and Methanol to Olefins (MTO) C₅₊ olefins. One or a combination of the aforementioned C₅₊ hydratable streams can be available at any given time when OCM/ETL is deployed, presenting an opportunity to boost the production of C₅₊ alcohols.

FCC light cracked naphtha can contain 60% olefins, and can be subject to a hydrotreating step to minimize olefins so as to meet gasoline specifications.

Steam cracker pyrolysis gasoline can contain up to 75% of olefins, di-olefins, cyclic olefins, and triple bond hydrocarbons. The stream typically goes through two steps of hydrogenation to saturate triple bond and di-olefinic molecules.

C₅₊ alcohols can be considered potentially superior oxygenates to conventional ones such as ethanol, since they contain less oxygen per unit mass or volume, allowing blending more of them compared to ethanol before reaching the maximum oxygen limit of gasoline. In addition, they are much less soluble in water, resulting in the ability to blend them into gasoline from the bulk plant, unlike ethanol which has to be blended at the station due to water ingression issues. The energy density of C₅₊ alcohols is substantially larger than that of ethanol, resulting in the consumption of less C₅₊ alcohol material to arrive at the same mileage attained by ethanol. Finally, the Reid vapor pressure of C₅₊ alcohols is extremely low compared to that of ethanol, being close to or less than 1.0 psi.

Hydrating C₅₊ olefins, di-olefins, cyclic olefins, and/or acetylenic compounds originating from FCC light naphtha, steam cracking pyrolysis gas, metathesis, ETL, delayed coker light naphtha, MTO, or Fischer-Tropsch units may substantially increase the amount of C₅₊ alcohols that are blendable into gasoline, thereby increasing gasoline volumes.

Concurrently, methodologies to reduce capital cost and the number of unit operations associated with the ETL process can be developed, as this can add to the technology competitiveness, diversity, and flexibility. One such methodology lies in catalytic distillation, which combines reaction and separation of products in the same vessel, and enables a high level of conversion of reactants due to continuous removal of products (as per Le Chatelier's principle), which drives the equilibrium of the reaction towards the products.

An aspect of the invention provides an ETL process modification/add-on, where the C₅₊ effluent, which may be composed of paraffins, isoparaffins, olefins, naphthenes, and aromatics, may be sent to a hydration catalytic distillation unit operating at hydration conditions, where the stream contacts water such that C₅₊ olefins may substantially convert to C₅₊ alcohols. In addition, C₅₊ olefins, di-olefins, cyclic olefins, and/or acetylenic compounds produced from FCC, steam cracker, metathesis, coker, MTO, or FT units may also be sent to the same hydration column/reactor, thereby boosting gasoline production.

FIG. 27 shows a schematic for C₅₊ hydration via catalytic distillation 2700. In this schematic, the unsaturated C₅₊ hydrocarbon stream 2701 and a water stream 2702 enters as feed into the catalytic distillation column 2703 where it may be put into contact with the hydration catalyst along with water that is concurrently introduced to the column, reacts with water and forms C₅₊ oxygenates. The temperature and pressure of the column may be selected such that formed C₅₊ oxygenates may condense into a liquid that moves downward in the column while unreacted C₅₊ hydrocarbon vapors may move up along with unconverted water. Water may be first condensed in a first condenser 2704 and recycled back to the column, while some of the unconverted C₅₊ hydrocarbon product may be condensed in a second condenser 2705 and refluxed back into the column to help maintain a liquid/vapor equilibrium/mixture as well as absorb any C₅₊ oxygenates entrained with the vapor stream. The C₅₊ oxygenates product stream may be partially vaporized in a reboiler 2706 and refluxed back as vapor stream that helps maintaining the vapor/liquid equilibrium/mixture in the column as well as strip any liquid C₅₊ hydrocarbon and/or water that may be falling below the reaction zone of the column. Refluxing higher amounts of C₅₊ hydrocarbons back into the column may increase the residence time of C₅₊ olefins around the hydration catalyst, which can lead to higher conversion of olefins with water and into C₅₊ oxygenates.

The hydration conditions can be selected from the range of 100 to 300° C., and pressures ranging from 1-100 bar, and water to olefin mole ratio of 0.01-20. Contact time of the reacting C5+ olefin and the hydration catalyst can be from 0.1-20 h⁻¹. The hydration catalyst can be a solid acid catalyst (e.g. ionic exchange resin, acidic zeolite, metal oxide).

As explained above, the temperature, pressure, water-unsaturate ratio, choice of hydration catalyst, and contact time can be varied to reach an acceptable level of conversion into C5+ oxygenates from the process. Operation of the reboiler and condenser units such as to regulate the reflux ratios of C5+ hydrocarbon liquid/vapor and C5+ oxygenates vapor back into the catalytic distillation column can be varied. Number of trays and/or height of packed catalyst bed used inside the column can be varied. Location of the catalyst bed inside the column can be varied. Location of the C5+ and water feeds into the column can be varied. Location of the column top product draw can be varied. Location of introducing the condenser reflux stream(s) back into the column can be varied. Location of the column bottom product draw can be varied. Location of introducing the reboiler reflux stream(s) back into the column can be varied.

### IV. Ni-Based ETL and Etherification Via Catalytic Distillation

In some cases, ETL technology in its current form takes OCM effluent or refinery offgas streams as feedstocks for the manufacture of higher hydrocarbons from the stream's light olefins (e.g. ethylene and propylene). The higher hydrocarbon product stream may comprise paraffins, isoparaffins, olefins, naphthenes, aromatics, or combinations thereof.

Ways to increase process versatility by altering the choice of product stream are needed to improve process flexibility and potentially profitability. One potential way is to gear the ETL process such as to maximize olefins production, with further conversion of olefins into higher value products such as ethers and oxygenates.

C₆₊ ethers are considered potentially superior oxygenates to conventional ones such as ethanol, since they contain less oxygen per unit mass or volume, allowing blending more of them compared to ethanol before reaching the maximum oxygen limit of gasoline. Also, some of the smaller ethers such as MTBE have had concerns associated with their contamination of underground water, promoting its ban in the USA. Finally, some of the higher ethers are usable as diesel fuel additives.

Concurrently, methodologies to reduce capital cost and the number of unit operations associated with the ETL process are needed, as this can add to the technology competitiveness, diversity, and flexibility. One such methodology lies in catalytic distillation, which combines reaction and separation of products in the same vessel, and enables a high level of conversion of reactants due to continuous removal of products (as per Le Chatelier's principle), which drives the equilibrium of the reaction towards the products.

In one aspect of the disclosure, the ETL process is based on the initial step of oligomerization of light olefins (e.g. ethylene, propylene, and/or butenes) into higher olefins, with minimal conversion to hydrocarbons other than olefins (e.g. paraffins, isoparaffins, naphthenes, and aromatics). This may be accomplished over supported catalysts geared towards oligomerization at moderate process conditions. Simultaneously, the reaction step of oligomerizing ethylene into C4+ olefins and the separation of olefins into a C₄ cut and a C₆₊ cut may be accomplished over a catalytic distillation unit, as shown in FIG. 28. Successively, the formed C₆₊ olefins may react with an alcohol over an etherification catalyst to form C₇₊ oxygenates, which may occur in the same catalytic distillation unit.

FIG. 28 shows an ETL process based on the initial step of oligomerization and catalytic distillation. In this schematic, ethylene 2801 enters as feed into the catalytic distillation column 2803 where it gets into contact with the oligomerization catalyst in a first catalytic bed, reacts, and forms C₄₊ olefins. The temperature and pressure of the column may be selected such that formed C₆₊ olefins may condense into a liquid that moves downward in the column while C₄ vapors may move up.

Unconverted ethylene may be routed back into the column entrance and butene product may be partially condensed in a condenser 2804 and refluxed back into the catalytic distillation column to help maintain a liquid/vapor equilibrium/mixture as well as absorb any C₆₊ olefins entrained with the vapor stream. The downward-flowing C₆₊ olefins may get in contact with an alcohol stream 2802 that is introduced into the column and over an etherification catalyst to react (till full extinction of the alcohol) and produce C₇₊ oxygenates that may move further down in the column. The C₇₊ oxygenate product stream is partially vaporized in a reboiler 2806 and refluxed back as vapor stream that helps maintaining the vapor/liquid equilibrium/mixture in the column as well as strip any liquid C₄ and/or alcohol that is falling below the reaction zone(s) of the column. Refluxing higher amounts of C₄ back into the column may increase the residence time of butene around the oligomerization catalyst, which can lead to higher conversion of butenes into higher olefins, potentially eliminating butenes production from the overall process (when operating in full-reflux mode). Additionally or alternatively, refluxing higher amounts of C₆₊ hydrocarbons back into the column may increase the residence time of C₆₊ olefins around the etherification catalyst, which can lead to higher conversion of olefins with alcohol and into C₇₊ oxygenates.

The oligomerization and etherification conditions can be selected from the range of 100 to 200° C., and pressures ranging from 10-80 bar, and alcohol to olefin mole ratio of 0.01-20. Contact time of the reacting C₆₊ olefin and the etherification catalyst, and that of ethylene and the oligomerization catalyst can be from 0.1-20 h⁻¹. The etherification catalyst can be a solid acid catalyst (e.g. ionic exchange resin, acidic zeolite, metal oxide).

An oxidizing agent, such as O₂, air, or water, can be fed along with the column feed (which may contain H₂), such as to minimize/limit the extent of ethylene/propylene hydrogenation over the oligomerization catalysts—a phenomenon that may take place over highly active oligomerization catalysts resulting in loss of olefins into paraffins, thereby reducing oligomer yield.

In some case, CO contained in ETL feeds may convert readily via FT reactions with H₂ into C₁-C₄ paraffins, minimizing the adverse impact it can have over the oligomerization metal (such as Ni) such as etching.

In some cases, a hydrotreating catalyst layer (or separate reaction zone) upstream of the ETL reactor/column can be employed to remove sulfur from certain ETL feeds. This can be in the form of a hydrotreating catalyst layer, composed of CoMo or NiMo based catalyst (which may react sulfur and not saturate olefins in the feed over the used process conditions), or in the form of a separate and upstream hydrotreating unit, which can be a MEROX type unit (employing a liquid catalyst) or a CoMo/NiMo based unit as described for the case of hydrotreating layer above.

The choice of active metal for effecting oligomerization of light olefins into higher olefins over the first catalyst bed can be any one or combination of Ni, Pd, Cr, V, Fe, Co, Ru, Rh, Cu, Ag, Re, Mo, W, Mn, and Pt, and with up to a total loading of 20% by weight of catalyst mass. Catalyst support can range between one or any combination of zeolites (such as ZSM-5, Beta, and ZSM-11), amorphous silica alumina, silica, alumina, mesoporous silica, mesoporous alumina, zirconia, titania, and pillared clay. Additional variables in the process include the operating conditions of the ETL catalytic distillation unit to suit optimal conversion and high oxygenates yield out of the ETL reactor/column (about 100-200° C. and about 10-80 bar) while effecting the condensation of part or all of formed higher olefins and oxygenates; choice of unit and associated operating conditions and catalyst employed for the upstream hydrotreating unit (if included) for removing sulfur; the ratio of oxidizing agent to feed hydrogen content to suppress olefin hydrogenation reactions; operation of the reboiler and condenser units such as to regulate the reflux ratios of C₄ liquid/vapor and C₆₊ vapor back into the catalytic distillation column; number of trays and/or height of packed catalyst beds used inside the column; location of the catalyst beds inside the column; location of the feeds into the column; location of the column top product draw; location of introducing the condenser reflux stream(s) back into the column; location of the column bottom product draw; location of introducing the reboiler reflux stream(s) back into the column; alcohol-olefin ratio, choice of etherification catalyst, and contact time can be varied to reach an acceptable level of conversion into C₇₊ oxygenates from the process; location of ethylene and alcohol feeds into the column.

### V. Ni-Based ETL and Hydration Via Catalytic Distillation

In some cases, ETL technology in its current form takes OCM effluent or refinery offgas streams as feedstocks for the manufacture of higher hydrocarbons from the stream's light olefins (e.g. ethylene and propylene). The higher hydrocarbon product stream may comprise paraffins, isoparaffins, olefins, naphthenes, aromatics, or combinations thereof.

Ways to increase process versatility by altering the choice of product stream are needed to improve process flexibility and potentially profitability. One potential way is to gear the ETL process such as to maximize olefins production, with further conversion of olefins into higher value products such as ethers and oxygenates.

C₆₊ alcohols are considered potentially superior oxygenates to conventional ones such as ethanol, since they contain less oxygen per unit mass or volume, allowing blending more of them compared to ethanol before reaching the maximum oxygen limit of gasoline. In addition, they are much less soluble in water, resulting in the ability to blend them into gasoline from the bulk plant, unlike ethanol which has to be blended at the station due to water ingression issues. The energy density of C₆₊ alcohols may be substantially larger than that of ethanol, resulting in the consumption of less C₆₊ alcohol material to arrive at the same mileage attained by ethanol. Finally, the RVP of C₆₊ alcohols may be low compared to that of ethanol, being close to or less than 1.0 psi.

Concurrently, methodologies to reduce capital cost and the number of unit operations associated with the ETL process are needed, as this can add to the technology competitiveness, diversity, and flexibility. One such methodology lies in catalytic distillation, which combines reaction and separation of products in the same vessel, and enables a high level of conversion of reactants due to continuous removal of products (as per Le Chatelier's principle), which drives the equilibrium of the reaction towards the products.

In one aspect of the disclosure, the ETL process is based on the initial step of oligomerization of light olefins (e.g. ethylene, propylene, and/or butenes) into higher olefins, with minimal conversion to hydrocarbons other than olefins (e.g. paraffins, isoparaffins, naphthenes, and aromatics). This may be accomplished over supported catalysts geared towards oligomerization at moderate process conditions. Simultaneously, the reaction step of oligomerizing ethylene into C₄₊ olefins and the separation of olefins into a C₄ cut and a C₆₊ cut may be accomplished over a catalytic distillation unit, as shown in FIG. 29. Successively, the formed C₆₊ olefins may react with water over a hydration catalyst to form C₆₊ oxygenates, which may occur in the same catalytic distillation unit.

FIG. 29 shows a process for catalytic distillation hydration and oligomerization with ETL. A stream containing ethylene 2901 and a stream containing water 2907 enters as feed into the catalytic distillation column 2903 where it may get into contact with the oligomerization catalyst in a first catalytic bed, reacts, and forms C₄₊ olefins. The temperature and pressure of the column my be selected such that formed C₆₊ olefins may condense into a liquid that moves downward in the column while C₄ vapors may move up. Unconverted ethylene may be condensed in a first condenser 2904 routed back into the column entrance and butene product may be partially condensed (in a second condenser 2905 following a first condenser that separates water that is recycled back into the column as feed) and refluxed back into the column to help maintain a liquid/vapor equilibrium/mixture as well as absorb any C₆₊ olefins entrained with the vapor stream. The downward-flowing C₆₊ olefins may get into contact with water that is introduced into the column and over a hydration catalyst to react and produce C₆₊ oxygenates that may move further down in the column. The C₆₊ oxygenate product stream may be partially vaporized in a reboiler 2906 and refluxed back as vapor stream that helps maintain the vapor/liquid equilibrium/mixture in the column as well as strip any liquid C₄ may be falling below the reaction zone(s) of the column. Refluxing higher amounts of C4 back into the column may increase the residence time of butene around the oligomerization catalyst, which can lead to higher conversion of butenes into higher olefins, potentially eliminating butenes production from the overall process (when operating in full-reflux mode). Refluxing higher amounts of C₆₊ hydrocarbons back into the column may increase the residence time of C₆₊ olefins around the hydration catalyst, which can lead to higher conversion of olefins with water and into C₆₊ oxygenates.

The oligomerization and hydration conditions can be selected from the range of 100 to 200° C., and pressures ranging from 10-80 bar, and alcohol to olefin mole ratio of 0.01-20. Contact time of the reacting C₆₊ olefin and the hydration catalyst, and that of ethylene and the oligomerization catalyst can be from 0.1-20 h⁻¹. The hydration catalyst can be a solid acid catalyst (e.g. ionic exchange resin, acidic zeolite, metal oxide).

An oxidizing agent, such as O₂, air, or water, can be fed along with the column feed (which may contain H₂), such as to minimize/limit the extent of ethylene/propylene hydrogenation over the oligomerization catalysts—a phenomenon that may take place over highly active oligomerization catalysts resulting in loss of olefins into paraffins, thereby reducing oligomer yield.

In some cases, CO contained in ETL feeds may convert readily via FT reactions with H₂ into C₁-C₄ paraffins, minimizing the adverse impact it can have over the oligomerization metal (such as Ni) such as etching.

In some cases, a hydrotreating catalyst layer (or separate reaction zone) upstream of the ETL reactor/column can be employed to remove sulfur from certain ETL feeds. This can be in the form of a hydrotreating catalyst layer, composed of CoMo or NiMo based catalyst (which may react sulfur and not saturate olefins in the feed over the used process conditions), or in the form of a separate and upstream hydrotreating unit, which can be a MEROX type unit (employing a liquid catalyst) or a CoMo/NiMo based unit as described for the case of hydrotreating layer above.

Aspects of this invention that can be varied include: the choice of active metal for effecting oligomerization of light olefins into higher olefins over the first catalyst bed can be any one or combination of Ni, Pd, Cr, V, Fe, Co, Ru, Rh, Cu, Ag, Re, Mo, W, Mn, and Pt, and with up to a total loading of 20% by weight of catalyst mass; Catalyst support can range between one or any combination of zeolites (such as ZSM-5, Beta, and ZSM-11), amorphous silica alumina, silica, alumina, mesoporous silica, mesoporous alumina, zirconia, titania, and pillared clay; the operating conditions of the ETL catalytic distillation unit to suit optimal conversion and high oxygenates yield out of the ETL reactor/column (about 100-200° C. and about 10-80 bar) while effecting the condensation of part or all of formed higher olefins and oxygenates—choice of unit and associated operating conditions and catalyst employed for the upstream hydrotreating unit (if included) for removing sulfur; The ratio of oxidizing agent to feed hydrogen content to suppress olefin hydrogenation reactions; Operation of the reboiler and condenser units such as to regulate the reflux ratios of C4 liquid/vapor and C₆₊ vapor back into the catalytic distillation column; Number of trays and/or height of packed catalyst beds used inside the column; Location of the catalyst beds inside the column—location of the feeds into the column; Location of the column top product draw; Location of introducing the condenser reflux stream(s) back into the column; Location of the column bottom product draw; Location of introducing the reboiler reflux stream(s) back into the column; Water-olefin ratio, choice of hydration catalyst, and contact time can be varied to reach an acceptable level of conversion into C₆₊ oxygenates from the process; Location of ethylene and water feeds into the column.

### VI. Dimerization/Alkylation Via Catalytic Distillation

Alkylation of olefins with isoparaffins may be used for the production of alkylate, a superior gasoline blendstock due to its unique characteristics such as high RON, no olefinic content, and low RVP, making it one of the most sought after streams for gasoline blenders. Processes for alkylation include solid acid based alkylation and alkylation process employing HF or sulfuric acid as the alkylation catalysts. These processes may have, however, some shortcomings such as the specification of feedstocks that go into them, such as being limited to isobutane and C₃₊ olefins as reactants.

Concurrently, methodologies to reduce capital cost and the number of unit operations associated with the ETL process are needed, as this can add to the technology competitiveness, diversity, and flexibility. One such methodology lies in catalytic distillation, which combines reaction and separation of products in the same vessel, and enables a high level of conversion of reactants due to continuous removal of products (as per Le Chatelier's principle), which drives the equilibrium of the reaction towards the products.

In one aspect of this disclosure, one of or a mixture of any of C₂-C₅ olefins may be introduced to a catalytic distillation unit, where a dimerization-alkylation catalyst may cause them to react upon contact with isobutane (iC₄) unit where production of higher olefins may be effected. In some cases, an olefin isomerization unit may be used upstream of the said catalytic distillation unit such that olefins (such as 1-butene) may be isomerized into a mixture of olefin isomers (such as 1-butene and cis-2-butene, and trans-2-butene).

FIG. 30 shows a schematic of dimerization/alkylation via catalytic distillation 3000. In this schematic, one or a mixture of any of C₂-C₅ olefins enters as feed 3003 into the catalytic distillation column 3002 in liquid phase, where it may get into contact with the dimerization-alkylation catalyst and a stream containing iC4 3001 which may also be introduced into the column, reacts, and forms alkylates (C₅₊). The temperature and pressure of the column may be selected such that formed C₈₊ alkylates may condense into a liquid that moves downward in the column while iC₄ and C₂—C olefins vapors may move up. By-product nC₄/nC₅ are lighter than alkylate, and they may be drawn out of the column as a side stream as shown in the schematic. Unconverted C₂-C₅ may be condensed in a condenser 3004 and routed back to the column along with fresh C₂-C₅ olefins and iC₄₋ The C₈₊ alkylate product stream may be partially vaporized in a reboiler 3005 and refluxed back as vapor stream that helps in maintaining the vapor/liquid equilibrium/mixture in the column as well as strip any liquid iC₄ that may be falling below the reaction zone of the column or nC₄/nC₅ by products.

The operating conditions and catalyst may include Ni, Pd, Cr, V, Fe, Co, Ru, Rh, Cu, Ag, Re, Mo, W, Mn, Pt, supported on any one or combination of zeolites, sulfated zirconia, tungstated zirconia, chlorided alumina, aluminum chloride (AlCls), silicon-aluminum phosphates, titaniosilicates (including VTM zeolite), polyphosphoric acid (including solid phosphoric acid, or SPA, catalysts, which are made by reacting phosphoric acid with diatomaceous earth), polytungstic acid, and supported liquid acids such as triflic acid on silica, sulfuric acid on silica, hydrogen fluoride on carbon, antimony fluoride on silica, aluminum chloride (AlCls) on alumina (Al₂O₃). The operating conditions, catalysts, and reactor type and configuration of the olefin isomerization unit (if included) which employs catalysts typically used for olefin isomerization such as alkaline oxides (including MgO) can be varied. Additional process variables include: The ratio of starting olefin to iC₄; Operation of the reboiler and condenser units such as to regulate the reflux ratios of C₂-C₅ olefins and iC₄ liquid/vapor and C₈₊ vapor back into the catalytic distillation column; Number of trays and/or height of packed catalyst bed used inside the column; Location of the catalyst bed inside the column; Location of the feed(s) into the column; Location of the column top product draw; Location of introducing the condenser reflux stream(s) back into the column; Location of the column bottom product draw; Location of introducing the reboiler reflux stream(s) back into the column; Location of the nC4/nC5 side draw stream.

### VII. 2-Bed Dimerization Followed by Alkylation

Alkylation of olefins with isoparaffins may be used for the production of alkylate, a superior gasoline blendstock due to its unique characteristics such as high RON, no olefinic content, and low RVP, making it one of the most sought after streams for gasoline blenders. Processes for alkylation include solid acid based alkylation and alkylation process employing HF or sulfuric acid as the alkylation catalysts. These processes may have, however, some shortcomings such as the specification of feedstocks that go into them, such as being limited to isobutane and C₃₊ olefins as reactants.

Concurrently, methodologies to reduce capital cost and the number of unit operations associated with the ETL process are needed, as this can add to the technology competitiveness, diversity, and flexibility. One such methodology lies in catalytic distillation, which combines reaction and separation of products in the same vessel, and enables a high level of conversion of reactants due to continuous removal of products (as per Le Chatelier's principle), which drives the equilibrium of the reaction towards the products.

In one aspect of the disclosure, one of or a mixture of any of C₂-C₅ olefins may be introduced to a catalytic distillation unit, where it may react over a dimerization catalyst to produce longer chain olefins. The formed higher olefins (e.g., C₄₌) may react with iC₄ which may be introduced into the column to form alkylate. In some cases, an olefin isomerization unit may be used upstream of the catalytic distillation unit such that olefins (such as 1-butene) may be isomerized into a mixture of olefin isomers (such as 1-butene and cis-2-butene, and trans-2-butene).

FIG. 31 shows a schematic for 2-bed dimerization followed by alkylation via catalytic distillation 3100. In this schematic, one or a mixture of any of C₂-C₅ olefins 3102 enters as feed into the catalytic distillation column 3103 in liquid or gas phase, where it may get into contact with a dimerization catalyst and convert into higher olefins (such as C₄₌). As formed olefins vapors move up in the column they may get into contact with iC₄ and an alkylation catalyst where alkylation reactions may proceed to form C₈₊ and nC₄/nC₅ by-products. The temperature and pressure of the column may be selected such that formed C₈₊ alkylates may condense into a liquid that moves downward in the column to a lower side stream 3106 while iC₄ and C₂-C₅ olefins vapors may move up. iC₄ may be condensed and recycled to the distillation tower using a condenser 3104. By-product nC₄/nC₅ are lighter than alkylate, and they may be drawn out of the column as an upper side stream 3105. Unconverted C₂-C₅ and iC₄ are condensed and routed back to the column. An optional re-boiler can be used to partially vaporize the C₈₊ alkylate product and recycle the vapor back into the column.

The operating conditions and catalyst of the dimerization bed may include Ni, Pd, Cr, V, Fe, Co, Ru, Rh, Cu, Ag, Re, Mo, W, Mn, Pt. The operating conditions and catalyst of the alkylation bed, with catalysts potentially including any one or combination of zeolites, sulfated zirconia, tungstated zirconia, chlorided alumina, aluminum chloride (AlCls), silicon-aluminum phosphates, titaniosilicates (including VTM zeolite), polyphosphoric acid (including solid phosphoric acid, or SPA, catalysts, which are made by reacting phosphoric acid with diatomaceous earth), polytungstic acid, and supported liquid acids such as triflic acid on silica, sulfuric acid on silica, hydrogen fluoride on carbon, antimony fluoride on silica, aluminum chloride (AlCls) on alumina (Al₂O₃). Operating conditions, catalysts, and reactor type and configuration of the olefin isomerization unit (if included), which employs catalysts typically used for olefin isomerization such as alkaline oxides (including MgO) can be varied. Ratio of starting olefin to iC₄-operation of the reboiler and condenser units (if included) such as to regulate the reflux ratios of C₂-C₅ olefins and iC₄ liquid/vapor and C₈₊ vapor back into the catalytic distillation column can be varied. Number of trays and/or height of packed catalyst beds used inside the column can be varied. Location of catalyst beds inside the column can be varied. Location of the feed(s) into the column can be varied. Location of the column top product draw can be varied. Location of introducing the condenser reflux stream(s) back into the column can be varied. Location of the column lower and upper side product draws can be varied. Location of introducing the reboiler reflux stream(s) (if any) back into the column can be varied.

### VIII. Ni-Based Oligomerization Followed by 2-Bed Alkylation Via Catalytic Distillation

In some cases, ETL technology in its current form takes OCM effluent or refinery offgas streams as feedstocks for the manufacture of higher hydrocarbons from the stream's light olefins (e.g. ethylene and propylene). The higher hydrocarbon product stream may comprise paraffins, isoparaffins, olefins, naphthenes, aromatics, or combinations thereof.

Ways to increase process versatility by altering the choice of product stream are needed to improve process flexibility and potentially profitability. One potential way is to gear the ETL process such as to maximize alkylate yields for the production of gasoline and diesel fuels.

Alkylation of olefins with isoparaffins may be used for the production of alkylate, a superior gasoline blendstock due to its unique characteristics such as high RON, no olefinic content, and low RVP, making it one of the most sought after streams for gasoline blenders. Processes for alkylation include solid acid based alkylation and alkylation process employing HF or sulfuric acid as the alkylation catalysts. These processes may have, however, some shortcomings such as the specification of feedstocks that go into them, such as being limited to isobutane and C₃₊ olefins as reactants.

Concurrently, methodologies to reduce capital cost and the number of unit operations associated with the ETL process are needed, as this can add to the technology competitiveness, diversity, and flexibility. One such methodology lies in catalytic distillation, which combines reaction and separation of products in the same vessel, and enables a high level of conversion of reactants due to continuous removal of products (as per Le Chatelier's principle), which drives the equilibrium of the reaction towards the products.

In one aspect of the disclosure, the ETL process is based on the initial step of oligomerization of light olefins (e.g. ethylene, propylene, and/or butenes) into higher olefins, with minimal conversion to hydrocarbons other than olefins (e.g. paraffins, isoparaffins, naphthenes, and aromatics). This may be accomplished over supported catalysts geared towards oligomerization at moderate process conditions. The C₄ olefin effluent from the previous step may be routed to a catalytic distillation unit, along with isobutane such that alkylation may be effected to produce a desired alkylate stream. The catalytic distillation unit may contain two alkylation catalyst beds where C₄ alkylation may take place by further alkylation of iC₈ and higher olefins (C₆₊) to produce a C₁₄₊ jet fuel and/or diesel blendstock.

Additionally, C₃ and C₄ olefins can be sourced from adjacent refinery/petrochemical units (such as FCC, MTO, FT, delayed cokers, or steam crackers) to form additional feed into the C4 alkylation bed in the distillation column, thereby increasing jet/diesel fuel production of out the process scheme

FIG. 32 is a schematic that demonstrates an example process scheme for a catalytic distillation and oligomerization 3200. In this schematic, a stream containing ethylene 3201 enters an ETL reactor 3202 to generate and ETL effluent. The effluent from the ETL reactor may enter as feed into the catalytic distillation column 3203 in liquid or gas phase, where C₂-C₄ olefins may move up in the column towards the top alkylation bed, get into contact with a stream containing iC₄ 3207 that is introduced into the column, and both react to form iC₈ (while by-product nC₄ is withdrawn as a side stream). iC₈ may move downward in the column, get into contact with C₆₊ olefins from ETL, and both react over a second alkylation bed towards the bottom of the column, producing C₁₄₊ hydrocarbons 3205. Unconverted C₂-C₄ and iC₄ (and any entrained nC₄) may be routed to a condenser 3204, where C₄s may be condensed out and recycled back into the column, while C₂₌ and water may be sent in vapor phase back into the ETL unit. A re-boiler 3206 may be used to partially vaporize the C₁₄₊ alkylate product and recycle the vapor back into the column, in order to strip any condensed unreacted C₆-C₈ hydrocarbons and send them back into the column.

An oxidizing agent, such as O₂, air, or water, can be fed along with the ETL unit feed (which may contain H₂), such as to minimize/limit the extent of ethylene/propylene hydrogenation over the oligomerization catalysts—a phenomenon that may take place over highly active oligomerization catalysts resulting in loss of olefins into paraffins, thereby reducing oligomer yield.

In some cases, CO contained in ETL feeds may convert readily via FT reactions with H₂ into C₁-C₄ paraffins, minimizing the adverse impact it can have over the oligomerization metal (such as Ni) such as etching.

In some cases, a hydrotreating catalyst layer (or separate reaction zone) upstream of the ETL reactor can be employed to remove sulfur from certain ETL feeds. This can be in the form of a hydrotreating catalyst layer, composed of CoMo or NiMo based catalyst (which may react sulfur and not saturate olefins in the feed over the used process conditions), or in the form of a separate and upstream hydrotreating unit, which can be a MEROX type unit (employing a liquid catalyst) or a CoMo/NiMo based unit as described for the case of hydrotreating layer above.

The choice of active metal for effecting oligomerization of light olefins into higher olefins can be any one or combination of Ni, Pd, Cr, V, Fe, Co, Ru, Rh, Cu, Ag, Re, Mo, W, Mn, and Pt, and with up to a total loading of 20% by weight of catalyst mass. Catalyst support can range between one or any combination of zeolites (such as ZSM-5, Beta, and ZSM-11), amorphous silica alumina, silica, alumina, mesoporous silica, mesoporous alumina, zirconia, titania, and pillared clay. The operating conditions of the ETL unit to suit optimal conversion and high olefin yield out of the ETL reactor (about 50-200° C. and about 10-80 bar). Choice of unit and associated operating conditions and catalyst employed for the upstream hydrotreating unit (if included) for removing sulfur can be varied. The ratio of oxidizing agent to feed hydrogen content to suppress olefin hydrogenation reactions can be varied. The operating conditions and catalyst of the alkylation beds may include Ni, Pd, Cr, V, Fe, Co, Ru, Rh, Cu, Ag, Re, Mo, W, Mn, Pt and supported on any one or combination of zeolites, sulfated zirconia, tungstated zirconia, chlorided alumina, aluminum chloride (AlCls), silicon-aluminum phosphates, titaniosilicates (including VTM zeolite), polyphosphoric acid (including solid phosphoric acid, or SPA, catalysts, which are made by reacting phosphoric acid with diatomaceous earth), polytungstic acid, and supported liquid acids such as triflic acid on silica, sulfuric acid on silica, hydrogen fluoride on carbon, antimony fluoride on silica, aluminum chloride (AlCls) on alumina (Al₂O₃). The ratio of iC₄ introduced to the column to olefin feed can be varied. The operation of the reboiler and condenser units (if included) such as to regulate the reflux ratios of olefins and iC₄ liquid/vapor and C₁₄₊ vapor back into the catalytic distillation column can be varied. The number of trays and/or height of packed catalyst beds used inside the column can be varied. The location of catalyst beds inside the column can be varied. The location of the feed(s) into the column can be varied. The location of the column top product draw can be varied. The location of introducing the condenser reflux stream(s) back into the column can be varied. The location of the column side product draw can be varied. The location of introducing the reboiler reflux stream(s) (if any) back into the column can be varied.

### Control Systems

The present disclosure also provides computer control systems that can be employed to regulate or otherwise control the methods and systems provided herein. A control system of the present disclosure can be programmed to control process parameters to, for example, effect a given product distribution, such as a lower concentration of unsaturated hydrocarbons (e.g., olefins) in a product stream out of an ETL reactor.

FIG. 6 shows a computer system 601 that is programmed or otherwise configured to regulate ETL, hydration and/or aromatization reactions, such as regulate fluid properties (e.g., temperature, pressure and stream flow rate(s)), mixing, heat exchange in the reactions. The computer system 601 can regulate, for example, fluid stream (“stream”) flow rates, stream temperatures, stream pressures, reaction unit temperature, reactor unit pressure, molar ratio between reactants, contact time of the reactant (or compounds) and reaction catalyst(s), and the quantity of products that are recycled.

The computer system 601 includes a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data.

The CPU 605 can execute a sequence of machine-readable instructions, which can be embodied in a program or software. The instructions may be stored in a memory location, such as the memory 610. Examples of operations performed by the CPU 605 can include fetch, decode, execute, and writeback. The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store programs generated by users and recorded sessions, as well as output(s) associated with the programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet. The computer system 601 can communicate with one or more remote computer systems through the network 630.

Methods as described herein can be implemented by way of machine (e.g., computer processor) executable code stored on an electronic storage location of the computer system 601, such as, for example, on the memory 610 or electronic storage unit 615. The machine executable or machine readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine have a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” in some cases in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, signals from a chip with time. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 605.

### Hydrocarbon Oligomerization Processes and Systems

An aspect of the present disclosure provides methods for forming C₂₊ compounds using oligomerization processes. Such methods can employ the integration of an oligomerization process in a non-oligomerization system or process, which can include retrofitting the non-oligomerization system or process with equipment to enable the formation of C₂₊ compounds using inputs from the non-oligomerization system or process.

In an oligomerization process, C₂₊ hydrocarbons are generated upon the reaction of olefinic hydrocarbons reacting with other olefins, alkanes, or aromatics to make longer hydrocarbon molecules. The reaction can be facilitated by a heterogeneous catalyst support such as zeolites, alumina, silica, alumina/silica mixtures, metal organic frameworks (MOF), sulfated zirconia, polyoxymetallates, titanosilicates, chlorided alumina, amorphous silica/alumina, alumina phosphates, and supported liquid acids. Additional elements may be introduced to the heterogenous catalyst support by way on ion exchange and wet impregnation techniques. These elements are co-catalysts with the heterogenous catalyst supports to facilitate the oligomerization reaction. Examples of elements introduced to the heterogenous support are: Nickel (Ni), Cobalt (Co), Manganese (Mn), Sodium (Na), Potassium (K), Calcium (Ca), Strontium (Sr), Barium (Ba), Titanium (Ti), Zirconium (Zr), Vanadium (V), Chromium (Cr), Tungstun (W), Iron (Fe), Palladium (Pd), Platinum (Pt), Zinc (Zn), Gallium (Ga), Boron (B), Phosphorus (P), Lanthanum (La), Cerium (Ce) and Neodymium (Nd).

FIG. 33 shows an oligomerization process 3300, as may be employed for use with methods (or processes) and systems of the present disclosure. The oligomerization process 3300 includes a source of olefins 3301, catalyst guard bed 3302, at least one oligomerization reactor 3303, and a separation system 3304. Inputs and outputs into respective units are indicated by arrows. The source of olefin, 3301, can be from and OCM reactor, the off-gas from an FCC reactor, and/or the off gas of a DCU reactor. The source of methane can include one or more separation units to separate olefins from any C₂₊ compounds and non-C₂₊ impurities.

During use, olefins from the source of olefin 3301 may be directed into the guard bed unit 3302, which may remove undesirable components or potential catalyst poisons contained in the feed stream.

Next, the olefin containing gas may be directed from the guard bed unit 3302 to the oligomerization unit 3303. In the oligomerization unit 3303, olefinic compounds are formed into higher molecular weight hydrocarbons. The hydrocarbons from the oligomerization unit 3303 can be directed to the separation unit 3304, which separates the hydrocarbons into streams each comprising a substrate of the C₂₊ compounds and in some cases non-C₂₊ impurities. In some cases, light olefin gases separated in unit 3304 may be directed back to oligomerization unit 3303 for further reaction.

The separation system 3304 can include at least 1, 2, 3, 4, 5, 6, 7, 8, 9, 10, 20, 30, 40, or 50 separation units, which can be in series and/or parallel. Each separation unit can be configured to effect the separation of an input stream into separate streams each comprising a subset of the components in the input stream. Examples of separation units include distillation units, absorption units, vapor-liquid separation units, knock out drums, and cryogenic separation units. In some examples, the separation system 3304 includes at least 1, 2, 3, 4, 5, 6, 7, 8, 9, 10, 20, 30, 40, or 50 distillations units.

In some cases, the source of olefins 101 has a C₂₊ olefin concentration that is less than about 50%, 40%, 30%, 20%, 10%, 5%, or 1%.

One oligomerization unit 3303 can include at least 1, 2, 3, 4, 5, 6, 7, 8, 9, 10, 20, 30, 40, or 50 oligomerization reactors. In some cases, at least one oligomerization unit 3303 includes at least 2, 3, 4, 5, 6, 7, 8, 9, 10, 20, 30, 40, or 50 oligomerization reactors in series. As an alternative, the at least one oligomerization reactor 3303 includes at least 2, 3, 4, 5, 6, 7, 8, 9, 10, 20, 30, 40, or 50 oligomerization reactors in parallel. As another alternative, the at least one oligomerization reactor 3303 includes at least 2, 3, 4, 5, 6, 7, 8, 9, 10, 20, 30, 40, or 50 oligomerization reactors, at least some of which are in series and some of which are in parallel. If multiple oligomerization reactors are employed in series, each oligomerization reactor can include the same or a different catalyst as another oligomerization reactor. For example, one oligomerization reactor can include a catalyst to effect formation of hydrocarbons having between two and ten carbon atoms, and another oligomerization reactor can include a catalyst to effect the formation of hydrocarbons having greater than ten carbon atoms.

An oligomerization reactor can include at least one heterogeneous catalyst or multiple heterogenous catalysts. The catalyst may be in the form of a honeycomb, packed (or fixed) bed, or fluidized bed. Oligomerization catalysts that can be employed for use with systems and methods of the present disclosure can comprise at least one metal or metallic material, such as a transition metal selected from Nickel (Ni), Cobalt (Co), Manganese (Mn), Sodium (Na), Potassium (K), Calcium (Ca), Strontium (Sr), Barium (Ba), Titanium (Ti), Zirconium (Zr), Vanadium (V), Chromium (Cr), Tungstun (W), Iron (Fe), Palladium (Pd), Platinum (Pt), Zinc (Zn), Gallium (Ga), Boron (B), Phosphorus (P), Lanthanum (La), Cerium (Ce), and Neodymium (Nd) which may be present in the form of an oxide, carbide, elemental metal, alloy, or a combination thereof. In some examples, the catalyst may comprise from about 1% to about 60% of metal material.

Oligomerization reactor conditions can be selected to provide a given selectivity and product distribution. In some cases, for catalyst selectivity towards aromatics, an ETL reactor can be operated at a temperature greater than or equal to about 300° C., 350° C., 400° C., 410° C., 420° C., 430° C., 440° C., 450° C., or 500° C., and a pressure greater than or equal to about 250 pounds per square inch (PSI) (absolute), 200 PSI, 250 PSI, 300 PSI, 350 PSI or 400 PSI. For catalyst selectivity towards jet or diesel fuel, an ETL reactor can be operated at a temperature greater than or equal to about 100° C., 150° C., 200° C., 210° C., 220° C., 230° C., 240° C., 250° C., or 300° C., and a pressure greater than or equal to about 350 PSI, 400 PSI, 450 PSI, or 500 PSI. For catalyst selectivity towards gasoline, an ETL reactor can be operated at a temperature greater than or equal to about 200° C., 250° C., 300° C., 310° C., 320° C., 330° C., 340° C., 350° C., or 400° C., and a pressure greater than or equal to about 250 PSI, 300 PSI, 350 PSI, or 400 PSI.

In some cases, the operating conditions of an ETL process are substantially determined by one or more of the following parameters: process temperature range, weight-hourly space velocity (mass flow rate of reactant per mass of solid catalyst), partial pressure of a reactant at the reactor inlet, concentration of a reactant at the reactor inlet, and recycle ratio and recycle split. The reactant can be a (light) olefin—e.g., an olefin that has a carbon number in the range C₂-C₇, C₂-C₆, or C₂-C₅.

Temperatures used in a gasoline process can be from about 150 to 600° C., 220° C. to 520° C., or 270° C. to 450° C. Lower temperature can result in insufficient conversion while higher temperatures can result in excessive coking and cracking of product. In an example, the WHSV can be between about 0.5 hr⁻¹ and 3 hr⁻¹, partial pressures can be between about 0.5 bar (absolute) and 3 bar, and concentrations at the reactor inlet can be between about 2% and 30%. Higher concentrations can yield difficult-to-manage temperature excursions, while lower concentrations can make it difficult to achieve sufficiently high partial pressures and separation of the products. A process can achieve longer catalyst lifetime and higher average yields when a portion of the effluent is recycled. The recycle can be determined by a recycle ratio (e.g., volume of recycle gas/volume of make-up feed) and the post-reactor vapor-liquid split which determines the composition of the recycle stream. There may be several degrees of freedom to the recycle split, but in some cases the composition of the recycle stream may be important, which is achieved by post-reactor separation (e.g., typical carbon number/boiling point range that is recycled vs. the carbon number/boiling point ranges that are removed by product and/or secondary process streams.

To achieve longer average chain lengths and to avoid cracking of elongated chains such as those found in jet fuel and distillates, ETL can be performed at reactor operating temperatures from about 150° C. to 500° C., 180° C. to 400° C., or 200° C. to 350° C. The slower kinetics may suggest a lower minimum WHSV of about 0.1 hr⁻¹. Longer chain lengths may be favored by high partial pressures, so the upper end for jet/distillates may be higher than for gasoline, in some cases as high as about 30 bar (absolute), 20 bar, 15 bar, or 10 bar.

More consistent production of aromatics can be achieved at high temperature ranges, such as a temperature up to about 200° C., 250° C., 300° C., 350° C., 400° C., 450° C., or 500° C. In an adiabatic or even in a pseudo-isothermal reactor, the ethylene/olefin feed can be diluted by an inert gas (e.g., N₂, Ar, methane, ethane, propane, butane or He). The inert gas can serve to moderate the temperature increase in the reactor bed, and maintain and stabilize contact time. The olefin concentration at the reactor inlet can be less than about 50%, 40%, 30%, 20%, or 10%. In some cases, the higher the molar heat capacity of the diluent, the higher the inlet concentration of olefins can be to achieve the same temperature rise.

The following is a list of suitable compounds that may be found in significant quantities in the process. Such compounds are listed in the order of increasing heat capacity: nitrogen, carbon dioxide, methane, ethane, propane, n-butane, iso-butane.

An effluent or product stream from an ETL reactor can be characterized by low water content. For example, an ETL product stream can comprise less than 60 wt %, 56 wt %, 55 wt %, 50 wt % 45 wt % 40 wt % 39 wt % 35 wt % 30 wt % 25 wt % 20 wt % 15 wt % 10 wt % 5 wt % 3 wt % or 1 wt % water. In some cases, at least a portion of the reactor effluent is recycled to the reactor inlet. As an alternative, at most a portion of the reactor effluent is recycled to the reactor inlet. The volumetric recycle ratio (i.e., flow rate of the recycle gas stream divided by flow rate of the make-up gas stream (e.g., fresh feed)) can be between about 0.1 and 30, 0.3 and 20, or 0.5 and 10.

A continuous process for making mixtures of hydrocarbons for use as gasoline can comprise feeding olefinic compounds to a reaction zone of an ETL reactor. The ETL reactor can include a catalyst that is selected for gasoline production, as described elsewhere herein. The process temperature can be between about 200° C. and 600° C., 250° C. and 500° C., or 300° C. and 450° C. The partial pressure of olefins in the feed can be between about 0.1 bar (absolute) to 10 bar, 0.3 bar to 5 bar, or 0.5 bar to 3 bar. The total pressure can be between about 1 bar (absolute) to 100 bar, 5 bar to 50 bar, or 10 bar to 50 bar. The weight hourly space velocity can be between about 0.1 hr⁻¹ to 20 hr⁻¹, 0.3 hr⁻¹ to 10 hr⁻¹, or 0.5 hr⁻¹ to 3 hr⁻¹.

For products in the distillate range (e.g., C₁₀₊ molecules, which can exclude gasoline in some cases), the catalyst composition can be selected as described elsewhere herein. The process temperature can be between about 100° C. and 600° C., 150° C. and 500° C., or 200° C. and 375° C. The partial pressure of olefins in the feed can be between about 0.5 bar (absolute) to 30 bar, 1 bar to 20 bar, or 1.5 bar to 10 bar. The total pressure can be between about 1 bar (absolute) to 100 bar, 5 bar to 50 bar, or 10 bar to 50 bar. The weight hourly space velocity can be between about 0.05 hr⁻¹ to 20 hr⁻¹, 0.1 hr⁻¹ to 10 hr⁻¹, or 0.1 hr⁻¹ to 1 hr⁻¹.

For products comprising mixtures of hydrocarbons substantially comprised of aromatics, the catalyst composition can be selected as described elsewhere herein. The process temperature can be between about 200° C. and 800° C., 300° C. and 600° C., or 400° C. and 500° C. The partial pressure of olefins in the feed can be between about 0.1 bar (absolute) to 10 bar, 0.3 bar to 5 bar, or 0.5 bar to 3 bar. The total pressure can be between about 1 bar (absolute) to 100 bar, 5 bar to 50 bar, or 10 bar to 50 bar. The weight hourly space velocity can be between about 0.05 hr⁻¹ to 20 hr⁻¹, 0.1 hr⁻¹ to 10 hr⁻¹, or 0.2 hr⁻¹ to 1 hr⁻¹.

The ETL process can generate a variety of long-chain hydrocarbons, including normal and isoparaffins, napthenes, aromatics and olefins, which may not be present in the feed to the ETL reactor. The catalyst can deactivate due to the deposition of carbonaceous deposits (“coke”) on the surfaces of the catalyst. As the deactivation progresses, the conversion of the process changes until a point is reached when the catalyst can be regenerated.

In some cases, in the early stages of a reaction cycle, the product distribution can contain large fractions of aromatics and short-chained alkanes. Later stages can feature increased fractions of olefins. All stages can feature various amounts isoparaffins, n-paraffins, naphthenes, aromatics, and olefins, including olefins other than feed olefins. The change in selectivity with time can be exploited by separating products. For example, the aromatics-rich effluent characteristic of the early stages of a reaction cycle may be readily separated from the effluent of a catalyst bed in a later stage of its cycle. This can result in high selectivities of individual products. An example of how the product distribution can change over time is given in FIG. 5, which is for a Ga-ZSM-5 catalyst.

The ETL process can generate various byproducts, such as carbon-containing byproducts (e.g., coke) and hydrogen. The selectivity for coke can be on the order of at least about 1%, 2%, 3%, 4%, or 5% over the course of an ETL process. Hydrogen production can vary with time, and the amount of hydrogen generated can be correlated with aromatics production.

In some cases, the time-averaged product of the process can yield a liquid with a composition that meets the specification of reformulated gasoline blendstock for oxygen blending (RBOB). In some cases, RBOB has at least about an 93 octane rating using the (RON+MON)/2 method, has less than about 1.3 vol % benzene as measured by ASTM D3606, has less than about 50 vol % aromatics as measured by ASTM D5769, has less than about 25 vol % olefins as measured by ASTM D1319 and/or D6550, has less than 80 ppm (wt) sulfur as measured by ASTM D2622, or any combination thereof. Such liquid can be employed for use as fuel or other combustion settings. This liquid can be partially characterized by the content of aromatics. In some cases, this liquid has an aromatics content from 10% to 80%, 20% to 70%, or 30% to 60%, and an olefins content from 1% to 60%, 5% to 40%, or 10% to 30%. Gasoline can comprise about 60% to 95%, 70% to 90%, or 80-90% of such liquid, with the remainder in some cases being an alcohol, such as ethanol.

In some situations, an ETL process is used to generate a mixture of hydrocarbons from light olefin compounds (e.g., ethylene). The mixture can be liquid at room temperature and atmospheric pressure. The process can be used to form a mixture of hydrocarbons having a hydrocarbon content that can be tailored for various uses. For example, mixtures typically characterized as gasoline or distillate (e.g., kerosene, diesel) blend stock, or aromatic compounds, can contribute at least 30%, 40%, 50%, 60%, or 70% by weight to the final fuel product.

The product selectivity of the ETL process can change with time. With such changes in selectivity, the product can include varying distributions of hydrocarbons. Separations units can be used to generate a product distribution which can be suitable for given end uses, such as gasoline.

Products of ETL processes of the present disclosure can include other elements or compounds that may be leached from reactors or catalysts of the system (e.g., OCM and/or ETL reactors). Examples of OCM catalysts and the elements comprising the catalyst that can be leached into the product can be found in U.S. Pat. No. 8,962,517 or U.S. Provisional Patent Application 61/988,063, each of which is incorporated by reference in its entirety. Such elements can include transition metals and lanthanides. Examples include, but are not limited to Mg, La, Nd, Sr, W, Ga, Al, Ni, Co, Ga, Zn, In, B, Ag, Pd, Pt, Be, Ca, and Sr. The concentration of such elements or compounds can be at least about 0.01 parts per billion (ppb), 0.05 ppb, 0.1 ppb, 0.2 ppb, 0.3 ppb, 0.4 ppb, 0.5 ppb, 0.6 ppb, 0.7 ppb, 0.8 ppb, 0.9 ppb, 1 ppb, 5 ppb, 10 ppb, 50 ppb, 100 ppb, 500 ppb, 1 part per million (ppm), 5 ppm, 10 ppm, or 50 ppm as measured by inductively coupled plasma mass spectrometry (ICPMS).

The composition of ETL products from a system can be consistent over several cycles of catalyst use and regeneration. A reactor system can be used and regenerated for at least about 10, 20, 30, 40, 50, 60, 70, 80, 90, or 100 cycles. After a number of regeneration cycles, the composition of the ETL product stream can differ from the composition of the first cycle ETL product stream by no more than about 0.1%, 0.2%, 0.3%, 0.4%, 0.5%, 0.6%, 0.7%, 0.8%, 0.9%, 1%, 2%, 3, 4%, 5%, 6%, 7%, 8%, 9%, 10%, 11%, 12%, 13%, 14%, 15%, 16%, 17%, 18%, 19%, or 20%.

FIG. 34 shows a system 3400 that is configured and adapted to generate hydrocarbons using an oligomerization process. The oligomerization process 3400 includes a source of olefins 3401, catalyst guard bed 3402, at least one oligomerization reactor 3403, a drying bed to move residual water 3404, and a separation system 3405. Inputs and outputs into respective units are indicated by arrows. The source of olefin 3401, can be from and OCM reactor, the off-gas from an FCC reactor, and/or the off gas of a DCU reactor. The source of olefin 3401, can be from and OCM reactor, the off-gas from an FCC reactor, and/or the off gas of a DCU reactor, or any olefin containing stream. The separation module 3404 can include at least 1, 2, 3, 4, 5, 6, 7, 8, 9, 10, 20, 30, 40, or 50 separation units, such as described above in the context of FIG. 34. In some examples, the first separation module can include one or more distillation units, cryogenic separation units, knock-out drum, liquid/vapor separator, and/or recycle split vapor (RSV) units.

During use, feed stream 3401 comprising C₂₊ olefins is directed to the guard bed module 3402, that can contain at least one guard bed. Next, the olefin containing gas is directed from the guard bed module 3402, to the oligomerization module 3403 that can contain at least one oligomerization reactor. Before entering the oligomerization reactor, the gas is brought to a desirable range of process pressure and process temperature. Feed stream 3401, pressure range can be from 1 barg to 100 barg and the temperature range can be from 50° C.-600° C. The feed pressure is raised to process pressure using a process gas compressor. The feed compression section can include at least 1, 2, 3, 4, 5, 6, 7, 8, 9, 10, 20, 30, 40, or 50 compressors. The feed stream temperature is raised through a series of heat exchangers. The feed heat exchanger section can include at least 1, 2, 3, 4, 5, 6, 7, 8, 9, 10, 20, 30, 40, or 50 heat exchangers. In the oligomerization unit 3403, olefinic compounds are formed into higher molecular weight hydrocarbons. The reactor design in the oligomerization module 3403, may be insulated to minimize heat exchange from the interior of the reactor to its surroundings. The gas exit temperature for the oligomerization process will be the temperature of the process plus any additional heat released from the chemical reactor. This type of reactor may be an adiabatic reactor. The exit gas temperature for an adiabatic oligomerization unit will be higher the inlet temperature for an exothermic reaction. An exothermic chemical reaction releases heat. In the oligomerization unit the exit gas temperature may range from 200-900° C. The increase in exit gas temperature from the oligomerization module, 3403, is dependent on the concentration of reactant, the percent conversion of the reactant in the reactor, and the heat capacity of the total gas mixture. Alternatively, the oligomerization module, 3403, may comprise reactors that allow heat exchange between the reactor and a cooling medium. The cooling medium may be a gas or liquid that is introduced to the oligomerization module to cool the process gas in the oligomerization reactor. This type of reactor may be an isothermal reactor. By cooling the process gas temperature in the reactor, the oligomerization module may benefit from increased olefin conversion per pass as well as better product selectivity to C₅₊ compounds.

The hydrocarbon containing stream is directed from the oligomerization unit, 3403, to a dryer unit 3404 to remove any residual water before continuing into the separations unit, 3405. The dryer module, 3404, can include at least 1, 2, 3, 4, 5, 6, 7, 8, 9, 10, 20, 30, 40, or 50 dryer units, such as described above in the context of FIG. 34. Before entering the dryer unit, the process gas from the oligomerization unit, 3403, will be cooled by a series of heat exchangers to bring the gas temperature to an acceptable level before entering the process gas dryer, 3404. Small quantities of water may be found in the product stream due to water impurities in the feed as well as small production of water in the oligomerization due to the reverse water gas shift reaction (rWGS). The reverse water gas shift reaction is the reaction of carbon dioxide (CO₂) and hydrogen (H₂) to produces carbon monoxide (CO) and water (H₂O). Water needs to be removed from the process stream before going into the separations unit, 3405, if an operation in unit 3404 is operating at or below approximately 15° C. Water freeze may freeze if operated at or below 0° C. In addition, water impurities in the process stream may react with hydrocarbons in the process gas stream to form clathrate hydrates. Clathrate hydrates are crystalline water-based solids physically resembling ice, in which small non-polar molecules (e.g., methane) or polar molecules with large hydrophobic moieties are trapped inside “cages” of hydrogen bonded, frozen water molecules. Formation of ice, consisting mainly of water, and/or clathrate hydrates, as described above, may be undesirable in the separations unit since the presence of either may limit or preclude entirely gas processing due to restricting or blocking gas flow of the unit. In the event, a unit operation in the separations unit, 3405, becomes plugged, by either ice, consisting mainly of water, and/or clathrate hydrates, the unit will have to removed from service and brought to an appropriate temperature to melt the blockage. Typically, temperatures greater than about 20° C. is sufficient to melt ice, consisting mainly of water, and/or clathrate hydrates.

A dryer unit in the dryer module 3404 may contain an adsorbent bed to remove water. The adsorbent bed may consist of a molecular sieve, zeolite, or a metal salt (e.g., calcium chloride, magnesium chloride, sodium sulfate, magnesium sulfate). As the adsorbent bed reaches water saturation the saturated adsorbent bed is taken offline and regenerated in-situ by raising the temperature of the bed to a sufficient temperature and flowing an inert gas over the bed to create a stream containing water, 3408. As one dryer bed is brought offline, a fresh adsorbent bed is simultaneously brought on-line to ensure continuous process gas drying. Alternatively, the adsorbent bed may need to be removed and recharged with new adsorbent material if required.

The separations unit 3405 produces a stream consisting mostly of C₅₊ products, 3407, and a stream containing mostly C₄₋ compounds, 3406. The 3406 stream contains some C₃ and C₄ olefinic compounds that can be recycled back to the reactor unit, 3403, for further reaction. In some cases, the concentration of the C₄₋ olefins is less than about 50%, 40%, 30%, 20%, 10%, 1%, 0.1 mol %. The recycle process is facilitated by a compressor, 3409, to bring the 3406 recycle stream pressure to the same process pressure as the feed stream. The ratio of recycle stream, 3406, volume flow rate to feed stream, volume flow rate may vary from 50:1 to 0.1:1.

FIG. 35 shows a system 3500 that is adapted to produce hydrocarbons using an oligomerization process. The process includes an olefin source, 3501, a guard bed, 3502, an oligomerization unit, 3503, a vapor/liquid separator, 3504, a process gas dryer, 3505, recycle gas compressor, 3508, and a product recovery unit, 3507. Once the oligomerization effluent exits the oligomerization reactor and the effluent is cooled using heat exchangers to about 25-200° C. and then processed through the vapor/liquid separator, 3504. The vapor/liquid separator, 3504, separates the process stream into 2 streams: (1) a vapor product and (2) a liquid product. The vapor product gas, 3506, can be recycled back to the oligomerization unit, 3503, via the recycle compressor, 3508. The vapor product gas, 3506, may comprise C₇₋ alkanes, C₇₋ olefins, water, carbon monoxide, carbon dioxide, methane, ethane, ethylene, propene, butenes, and napthenes. The liquid product stream, 3511, may be collected and processed further to remove undesirable compounds such as C₄₋ or water. The vapor/liquid separator, 3504, may be a two-phase separator that separates gas products from liquid products. In a further embodiment, the vapor/liquid separator may be a three-phase separator that separates gas products, hydrocarbon liquid products, and water products.

Recycling can have various benefits, such as: 1) further reaction of shorter chain hydrocarbon products to form higher molecular weight products, 2) increasing catalyst lifetime, and 3) diluting the C₂H₄ feed stream to control the reactor process conditions of reactant concentration and adiabatic temperature rise.

In some cases, an inlet feed stream that is diluted with recycle product stream allows for a smaller adiabatic temperature rise in the reactor and reduced C₂H₄ concentration into the reactor. A lower adiabatic temperature rise, and therefore peak reactor temperature, can alter the effluent product stream composition. Higher peak reactor temperatures, for instance, can increase the yield and selectivity of aromatic products.

Different amounts of ethylene in an ETL product stream can be recycled. In some cases, at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99%, or 100% of ethylene in an ETL product stream is recycled. In some cases, at most about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99%, or 100% of ethylene in an ETL product stream is recycled.

An ETL process can be characterized by a single pass conversion or single pass conversion of C₂₊ compounds to C₃₊ compounds of at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.9%, or 99.99%.

FIG. 36 shows guard bed module, 3600, adapted to lower and/or remove undesirable impurities and undesirable components in the olefin containing feed stream to the oligomerization unit. Guard beds 3602A-B are designed to lower and/or remove impurities in the olefin containing stream. The impurities may include: arsines, phosphorous containing compounds (e.g. phosphines, phosphates), alkali metal (e.g. lithium, sodium, potassium) containing compounds (e.g. alkali metal oxides, alkali metal carbonates, alkali metal phosphates), alkali earth metal (e.g. magnesium, calcium, barium) containing compounds (e.g. alkali metal oxides, alkali earth metal carbonates, alkali earth metal phosphates), transition metal (eg. nickel, cobalt, titanium) containing compounds (e.g. transition metal oxides, transition metal carbonates, transition metal phosphates), and nitrogen containing compounds (e.g. amines, pyridines, imidazoles, pyrimidines). The guard bed section can include at least 1, 2, 3, 4, 5, 6, 7, 8, 9, 10, 20, 30, 40, or 50 guard beds. The guard beds may be operated such that when one bed needs to be removed from service another guard bed is ready to be brought online to ensure continuous service. The adsorbent materials in the guard beds may include: activated carbon; amorphous silica/alumina; alpha alumina; gamma alumina; amorphous silica; silica/alumina molecular sieves; silica molecular sieves; amorphous alumina/phosphates; and alumina/phosphates molecular sieves. These materials may be formed into various shapes and loaded into the guarded bed vessel. Shapes and sizes for the adsorbent material for guard beds, 3602A-B, may include: spheres; trilobes; quadralobes; and cylinders in the range of about 1 mm-20 mm in diameter and about 1 mm-50 mm in length.

In an example, two guard beds are placed upstream of four or five parallel ETL reactor beds. The two guard beds are designed in a lead-lag configuration. The inlet temperature of the guard bed may be about 40° C., about 60° C., about 80° C., or about 100° C. lower than the inlet to the ETL reactors and the space velocity may be at least about 5x, at least about 10×, at least about 20× or at least about 50× greater than the space velocity of the ETL reactors. The ETL reactors are on a schedule where each parallel reactor is regenerated and decoked every three weeks. But the guard bed is regenerated and decoked every 36 hours.

The guard bed module, may comprise a section for hydrogen (H₂) removal, 3602C. The hydrogen removal section consists of adsorption beds and a compressor may selectively remove hydrogen to lower the hydrogen concentration of feed stream 3601 prior to entering the oligomerization module, 3604. The feed gas exiting the guard beds 3602A-B may be compressed to 2-50 barg and then enters the 3602C adsorption beds. Non-H₂ components in the feed stream are preferentially adsorbed on the adsorbent and H₂ is allowed to flow the bed to produce a purity H₂ stream. Once adsorption equilibrium is reached the vessel is depressurized to produce a tail gas stream with lower H₂ concentration. Removing H₂ prior to the oligomerization module may be desirable due to the deleterious effect of H₂ for C₅₊ product selectivity in the overall process.

FIG. 36 is an example contour plot of the effect of H₂ concentration in the oligomerization process feed on the C₅₊ process yield. As the ethylene mol fraction and the hydrogen mol fraction increases, the C₅₊ yield decreases. The presence of H₂ in the oligomerization unit may promote side reactions such as hydrogenation and cracking that produce lower carbon chain hydrocarbons (e.g. ethane, propane, butane). The H₂ removal unit, 3602C, may be designed and operated to remove 99+% of the H₂ in the feed stream or to remove a fraction of the H₂ in the feed stream (e.g. 80%, 70%, 60%, 50%, 40%, 30%, 20%, 10%, 9%, 8%, 7%, 6%, 5%, 4%, 3%, 2%, 1%). The H₂ removal unit, 3602C, is situated upstream of the recycle stream, 3606, to minimize the amount of process gas flow through the H₂ removal unit. Alternatively, the H₂ removal unit, 3602 C, may be situated on the process stream 3606, after the recycle compressor, 3608, and before the oligomerization module, 3604.

### Catalyst Regeneration Processes and Methods

ETL catalysts may need to be regenerated from a state of low ethylene conversion (e.g., 20% or less) to high ethylene conversion, such as, e.g., greater than 20%, 30%, 40%, 50%, 60%, or 70%. Regeneration can occur by heating the catalyst bed to an appropriate temperature while introducing a portion of diluted air. The oxygen in air can be used to remove coke by combustion and thus renew catalyst activity. Too much oxygen can cause uncontrolled combustion, a highly exothermic process, and the resultant catalyst bed temperature rise may cause irreversible catalyst damage. As a consequence, the amount of air that is permitted during adiabatic reactor regeneration is limited and monitored.

The ETL catalyst can be regenerated in the presence of any suitable fluid, such as air, nitrogen (N₂), carbon dioxide (CO₂), methane (CH₄), natural gas, hydrogen (H₂), or any combination thereof. Specifically, air can be diluted by mixing with fresh nitrogen, air can be diluted by mixing with recycled nitrogen, air can be diluted by mixing with carbon dioxide, air can be diluted by mixing with methane, air can be diluted by mixing with natural gas, or combinations thereof. The fluid can be freshly produced, or recycled from another part of the process. In some cases, the fluid (i.e., N₂) can be provided by an air separation unit (ASU). However, some processes that are to be retrofitted with an ETL process do not have an ASU (e.g., midstream gas processing plants) and installation of an ASU may be excessively costly. Therefore, the present disclosure provides for systems and methods for regenerating the ETL catalyst using CO₂, CH₄, natural gas and/or H₂.

The catalyst regeneration time for an adiabatic reactor can be largely dictated by the amount of oxygen that can be permitted in the reactor. The greater heat transfer properties of the disclosed multi-tubular reactors can permit greater concentrations of oxygen during catalyst regeneration to hasten catalyst regeneration while ensuring that the catalyst bed temperature does not reach the point of irreversible catalyst deactivation.

Since ETL catalysts can deactivate over time through coke deposition, the fixed bed reactors can be taken off-line and regenerated, such as by an oxidative or non-oxidative process, as described elsewhere herein. Once regenerated to full activity the ETL reactors can be put back on-line to process more feedstock.

Systems and methods of the present disclosure can employ the use of ETL continuous catalyst regeneration reactors. Continuous catalyst regeneration reactors (CCRR) can be attractive for processes where the catalyst deactivates over time and need to be taken off-line to be regenerated. By regenerating the catalyst in a continuous fashion less catalyst, fewer reactors for the process as well as fewer required operations are to regenerate the catalyst. There are two classes of deployments for CCRR reactors: (1) moving bed reactors and (2) fluidized bed reactors. In moving bed CCRR design, the pelletized catalyst bed moves along the reactor length and is removed and regenerated in a separate vessel. Once the catalyst is regenerated the catalyst pellets are put back in the ETL conversion reactor to process more feedstock. The online/regeneration process can be continuous and can maintain a constant flow of active catalyst in the ETL reactor. In fluidized bed ETL reactors, ETL catalyst particles are “fluidized” by a combination of ETL process gas velocity and catalyst particle weight. During bed fluidization, the bed expands, swirls, and agitates during reactor operation. The advantages of an ETL fluidized bed reactor are excellent mixing of process gas within the reactor, uniform temperature within the reactor, and the ability to continuously regenerate the coked ETL catalyst.

The ETL catalyst can be regenerated with methane or natural gas. The regeneration stream can have oxygen (O₂) or other oxidizing agent. The concentration of oxygen in the regeneration stream can be below the limiting oxygen concentration (LOC), such that the mixture is not flammable. In some embodiments, the concentration of O₂ in the regeneration stream is less than about 6%, less than about 5%, less than about 4%, less than about 3%, less than about 2%, or less than about 1%. In some cases, the concentration of O₂ in the regeneration stream is between 0% and about 3%. An advantage of regenerating the ETL catalyst with methane or natural gas is that, following flowing over the ETL catalyst for regeneration, the stream can be used in the OCM and/or ETL process (e.g., the stream can be combusted to provide energy). The use of methane and/or natural gas to regenerate the ETL catalyst may not introduce any new components into the process to achieve catalyst regeneration, which can lead to an efficient use of materials. In some cases, the use of methane and/or natural gas makes the economics of the process insensitive, or less dependent on, the period of time that the ETL catalyst can operate between regeneration cycles.

FIG. 37 shows the catalyst regeneration module that is configured and adapted to regenerate the oligomerization catalyst. First, the feed module, 3701, purges at least one reactor in the oligomerization module, 3704, with at least 1 bed volume equivalent of nitrogen (N₂) that has been heated in a range between 200-600° C. In some cases the oligomerization vessel may be purged with 2-5 bed volume equivalents of nitrogen N₂ gas, 6-8 bed volume equivalents of N₂ gas, or 9-10 bed volume equivalents of N₂ gas that has been heated in a range between 200-600° C. Once the vessel has been charged with heated N₂ gas, a flow of air, 3702, may be heated to a range between 200° C.-600° C., to remove the catalyst coke. The amount of air flow, 3702, is controlled to keep the oxygen (O₂) concentration between 0.1-21 mol %. The air flow, 3702, can be introduced at the bottom of the oligomerization reactor and flow from bottom of the reactor to the top of reactor against the force of gravity. Alternatively, the air flow, 3702, can be introduced at the top of the reactor and flow from the top of the reactor to the bottom of the reactor in the direction of gravity. Process conditions can be selected to keep the increase in temperature of the ETL catalyst less than or equal to about 700° C., 650° C., 600° C., 550° C., 500° C. or less during the regeneration. This can help prevent catalyst damage during the regeneration process. Oxidative regeneration reactor inlet temperatures can range from about 100° C. to 800° C., 150° C. to 700° C., or 200° C. to 600° C. Inlet gas temperatures can be ramped from low to high temperatures to safely control the regeneration process. During oxidative regeneration, process gas pressures can range from about 1 bar (gauge, or “barg”) to 100 barg, 1 barg to 80 barg, or 1 barg to 50 barg. The oxidative regeneration effluent, 3703, is sent to the compressor or blower unit, 3708, then sent back to the oligomerization reactor to be added to the air stream, 3702. The compressor or blower increase the recycle stream, 3703, differential pressure by 1-10 barg. The volumetric ratio of recycle stream, 3703, to air stream 3702 is controlled to maintain the desired O₂ concentration in the oxidative regeneration process gas during the regeneration process. The recycle stream, 3703, comprises CO₂, H₂O, CO, and O₂ components. The recycle steam, 3703, may go through dryer units, 3705A or 3705B, configured to remove H₂O from the recycle stream. The dryer may be positioned either before or after the compressor/blower unit. Removing water in recycle stream, 3703, avoids build up of H₂O concentration in the recycle loop. In some instances, the dryer unit is precluded. As H₂O builds up in the recycle stream, 3703, the catalyst is exposed higher H₂O concentration which may accelerate the deactivation of the oligomerization zeolite catalyst through de-alumination of the catalyst active site. The purge stream, 3704, controls the process pressure during the oxidative regeneration process.

Non-oxidative catalyst regeneration may also be used for the regeneration process. Specifically, hydrogen (H₂) and/or hydrocarbons can be used to regenerate the catalyst bed to improve catalyst activity of the ETL catalyst. Hydrogen or hydrocarbon gases can be directed over the catalyst bed at a temperature from about 100° C. to 800° C., 150° C. to 600° C., or 200° C. to 500° C. This can aid in removing or decreasing the concentration of carbon-containing material from the catalyst bed.

In addition, hydrogen in a feedstock stream into an ETL reactor can enhance ETL catalyst lifetime. Hydrogen gas (H₂) can be directed into an ETL reactor and over an ETL catalyst, which can reduce the concentration of carbon-containing material (e.g., coke) that may be present on the catalyst and prohibit the deposition of carbon-containing material by hydrocracking reactions, for example, by breaking up larger molecules that may be eventually turned into coke and decrease catalyst activity.

### Catalysts for the Conversion of Olefins to Liquids

The present invention also provides catalysts and catalyst compositions for ethylene conversion processes, in accordance with the processes described herein. In some embodiments, the disclosure provides modified zeolite catalysts and catalyst compositions for carrying out a number of desired ethylene conversion reaction processes. In some cases, provided are impregnated or ion exchanged zeolite catalysts useful in conversion of ethylene to higher hydrocarbons, such as gasoline or gasoline blendstocks, diesel and/or jet fuels, as well as a variety of different aromatic compounds. For example, where one is using ethylene conversion processes to convert OCM product gases to gasoline or gasoline feedstock products or aromatic mixtures, one may employ modified ZSM catalysts, such as ZSM-5 catalysts modified with Ga, Zn, Al, or mixtures thereof. In some cases, Ga, Zn and/or Al modified ZSM-5 catalysts are preferred for use in converting ethylene to gasoline or gasoline feedstocks. Modified catalyst base materials other than ZSM-5 may also be employed in conjunction with the invention, including, e.g., Y, ferrierite, mordenite, and additional catalyst base materials described herein. The amount of active sites for these base materials is proportional to the SiO₂/Al₂O₃ ratio. The SiO₂/Al₂O₃ ratio for oligomerization catalyst can range from 2-1000, 20-800, and 80-280.

In some cases, ZSM catalysts, such as ZSM-5 are modified with Co, Fe, Ce, or mixtures of these and are used in ethylene conversion processes using dilute ethylene streams that include both carbon monoxide and hydrogen components (See, e.g., Choudhary, et al., Microporous and Mesoporous Materials 2001, 253-267, which is incorporated herein by reference). In particular, these catalysts can be capable of co-oligomerizing the ethylene and H₂ and CO components into higher hydrocarbons, and mixtures useful as gasoline, diesel or jet fuel or blendstocks of these. In such embodiments, a mixed stream that includes dilute or non-dilute ethylene concentrations along with CO/H₂ gases can be passed over the catalyst under conditions that cause the co-oligomerization of both sets of feed components. Use of ZSM catalysts for conversion of syngas to higher hydrocarbons can be described in, for example, Li, et al., Energy and Fuels 2008, 22:1897-1901, which is incorporated herein by reference in its entirety.

The present disclosure provides various catalysts for use in converting olefins to liquids. Such catalysts can include an active material on a solid support. The active material can be configured to catalyze an ETL process to convert olefins to higher molecular weight hydrocarbons.

ETL reactors of the present disclosure can include various types of ETL catalysts. In some cases, such catalysts are zeolite and/or amorphous catalysts. Examples of zeolite catalysts include ZSM-5, Zeolite Y, Beta zeolite and Mordenite. Examples of amorphous catalysts include solid phosphoric acid and amorphous aluminum silicate. Such catalysts can be doped, such as using metallic and/or semiconductor dopants. Examples of dopants include, without limitation, Ni, Pd, Pt, Zn, B, Al, Ga, In, Be, Co, Mg, Ca and Sr. Such dopants can be situated at the surfaces, in the pore structure of the catalyst and/or bulk regions of such catalysts.

Catalyst can be doped with materials that are selected to effect a given or predetermined product distribution. For example, a catalyst doped with Mg or Ca can provide selectivity towards olefins for use in gasoline. As another example, a catalyst doped with Zn or Ga (e.g., Zn-doped ZSM-5 or Ga-doped ZSM-5) can provide selectivity towards aromatics. As another example, a catalyst doped with Ni (e.g., Ni-doped zeolite Y) can provide selectivity towards diesel or jet fuel.

Catalysts can be situated on solid supports. Solid supports can be formed of insulating materials, such as TiOx or AlOx, wherein ‘x’ is a number greater than zero, or ceramic materials.

Catalyst of the present disclosure can have various cycle lifetimes (e.g., the average period of time between catalyst regeneration cycles). In some cases, ETL catalysts can have lifetimes of at least about 50 hours, 100 hours, 110 hours, 120 hours, 130 hours, 140 hours, 150 hours, 160 hours, 170 hours, 180 hours, 190 hours, 200 hours, 210 hours, 220 hours, 230 hours, 240 hours, 250 hours, 300 hours, 350 hours, or 400 hours. At such cycle lifetimes, olefin conversion efficiencies less than about 90%, 85%, 80%, 75%, 70%, 65%, or 60% may be observed.

Catalysts of the present disclosure can be regenerated through various regeneration procedures, as described elsewhere herein. Such procedures can increase the total lifetimes of catalysts (e.g., length of time before the catalyst is disposed of). An example of a catalyst regeneration process is provided in Lubo Zhou, “BP-UOP Cyclar Process,” Handbook of Petroleum Refining Processes, The McGraw-Hill Companies (2004), pages 2.29-2.38, which is entirely incorporated herein by reference.

In some embodiments, ETL catalysts can be comprised of base materials (first active components) and dopants (second active components). The dopants can be introduced to the base materials through appropriate methods and procedures, such as vapor or liquid phase deposition. Dopants can be selected from a variety of elements, including metallic, non-metallic or amphoteric in forms of elementary substance, ions or compounds. A few representative doping elements are Ga, Zn, Al, In, Ni, Mg, B and Ag. Such dopants can be provided by dopant sources. For example, silver can be provided by way of AgCl or sputtering. The selection of doping materials can depend on the target product nature, such as product distribution. For example, Ga is favorable for aromatics-rich liquid production while Mg is favorable for aromatics-poor liquid production.

Base materials can be selected from crystalline zeolite materials, such as ZSM-5, ZSM-11, ZSM-22, Y, beta, mordenite, L, ferrierite, MCM-41, SAPO-34, SAPO-11, TS-1, SBA 15 or amorphous porous materials, such as amorphous silicoaluminate (ASA) and solid phosphoric acid catalysts. The cations of these materials can be NH₄, H or others. The surface areas of these materials can be in a range of 1 m²/g to 10,000 m²/g, 10 m²/g to 5,000 m²/g, or 100 m²/g to 1,000 m²/g. The base materials can be directly used for synthesis or undergo some chemical treatment, such as desilication (de-Si) or dealumination (de-Al) to further modify the functionalities of these materials.

The base materials can be directly used for synthesis or undergo chemical treatment, such as desilication (de-Si) or dealumination (de-Al), to get derivatives of the base materials. Such treatment can improve the catalyst lifetime performance by creating larger pore volumes, such as pores having diameters greater than or equal to about 1 nanometer (nm), 2 nm, 3 nm, 4 nm, 5 nm, 10 nm, 20 nm, 30 nm, 40 nm, 50 nm, or 100 nm. In some cases, mesopores having diameters between about 1 nm and 100 nm, or 2 nm and 50 nm are created. In some examples, silica or alumina, or a combination of silica and alumina, can be etched from the base material to make a larger pore structure in the base catalyst that can enhance diffusion of reactants and products into the catalyst material. Pore diameter(s) and volume, in addition to porosity, can be as determined by adsorption or desorption isotherms (e.g., Brunauer-Emmett-Teller (BET) isotherm), such as using the method of Barrett-Joyner-Halenda (BJH). See Barrett E. P. et al., “The determination of pore volume and area distributions in porous substances. I. Computations from nitrogen isotherms,” J. Am. Chem. Soc. 1951. V. 73. P. 373-380. Such method can be used to calculate material porosity and mesopore volumes, in some cases volumes that are 3-7 times larger than their original materials. In general, any changes in catalyst structure, composition and morphology can be measured by technologies of BET, SEM and TEM, etc.

There are various approaches for doping catalysts. In an example, the doping components can be added to the base materials and their derivatives through impregnation, in some cases using incipient wetness impregnation (IWI), ion exchange or framework substitution in a zeolite synthesis operation. In some cases, IWI can include i) mixing a salt solution of the doping component with base material, for which the amount of salt is calculated based on doping level, ii) drying the mixture in an oven, and iii) calcining the product at a certain temperature for a certain time, typically 550-650° C., 6-10 hrs. Ion exchange catalyst synthesis can include i) mixing a salt solution, which can contain at least 1.5, 2, 3, 4, 5, 6, 7, 8, 9, or 10 times excess amount of the doping component, with base material, ii) heating the mixture, such as, for example, at a temperature from about 50° C. to 100° C., 60° C. to 90° C., or 70° C. to 80° C. for a time period of at least about 10 minutes, 30 minutes, 1 hour, 2 hours, 3 hours, 4 hours, 5 hours, 6 hours, 7 hours, 8 hours, 9 hours, 10 hours, 11 hours, or 12 hours, to conduct a first ion exchange, iii) separating the first ion exchange mother solution, iv) adding a new salt solution and repeating ii) and iii) to conduct a second ion exchange, v) washing the wet solid with deionized water to remove or lower the concentration of soluble components, vi) drying the raw product, such as air drying or in an oven, and vii) calcining the raw product at a temperature from about 450° C. to 800° C., 500° C. to 750° C., or 550° C. to 650° C. for a time period from about 1 hour to 24 hours, 4 hours to 12 hours, or 6 hours to 10 hours.

### Catalyst Forming

In some situations, powder catalysts prepared according to methods of the present disclosure may need to be formed prior to prepared in predetermined forms (or form factors) prior to use. In some examples, the forms can be selected from cylinder extrudates, rings, trilobe, and pellets. The sizes of the forms can be determined by reactor size. For example, for a 1″-2″ internal diameter (ID) reactor, 1.7 mm to 3.0 mm extrudates or equivalent size for other forms can be used. Larger forms can be used for different commercial scales (such as 5 mm forms). The ETL reactor inner diameter (ID) can be any diameter, including ranging from 2 inches to 10 feet, from 1 foot to 6 feet, and from 3 feet to 4 feet. In commercial reactors, the diameters of the catalyst (e.g., extrudate) can be greater than about 3 mm, greater than about 4 mm, greater than about 5 mm, greater than about 7 mm, greater than about 10 mm, greater than about 15 mm, or greater than about 20 mm. Binding materials (binder) can be used for forming the catalysts and improving catalyst particle strength. Various solid materials that are inert towards olefins (e.g., ethylene), such as Boehmite, alumina, silicate, Bentonite, or kaolin, can be used as binders.

Other binder material may be used to catalyze coke combustion in the catalyst regeneration process. These materials are capable of lowering the catalyst coke combustion process temperature below the temperature required for un-catalyzed catalyst coke combustion process. Lowering the catalyst coke combustion temperature may achieve a more conservative catalyst regeneration process and may be beneficial to the catalyst lifetime. Catalyst activity can be reduced by temperatures over about 650° C. especially in the presence of water. Catalyst activity can be reduced by exposure to water for extended periods of time. The combination of high temperature and water (e.g. steam) may over time during many regeneration cycles irreversibly deactivate the catalyst, requiring a fresh catalyst charge in the oligomerization reactors. Lowering the required catalyst regeneration temperature can be achieved through judicious choice of catalyst binders to act as catalyst for the coke combustion process. These catalyst binders may include but not limited to: cerium oxide (CeO₂, Ce₂O₃); zirconium oxide (ZrO₂); praseodymium oxide (Pr₂O₃, PrO₂); titanium oxide (TiO₂); and mixtures thereof. The binder material may have surface areas that range from <1 m²/g binder to <10 m²/g binder; 10 m²/g binder to <100 m²/g binder; 100 m²/g binder to <1000 m²/g binder.

A wide range of catalyst:binder ratio can be used, such as, from about 95:5 to 30:70, or 90:10 to 50:50. In some cases, a ratio of 80:20 is used for bench scale and pilot reactor catalyst synthesis. For formed catalysts, the crush strengths can be in the range of about 1 N/mm to 60 N/mm, 5 N/mm to 30 N/mm, or 7 N/mm to 15 N/mm.

Catalyst binders may also be used to activate O₂ present in the oligomerization process feed gas, 209, for continuous removal coke compounds on the catalyst surface and/or activating O₂ present in the process feed gas, 209, for increasing the selectivity for C₅₊ compounds and aromatic compounds (e.g. benzene, toluene, xylenes, mesitylenes). The binder promotes the oxidative dehydrogenation reaction of alkanes and napthenes to produce C₅₊ compounds and/or aromatic compounds respectively in the presence of O₂. These catalyst binders may include but not limited to: cerium oxide (CeO₂, Ce₂O₃); zirconium oxide (ZrO₂); praseodymium oxide (Pr₂O₃, PrO₂); titanium oxide (TiO₂); and mixtures thereof. The binder material may have surface areas that range from <1 m²/g binder to <10 m²/g binder; 10 m²/g binder to <100 m²/g binder; 100 m²/g binder to <1000 m²/g binder.

A wide range of catalyst:binder ratio can be used, such as, from about 95:5 to 30:70, or 90:10 to 50:50. In some cases, a ratio of 80:20 is used for bench scale and pilot reactor catalyst synthesis. For formed catalysts, the crush strengths can be in the range of about 1 N/mm to 60 N/mm, 5 N/mm to 30 N/mm, or 7 N/mm to 15 N/mm.

Catalysts prepared according to methods of the present disclosure can be tested for the production of various hydrocarbon products, such as gasoline and/or aromatics production. In some cases, such catalysts are tested for the production of both gasoline and aromatics.

In an example, a short-term test condition for gasoline production is 300° C., atmospheric pressure, WHSV=0.65 hr⁻¹, N₂ 50% and C₂H₄ 50%, two hour runs. In another example, a short-term test condition for aromatics production is 450° C., atmospheric pressure, WHSV=1.31 hr⁻¹, N₂ 50% and C₂H₄ 50%, two hour runs. In addition to conducting the two hour short-term test to obtain the initial catalytic activity data, for some selected catalysts, the long-term test (lifetime test) are also performed to obtain data of catalyst lifetime, catalyst capacity as well as average product composition over the lifetime runs.

In an example, the results on an initial catalytic activity test at gasoline production conditions is C₂H₄ conversion greater than about 99%, C₅₊ C mole selectivity greater than about 65% (e.g., 65%-70%), and C₅₊ C mole yield greater than about 65% (e.g., 65%-70%). Catalyst lifetime performance in one cycle run at gasoline conditions can be at least about 189 hours, cut at conversion down to 80%; catalyst capacity is about 182 g-C₂H₄ converted per g-catalyst with C mole yield of C₅₊+C₃₌C₄₌ greater than about 70%. With recycling, C₃₌ and C₄₌ can be accounted as liquid products.

In another example, the results on an initial catalytic activity at aromatics production conditions is C₂H₄ conversion greater than about 99%, C₅₊ C mole selectivity greater than about 75% (e.g., 75-80%), C₅₊ C mole yield greater than about 75% (e.g., 75-80%) and aromatics in C₅₊ greater than about 90%. Catalyst lifetime performance in one cycle run at aromatics production conditions can be at least about 228 hours, cut at conversion down to 82%, catalyst capacity 143 g-C₂H₄ converted/g-catalyst with average C₅₊ yield around 72% and aromatics yield around 62%.

An ETL catalysts can have a porosity that is selected to optimize catalyst performance, including selectivity, lifetime, and product output. The porosity of an ETL catalyst can be between about 4 Angstroms to about 1 micrometer, from 0.01 nm to 500 nm, from 0.1 nm to 100 nm, or from 1 nm to 10 nm as measured by pore symmetry (e.g., nitrogen porosimetry). An ETL catalyst can have a base material with a set of pores that have an average pore size (e.g., diameter) from about 4 Angstroms to 100 nm, or 4 Angstroms to 10 nm, or 4 Angstroms to 10 Angstroms.

The catalytic materials may also be employed in any number of forms. In this regard, the physical form of the catalytic materials may contribute to their performance in various catalytic reactions. In particular, the performance of a number of operating parameters for a catalytic reactor that impact its performance can be significantly impacted by the form in which the catalyst is disposed within the reactor. The catalyst may be provided in the form of discrete particles, e.g., pellets, extrudates or other formed aggregate particles, or it may be provided in one or more monolithic forms, e.g., blocks, honeycombs, foils, lattices, etc. These operating parameters include, for example, thermal transfer, flow rate and pressure drop through a reactor bed, catalyst accessibility, catalyst lifetime, aggregate strength, performance, and manageability.

In some cases, it is also desirable that the catalyst forms used will have crush strengths that meet the operating parameters of the reactor systems. In particular, a catalyst particle crush strength should generally support both the pressure applied to that particle from the operating conditions, e.g., gas inlet pressure, as well as the weight of the catalyst bed. In general, it may be desirable that a catalyst particle have a crush strength that is greater than about 1 N/mm², 2 N/mm², 3 N/mm², 4 N/mm², 5 N/mm², 6 N/mm², 7 N/mm², 8 N/mm², 9 N/mm², 10 N/mm², or more. As will be appreciated, crush strength may be increased through the use of catalyst forms that are more compact, e.g., having lower surface to volume ratios. However, adopting such forms may adversely impact performance. Accordingly, forms are chosen that provide the above described crush strengths within the desired activity ranges, pressure drops, etc. Crush strength may also be impacted through use of binder and preparation methods (e.g., extrusion or pelleting).

For example, in some embodiments the catalytic materials are in the form of an extrudate or pellet. Extrudates may be prepared by passing a semi-solid composition comprising the catalytic materials through an appropriate orifice or using molding or other appropriate techniques. Pellets may be prepared by pressing a solid composition comprising the catalytic materials under pressure in the die of a tablet press. Other catalytic forms include catalysts supported or impregnated on a support material or structure. In general, any support material or structure may be used to support the active catalyst. The support material or structure may be inert or have catalytic activity in the reaction of interest. For example, catalysts may be supported or impregnated on a monolith support. In some embodiments, the active catalyst is actually supported on the walls of the reactor itself, which may serve to minimize oxygen concentration at the inner wall or to promote heat exchange by generating heat of reaction at the reactor wall exclusively (e.g., an annular reactor in this case and higher space velocities).

The stability of the catalytic materials is defined as the length of time a catalytic material will maintain its catalytic performance without a significant decrease in performance (e.g., a decrease that is greater than about 1%, 5%, 10%, 15%, 20%, or more in hydrocarbon or soot combustion activity). In some embodiments, the catalytic materials have stability under conditions required for the hydrocarbon combustion reaction of >1 hr, >5 hrs, >10 hrs, >20 hrs, >50 hrs, >80 hrs, >90 hrs, >100 hrs, >150 hrs, >200 hrs, >250 hrs, >300 hrs, >350 hrs, >400 hrs, >450 hrs, >500 hrs, >550 hrs, >600 hrs, >650 hrs, >700 hrs, >750 hrs, >800 hrs, >850 hrs, >900 hrs, >950 hrs, >1,000 hrs, >2,000 hrs, >3,000 hrs, >4,000 hrs, >5,000 hrs, >6,000 hrs, >7,000 hrs, >8,000 hrs, >9,000 hrs, >10,000 hrs, >11,000 hrs, >12,000 hrs, >13,000 hrs, >14,000 hrs, >15,000 hrs, >16,000 hrs, >17,000 hrs, >18,000 hrs, >19,000 hrs, >20,000 hrs, >1 yrs, >2 yrs, >3 yrs, >4 yrs, >5 yrs or more.

The ETL catalyst can require a high density of active sites to be effective in some cases. Low active site density can lead to poor catalyst activity or performance. Another aspect of the present disclosure provides a catalyst for converting olefins to liquid hydrocarbons, the catalyst comprising: (a) a zeolite base material; (b) a binder; and (c) a dopant material, where the catalyst has an active site density of at least about 400 micro-moles (mol) of active sites per gram (g) of catalyst as measured by ammonia temperature programmed desorption (TPD). TPD is an acid-base titration that can be used to quantify the amount of active sites in a sample of catalyst and is a routinely used procedure in the field of catalysis.

In some embodiments, the catalyst is capable of converting at least about 99% of olefins to liquid hydrocarbons at an olefin weight hourly space velocity (WHSV) of at least about 0.7 at a reaction temperature of about 300° C.

In some cases, the active site density of the catalyst is about 350 micro-moles per gram (mol/g), about 375 μmol/g, about 400 μmol/g, about 425 μmol/g, about 450 μmol/g, about 500 mol/g, about 525 μmol/g, about 550 μmol/g, about 575 μmol/g, about 600 μmol/g, about 650 mol/g, about 700 μmol/g, about 750 μmol/g, about 800 μmol/g, about 900 μmol/g, about 1000 mol/g, about 1200 μmol/g, about 1500 μmol/g, about 2000 μmol/g, or about 5000 μmol/g. In some instances, the active site density of the catalyst is at least about 350 micro-moles per gram (mol/g), at least about 375 μmol/g, at least about 400 μmol/g, at least about 425 μmol/g, at least about 450 mol/g, at least about 500 μmol/g, at least about 525 μmol/g, at least about 550 μmol/g, at least about 575 μmol/g, at least about 600 μmol/g, at least about 650 μmol/g, at least about 700 μmol/g, at least about 750 μmol/g, at least about 800 μmol/g, at least about 900 μmol/g, at least about 1000 mol/g, at least about 1200 μmol/g, at least about 1500 μmol/g, at least about 2000 μmol/g, or at least about 5000 μmol/g.

### Catalyst Poisoning

Catalysts of the present disclosure can be poisoned during the course of catalytically generating a given product. ETL catalysts, for instance, can be poisoned upon generating higher molecular weight hydrocarbons from olefins (e.g., ethylene). The present disclosure provides various approaches for avoiding such poisons.

Alkynes can be oligomerized over ETL catalysts, such as zeolites or acid catalysts. During alkyne oligomerization, the alkynes can be rapidly transformed into polyaromatic molecules, precursors to coke, which can deactivate the catalyst. The selectivity for acetylene to make coke can deactivate the ETL catalyst at a faster rate than an alkene and the catalyst may need to be taken off line to be regenerated. Any molecule containing an alkyne functional group can deactivate the ETL catalyst at a faster rate than an alkene group. One example is acetylene, an alkyne produced in small quantities within the OCM process.

An approach for eliminating alkynes from feedstock to an ETL catalyst is to convert the alkynes to other material that may not poison the ETL catalyst. For example, alkynes can be selectively hydrogenated to make olefins using a variety of transition metal catalysts without hydrogenating the olefins into alkanes. Examples of these catalysts are Pd, Fe, Co, Ni, Zn, and Cu containing catalysts. Such catalysts can be incorporated in or more reactors upstream of ETL catalysts.

Dienes can be oligomerized over ETL catalysts, such as zeolites or acid catalysts. However during diene oligomerization, dienes can be rapidly transformed into polydienes molecules, precursors to coke, which can deactivate the ETL catalyst. The selectivity for dienes to make coke can rapidly deactivate the ETL catalyst and the catalyst may need to be taken off line to be regenerated. Any molecule containing a diene functional group can rapidly deactivate the ETL catalyst. An example is butadiene, a diene produced in small quantities within the OCM process.

An approach for eliminating dienes from feedstock to an ETL catalyst is to convert the dienes to other material that may not poison the ETL catalyst. For example, dienes can be selectively hydrogenated to make olefins using a variety of transition metal catalysts without hydrogenating the olefins into alkanes. Examples of these catalysts are Pd, Fe, Co, Ni, Zn, and Cu containing catalysts.

Bases can react to neutralize the acid functionality that catalyzes ETL reactions. If enough base reacts with the ETL catalyst, the catalyst may no longer be active toward oligomerization and may need to be regenerated. Bases include nitrogen containing compounds, particularly ammonia, amines, pyridines, pyroles, and other organic nitrogen containing compounds. Metal hydroxide compounds such as lithium, sodium, potassium, cesium hydroxides and group IIA metal hydroxides may deactivate the catalyst as well as carbonates of group IA and IIA metals.

Bases can be removed from feedstock to an ETL reactor by, for example, contacting the feedstock stream with water. This can remove or decrease the concentration of bases, such as amines, carbonates, and hydroxides.

Sulfur-containing compounds can deactivate ETL catalysts, particularly if the catalysts are doped with transition metal compounds. Sulfur can irreversible bind to the catalyst or metal dopant to deactivate the catalyst toward oligomerization. Organic sulfur compounds such as thiols, disulfides, thiolethers, thiophenes and others mercaptan compounds can be detrimental to the ETL catalyst.

Sulfur-containing compounds can be removed from feedstock to an ETL reactor by gas scrubbing, such as, for example, amine gas scrubbing. Amines can react with sulfur compounds (e.g., H₂S) to remove such compounds from gas streams. Other ways of removing sulfur compounds are by molecular sieves or hydrotreating. Examples of approaches for removing sulfur-containing compounds from a gas stream are provided in Nielsen, Richard B., et al. “Treat LPGs with amines,” Hydrocarbon Process 79 (1997): 49-59, which is entirely incorporated herein by reference.

The impact that certain non-ethylene gases can have on ETL catalysts is summarized in Table 1.

TABLE 1
Impact of non-ethylene gases on ETL catalyst

| Feedstock | General Catalyst Impact |
|---|---|
| N₂ | Inert |
| Methane | Inert |
| CO₂ | Inert in small quantities |
| H₂ | Coke suppressant but can hydrogenate olefins |
| | in large quantities and facilitate cracking of |
| | C₅₊ product |
| H₂O | Coke suppressant but can deactivate catalyst |
| | in large quantities |
| ethane | Inert |
| propylene | Oligomerizes to C5+ |
| butylene | Oligomerizes to C5+ |
| acetylene | Coke accelerator |
| Dienes | Coke accelerator |
| CO | Inert in small quantities |
| amines | Lowers catalyst activity |
| Metal oxides | Lowers catalyst activity |
| phosphines | Lowers catalyst activity |
| arsines | Lowers catalyst activity |

The present disclosure also provides reactor systems for carrying out ethylene conversion processes. A number of ethylene conversion processes can involve exothermic catalytic reactions where substantial heat is generated by the process. Likewise, for a number of these catalytic systems, the regeneration processes for the catalyst materials likewise involve exothermic reactions.

As such, reactor systems for use in these processes can generally be configured to effectively manage excess thermal energy produced by the reactions, in order to control the reactor bed temperatures to most efficiently control the reaction, prevent deleterious reactions, and prevent catalyst or reactor damage or destruction.

### ETL Separations

Separations for ETL processes of the present disclosure can be carried out in three places within the ETL scheme: before the ETL reactor, within the ETL reactor and downstream of the ETL reactor. In each of these three places, different separations technologies can be employed.

To process the ETL reactor feed, traditional gas separations equipment can be used. These separations may include pressure swing adsorption, temperature swing adsorption and membrane-based separation. The reactor feed may also be augmented by utilizing cryogenic separations equipment found in a traditional midstream gas plant.

To make changes to the composition within the reactor, different types of catalysts can be co-mixed or layered within the catalyst bed or reactor vessel. Different types of zeolite catalysts (for example a ZSM-5 and a SAPO 34 in a 60%/40% mixture or in a 50%/50% mixture) may create different hydrocarbon profiles at the reactor vessel outlet. Also within this vessel, there may be a combination of multiple beds with appropriate quenches built in to affect the final product composition.

To separate the reactor outlet mixtures, a combination of flash separation, hydrogenation, isomerization and distillation can be used. Flash separation may remove most of the light fractions of the hydrocarbon liquid product. This can affect product qualities like Reid Vapor Pressure. Hydrogenation, isomerization and distillation can then be used, much like traditional refining processes, to create a fungible product.

ETL separation can be implemented upstream of an ETL reactor. Membranes used in conjunction with the ETL process can be used on the process feedstock to enrich components prior to directing the feedstock to the ETL reactor. Ethylene may be a component that can be enriched. Other components of the feedstock may also be enriched, such as H₂ and/or CO₂. In some cases, CO may be rejected.

For example, CO in the feedstock may be a catalyst poison. CO can be removed prior to directing the feedstock to the ETL reactor. Hydrogen may be an advantageous species to have in the feedstock because it can reduce coking rates, thus lengthening on-stream time between de-coke cycles.

In some cases, a membrane separation unit upstream of an ETL reactor may be employed. The membrane unit can remove at least about 20%, 30%, 40%, 50% or 60% of one component, or increase the amount of ethylene from at least about 1%, 2%, 3%, 4% or 5% to at least about 10%, 15%, 20%, 30%, or 40%.

As another example, ethylene can be enriched using a membrane that has a certain chemical affinity to ethylene. For oxygen separations membranes, cobalt can be used within the membranes to chemically pull oxygen through the membranes. Chemically-modified membranes can be used to effect such separation.

Another technique that can be employed for upstream separation is pressure swing adsorption (PSA). Pressure swing adsorption can be used to remove substantially all of a certain poison, or enrich ethylene to near purity. In some cases, PSA may be used in place of, or in addition, membrane. The PSA unit can include at least 2, 3, 4, 5, 6, 7, 8, 9, or 10 vessels that contain an adsorbent. This adsorbent may be a combination of zeolites, molecular sieves or activated carbon, Metal Organic Frameworks (MOF) for example. Each vessel can contain one or more adsorbents co-mixed or layered within the vessel.

Metal Organic Frameworks are a class of porous materials comprised of inorganic units linked with coordinating organic units. MOFs have a large internal surface area and can be tuned to a desired physical or chemical property by judicious selection of the inorganic unit and the organic linker unit. Due to the high internal surface area and strong adsorption sites (e.g. exposed metal cations), MOFs have applications in gas separation, chemical catalysis, and sensors. For example in gas separation, the high density of exposed metal sites leads to a high capacity for gas adsorption of gas molecules (e.g. ethylene, ethane, CO₂) per mass of MOF. MOF applications in hydrocarbon separations can be found in the following references: Geier et al. Chem. Sci. 4:2054 (2013); Blocj eta. Science 335:1606). The inorganic unit and organic unit in MOFs can also be tuned to be selectively store hydrogen (H₂) gas. H₂ separation and storage can be found in the following references: Zhou et al. J. Am. Chem. Soc. 130:15268 (2008). Liu et al. Langmuir 24:4772 (2008). Methane (CH₄) separation and storage can be found in the following references: Wu et al. J Am. Chem. Soc. 131:4995; Makal et al. Chem. Soc. Rev. 41:7761. Carbon dioxide (CO₂) separation and storage can be found in the following references: Dietzel et al. Chem. Commun. 5125 (2008); Caskey et al. J. Am. Chem. Soc. 130: 10870 (2009).

MOFs may comprise repeating cores which comprise: a plurality of metals, metal ions, and/or metal containing complexes that are linked together by forming covalent bonds with linking clusters of a plurality of linking moieties. One or more metals, metal ions, and/or metal containing complexes, that can be used in the synthesis of a MOF, exchanged post synthesis of a MOF, and/or added to a MOF by forming a coordination comples with post framework reactant linking clusters, including, but not limited to:
Group I” elements include lithium (Li), sodium (Na), potassium (K), rubidium (Rb), cesium (Cs), and francium (Fr).
“Group II” elements include beryllium (Be), magnesium (Mg), calcium (Ca), strontium (Sr), barium (Ba), and radium (Ra).
“Group III” elements include scandium (Sc) and yttrium (Y).
“Group IV” elements include titanium (Ti), zirconium (Zr), halfnium (Hf).
“Group V” elements include vanadium (V), niobium (Nb), tantalum (Ta).
“Group VI” elements include chromium (Cr), molybdenum (Mo), tungsten (W).
“Group VII” elements include manganese (Mn), technetium (Tc), rhenium (Re).
“Group VIII” elements include iron (Fe), ruthenium (Ru), osmium (Os).
“Group IX” elements include cobalt (Co), rhodium (Rh), iridium (Ir).
“Group X” elements include nickel (Ni), palladium (Pd), platinum (Pt).
“Group XI” elements include copper (Cu), silver (Ag), gold (Au).
“Group XII” elements include zinc (Zn), cadmium (Cd), mercury (Hg).
“Lanthanides” include lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), yitterbium (Yb), and lutetium (Lu).
“Actinides” include actinium (Ac), thorium (Th), protactinium (Pa), uranium (U), neptunium (Np), plutonium (Pu), americium (Am), curium (Cm), berklelium (Bk), californium (Cf), einsteinium (Es), fermium (Fm), mendelevium (Md), nobelium (No), and lawrencium (Lr).

MOFs may contain a plurality of pores which can be used for gas adsorption. In one variation, the plurality of pores has a unimodal size distribution. In another variation, the plurality of pores has a multimodal (e.g. bimodal) size distribution.

MOF gas storage or separation material may store or separate the following gases, but not limited to, ammonia, argon, carbon dioxide, carbon monoxide, hydrogen, methane, ethylene, ethane, H₂, propane, propenes, butenes, butanes, and combinations thereof.

MOF material powders may be formed into various shapes and sizes using extrustion or pelleting techniques before being placed in storage or separations process vessels. Shapes and sizes for the adsorbent material for guard beds, (e.g., guard beds 3602A-B in FIG. 36), include: spheres; trilobes; quadralobes; and cylinders in the range of about 1 mm-20 mm in diameter and about 1 mm-50 mm in length.

Binding materials (binder) can be used for forming the catalysts and improving catalyst particle strength. Various solid materials that are inert towards olefins (e.g., ethylene), such as Boehmite, alumina, silicate, Bentonite, or kaolin, can be used as binders. In addition, organic compounds and polymers may be used as binders for forming MOFs (e.g. starch, styrene, polyvinylpyrrolidone, polyethyleneglycol).

The PSA units can operate at ETL reactor pressures (e.g., about 5-50 bar) and blow down to atmospheric pressure. Activated carbon, 3A, 4A, 5A molecular sieves, zeolites, Metal Organic Frameworks, and Metal Organic Frameworks that have subjected to pyrolysis can be used in these beds. The vessels can be operated such that the wanted gases (e.g., ethylene) pass through the beds at high pressure, and unwanted gases (e.g., CO, CO₂ or methane) are blown down out of the bed at low pressure. Alternatively, the PSA vessels can be operated such that the unwanted gases (e.g., CO, H₂, CO₂ or methane) pass through the beds at high pressure, and wanted gases (e.g., ethylene) are blown down out of the bed at low pressure.

As an example, the specific choice of sorbent can determine the species that passes through at high pressure or is exhausted at low pressure. In some cases, a PSA can use layered sorbents, such as to effect methane and nitrogen separation. Such layering within the bed allows methane to be the blow down gas, rather than nitrogen.

PSA technology can also be used in other situations. Multiple beds can be used in series to further enrich the wanted process gases. PSA units with at least 2, 3, 4, 5, 6, 7, 8, 9, 10, 20, or 30 vessels may be employed. The PSA can be operated at high frequencies, which can further promote better separation.

Another separation technique that can be employed for use with ETL is temperature swing adsorption (TSA). In TSA, temperature changes are used to effect separation. TSA can be used to separation hydrocarbons mixtures after the ETL reactor. When gas mixtures are close to changing phases, TSA can be helpful in removing the heavy fraction from the light fraction.

The present disclosure also provides in-reactor separations (product augmentation) approaches. Some of the separations goals can be achieved within the catalyst bed, or within the reactor vessel itself, using reactive separations, for example. In reactive separation, a first molecule can be reacted to form a larger or smaller molecule that may be separated from a given stream.

In some cases, gas phase ethylene can be condensed to a liquid via reaction. This augmentation can take two forms within the catalyst bed: it can augment the product to bring it to within a given specification, or it can augment the product to remove downstream equipment. As an example of bringing products into specification, a hydrogenation catalyst can be co-mixed or layered within the bed, or as a second bed within a reactor vessel. This catalyst can utilize the available hydrogen to decrease the olefin content of the final product. Since fungible gasoline (and many other products) can have an olefin specification to prevent gumming, this in situ separation can remove a large amount of olefin content from the resulting liquid, bringing it to within a given specification.

A co-mixed bed with multiple types of different zeolite can affect the overall product composition. For example, a low-aromatic producing catalyst can be added in an 80%/20% mixture to a typical ETL catalyst. The resulting product stream can be lower in aromatics, and can bring an off-spec product to within a given specification.

As another approach, a downstream (in vessel) isomerization bed can be used to remove unwanted isomers, like durene. Hydrocarbon compounds of any appropriate carbon number, such as hydrocarbon compounds with four or more carbon atoms (C₄₊ compounds), can be isomerized. If a downstream unit is necessary to isomerize components like durene, or remove components, such as high boiling point components, an in-bed reactor approach can be employed.

In some situations, a mixture of zeolites that have been augmented via a process may also provide for a desirable separation. Such mixture can be used to provide for product augmentation.

The present disclosure also provides separations approaches downstream of an ETL reactor. Downstream separations equipment for an ETL process can be similar to equipment employed for use in refineries. In some cases, downstream unit operations can include flash separation, isomerization, hydrogenation and distillation, which can aid in bringing the final product to within a given specification.

Isomerization equipment can convert unwanted iso-durene into a more volatile form. Hydrogenation equipment can reduce the amount of olefins/aromatics in the final product. Distillation can separate material on the basis of boiling point. These units can be readily used to create a product having a product distribution as desired.

Isomerization equipment can be used to upgrade the octane rating of a hydrocarbon product composition. For example, n-hexane can be isomerized to i-hexane. N-pentane (62 octane) can be isomerized to 2-methyl-butane (93 octane). Hexane (25 octane) can be isomerized to 2-methyl-pentane (73 octane).

Alkylation and dimerization units can upgrade lighter fractions, such as butanes, into more valuable, higher octane products. If the ETL reactor produces a large amount of butenes compared to butanes, then dimerization can be used to convert the butene into isooctene/isooctane.

A catalytic reformer unit can upgrade light naphtha fraction to a reformate. This unit works by combining molecules and producing hydrogen. If well-placed, the hydrogen produced in this unit can be utilized in a downstream unit.

Depending on the size and scale of the ETL reactor, vacuum distillation can be employed to further refine the hydrocarbon product outputted by the ETL reactor. If such products are valuable as lubricants, oils and waxes, then the extra step to vacuum distill these products can be advantageous. In some cases, the amount of heavy components produced in the ETL reactor is less than 20%, 15%, 10%, 5% or 1%, but the value generated out of those products can be substantial.

Another approach for separating hydrocarbons is cryogenic separation. Such separation can be used to capture C₄ and C₅₊ compounds from an ETL reactor effluent product stream. In some cases, a cryogenic separation unit can include a cold box that may not use traditional deep cryogenic temperatures and may not require traditional unit operations of demethanizer and deethanizer. Such cryogenic separation unit may not produce high purity methane, ethane, or propane products. However, it may produce a mixed (in some cases primarily methane) stream with impurity ethane, propane, other light hydrocarbons and inert gases that are acceptable for use in other settings, such as reinjection to pipeline gas, as residue gas, or used to meet fuel requirements for power plants or feedstocks for syngas plants for the production of methanol or ammonia.

In some examples, a cryogenic separation unit can operate at a temperature from about −100° C. to −20° C., −90° C. to −40° C., or −80° C. to −50° C. Such temperatures can be obtained through methods that use the turboexpansion of high pressure pipeline natural gas or turboexpansion of moderate pressure high methane content feedstock gas, which may be typical of OCM reactor inlet requirements where additional cooling may be accomplished using traditional process plant refrigeration loops, including propane refrigeration or other mixed refrigerants.

In some cases, there may be substantial recovery of pressure-reduced power by coupling of turboexpander and residue gas compressors depending on final destination and usage of lighter nonreacted and unrecoverable hydrocarbons and other components.

In an example OCM-ETL system, gas is expanded and/or additional refrigeration cooled and fed to a cryogenic cold box unit, where heat is exchanged with multiple downstream product streams. It can then be fed to an OCM reaction and heat recovery section. Pressure can be increased through multiple process gas compressors, then heated for ETL and then ETL reaction section. Unrefrigerated liquids recovery can be accomplished using air and cooling water utilities before the product gas enters the cryogenic cold box unit, where it is cooled, pressure reduced for cooling effects, and additional condensed liquids removed via a liquid-liquid separator. Separated liquids can reenter the cryogenic cold box unit, where they are heat exchanged prior to being fed to a depropanizer unit which removes impurity propane and other light compounds from final C₄₊ product. Separated gas from the liquid-liquid separator also renter the cryogenic cold box unit where they are heat exchanged prior to being mixed with depropanizer overhead product gas and then fed to residue gas compressors based on final residue gas users. The depropanizer reflux condensation is also provided by sending this gas stream through the cryogenic cold box unit.

In some cases, a debutanizer column can be installed with bottoms product from depropanizer as feed. Its use can be to provide RVP control of final C₄₊ product. In some cases, RVP control may be precluded, other purifications or chemical conversions may be employed.

### ETL Reactor Feedstock

Olefin-to-liquids (e.g., ETL) processes of the present disclosure can be performed using feedstocks comprising one or more olefins, such as pure ethylene or diluted ethylene. Ethylene can be mixed with non-hydrocarbon molecules or other hydrocarbons, including olefins, paraffins, naphthenes, and aromatics. When a feedstock comprising these materials is directed over an ETL catalyst, such as a zeolite catalyst bed at temperatures of at least about 150° C., 200° C., 250° C., or 300° C., the reactants can oligomerize to form a combination of longer chain isomers of olefins and paraffins, naphthenes, and aromatics. The product slate can include hydrocarbons with carbon numbers between 1 and 19 (i.e., C₁-C₁₉).

The concentration of ethylene (or other olefin(s)) can be changed by adjusting the partial pressure of ethylene (or other olefin(s)) at constant total pressure by dilution with an inert gas, such as nitrogen or methane, or by adding an inert gas to increase the total pressure while keeping the partial pressure of ethylene constant. A change in concentration due to changes in the total pressure may not lead to significant variations in the process unless the system is operated in an adiabatic mode, in which temperature spikes introduce additional variability.

In an isothermal reactor operation, a change in concentration via adjustments in the partial pressure of ethylene can prompt increases in liquid content and reduction of olefins at the benefit of paraffins and aromatics. The changes observed in product slate and liquid formation can depend on the temperature regime and the class of molecules formed in that regime (e.g., isoparaffins and aromatics at temperatures below or above about 400° C., respectively). For example, increasing the concentration of ethylene from 5% to 15% at a constant total pressure of 1 bar and a WHSV of 1 g ethylene/g catalyst/hour can result in a change from 15% to 45% liquids at 300° C.

As the temperature increases, the starting liquid percent increases, yet the net change upon an increase in concentration diminishes. For example, at 390° C., increasing the concentration of ethylene from 5% to 15% at a constant total pressure of 1 bar can result in a change of 45% to 65% liquids. The composition of the product can also change with increasing concentration of ethylene. The trend is uniform with temperature: as the concentration increases, the content of olefins decreases at the benefit of paraffin isomers, naphthenes, and aromatics. As the temperature is increased to at least about 300° C., 350° C., 400° C. or 450° C. and the product slate is heavily aromatic, changes in the partial pressure of ethylene may not change the product slate but can cause a decrease in the liquid content.

In an adiabatic operation, the concentration of ethylene may result in a change in the liquid and product slate, which is coupled to the variations in temperature zones across the reactor bed. In this mode, the rate of heat transfer from a differential volume unit of the reactor bed is a function of the heat capacity of the catalyst and gaseous molecules in the stream—in particular the inert species. Thus, decreasing the concentration of ethylene helps increase the heat dissipation and the temperature in the volume unit. In general, as the concentration of ethylene is increased, the temperature in the bed can increase and the content of aromatics and net liquids can also increase at the expense of paraffins, isoparaffins, olefins, and naphthenes. When the temperature reaches at least about 300° C., 350° C., 400° C. or 450° C., the net amount of liquid can decrease as cracking of the liquid molecules becomes more prevalent.

In some cases, the addition of other hydrocarbons from a recycle, refinery or midstream operation combined with the ethylene feedstock may have a positive effect on the formation of liquids. The ETL process is an oligomerization reaction, in which hydrocarbons are combined to form longer chain hydrocarbons. Thus, introducing hydrocarbons with C₃₊ olefin chain length in addition to the C₂ ethylene promotes the formation of liquid. As long as the reaction conditions or inherent nature of the catalyst itself precludes cracking (β-scission) of the hydrocarbon, the addition of longer chain hydrocarbons in the feed may yield an oligomerized product that is the sum of the two molecules. In other words, the barrier to producing longer chain molecules is reduced by minimizing the number of molecular units at the start of the reactor (C₂+C₂+C₂+C₂=C₈ vs. C₂+C₆=C₈).

Gas molecules that can be co-fed with ethylene can come from a recycle stream, natural gas liquids, midstream operations, or refinery effluents comprising ethane, propylene, propane, butene isomers, and butane isomers, and other C₄₊ olefins. The general product slate can be more or less unchanged by introducing propylene, isobutene, and trans-2-butene (with similar expectations for other butene isomers). At a constant volumetric flowrate of hydrocarbon species, substitution of a longer chain hydrocarbon for a shorter chain hydrocarbon (e.g., propylene replacing ethylene) can result in a higher content of liquid formed.

For example, at T=300° C. with 0.15 bar partial pressure of hydrocarbon, 1 bar total pressure, a 50:50 mixture of propylene or isobutene with ethylene increases the liquid yield by 10%-20% in comparison to a pure ethylene feedstock (an increase in liquids can be due to an increase in liquid (C₅₊) isoparaffins). When the temperature is 390° C. or higher and aromatic molecules are the dominant product species, the impact of hydrocarbon length has less effect on the liquid formation. Regardless, we have found that the presence of propylene or isobutene in the feed promotes the formation of liquids (aromatics) to an extent (a few percentage points) that is greater than using an isolated pure feeds.

Additional paraffins (e.g., ethane, propane, and butane) can influence may impact an ETL reaction and product distribution. The introduction of n-paraffins may yield an increase in isoparaffin content due to isomerization of the molecules on the acid zeolite catalyst. As the temperature and rate of dehydrogenation increases, the impact of introduced paraffins may mirror the behavior observed by adding olefins. Co-feeding C₅₊ hydrocarbons with ethylene may also improve the liquid conversion performance of the ETL process due to the nature of the oligomerization process.

Additional details of the ETL process can be found in U.S. Pat. Nos. 9,321,702B2, 9,328,297B1, and 9,598,328B2, each of which is incorporated herein by reference in its entirety.

### ETL Using FCC Off-Gas to Produce C₅₊ and Olefin Free Fuel Oil

Fluid catalytic cracking (FCC) is one of the most important conversion processes used in petroleum refineries. It is widely used to convert the high-boiling, high-molecular weight hydrocarbon fractions of petroleum crude oils into more valuable gasoline, olefinic gases, and other products. Cracking of petroleum hydrocarbons was originally done by thermal cracking, which has been almost completely replaced by catalytic cracking because it produces more gasoline with a higher octane rating. It also produces byproduct gases that have more carbon-carbon double bonds (i.e. more olefins), and hence more economic value, than those produced by thermal cracking.

The feedstock to FCC is usually that portion of the crude oil that has an initial boiling point of 340° C. or higher at atmospheric pressure and an average molecular weight ranging from about 200 to 600 or higher. This portion of crude oil is often referred to as heavy gas oil or vacuum gas oil (HVGO). In the FCC process, the feedstock is heated to a high temperature and moderate pressure, and brought into contact with a hot, powdered catalyst. The catalyst breaks the long-chain molecules of the high-boiling hydrocarbon liquids into much shorter molecules, which are collected as a vapor.

The reaction product vapors (at 535° C. and a pressure of 1.72 bar) flow from the top of the reactor to the bottom section of the distillation column (commonly referred to as the main fractionator) where they are distilled into the FCC end products of cracked petroleum naphtha, fuel oil, and offgas. After further processing for removal of sulfur compounds, the cracked naphtha becomes a high-octane component of the refinery's blended gasolines.

The main fractionator offgas is sent to what is called a gas recovery unit where it is separated into butanes and butylenes, propane and propylenes, and lower molecular weight gases (hydrogen, methane, ethylene and ethane). Some FCC gas recovery units may also separate out some of the ethane and ethylene.

A delayed coker unit (DCU) also produces offgas that contains olefins, in a process similar to FCC. A DCU is a type of coker whose process consists of heating a residual oil feed to its thermal cracking temperature in a furnace with multiple parallel passes. This cracks the heavy, long chain hydrocarbon molecules of the residual oil into coker gas oil and petroleum coke.

Another possible source of refinery offgas includes a propane dehydrogenation (PDH) unit. Propane dehydrogenation (PDH) converts propane into propene and by-product hydrogen. The propene from propane yield is about 85 mole %. Reaction by-products (mainly hydrogen) are usually used as fuel for the propane dehydrogenation reaction.

Another possible source or refinery offgas is an oxidative dehydrogenation (ODH) unit. Dehydrogenation is a chemical reaction that involves the removal of hydrogen from an organic molecule. It is the reverse of hydrogenation. Dehydrogenation is an important reaction because it converts alkanes, which are relatively inert and thus low-valued, to olefins, which are reactive and thus more valuable.

There are alternative sources of refinery offgas that can be used in ETL processes, sources that depend on the individual refinery.

The offgas can be used as a fuel gas, in which it can be used as heat in the distillation column reboiler or elsewhere in the refinery. However, due to the olefin content of these streams, it is not suitable for use in gas turbines in order to generate electricity.

An aspect of the present invention are methods to utilize ethylene-to-liquids (ETL) technology in order to produce olefin-free fuel gas and C₅₊ hydrocarbons from the olefin rich offgas.

FIG. 38 shows a process by which clean fuel gas and C₅₊ hydrocarbons can be generated from FCC or DCU offgas 3800. An offgas stream 3801, coming from an FCC unit, a DCU, or another refinery offgas stream, is injected into a pretreatment bed subsystem 3802. The pretreatment bed subsystem can be used to remove contaminants that may otherwise damage or poison an ETL catalyst. Some contaminants may be sulfur-containing species. The gas then exits the pretreatment system and enters an ETL subsystem 3803. The ETL subsystem converts the ethylene and other olefins from the offgas stream into C₅₊ hydrocarbons. This stream may contain water, in which case it is injected into a drying unit 3804 in order to remove the water. Once water is removed, the dried ETL effluent stream is injected into a separations subsystem 3805 that can produce a stream containing C₅₊ hydrocarbons 3807 and a stream containing light gases 3806. The light gas stream can then be used as a fuel gas without olefins.

The type of pretreatment bed that is used depends on the composition of the offgas stream. For example, offgases that are rich in sulfur containing species (sour) may require a different guard bed than one that is poor in sulfur containing species (sweet). Additionally, the molecular identity of the sulfur containing species may also affect the pretreatment system (e.g. organic sulfur vs. H₂S). If there are multiple offgas streams from different sources (e.g. FCC offgas, oxidative-couping of methane offgas, oxidative dehydrogenation offgas, propane dehydrogenation offgas), one of those offgas streams can be injected into a process such as 3800 after the pretreatment bed 3802. The offgas can have between 1% and 20% light olefins after refinery FCC fractionation, and can contain up to 25% or more hydrogen.

The ETL subsystem can comprise one or more ETL reactors and one or more catalyst regeneration systems. In order to assist temperature control in the ETL reactor(s), a portion of the ETL effluent stream may be recycled into the ETL reactor(s).

Additionally, there can be synergies between using both ETL and OCM technology with refinery offgas streams. OCM can generate additional ethylene for use in ETL. This way, refinery offgas as well as OCM product gas can used as a feedstock for ETL, and some of the ETL products can be used as a feedstock for OCM.

FIG. 39 shows a process in which ETL and OCM are used with refinery offgas as a feedstock 3900. Here, a refinery offgas source 3901 is injected into a pretreatment bed 3902 in order to remove impurities that may poison or damage the ETL catalyst. The pretreated refinery offgas is then injected into an ETL subsystem 3903 that can convert ethylene and other light olefins into an ETL effluent stream containing C₅₊ hydrocarbons. The ETL effluent stream may contain water, in which case it is injected into a drying system 3904 to remove water and produce a dry ETL effluent stream. The dry ETL effluent stream is then injected into a separation subsystem 3905 that can produce a stream containing light gases 3906 (e.g. methane, hydrogen) from a stream containing ethane and heavier gases. The separation subsystem 3905 can be a demethanizer column, an adsorption system, a membrane system, or combinations thereof. The stream containing ethane and heavier gases is then injected into a subsequent separation subsystem 3907 that produces a stream containing ethane 3908 and a stream containing heavier gases. The separation subsystem 3907 can be a deethanizer column, an adsorption system, a membrane system, or combinations thereof. The stream containing heavier gases is then injected into a separation subsystem 3909 that produces a stream containing C₅₊ hydrocarbons 3910 and a stream containing propane 3911. The stream containing ethane 3908 and the stream containing propane 3911 are injected into an oxidative coupling of methane (OCM) subsystem 3912 to produce and OCM effluent 3913. The OCM subsystem 3912 can include one or more pretreatment subsystems, one or more OCM reactors, one or more heat exchangers, one or more process gas compressors, one or more amine scrubbers, and/or one or more additional separation subsystems. Some of the refinery offgas 3901 can also be used as a feed for the OCM subsystem. Additional natural gas, ethane, and/or propane can be added to the OCM subsystem. The OCM effluent gas is then injected into the ETL subsystem 3902.

### Alkylation and Dimerization Via Catalytic Distillation

Alkylation of olefins with isoparaffins can be used for the production of alkylate, a superior gasoline blendstock due to its unique characteristics such as high RON, no olefinic content, and low RVP, making it one of the most sought-after streams for gasoline blenders. Processes for alkylation include solid acid based alkylation and alkylation process employing HF or sulfuric acid as the alkylation catalysts. These processes may have, however, some shortcomings such as the specification of feedstocks that go into them, such as being limited to isobutane and C₃₊ olefins as reactants.

Example catalysts that can be effective in ethylene dimerization as well as in C₄ alkylation can be found in U.S. Pat. No. 9,079,815 and International Patent Publication No. WO/2016/210006, each of which is entirely incorporated herein by reference.

Concurrently, methodologies to reduce capital cost and the number of unit operations associated with the ETL process are needed, as this can add to the technology competitiveness, diversity, and flexibility. One such methodology lies in catalytic distillation, which combines reaction and separation of products in the same vessel, and enables a high level of conversion of reactants due to continuous removal of products (as per Le Chatelier's principle), which drives the equilibrium of the reaction towards the products. Literature that shows examples of the use and design of catalytic distillation units to carry our chemical transformations and separations is provided in U.S. Pat. Nos. 4,232,177, 5,003,124, 5,055,627, 5,057,468, and U.S. Patent Pub. No. 2006/0235246.

In an aspect of the present disclosure, one of or a mixture of any of C₂-C₅ olefins may be introduced to a catalytic distillation unit, where it reacts over a dimerization catalyst to produce longer chain olefins. The formed higher olefins (e.g., C₄₌) may react with iC₄ which may be introduced into the column to form alkylate. In some cases, an olefin isomerization unit may be used upstream of the catalytic distillation unit such that olefins (such as 1-butene) are isomerized into a mixture of olefin isomers (such as 1-butene and cis-2-butene, and trans-2-butene).

FIG. 40 shows a schematic for alkylation and dimerization via catalytic distillation 4000. In this schematic, a feed containing one or a mixture of any of C₂-C₅ olefins 4002 and a feed containing isobutane (iC₄) 4001 are injected into the catalytic distillation column 4003 in liquid or gas phase, where it may get into contact with a dimerization catalyst and converts into higher olefins (such as C₄₌). As formed olefins vapors move up in the column they get into contact with iC₄ and an alkylation catalyst where alkylation reactions proceed to form C₈₊ and nC₄/nC₅ by-products. The temperature and pressure of the column may be selected such that formed C₈₊ alkylates may condense into a liquid that moves downward in the column to a lower side stream 4006 while iC₄ and C₂-C₅ olefins vapors move up. By-product nC₄/nC₅ are lighter than alkylate and they may be drawn out of the column as an upper side stream 4005. Unconverted C₂-C₅ and iC₄ may be condensed and routed back to the column in a condenser 4004. In some cases, a re-boiler can be used to partially vaporize the C₈₊ alkylate product and recycle the vapor back into the column, but this is not required.

The operating conditions and catalyst of the dimerization bed, per those disclosed in U.S. Pat. No. 9,079,815 and International Patent Publication No. WO/2016/210006, each of which is entirely incorporated herein by reference, with catalysts that may include Ni, Pd, Cr, V, Fe, Co, Ru, Rh, Cu, Ag, Re, Mo, W, Mn, Pt—having a hydrogenation function introduced into the dimerization catalyst such that catalyst regeneration can proceed as per the simple methods disclosed in the above-mentioned patent/publication. The operating conditions and catalyst of the alkylation bed, with catalysts potentially including any one or combination of zeolites, sulfated zirconia, tungstated zirconia, chlorided alumina, aluminum chloride (AlCls), silicon-aluminum phosphates, titaniosilicates (including VTM zeolite), polyphosphoric acid (including solid phosphoric acid, or SPA, catalysts, which are made by reacting phosphoric acid with diatomaceous earth), polytungstic acid, and supported liquid acids such as triflic acid on silica, sulfuric acid on silica, hydrogen fluoride on carbon, antimony fluoride on silica, aluminum chloride (AlCls) on alumina (Al₂O₃), or the catalyst(s) disclosed in U.S. Pat. No. 9,079,815 and International Patent Publication No. WO/2016/210006. The operating conditions, catalysts, and reactor type and configuration of the olefin isomerization unit (if included), which employs catalysts typically used for olefin isomerization such as alkaline oxides (including MgO) can be varied. The ratio of starting olefin to iC₄ can be varied. Operation of the reboiler and condenser units (if included) such as to regulate the reflux ratios of C₂-C₅ olefins and iC₄ liquid/vapor and C₈₊ vapor back into the catalytic distillation column can be varied. The number of trays and/or height of packed catalyst beds used inside the column can be varied. The location of catalyst beds inside the column can be varied. The location of the feed(s) into the column can be varied. The location of the column top product draw can be varied. The location of introducing the condenser reflux stream(s) back into the column can be varied. The location of the column lower and upper side product draws can be varied. The location of introducing the reboiler reflux stream(s) (if any) back into the column can be varied.

### ETL-Based Oligomerization Followed by Alkylation Via Catalytic Distillation

In another aspect of the present disclosure, the ETL process is based on the initial step of oligomerization of light olefins (e.g. ethylene, propylene, and/or butenes) into higher olefins, with minimal conversion to hydrocarbons other than olefins (e.g. paraffins, isoparaffins, naphthenes, and aromatics). This may be accomplished over supported catalysts geared towards oligomerization at moderate process conditions. The C₄ olefin effluent from the previous step may be routed to a catalytic distillation unit, along with isobutane such that alkylation is effected to produce a desired alkylate stream. The catalytic distillation unit may comprise two or more alkylation catalyst beds where C₄ alkylation may take place by further alkylation of iC₈ and higher olefins (C₆₊) to produce a C₁₄₊ jet fuel and/or diesel blendstock. The example alkylation catalyst beds can employ conditions and catalysts as disclosed in U.S. Pat. No. 9,079,815 and International Patent Publication No. WO/2016/210006.

Additionally, C₃ and C₄ olefins can be sourced from adjacent refinery/petrochemical units (such as FCC, MTO, FT, delayed cokers, or steam crackers) to form additional feed into the C₄ alkylation bed in the distillation column, thereby increasing jet/diesel fuel production of out the process scheme.

FIG. 41 shows a schematic for ETL-based oligomerization followed by alkylation via catalytic distillation 4100. In this schematic, a stream containing ethylene 4101 is injected into an ETL reactor 4102. The effluent from the ETL reactor enters as feed into the catalytic distillation column 4103 in liquid or gas phase, along with a feed containing isobutane (iC₄) 4107. In the catalytic distillation column 4103, C₂-C₄ olefins may move up in the column towards the top alkylation bed, get into contact with iC₄ that is introduced also into the column, and both react to form iC₈ (while by-product nC₄ is withdrawn as a side stream). iC₈ may move downward in the column, get into contact with C₆₊ olefins from ETL, and both react over a second alkylation bed towards the bottom of the column, producing C₁₄₊ hydrocarbons. Unconverted C₂-C₄ and iC₄ (and any entrained nC₄) may be routed to a condenser 4104, where C₄s are condensed out and recycled back into the column, while C₂₌ and water are sent in vapor phase back into the ETL unit. A re-boiler 4105 may be used to partially vaporize the C₁₄₊ alkylate product 4106 and recycle the vapor back into the column, in order to strip any condensed unreacted C₆-C₈ hydrocarbons and send them back into the column. Butane can also be a product stream of the column 4108.

An oxidizing agent, such as O₂, air, or water, can be fed along with the ETL unit feed (which may contain H₂), such as to minimize/limit the extent of ethylene/propylene hydrogenation over the oligomerization catalysts—a phenomenon that takes place over highly active oligomerization catalysts resulting in loss of olefins into paraffins, thereby reducing oligomer yield. U.S. Pat. No. 4,717,782 discloses a method to introduce water along the oligomerization unit feed to effectively inhibit hydrogenation activity under a hydrogen atmosphere.

In some cases, CO contained in ETL feeds may convert readily via FT reactions with H₂ into C₁-C₄ paraffins, minimizing the adverse impact it can have over the oligomerization metal (such as Ni) such as etching.

A hydrotreating catalyst layer (or separate reaction zone) upstream of the ETL reactor can be employed to remove sulfur from certain ETL feeds. This can be in the form of a hydrotreating catalyst layer, composed of CoMo or NiMo based catalyst (which may react sulfur and not saturate olefins in the feed over the used process conditions), or in the form of a separate and upstream hydrotreating unit, which can be a MEROX type unit (employing a liquid catalyst) or a CoMo/NiMo based unit as described for the case of hydrotreating layer above.

The choice of active metal for effecting oligomerization of light olefins into higher olefins can be any one or combination of Ni, Pd, Cr, V, Fe, Co, Ru, Rh, Cu, Ag, Re, Mo, W, Mn, and Pt, and with up to a total loading of 20% by weight of catalyst mass. Catalyst support can range between one or any combination of zeolites (such as ZSM-5, Beta, and ZSM-11), amorphous silica alumina, silica, alumina, mesoporous silica, mesoporous alumina, zirconia, titania, and pillared clay. The operating conditions of the ETL unit to suit optimal conversion and high olefin yield out of the ETL reactor (about 50-200° C. and 10-80 bar). Choice of unit and associated operating conditions and catalyst employed for the upstream hydrotreating unit (if included) for removing sulfur. The ratio of oxidizing agent to feed hydrogen content to suppress olefin hydrogenation reactions. The operating conditions and catalyst of the alkylation beds, per those disclosed in U.S. Pat. No. 9,079,815 and International Patent Publication No. WO/2016/210006 (each of which is entirely incorporated herein by reference) units, with catalysts which may include Ni, Pd, Cr, V, Fe, Co, Ru, Rh, Cu, Ag, Re, Mo, W, Mn, Pt and supported on any one or combination of zeolites, sulfated zirconia, tungstated zirconia, chlorided alumina, aluminum chloride (AlCls), silicon-aluminum phosphates, titaniosilicates (including VTM zeolite), polyphosphoric acid (including solid phosphoric acid, or SPA, catalysts, which are made by reacting phosphoric acid with diatomaceous earth), polytungstic acid, and supported liquid acids such as triflic acid on silica, sulfuric acid on silica, hydrogen fluoride on carbon, antimony fluoride on silica, aluminum chloride (AlCls) on alumina (Al₂O₃). The ratio of iC₄ introduced to the column to olefin feed can be varied. The operation of the reboiler and condenser units (if included) such as to regulate the reflux ratios of olefins and iC₄ liquid/vapor and C₁₄₊ vapor back into the catalytic distillation column can be varied. The number of trays and/or height of packed catalyst beds used inside the column can be varied. The location of catalyst beds inside the column can be varied. The location of the feed(s) into the column can be varied. The location of the column top product draw can be varied. The location of introducing the condenser reflux stream(s) back into the column can be varied. The location of the column side product draw can be varied. The location of introducing the reboiler reflux stream(s) (if any) back into the column can be varied.

### EXAMPLES

### Example 1: Synthesis of Mesostructured Zeolites

FIG. 7 illustrates a sample procedure for producing mesostructured zeolites. As shown in the figure, firstly, 90 milliliter (mL) of 0.2 molar (M) NaOH solution is prepared. 3.675 grams (g) cetyltrimethylammonium bromide (CTAB) is then added to the NaOH solution. Temperature is kept at 40° C. to dissolve CTAB. Next, Ig of ZSM-5 is added to the solution, dispersed and stirred for about 2 hours (hr). Upon addition of the ZSM-5, 2 wt % Gallium (Ga) may be added to the solution. The solution is then heated at 100° C. for 24 hours (hrs) with stirring in polypropylene bottle.

Subsequently, pH of the solution is adjusted to 9 using H₂SO₄, and the solution is stirred overnight for 24 hrs. The solution is then heated to 100° C. for 24 hrs, followed by heating, washing and drying of the ZSM-5 at 80° C. overnight. The ZSM-5 is then calcined at 550° C. for 6 hrs, followed by ion exchange thrice using 0.05M NH₄NO₃ solution at 80° C. for 2 hrs.

Next, the meso-zeolite is again calcined under 550° C. for 2 hrs. 2 wt % Ga is then loaded by incipient impregnation technique. Finally, the zeolite is dried and calcined at 600° C. for 10 hrs at 2° C./min, resulting in the final mesostructured zeolite ready for use.

A list of sample zeolites synthesized by the methods of the present disclosure is shown in the below Table 2.

TABLE 2

| | | NaOH | Ga |
|---|---|---|---|
| Sample | SAR | Molarity | Introduction |
| RR1 | 80 | 0.2 | BOTH |
| RR2 | 30 | 0.2 | IWI |
| RR3 | 30 | 0.3 | WHILE MESOSTRUCTURING |
| RR4 | 30 | 0.4 | BOTH |
| RR5 | 30 | 0.4 | IWI |
| RR6 | 80 | 0.3 | IWI |
| RR7 | 80 | 0.3 | WHILE MESOSTRUCTURING |
| RR8 | 280 | 0.2 | BOTH |
| RR9 | 280 | 0.2 | WHILE MESOSTRUCTURING |
| RR10 | 280 | 0.3 | IWI |
| RR11 | 80 | 0.4 | BOTH |
| RR12 | 280 | 0.4 | WHILE MESOSTRUCTURING |

Synthesized mesostructured zeolites have been characterized using techniques including Brunauer-Emmett-Teller (BET), thermogravimetric analysis (TGA) and XRD with results shown in Table 3 and FIGS. 8A-8C and 9A-9C.

TABLE 3

| | | | | Parent | New | New Pore | Mesopore |
|---|---|---|---|---|---|---|---|
| | | NaOH | Ga | BET SA | BET SA | Volume | Volume |
| Sample | SAR | Molarity | Introduction | (m²/g) | (m²/g) | (cm³/g) | (cm³/g) |
| RR1 | 80 | 0.2 | BOTH | 470 | 756 | 1.11 | 0.60 |
| RR2 | 30 | 0.2 | IWI | 443 | 588 | 0.757 | 0.37 |
| RR3 | 30 | 0.3 | WHILE MESOSTRUCTURING | 443 | 762 | 1.12 | 0.60 |
| RR4 | 30 | 0.4 | BOTH | 443 | 783 | 1.00 | 0.69 |
| RR5 | 30 | 0.4 | IWI | 443 | 785 | 1.04 | 0.72 |
| RR6 | 80 | 0.3 | IWI | 470 | 853 | 1.38 | 0.74 |
| RR7 | 80 | 0.3 | WHILE MESOSTRUCTURING | 470 | 848 | 1.05 | 0.77 |
| RR8 | 280 | 0.2 | BOTH | 437 | 751 | 0.90 | 0.66 |
| RR9 | 280 | 0.2 | WHILE MESOSTRUCTURING | 437 | 715 | 0.87 | 0.60 |
| BR10 | 280 | 0.3 | IWI | 437 | 903 | 1.08 | 0.85 |
| RR11 | 80 | 0.4 | BOTH | 470 | 843 | 1.01 | 0.77 |
| RR12 | 280 | 0.4 | WHILE MESOSTRUCTURING | 437 | 866 | 1.01 | 0.80 |

### Example 2: Catalyst Performance Under ETL Conditions

FIGS. 10A-10C and 11A-11C illustrate catalyst performance under different ETL conditions. As shown in the figures, the mesostructured zeolites with relatively high SAR and Ga-modified framework have better performance and longer lifetimes as compared to non-modified or other modified zeolites.

### Example 3: Further Processing of Meso-Structured Catalysts

Mesostructured zeolites are conditioned using a step in including steaming, calcination, reduction or combinations thereof prior to being subjected to reaction conditions such as ETL. FIG. 12 shows a list of sample mesostructured zeolites steamed under certain conditions. Performance of such formed zeolites under differing ETL conditions is illustrated in FIGS. 13A-13C and 14A-14C.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents. It is intended that the following claims define the scope of the invention and that methods and structures within the scope of these claims and their equivalents be covered thereby.

What is claimed is:

1. A method for generating hydrocarbon compounds with eight or more carbon atoms (C₈₊ compounds), comprising:
(a) directing a feed stream comprising unsaturated C₂₊ hydrocarbon compounds into an oligomerization unit that permits at least a portion of said unsaturated C₂₊ hydrocarbon compounds to react in an oligomerization process to yield an effluent comprising unsaturated C₃₊ hydrocarbon compounds; and
(b) directing at least a portion of said effluent from said oligomerization unit and a stream comprising isoparaffins into an alkylation unit downstream of and separate from said oligomerization unit, which alkylation unit permits at least a portion of said unsaturated C₃₊ hydrocarbon compounds and said isoparaffins to react in an alkylation process to yield a product stream comprising said C₈₊ compounds.

2. The method of claim 1, further comprising: directing methane and an oxidizing agent into an oxidative coupling of methane (OCM) unit that facilitates an OCM reaction to generate an OCM product stream comprising ethylene (C₂H₄), and wherein at least a portion of said feed stream is provided by the OCM product stream.

3. The method of claim 1, wherein the oligomerization unit comprises a dimerization unit and the oligomerization process comprises a dimerization process, and wherein the dimerization process operates at a temperature of 20° C. to 200° C. and a pressure of 100 psia to 400 psia.

4. The method of claim 1, wherein the product stream is an alkylate stream comprising an alkylate product comprising saturated C₈₊ hydrocarbon compounds, isomers thereof, or both saturated C₈₊ hydrocarbon compounds and isomers thereof.

5. The method of claim 4, wherein the alkylate product comprises at least 95 wt % saturated C₈₊ hydrocarbon compounds, isomers thereof, or both saturated C₈₊ hydrocarbon compounds and isomers thereof.

6. The method of claim 4, wherein the alkylate product has a research octane number of 90 to 99.

7. The method of claim 4, wherein the alkylate product has a motor octane number of 80 to 95.

8. The method of claim 1, further comprising: directing at least a portion of said effluent from said oligomerization unit to an isomerization unit, wherein at least a portion of said unsaturated C₃₊ hydrocarbon compounds in the at least a portion of said effluent from said oligomerization unit react in an isomerization process to yield an isomerization product stream comprising a mixture of unsaturated C₃₊ hydrocarbon compounds and isomers thereof.

9. The method of claim 8, further comprising: directing the isomerization product stream into the alkylation unit.

10. The method of claim 1, further comprising: prior to step (a), directing the feed stream into an isomerization unit upstream of the oligomerization unit, wherein at least a portion of said unsaturated C₂₊ hydrocarbon compounds react in an isomerization process to yield an isomerized feed stream comprising a mixture of unsaturated C₂₊ hydrocarbon compounds and isomers thereof.

11. The method of claim 1, wherein the isoparaffins comprise isobutane, isopentane, or combinations thereof.

12. The method of claim 1, further comprising: directing one or more additional feed streams comprising saturated C₃₊ hydrocarbon compounds into the alkylation unit, wherein the one or more additional feed streams are generated by one or more additional processing units, wherein the one or more additional processing units comprise a fluid catalytic cracking unit, a methanol-to-olefins unit, a Fischer-Tropsch unit, a delayed coker unit, a steam cracker, or combinations thereof.

13. The method of claim 1, further comprising:
(i) directing at least a portion of said effluent from said oligomerization unit into a separations unit to separate at least a portion of said unsaturated C₃₊ hydrocarbon compounds and at least a portion of unreacted ethylene from the at least a portion of said effluent from said oligomerization unit;
(ii) directing at least a portion of the separated unsaturated C₃₊ hydrocarbon compounds from the separations unit into a fractionation unit to separate at least one impurity from the separated unsaturated C₃₊ hydrocarbon compounds and thereby yield a first stream comprising at least a portion of the at least one impurity and a second stream comprising at least a portion of the separated unsaturated C₃₊ hydrocarbon compounds with a reduced concentration of the at least one impurity; and
(iii) directing the second stream into the alkylation unit.

14. The method of claim 13, further comprising: prior to step (i) directing at least a portion of said effluent from said oligomerization unit into an additional separations unit to separate isoparaffins from the at least a portion of said effluent from said oligomerization unit, and directing the isoparaffins separated in the additional separations unit to the alkylation unit.

15. The method of claim 14, wherein the isoparaffins comprise isobutane, isopentane, or combinations thereof.

16. The method of claim 15, wherein the isoparaffins comprise at least 70 wt % isopentane and less than 20 wt % isobutane.

17. The method of claim 1, further comprising:
(i) directing at least a portion of the product stream from the alkylation unit into a separations unit to yield a separations product stream comprising C₆₊ hydrocarbon compounds; and
(ii) directing at least a portion of the separations product stream from the separations unit into a second alkylation unit that permits at least a portion of the C₆₊ hydrocarbon compounds to react in a second alkylation process to yield a second product stream comprising C₁₄₊ compounds.

18. The method of claim 17, wherein C₆₊ compounds comprise (i) C₈₊ isoparaffins and (ii) unsaturated C hydrocarbon compounds.

19. A system for producing C₈₊ hydrocarbon compounds comprising:
(a) an oligomerization unit comprising an oligomerization catalyst, wherein the oligomerization unit is configured to receive a feed stream comprising unsaturated C₂₊ hydrocarbon compounds and at least a portion of the unsaturated C₂₊ hydrocarbon compounds react in an oligomerization process to yield an effluent comprising unsaturated C₃₊ hydrocarbon compounds;
(b) an alkylation unit downstream of and in fluid communication with the oligomerization unit, wherein the alkylation unit is configured to receive at least a portion of the effluent and a stream comprising isoparaffins and at least a portion of said unsaturated C₃₊ hydrocarbon compounds and said isoparaffins react in an alkylation process to yield a product stream comprising said C₈₊ hydrocarbon compounds.

20. The system of claim 19, further comprising: an oxidative coupling of methane (OCM) unit upstream of and in fluid communication with the oligomerization unit, wherein the OCM unit is configured to receive methane and an oxidizing agent and to react the methane and oxidizing agent to generate an OCM product stream comprising unsaturated C₂₊ hydrocarbon compounds, and wherein at least a portion of said feed stream is provided by said OCM product stream.
